(19) United States
(12) Patent Application Publication
(10) Pub. No.: US 2023/0124929 A1
(43) Pub. Date: Apr. 20, 2023

(54) NITROGEN-CONTAINING COMPOUND, ORGANIC ELECTROLUMINESCENT DEVICE, AND ELECTRONIC APPARATUS

(71) Applicant: Shaanxi Lighte Optoelectronics Material Co., Ltd., Xi'an (CN)

(72) Inventors: Jinping WANG, Xi'an (CN); Zhen XUE, Xi'an (CN); Shan YAN, Xi'an (CN); Zhiwei CHEN, Xi'an (CN)

(21) Appl. No.: 17/788,064

(22) PCT Filed: Dec. 24, 2020
(86) PCT No.: PCT/CN2020/139067

§ 371 Date: Jun. 22, 2022

(30) Foreign Application Priority Data
Dec. 31, 2019 (CN) 201911420649.8
Dec. 15, 2020 (CN) 202011477115.1

(51) Int. Cl. H01L 51/00; C07C 211/61; C09K 11/06; C07D 307/91; C07D 209/86; C07D 209/88; C07D 409/12; C07D 333/76; C07D 263/57; C07D 251/24; C07D 239/74

(52) U.S. Cl. CPC H01L 51/006; C07C 211/61; C07D 209/86; C07D 209/88; C07D 239/74; C07D 251/24; C07D 263/57; C07D 307/91; C07D 333/76; C07D 409/12; C09K 11/06; H01L 51/0061; H01L 51/5064

(57) ABSTRACT

Provided are a nitrogen-containing compound, an organic electroluminescent device and an electronic apparatus. The nitrogen-containing compound has a structure as shown in chemical formula 1; Ar₁ and Ar₂ are the same or different, and each is independently selected from a substituted or unsubstituted C6-C30 aryl, or a substituted or unsubstituted C3-C30 heteroaryl; and R₀ is selected from hydrogen, a C1-C6 alkyl, a substituted or unsubstituted C6-C30 aryl, or a substituted or unsubstituted C3-C30 heteroaryl. The nitrogen-containing compound acts as a hole transporting material or a host material of a luminescent layer and can effectively improve the performance of organic electroluminescent devices.

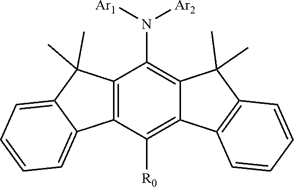

### CROSS REFERENCE TO RELATED APPLICATIONS

The present application claims the priority of Chinese patent application No. CN201911420649.8 filed on Dec. 31, 2019 and the priority of Chinese patent application No. CN202011477115.1 filed on Dec. 15, 2020, which are incorporated herein by reference in their entirety.

### TECHNICAL FIELD

The present disclosure relates to the field of organic electroluminescence, in particular to a nitrogen-containing compound, an organic electroluminescent device and an electronic apparatus.

### BACKGROUND

Organic optoelectronic materials are organic materials with the characteristics of generation, conversion, transport and the like of photons and electrons. At present, the controllable photoelectric properties of the organic optoelectronic materials have already been applied to organic light-emitting diodes (OLEDs), organic photovoltage (OPV), organic field effect transistors (OFETs), biological/chemical/optical sensors, storages, and even organic lasers.

Organic light-emitting diode (OLED) displays are deemed to be very likely to become next generation displays. Due to active light emitting, compared with liquid crystal displays, organic light-emitting diode displays have the advantages of low energy consumption, high response speed, wide visual angle, thinner device structure, outstanding low-temperature characteristics, even capability of being made into a flexible display screen and the like. At present, the OLED is successfully applied, but many bottlenecks need to be solved in the organic light-emitting display technology, and particularly, the challenges of impure chromaticity of the light display, low efficiency and short material service life need to be faced on light display.

The basic structural unit of the OLED display is OLED device, and the OLED device can be divided into a fluorescent device and a phosphorescent device according to different light-emitting mechanisms. The fluorescent OLED based on singlet luminescence, as the first-generation luminescent material, has a theoretical internal quantum efficiency of only 25%, and the efficiency of the fluorescent OLED cannot be further improved. The phosphorescent OLED is called the second generation, and the internal quantum efficiency of the phosphorescent OLED can reach 100%. The phosphorescent material enhances intersystem crossing due to strong spin-orbit coupling of a heavy atom center, singlet excitons and triplet excitons formed by electrical excitation can be effectively utilized to emit light, so that the internal quantum efficiency of the device reaches 100%, but the phosphorescent material has the problems of high price, poor material stability, short service life, serious device efficiency roll-off, weak blue light phosphorescence and the like, so that the application of the phosphorescent material in the OLED is limited.

In 2009, a class of carbazolylbenzonitrile derivatives was designed and synthesized by Prof. Adachi from Kyushu University in Japan, and then a new thermally activated delayed fluorescence (TADF) material based on triplet-singlet transition is found, its internal quantum efficiency is close to 100%, and the material is a third-generation organic light-emitting material developed after the organic fluorescent material and the organic phosphorescent material. The material generally has a small singlet-triplet energy level difference (ΔE_{ST}), and triplet excitons can be converted into singlet excitons through reverse intersystem crossing to emit light. Singlet excitons and triplet excitons formed under electrical excitation can be fully utilized, and the internal quantum efficiency of the device can reach 100%. Meanwhile, the material is controllable in structure, stable in property, low in price, free of precious metal, and has wide application prospect in the field of OLED. However, the correlation between the structure and the photophysical property of the material and the device efficiency is not clear, the development of the efficient delayed fluorescent material is limited, and the problems with the existing TADF material are caused, for example, the type is single, the device efficiency is low and the material service life is short. The requirement of the efficient organic light-emitting diode cannot be met.

### SUMMARY

In terms of the problems in the prior art, the present disclosure aims to provide a nitrogen-containing compound, an organic electroluminescent device and an electronic apparatus. The nitrogen-containing compound can improve the performance of organic electroluminescent device.

In the first aspect, the present disclosure provides a nitrogen-containing compound, having a structure as shown in chemical formula 1:

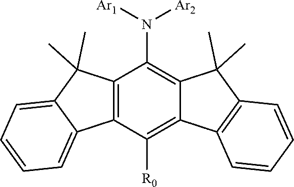

wherein Ar₁ and Ar₂ are the same or different, and are each independently selected from a substituted or unsubstituted C6-C30 aryl, or a substituted or unsubstituted C3-C30 heteroaryl; and

R₀ is selected from hydrogen, a C1-C6 alkyl, a substituted or unsubstituted C6-C30 aryl, or a substituted or unsubstituted C3-C30 heteroaryl.

In the second aspect, the present disclosure provides an organic electroluminescent device, including an anode, a cathode and a functional layer located between the anode and the cathode, and the functional layer includes the nitrogen-containing compound described in the first aspect of the present disclosure.

In the third aspect, the present disclosure provides an electronic apparatus, including the organic electroluminescent device described in the second aspect of the disclosure.

The nitrogen-containing compound provided by the present disclosure takes 10,10,12,12-tetramethyl-10,12-dihydroindeno[2,1-b]fluorene as the parent nucleus, and an arylamine group is bound to the position 11 of the parent nucleus, so that the steric hindrance of the molecular structure is relatively larger, the distortion angle is increased, so the nitrogen-containing compound has relatively higher stability and can improve the performance of the organic electroluminescent device. As a hole transporting material, the nitrogen-containing compound can reduce the driving voltage of a device and prolong the service life of the device, and can also meet higher photoelectric efficiency; and as a host material of a luminescent layer, the nitrogen-containing compound can effectively improve the photoelectric efficiency of an OLED, prolong the service life of the OLED device, and enable the device to meet lower driving voltage at the same time.

### BRIEF DESCRIPTION OF THE DRAWINGS

FIG. 1 is a structural schematic diagram of an organic electroluminescent device in one embodiment of the present disclosure; and

FIG. 2 is a schematic diagram of an electronic apparatus in one embodiment of the present disclosure.

The reference signs of main elements in the drawings are described as follows:

100, anode; 200, cathode; 300, functional layer; 310, hole injectioning layer; 320, hole transporting layer; 321, first hole transporting layer; 322, second hole transporting layer; 330, luminescent layer; 340, electron transporting layer; 350, electron injection layer; 10, mobile phone display panel; and 20, electronic apparatus.

### DETAILED DESCRIPTION

In one aspect, the present disclosure provides a nitrogen-containing compound, having a structure as shown in Chemical formula 1:

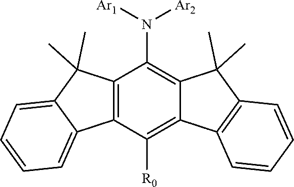

wherein Ar₁ and Ar₂ are the same or different, and are each independently selected from a substituted or unsubstituted C6-C30 aryl, or a substituted or unsubstituted C3-C30 heteroaryl; and R₀ is selected from hydrogen, a C1-C6 alkyl, a substituted or unsubstituted C6-C30 aryl, or a substituted or unsubstituted C3-C30 heteroaryl.

In the present disclosure, when Ar₁ and Ar₂ are aryl or heterocyclic aryl, and R₀ is alkyl such as tert-butyl, hydrogen or aryl, or electron-rich heteroaryl such as dibenzofuranyl, dibenzothiopheneyl and the like, the molecular structure has lower ionization energy, and the N atom on a tertiary amine group (tertiary amine group) connected to the specific position of the parent nucleus has very strong electron donating ability, which is easy to oxidize into cation free radical (hole) so as to show electropositivity and the characteristic of hole migration, and such molecules have high hole mobility. In addition, the nitrogen-containing compound takes 10,10,12,12-tetramethyl-10,12-dihydroindeno[1,2-b]fluorene as a core, and can be used as a thermally activated delayed fluorescence compound material; and when R₀ is electron-deficient nitrogen-containing heteroaryl such as triazinyl and the like, a “D-π-A type” organic small molecule compound formed by the parent nucleus, together with the arylamine group and R₀, which are respectively connected to the specific positions on the parent nucleus, is particularly suitable for being used as a host material.

In the present disclosure, the term such as “substituted or unsubstituted” means that a functional group described following the term may have or do not have a substituent (hereinafter, the substituent is collectively referred to as Rc in order to describe easilly). For example, the “substituted or unsubstituted aryl” refers to an aryl with substituent Rc or an unsubstituted aryl. The above-mentioned substituent, namely Rc may be, for example, deuterium, halogen group, cyano, alkyl, alkoxy, alkylthio, haloalkyl, deuterated alkyl, cycloalkyl, trialkylsilyl, triphenylsilyl, diarylphosphinyl, aryloxy, or the like. In the present disclosure, the “substituted” functional group can be substituted by one or two or more substituents Rc.

In the present disclosure, the number of carbon atoms of substituted or unsubstituted group refers to the number of all carbon atoms. For example, if Ar₁ is a substituted aryl having 12 carbon atoms, the number of all carbon atoms of the aryl and substituents on the aryl is 12.

The description modes “each...is independently”, “...is respectively and independently” and “...is independently selected from” adopted in the present disclosure can be interchanged, and should be understood in a broad sense, which means that in different groups, specific options expressed between the same symbols are not influenced with each other, or in a same group, specific options expressed between the same symbols are not influenced with each other. For example, in the description of

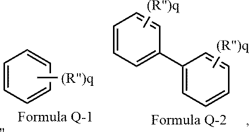

wherein each q is independently 0, 1, 2 or 3, and each R” is independently selected from hydrogen, fluorine, or chlorine”, its meaning is as follows: Formula Q-1 indicates that the benzene ring has q substituents R”, and each R” can be the same or different, and options of each R” are not influenced with each other; Formula Q-2 indicates that each benzene ring of the biphenyl has q substituents R”, the number q of the substituents R” on the two benzene rings can be the same or different, and each R” can be the same or different, and options of each R” are not influenced with each other.

In the present disclosure, the term “optionally” means that the subsequently described event or environment may, but does not have to, occur, and the description includes an occasion where the event or environment occurs or does not occur. For example, “R₄ and R₅ optionally form a ring” means that the two substituents R₄ and R₅can form a ring but do not have to form a ring, including: a situation that R₄ and R₅ form a ring and a situation that R₄ and R₅ do not form a ring.

The non-localized connecting bond in the present disclosure is a single bond “———_{#}” stretching out of a ring system, which means that one end of the connecting bond can be connected with any position in the ring system through which the bond stretching out of, and the other end of the connecting bond is connected with the remaining part of a compound molecule. For example, as shown in the following formula (X′), phenanthryl represented by the formula (X′) is connected with other positions of a molecule through one non-localized connecting bond stretching out of the middle of a benzene ring on one side, and its meaning includes any possible connecting mode represented by formulas (X′-1)-(X′-4).

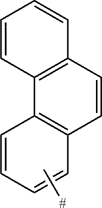

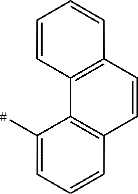

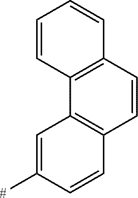

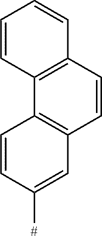

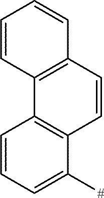

In the present disclosure, the expression of a combination of C with a number can be summarized as ‘Cj’, j represents the number, for example, when j is 3, ‘Cj’ is ‘C3’, and ‘Cj’ represents the number of carbon atoms. For example, ‘C3’ represents that the number of carbon atoms is 3, and ‘C6-C30’ represents that the number of carbon atoms is 6 to 30.

In the present disclosure, “aryl” refers to an optional functional group or a substituent derived from an aromatic hydrocarbon ring. The aryl may be a monocyclic aryl or a polycyclic aryl, in other words, the aryl can be a monocyclic aryl, a fused cyclic aryl, a group formed by two or more monocyclic aryl conjugatedly connected via carbon-carbon bond, monocyclic aryl and fused cyclic aryl conjugatedly connected via carbon-carbon bond, and two or more fused cyclic aryl conjugatedly connected via carbon-carbon bond. In other words, the group formed by two or more aromatic groups conjugatedly connected via carbon-carbon bond can also be regarded as the aryl of the present disclosure. The fused cyclic aryl may include, for example, bicyclic fused aryl (e.g., naphthyl), tricyclic fused aryl (e.g., phenanthryl, fluorenyl, and anthryl), and the like. The aryl does not contain heteroatoms such as B, N, O, S, P, Se or Si. For example, in the present disclosure, biphenyl, terphenyl, etc. are aryl. Specific examples of the aryl include, but not limited to, phenyl, naphthyl, fluorenyl, spiro-fluorenyl, anthryl, phenanthryl, biphenyl, terphenyl, quaterphenyl, benzo[9,10]phenanthryl, benzofluoranthenyl, chrysenyl and the like.

The substituted aryl means that one or two or more hydrogen atoms of the aryl are substituted by groups such as deuterium atom, halogen group, -CN, alkyl (such as a C1-C6 alkyl), cycloalkyl (such as a C3-C10 cycloalkyl), alkoxy (such as a C1-C6 alkoxy), trialkylsilyl (such as a C3-C10 trialkylsilyl) and the like. It should be understood that the number of carbon atoms in the substituted aryl refers to the total number of carbon atoms in the aryl and substituents thereon. For example, a substituted C6-C30 aryl means that the total number of carbon atoms of the aryl and the substituents on the aryl is 6 to 30.

In the present disclosure, “heteroaryl” refers to a monovalent aromatic ring containing at least one heteroatom in the ring or derivative thereof, and the heteroatom may be at least one of B, O, N, P, Si, Se and S. The heteroaryl may be a monocyclic heteroaryl or a polycyclic heteroaryl. In other words, the heteroaryl may be a single aromatic ring system or plurality aromatic ring systems conjugatedly connected via carbon-carbon bond, and any aromatic ring system is an aromatic monocyclic ring or an aromatic fused cyclic ring. Specific examples of the heteroaryl include, but not limited to, thienyl, furyl, pyrrolyl, imidazolyl, thiazolyl, oxazolyl, oxadiazolyl, triazolyl, pyridyl, bipyridyl, pyrimidinyl, triazinyl, acridinyl, pyridazinyl, pyrazinyl, quinolyl, quinazolinyl, quinoxalinyl, phenoxazinyl, phthalazinyl, pyridinopyrimidyl, pyridinopyrazinyl, pyrazinopyrazinyl, isoquinolyl, indolyl, carbazolyl, benzoxazolyl, benzimidazolyl, benzothiazolyl, benzocarbazolyl, benzothienyl, dibenzothienyl, thienothienyl, benzofuryl, phenanthrolinyl, isoxazolyl, thiadiazolyl, benzothiazolyl, phenothiazinyl, silafluorenyl, dibenzofuryl, N-arylcarbazolyl (such as N-phenylcarbazolyl), N-heteroarylcarbazolyl (such as N-pyridylcarbazolyl), N-alkylcarbazolyl (such as N-methylcarbazolyl), dibenzofuranyl substituted by phenyl, phenyl substituted by dibenzofuryl, 4,6-diaryl-1,3,5-triazin-2-yl and the like. Among them, thienyl, furyl, phenanthrolinyl and the like are heteroaryl gronps of the single aromatic ring system, and N-arylcarbazolyl, N-heteroarylcarbazolyl, dibenzofuryl substituted by phenyl, phenyl substituted by dibenzofuryl and the like are heteroaryl gronps of the plurality aromatic ring systems conjugatedly connected via carbon-carbon bonds.

The substituted heteroaryl means that one or more hydrogen atoms in the heteroaryl are substituted by groups such as deuterium atom, halogen group, -CN, alkyl (such as a C1-C6 alkyl), cycloalkyl (such as a C3-C10 cycloalkyl), alkoxy (such as a C1-C6 alkoxy), alkylthio and the like. It should be understood that the number of carbon atoms of the substituted heteroaryl refers to the total number of carbon atoms of the heteroaryl and substituents thereon. For example, a C3-C30 substituted heteroaryl means that the total number of carbon atoms of the heteroaryl and substituents on the heteroaryl is 3 to 30.

In the present disclosure, the number of ring-forming carbon atoms refers to the number of carbon atoms positioned on all aromatic rings of the substituted or unsubstituted aryl and substituted or unsubstituted heteroaryl, and it should be noted that when the structures of the substituted or unsubstituted aryl and the substituted or unsubstituted heteroaryl include a plurality of aromatic rings, the number of carbon atoms on all aromatic rings is considered to be within the number of ring-forming carbon atoms, and the number of carbon atoms of other substituents (such as methyl and cyano) on the aromatic rings is not counted. For example, the number of ring-forming carbon atoms of fluorenyl is 13, the number of ring-forming carbon atoms of 9,9-dimethylfluorenyl is 15, the number of ring-forming carbon atoms of diphenylfluorenyl is 25, and the number of ring-forming carbon atoms of phenyl substituted by methyl is 6.

In the present disclosure, cycloalkyl can be used as the substituent of aryl and heteroaryl, the number of carbon atoms can be 3 to 10, preferably 5 to 10, and specific examples of the cycloalkyl include, but not limited to, cyclopentyl, cyclohexyl, adamantyl and the like.

In the present disclosure, the halogen group may include fluorine, bromine, chlorine, iodine and the like.

In the present disclosure, the C1-C6 alkyl includes a C1-C3 linear alkyl and a C3-C6 branched alkyl, the number of carbon atoms may be, for example, 1, 2, 3, 4, 5 or 6, and specific examples of the C1-C6 alkyl include, but are not limited to, methyl, ethyl, n-propyl, isopropyl, n-butyl, tert-butyl, n-amyl, n-hexyl and the like.

Optionally, Ar₁ and Ar₂ are the same or different, and are each independently selected from a substituted or unsubstituted C6-C30 aryl, or a substituted or unsubstituted C3-C30 heteroaryl; and R₀ is selected from a C1-C6 alkyl, a substituted or unsubstituted C6-C30 aryl, or a substituted or unsubstituted C3-C30 heteroaryl.

In the present disclosure, when Ar₁, Ar₂ and R₀ are each independently aryl, the number of carbon atoms of Ar₁, Ar₂ and R₀ may be respectively and independently 6, 7, 8, 9, 10, 11, 12, 13, 14, 15, 16, 17, 18, 19, 20, 21, 22, 23, 24, 25, 26, 27, 28, 29 or 30. When Ar₁, Ar₂ and R₀ are each independently heteroaryl, the number of carbon atoms of Ar₁, Ar₂ and R₀ may be each independently 3, 4, 5, 6, 7, 8, 9, 10, 11, 12, 13, 14, 15, 16, 17, 18, 19, 20, 21, 22, 23, 24, 25, 26, 27, 28, 29 or 30.

According to one exemplary embodiment, the substituents of Ar₁, Ar₂ and R₀ are each independently selected from deuterium, a C1-C6 alkyl, a C3-C10 cycloalkyl, a C1-C6 alkoxy, a C1-C6 alkylthio, cyano, or halogen group.

Optionally, the substituents of Ar₁, Ar₂ and R₀ are each independently selected from deuterium, methyl, ethyl, n-propyl, isopropyl, tert-butyl, methoxy, ethoxy, methylthio, ethylthio, cyclopentyl, cyclohexyl, adamantyl, cyano, or fluorine. The number of the substituents of Ar₁, Ar₂ and R₀ can be one or two or more, and when the number of the substituents is two or more, each substituent can be the same or different.

Optionally, Ar₁, Ar₂ and R₀ are each independently selected from an aryl with 6 to 25 ring-forming carbon atoms, or a heteroaryl with 3 to 25 ring-forming carbon atoms.

According to one specific embodiment, Ar₁ and Ar₂ are each independently selected from a substituted or unsubstituted C6-C30 aryl, or a substituted or unsubstituted C3-C30 heteroaryl, and R₀ is a C1-C6 alkyl. Optionally, R₀ is selected from methyl or tert-butyl.

Optionally, Ar₁, Ar₂ and R₀ are each independently selected from a substituted or unsubstituted C6-C30 aryl.

Optionally, Ar₁ and Ar₂ are each independently selected from a substituted or unsubstituted C6-C30 aryl, and R₀ is a substituted or unsubstituted C3-C30 heteroaryl.

Optionally, Ar₁ and Ar₂ are each independently selected from a substituted or unsubstituted C6-C20 aryl, or a substituted or unsubstituted C6-C20 heteroaryl; and R₀ is hydrogen, a C1-C4 alkyl, a substituted or unsubstituted C6-C18 aryl, or a substituted or unsubstituted C6-C22 heteroaryl.

As described above, Ar₁, Ar₂ and R₀ may be each independently selected from a substituted or unsubstituted C6-C30 aryl, or a substituted or unsubstituted C3-C30 heteroaryl. In one embodiment, Ar₁, Ar₂ and R₀ are each independently selected from the following groups:

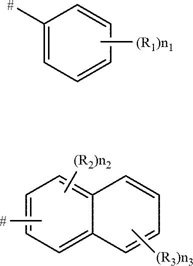

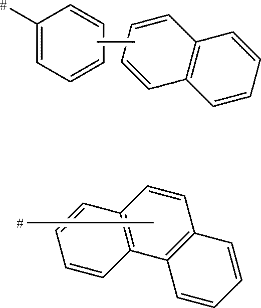

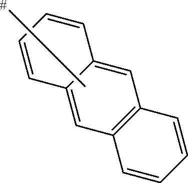

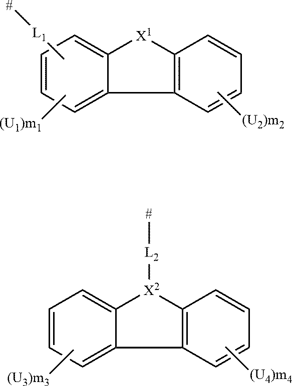

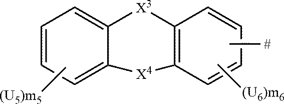

X¹, X³ and X⁴ are each independently selected from O, S, C(R₄R₅), N(Rs), or Si(R₆R₇); and X² represents N atom;

R₁ to R₃ are each independently selected from phenyl, biphenyl, hydrogen, halogen group, cyano, a C1-C6 alkyl, a C3-C10 cycloalkyl, a C1-C6 alkoxy, or a C1-C6 alkylthio;

n₁ represents the number of R₁ and is specifically selected from 1, 2, 3, 4, or 5; n₂ represents the number of R₂ and is specifically selected from 1, 2, or 3; and n₃ represents the number of R₃ and is specifically selected from 1, 2, 3, or 4;

U₁ to U₆ are each independently selected from hydrogen, halogen group, cyano, a C1-C6 alkyl, a C3-C10 cycloalkyl, or a C1-C6 alkoxy; m₁ and m₆ are each independently selected from 1, 2, or 3; m₂ to m₅ are each independently selected from 1, 2, 3, or 4; mₖ respectively represents the number of Uₖ (k represents a variable and is specifically selected from any integer of 1 to 6, for example, when k is equal to 1, mₖ refers to m₁, and Uₖ refers to U₁, and for another example, when k is equal to 6, mₖ refers to m₆, Uₖ refers to U₆);

R₄ to R₈ are each independently selected from hydrogen, deuterium, halogen group, cyano, a C1-C6 alkyl, a C6-C18 aryl, a C3-C18 heteroaryl, a C3-C10 cycloalkyl, or a C1-C6 alkoxy, and R₄ and R₅optionally form a ring, and R₆ and R₇ optionally form a ring;

L₁ and L₂ are each independently selected from single bond, phenylene, naphthylene, anthrylene, or phenanthrylene; and # represents a position for connection.

In the present disclosure, the ring formed by R₄ and R₅, and the ring formed by R₆ and R₇ may be, for example, a saturated or unsaturated C3-C10 cyclic group.

Optionally, Ar₁, Ar₂ and R₀ are each independently selected from the following groups:

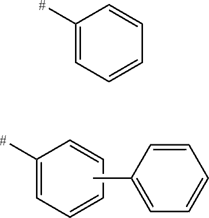

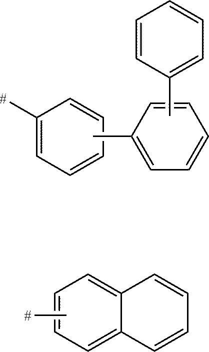

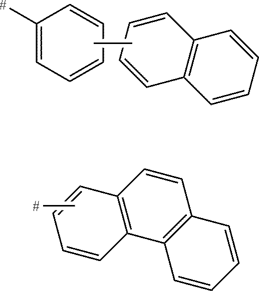

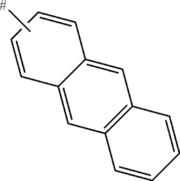

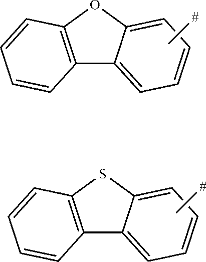

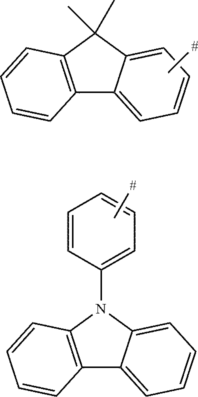

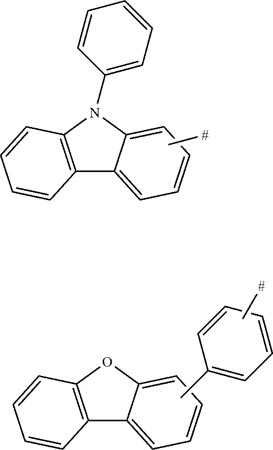

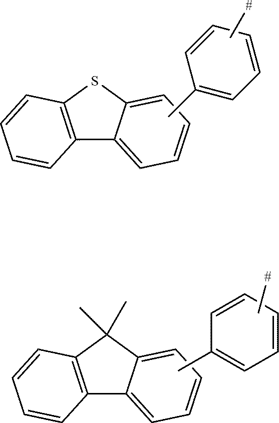

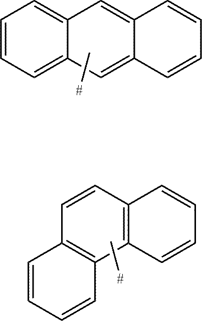

# represents a position for connection.

In one embodiment, R₀ is selected from the following groups:

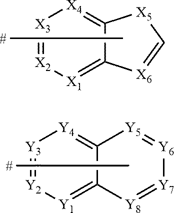

wherein X₁, X₂, X₃ and X₄ are each independently selected from CH or N atom, X₅ is selected from O atom or S atom, and X₆ is selected from CH or N; and at least one of X₁ to X₄ and X₆ is CH; Y₁ to Y₈ are each independently selected from CH or N atom, and at least one of Y₁ to Y₈ is N atom.

Optionally, R₀ is selected from the following groups:

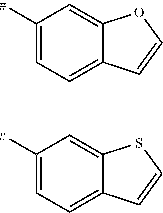

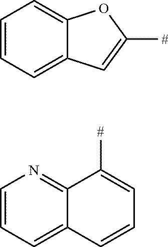

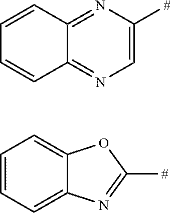

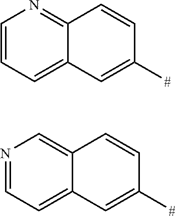

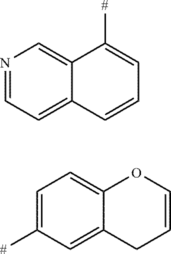

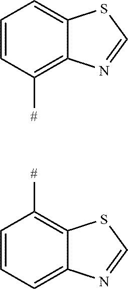

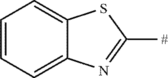

In another embodiment, R₀ is selected from the following groups:

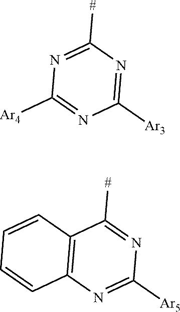

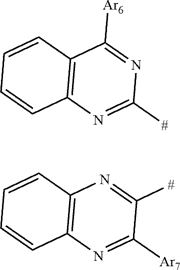

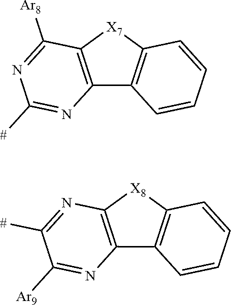

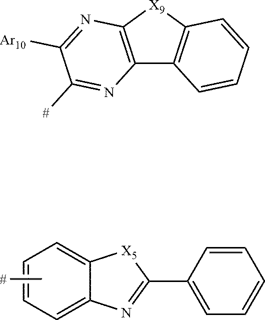

wherein X₅, and X₇ to X₉ are each independently selected from O atom or S atom; and

Ar₃ to Ar₁₀ are the same or different, and are each independently selected from a substituted or unsubstituted C6-C15 aryl, or a substituted or unsubstituted C6-C15 heteroaryl; the substitution refers to substitution by a group selected from deuterium, fluorine, methyl, or tert-butyl. When Ar₃ to Ar₁₀ have substituents, the number of the substituents may be one or two or more, and when the number of the substituents is two or more, each substituent can be the same or different.

Optionally, Ar₃ to Ar₁₀ are each independently selected from substituted or unsubstituted phenyl, substituted or unsubstituted naphthyl, substituted or unsubstituted biphenyl, substituted or unsubstituted dibenzofuranyl, or substituted or unsubstituted dibenzothienyl.

In some embodiments, Ar₁ and Ar₂ are each independently selected from substituted or unsubstituted phenyl, substituted or unsubstituted naphthyl, substituted or unsubstituted biphenyl, substituted or unsubstituted fluorenyl, substituted or unsubstituted anthryl, substituted or unsubstituted phenanthryl, substituted or unsubstituted dibenzofuranyl, substituted or unsubstituted dibenzothienyl, or N-phenylcarbazolyl; or a new group formed by connecting any two above groups via a single bond (such as a group formed by connecting phenyl and naphthyl via a single bond, a group formed by connecting phenyl and 9,9-dimethylfluorenyl via a single bond, a group formed by connecting phenyl and phenanthryl via a single bond, a group formed by connecting phenyl and dibenzofuranyl via a single bond, a group formed by connecting phenyl and dibenzothienyl via a single bond and the like); the substituents of Ar₁ and Ar₂ are each independently selected from deuterium, methyl, ethyl, n-propyl, isopropyl, tert-butyl, methoxy, ethoxy, methylthio, cyclopentyl, cyclohexyl, cyano, or fluorine, the number of the substituents is one or two or more, and when the number of the substituents is two or more, each substituent is the same or different.

Optionally, Ar₁ and Ar₂ are each independently selected from the following groups:

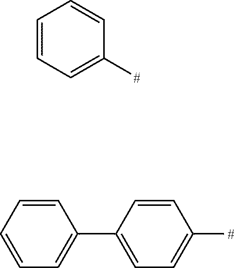

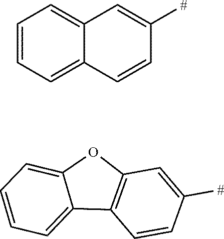

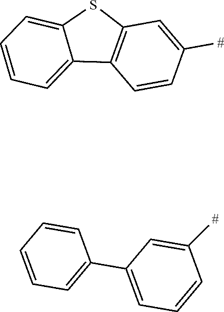

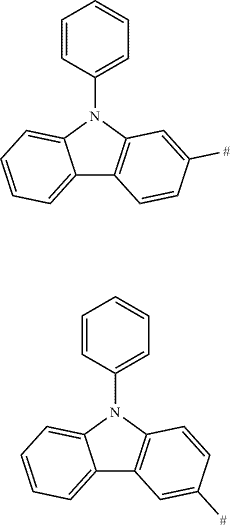

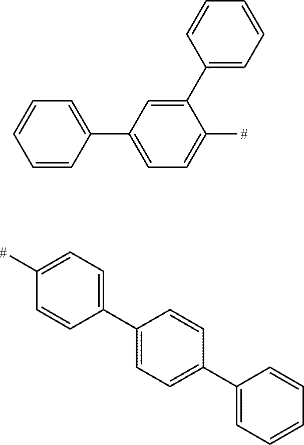

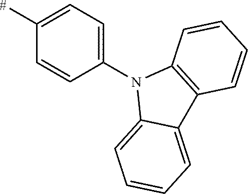

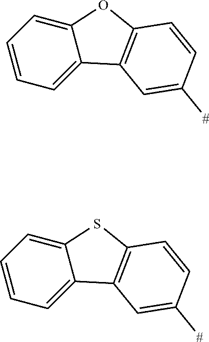

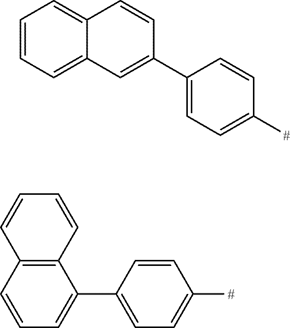

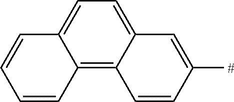

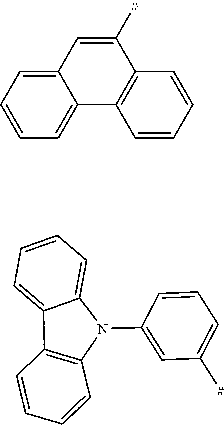

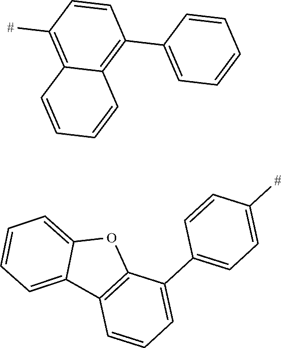

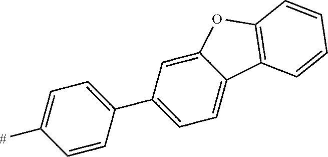

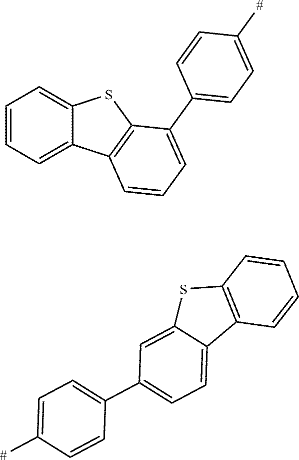

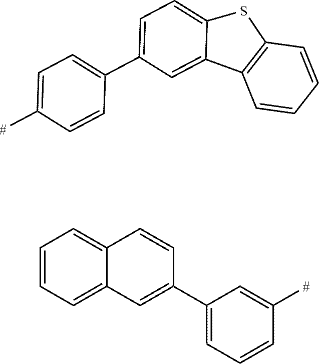

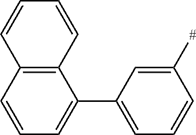

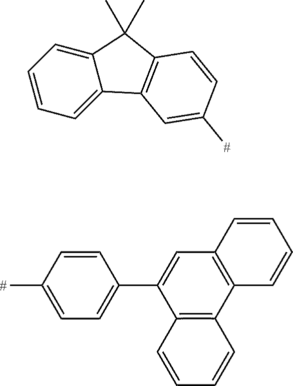

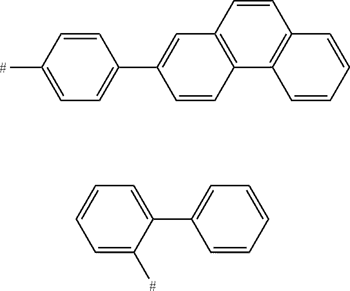

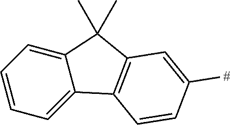

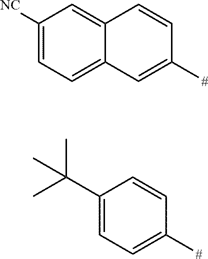

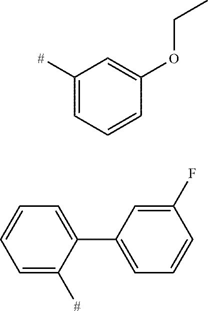

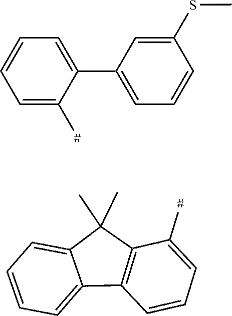

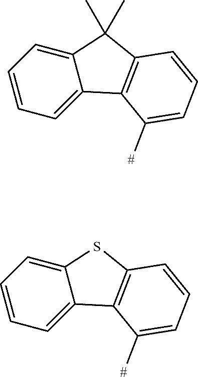

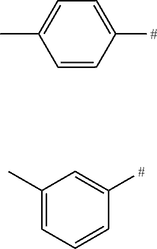

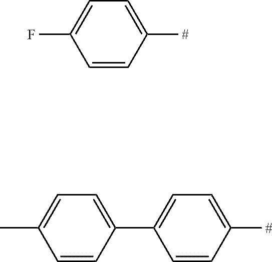

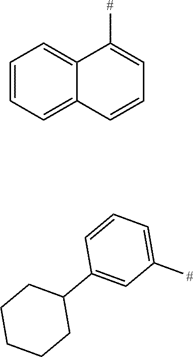

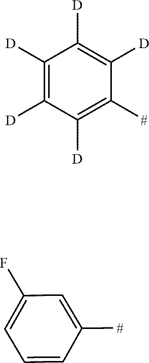

Optionally, R₀ is selected from hydrogen, methyl, ethyl, isopropyl, tert-butyl, or the following groups:

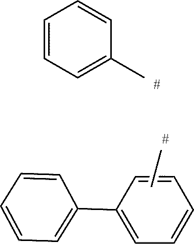

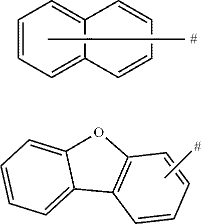

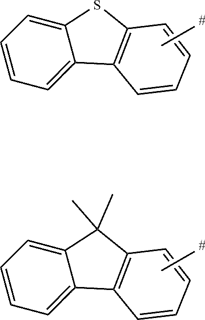

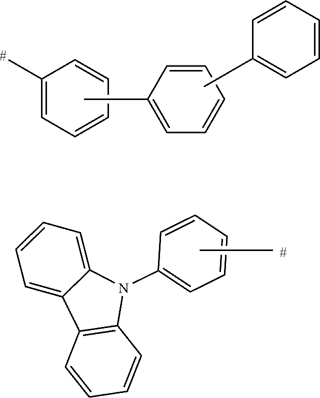

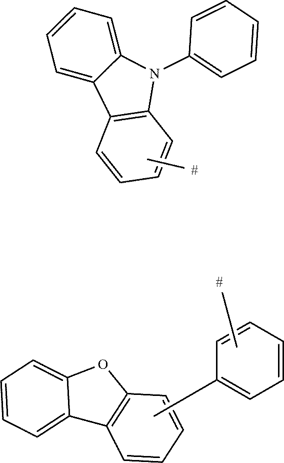

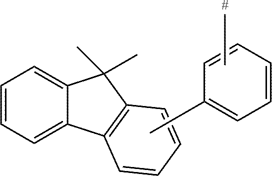

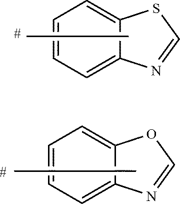

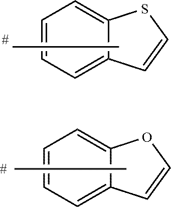

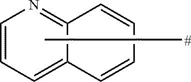

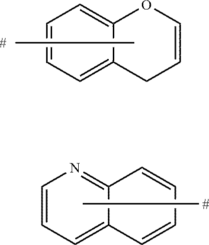

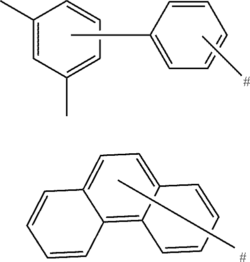

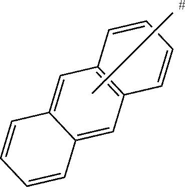

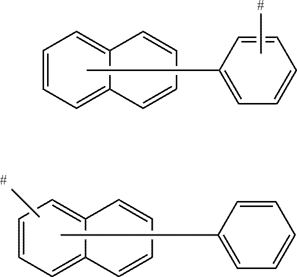

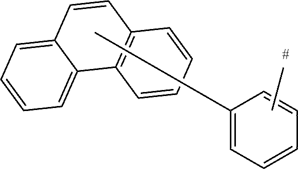

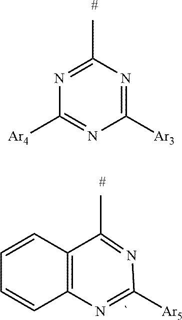

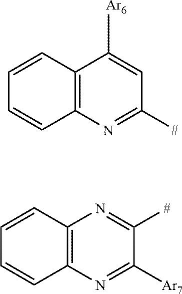

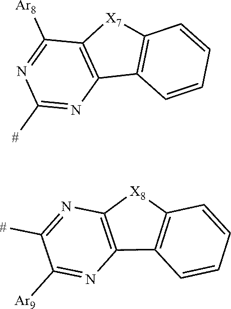

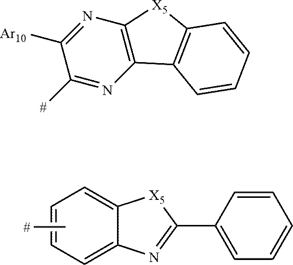

wherein the definitions of X₅, X₇ to X₉ and Ar₃ to Ar₁₀ as are described above.

Further optionally, R₀ is selected from hydrogen, methyl, or tert-butyl, or selected from the following groups:

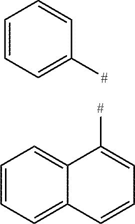

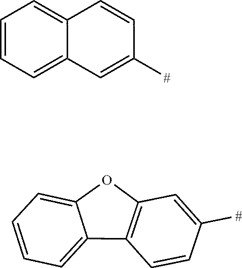

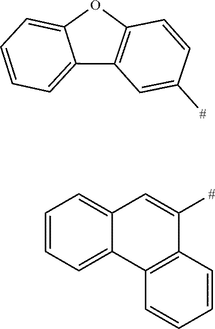

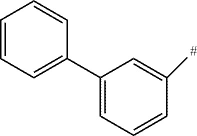

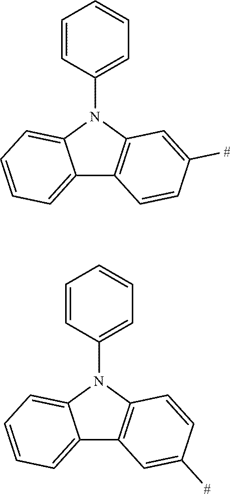

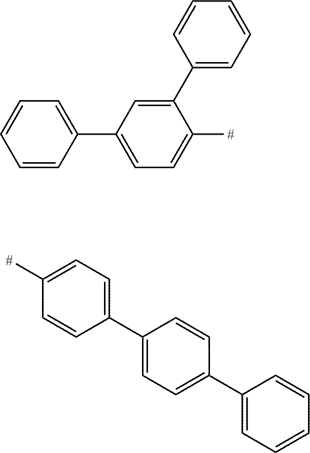

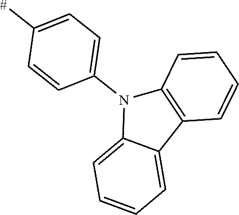

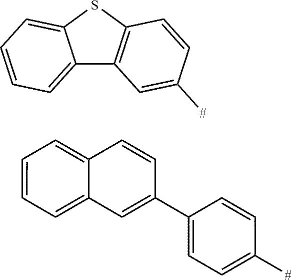

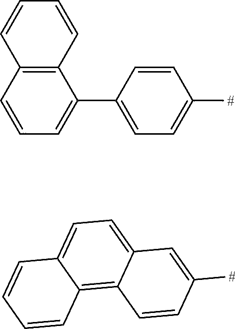

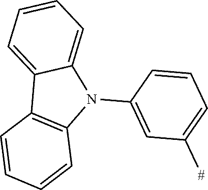

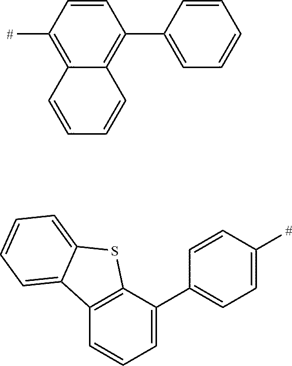

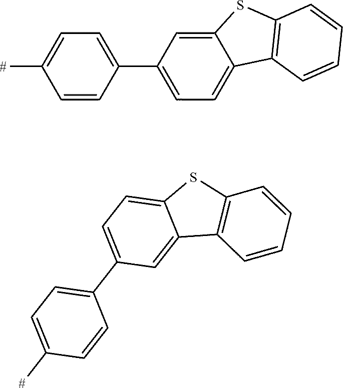

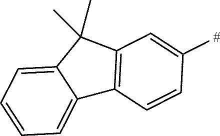

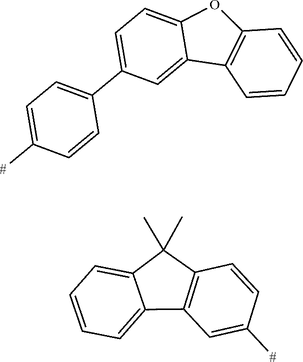

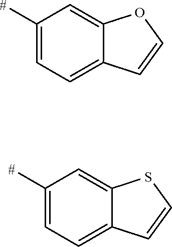

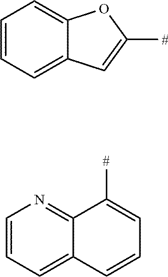

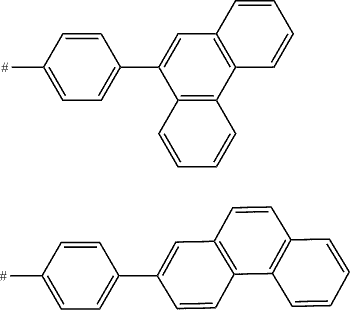

Optionally, the nitrogen-containing compound is selected from the following compounds:

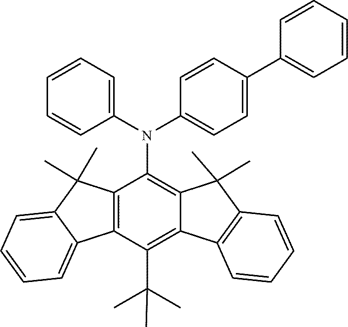 [SMILES: CC(C)(C)c1c2c(c(N(c3ccccc3)c3ccc(-c4ccccc4)cc3)c3c1-c1ccccc1C3(C)C)C(C)(C)c1ccccc1-2]

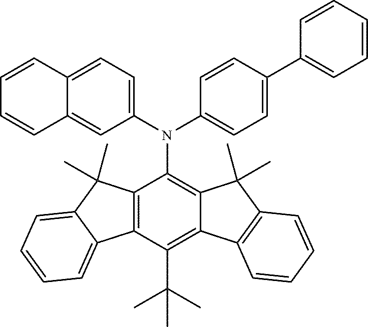 [SMILES: CC(C)(C)c1c2c(c(N(c3ccc(-c4ccccc4)cc3)c3ccc4ccccc4c3)c3c1-c1ccccc1C3(C)C)C(C)(C)c1ccccc1-2]

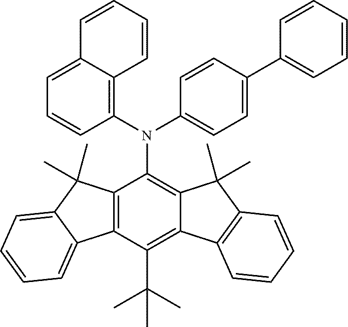 [SMILES: CC(C)(C)c1c2c(c(N(c3ccc(-c4ccccc4)cc3)c3cccc4ccccc34)c3c1-c1ccccc1C3(C)C)C(C)(C)c1ccccc1-2]

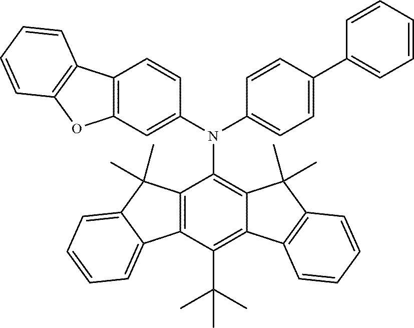 [SMILES: CC(C)(C)c1c2c(c(N(c3ccc(-c4ccccc4)cc3)c3ccc4c(c3)oc3ccccc34)c3c1-c1ccccc1C3(C)C)C(C)(C)c1ccccc1-2]

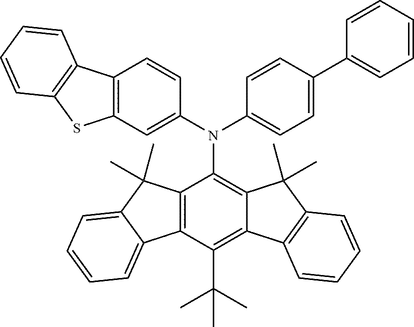 [SMILES: CC(C)(C)c1c2c(c(N(c3ccc(-c4ccccc4)cc3)c3ccc4c(c3)sc3ccccc34)c3c1-c1ccccc1C3(C)C)C(C)(C)c1ccccc1-2]

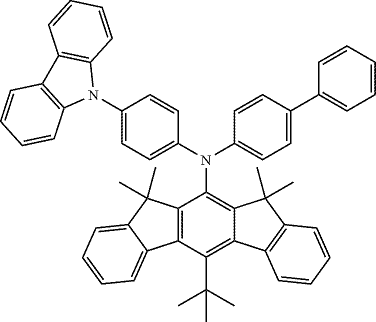 [SMILES: CC(C)(C)c1c2c(c(N(c3ccc(-c4ccccc4)cc3)c3ccc(-n4c5ccccc5c5ccccc54)cc3)c3c1-c1ccccc1C3(C)C)C(C)(C)c1ccccc1-2]

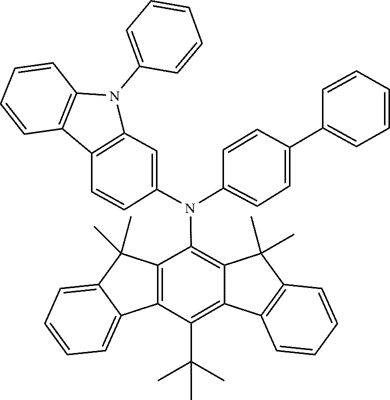 [SMILES: CC(C)(C)c1c2c(c(N(c3ccc(-c4ccccc4)cc3)c3ccc4c5ccccc5n(-c5ccccc5)c4c3)c3c1-c1ccccc1C3(C)C)C(C)(C)c1ccccc1-2]

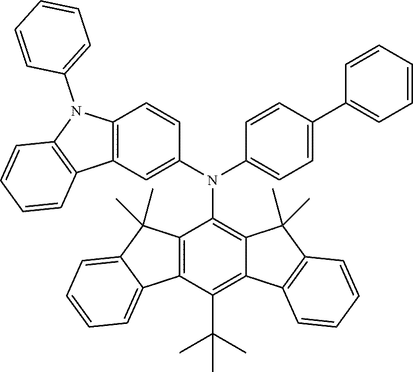 [SMILES: CC(C)(C)c1c2c(c(N(c3ccc(-c4ccccc4)cc3)c3ccc4c(c3)c3ccccc3n4-c3ccccc3)c3c1-c1ccccc1C3(C)C)C(C)(C)c1ccccc1-2]

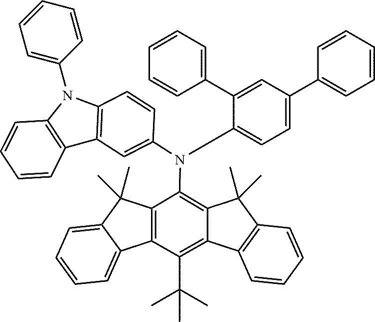 [SMILES: CC(C)(C)c1c2c(c(N(c3ccc4c(c3)c3ccccc3n4-c3ccccc3)c3ccc(-c4ccccc4)cc3-c3ccccc3)c3c1-c1ccccc1C3(C)C)C(C)(C)c1ccccc1-2]

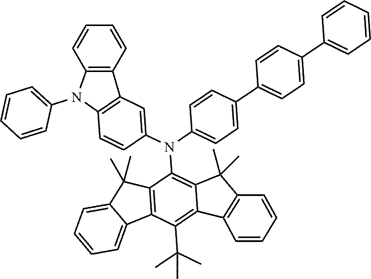 [SMILES: CC(C)(C)c1c2c(c(N(c3ccc(-c4ccc(-c5ccccc5)cc4)cc3)c3ccc4c(c3)c3ccccc3n4-c3ccccc3)c3c1-c1ccccc1C3(C)C)C(C)(C)c1ccccc1-2]

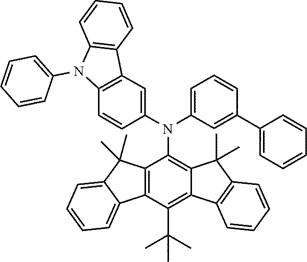 [SMILES: CC(C)(C)c1c2c(c(N(c3cccc(-c4ccccc4)c3)c3ccc4c(c3)c3ccccc3n4-c3ccccc3)c3c1-c1ccccc1C3(C)C)C(C)(C)c1ccccc1-2]

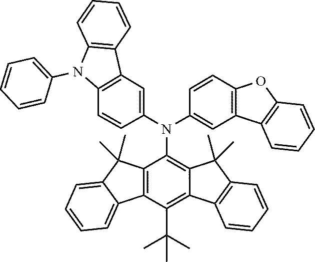 [SMILES: CC(C)(C)c1c2c(c(N(c3ccc4oc5ccccc5c4c3)c3ccc4c(c3)c3ccccc3n4-c3ccccc3)c3c1-c1ccccc1C3(C)C)C(C)(C)c1ccccc1-2]

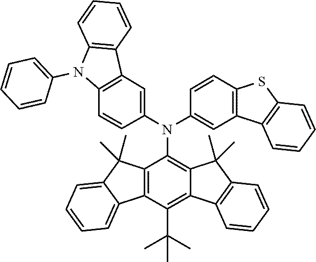 [SMILES: CC(C)(C)c1c2c(c(N(c3ccc4sc5ccccc5c4c3)c3ccc4c(c3)c3ccccc3n4-c3ccccc3)c3c1-c1ccccc1C3(C)C)C(C)(C)c1ccccc1-2]

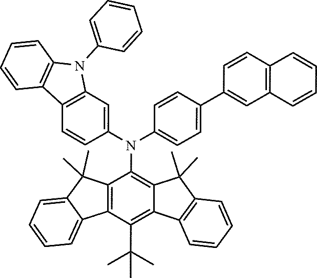 [SMILES: CC(C)(C)c1c2c(c(N(c3ccc(-c4ccc5ccccc5c4)cc3)c3ccc4c5ccccc5n(-c5ccccc5)c4c3)c3c1-c1ccccc1C3(C)C)C(C)(C)c1ccccc1-2]

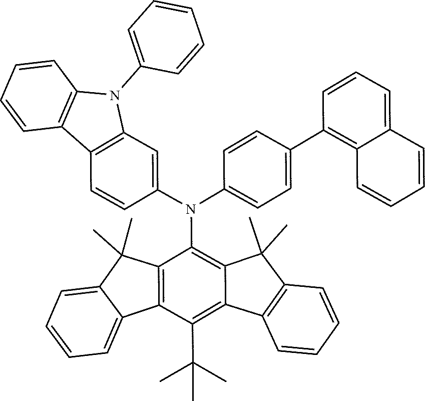 [SMILES: CC(C)(C)c1c2c(c(N(c3ccc(-c4cccc5ccccc45)cc3)c3ccc4c5ccccc5n(-c5ccccc5)c4c3)c3c1-c1ccccc1C3(C)C)C(C)(C)c1ccccc1-2]

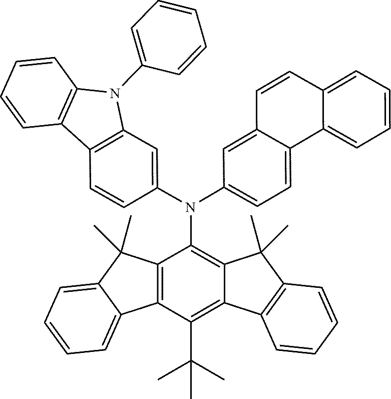 [SMILES: CC(C)(C)c1c2c(c(N(c3ccc4c(ccc5ccccc54)c3)c3ccc4c5ccccc5n(-c5ccccc5)c4c3)c3c1-c1ccccc1C3(C)C)C(C)(C)c1ccccc1-2]

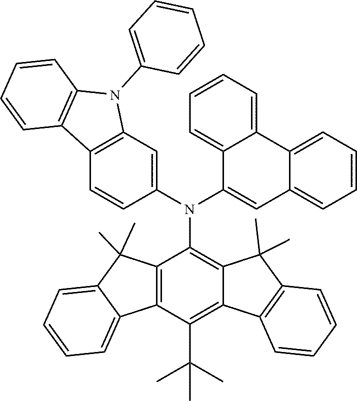 [SMILES: CC(C)(C)c1c2c(c(N(c3ccc4c5ccccc5n(-c5ccccc5)c4c3)c3cc4ccccc4c4ccccc34)c3c1-c1ccccc1C3(C)C)C(C)(C)c1ccccc1-2]

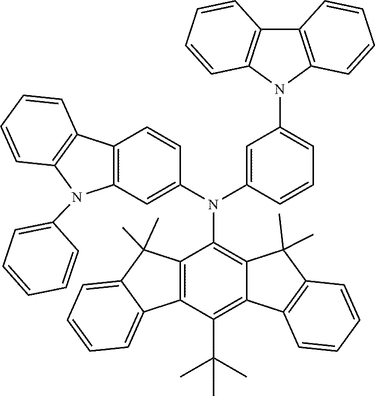 [SMILES: CC(C)(C)c1c2c(c(N(c3cccc(-n4c5ccccc5c5ccccc54)c3)c3ccc4c5ccccc5n(-c5ccccc5)c4c3)c3c1-c1ccccc1C3(C)C)C(C)(C)c1ccccc1-2]

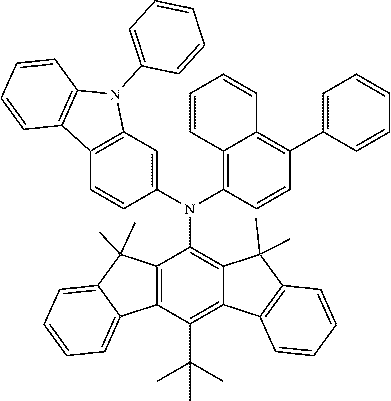 [SMILES: CC(C)(C)c1c2c(c(N(c3ccc4c5ccccc5n(-c5ccccc5)c4c3)c3ccc(-c4ccccc4)c4ccccc34)c3c1-c1ccccc1C3(C)C)C(C)(C)c1ccccc1-2]

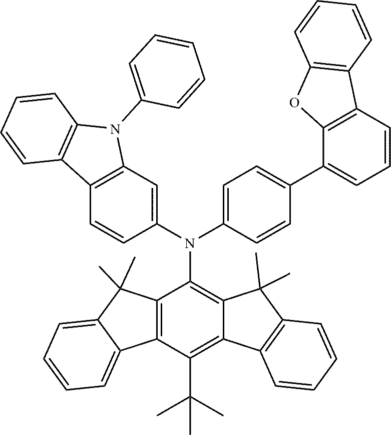 [SMILES: CC(C)(C)c1c2c(c(N(c3ccc(-c4cccc5c4oc4ccccc45)cc3)c3ccc4c5ccccc5n(-c5ccccc5)c4c3)c3c1-c1ccccc1C3(C)C)C(C)(C)c1ccccc1-2]

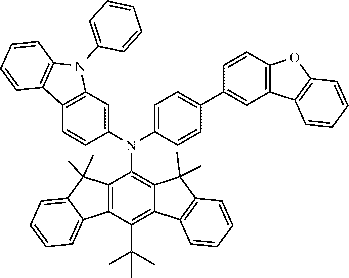 [SMILES: CC(C)(C)c1c2c(c(N(c3ccc(-c4ccc5oc6ccccc6c5c4)cc3)c3ccc4c5ccccc5n(-c5ccccc5)c4c3)c3c1-c1ccccc1C3(C)C)C(C)(C)c1ccccc1-2]

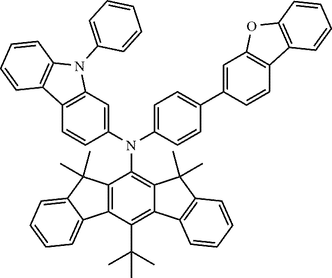 [SMILES: CC(C)(C)c1c2c(c(N(c3ccc(-c4ccc5c(c4)oc4ccccc45)cc3)c3ccc4c5ccccc5n(-c5ccccc5)c4c3)c3c1-c1ccccc1C3(C)C)C(C)(C)c1ccccc1-2]

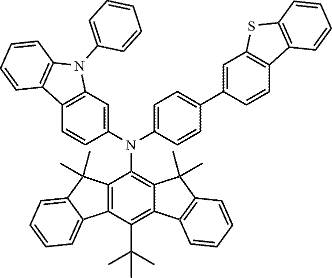 [SMILES: CC(C)(C)c1c2c(c(N(c3ccc(-c4ccc5c(c4)sc4ccccc45)cc3)c3ccc4c5ccccc5n(-c5ccccc5)c4c3)c3c1-c1ccccc1C3(C)C)C(C)(C)c1ccccc1-2]

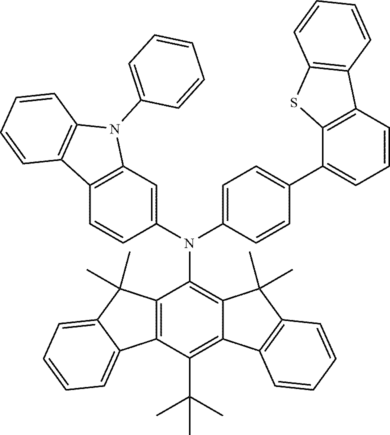 [SMILES: CC(C)(C)c1c2c(c(N(c3ccc(-c4cccc5c4sc4ccccc45)cc3)c3ccc4c5ccccc5n(-c5ccccc5)c4c3)c3c1-c1ccccc1C3(C)C)C(C)(C)c1ccccc1-2]

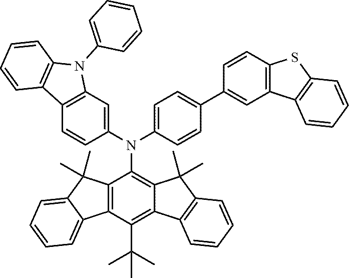 [SMILES: CC(C)(C)c1c2c(c(N(c3ccc(-c4ccc5sc6ccccc6c5c4)cc3)c3ccc4c5ccccc5n(-c5ccccc5)c4c3)c3c1-c1ccccc1C3(C)C)C(C)(C)c1ccccc1-2]

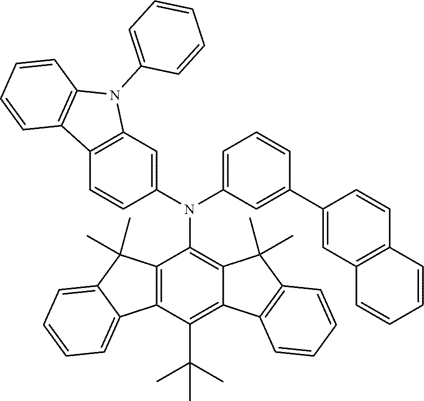 [SMILES: CC(C)(C)c1c2c(c(N(c3cccc(-c4ccc5ccccc5c4)c3)c3ccc4c5ccccc5n(-c5ccccc5)c4c3)c3c1-c1ccccc1C3(C)C)C(C)(C)c1ccccc1-2]

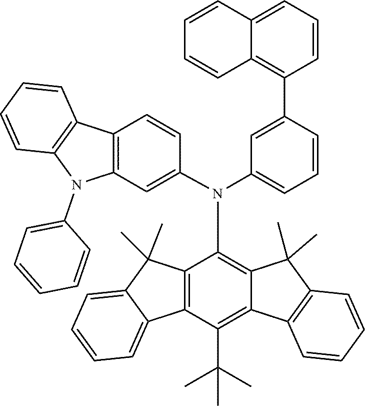 [SMILES: CC(C)(C)c1c2c(c(N(c3cccc(-c4cccc5ccccc45)c3)c3ccc4c5ccccc5n(-c5ccccc5)c4c3)c3c1-c1ccccc1C3(C)C)C(C)(C)c1ccccc1-2]

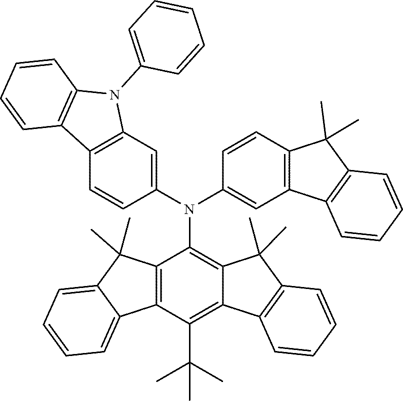 [SMILES: CC(C)(C)c1c2c(c(N(c3ccc4c(c3)-c3ccccc3C4(C)C)c3ccc4c5ccccc5n(-c5ccccc5)c4c3)c3c1-c1ccccc1C3(C)C)C(C)(C)c1ccccc1-2]

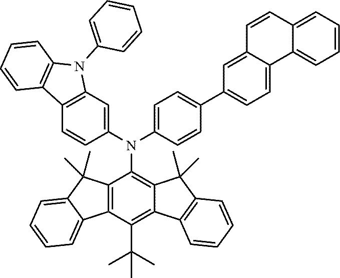 [SMILES: CC(C)(C)c1c2c(c(N(c3ccc(-c4ccc5c(ccc6ccccc65)c4)cc3)c3ccc4c5ccccc5n(-c5ccccc5)c4c3)c3c1-c1ccccc1C3(C)C)C(C)(C)c1ccccc1-2]

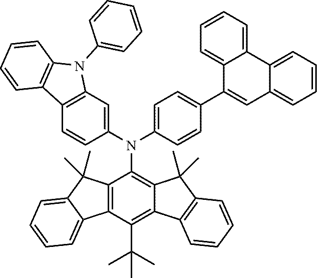 [SMILES: CC(C)(C)c1c2c(c(N(c3ccc(-c4cc5ccccc5c5ccccc45)cc3)c3ccc4c5ccccc5n(-c5ccccc5)c4c3)c3c1-c1ccccc1C3(C)C)C(C)(C)c1ccccc1-2]

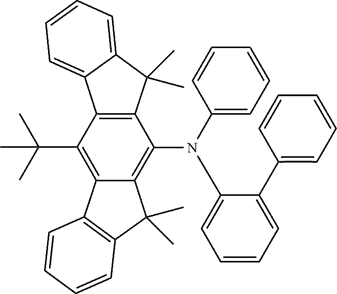 [SMILES: CC(C)(C)c1c2c(c(N(c3ccccc3)c3ccccc3-c3ccccc3)c3c1-c1ccccc1C3(C)C)C(C)(C)c1ccccc1-2]

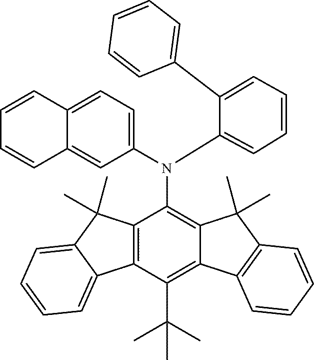 [SMILES: CC(C)(C)c1c2c(c(N(c3ccc4ccccc4c3)c3ccccc3-c3ccccc3)c3c1-c1ccccc1C3(C)C)C(C)(C)c1ccccc1-2]

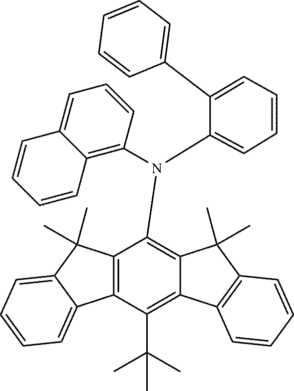 [SMILES: CC(C)(C)c1c2c(c(N(c3ccccc3-c3ccccc3)c3cccc4ccccc34)c3c1-c1ccccc1C3(C)C)C(C)(C)c1ccccc1-2]

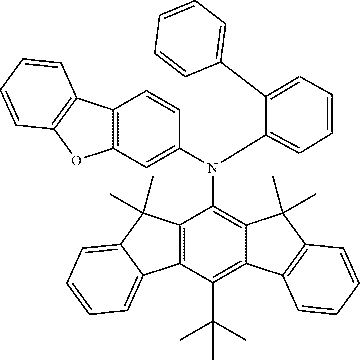 [SMILES: CC(C)(C)c1c2c(c(N(c3ccc4c(c3)oc3ccccc34)c3ccccc3-c3ccccc3)c3c1-c1ccccc1C3(C)C)C(C)(C)c1ccccc1-2]

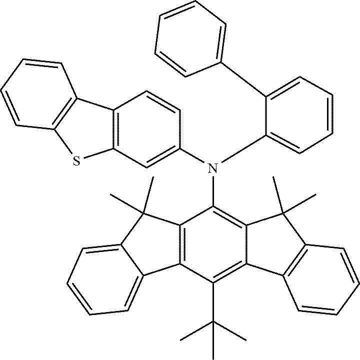 [SMILES: CC(C)(C)c1c2c(c(N(c3ccc4c(c3)sc3ccccc34)c3ccccc3-c3ccccc3)c3c1-c1ccccc1C3(C)C)C(C)(C)c1ccccc1-2]

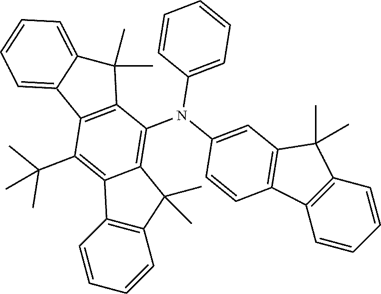 [SMILES: CC(C)(C)c1c2c(c(N(c3ccccc3)c3ccc4c(c3)C(C)(C)c3ccccc3-4)c3c1-c1ccccc1C3(C)C)C(C)(C)c1ccccc1-2]

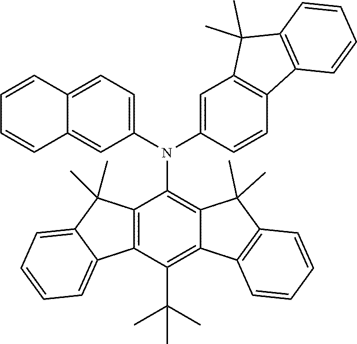 [SMILES: CC(C)(C)c1c2c(c(N(c3ccc4c(c3)C(C)(C)c3ccccc3-4)c3ccc4ccccc4c3)c3c1-c1ccccc1C3(C)C)C(C)(C)c1ccccc1-2]

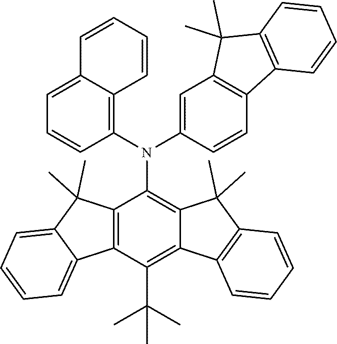 [SMILES: CC(C)(C)c1c2c(c(N(c3ccc4c(c3)C(C)(C)c3ccccc3-4)c3cccc4ccccc34)c3c1-c1ccccc1C3(C)C)C(C)(C)c1ccccc1-2]

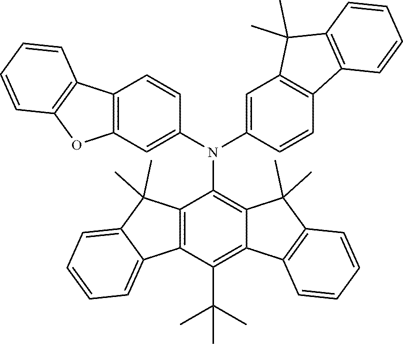 [SMILES: CC(C)(C)c1c2c(c(N(c3ccc4c(c3)C(C)(C)c3ccccc3-4)c3ccc4c(c3)oc3ccccc34)c3c1-c1ccccc1C3(C)C)C(C)(C)c1ccccc1-2]

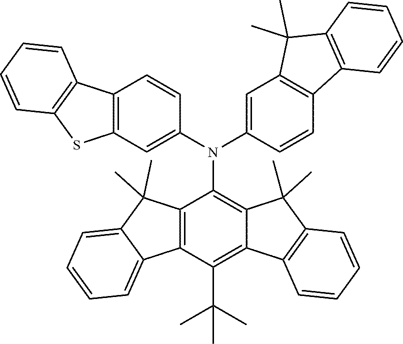 [SMILES: CC(C)(C)c1c2c(c(N(c3ccc4c(c3)C(C)(C)c3ccccc3-4)c3ccc4c(c3)sc3ccccc34)c3c1-c1ccccc1C3(C)C)C(C)(C)c1ccccc1-2]

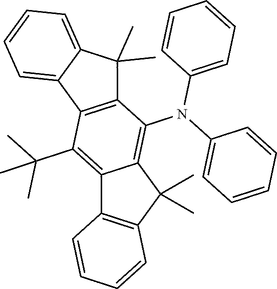 [SMILES: CC(C)(C)c1c2c(c(N(c3ccccc3)c3ccccc3)c3c1-c1ccccc1C3(C)C)C(C)(C)c1ccccc1-2]

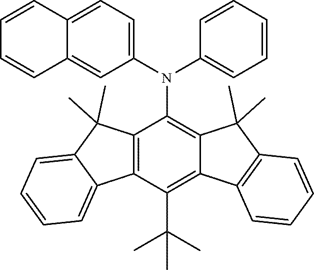 [SMILES: CC(C)(C)c1c2c(c(N(c3ccccc3)c3ccc4ccccc4c3)c3c1-c1ccccc1C3(C)C)C(C)(C)c1ccccc1-2]

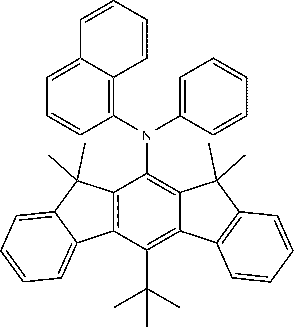 [SMILES: CC(C)(C)c1c2c(c(N(c3ccccc3)c3cccc4ccccc34)c3c1-c1ccccc1C3(C)C)C(C)(C)c1ccccc1-2]

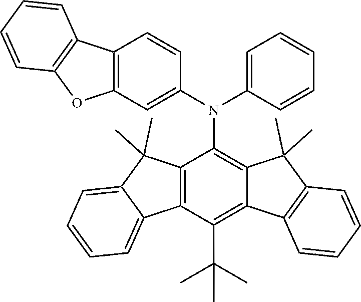 [SMILES: CC(C)(C)c1c2c(c(N(c3ccccc3)c3ccc4c(c3)oc3ccccc34)c3c1-c1ccccc1C3(C)C)C(C)(C)c1ccccc1-2]

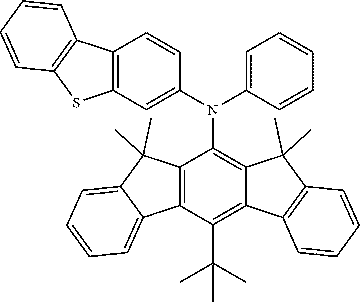 [SMILES: CC(C)(C)c1c2c(c(N(c3ccccc3)c3ccc4c(c3)sc3ccccc34)c3c1-c1ccccc1C3(C)C)C(C)(C)c1ccccc1-2]

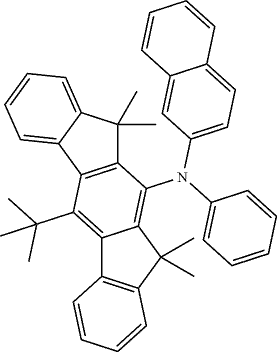 [SMILES: CC(C)(C)c1c2c(c(N(c3ccccc3)c3ccc4ccccc4c3)c3c1-c1ccccc1C3(C)C)C(C)(C)c1ccccc1-2]

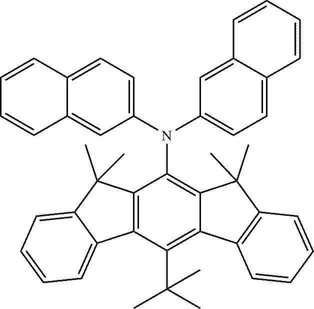 [SMILES: CC(C)(C)c1c2c(c(N(c3ccc4ccccc4c3)c3ccc4ccccc4c3)c3c1-c1ccccc1C3(C)C)C(C)(C)c1ccccc1-2]

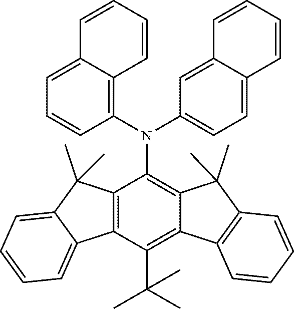 [SMILES: CC(C)(C)c1c2c(c(N(c3ccc4ccccc4c3)c3cccc4ccccc34)c3c1-c1ccccc1C3(C)C)C(C)(C)c1ccccc1-2]

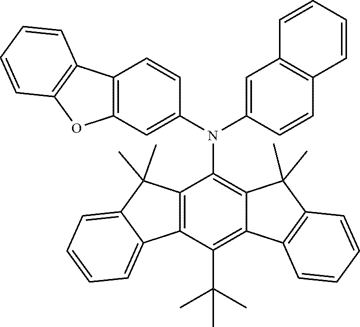 [SMILES: CC(C)(C)c1c2c(c(N(c3ccc4ccccc4c3)c3ccc4c(c3)oc3ccccc34)c3c1-c1ccccc1C3(C)C)C(C)(C)c1ccccc1-2]

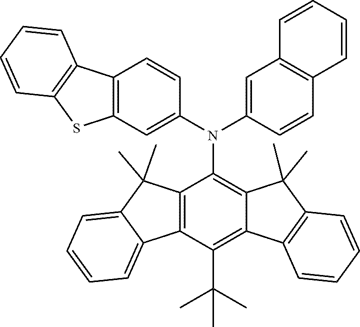 [SMILES: CC(C)(C)c1c2c(c(N(c3ccc4ccccc4c3)c3ccc4c(c3)sc3ccccc34)c3c1-c1ccccc1C3(C)C)C(C)(C)c1ccccc1-2]

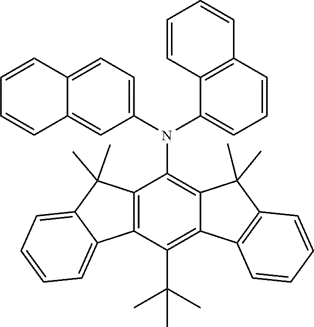 [SMILES: CC(C)(C)c1c2c(c(N(c3ccc4ccccc4c3)c3cccc4ccccc34)c3c1-c1ccccc1C3(C)C)C(C)(C)c1ccccc1-2]

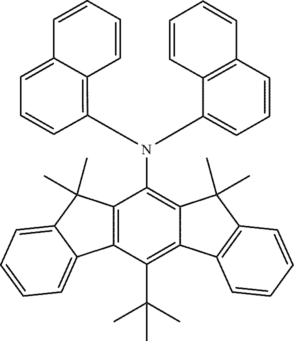 [SMILES: CC(C)(C)c1c2c(c(N(c3cccc4ccccc34)c3cccc4ccccc34)c3c1-c1ccccc1C3(C)C)C(C)(C)c1ccccc1-2]

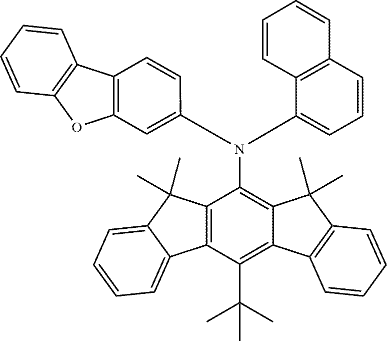 [SMILES: CC(C)(C)c1c2c(c(N(c3ccc4c(c3)oc3ccccc34)c3cccc4ccccc34)c3c1-c1ccccc1C3(C)C)C(C)(C)c1ccccc1-2]

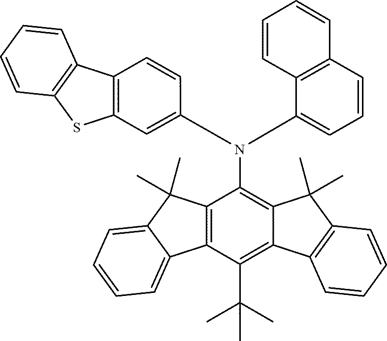 [SMILES: CC(C)(C)c1c2c(c(N(c3ccc4c(c3)sc3ccccc34)c3cccc4ccccc34)c3c1-c1ccccc1C3(C)C)C(C)(C)c1ccccc1-2]

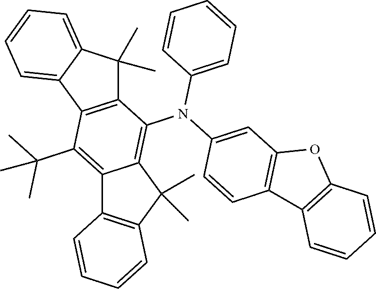 [SMILES: CC(C)(C)c1c2c(c(N(c3ccccc3)c3ccc4c(c3)oc3ccccc34)c3c1-c1ccccc1C3(C)C)C(C)(C)c1ccccc1-2]

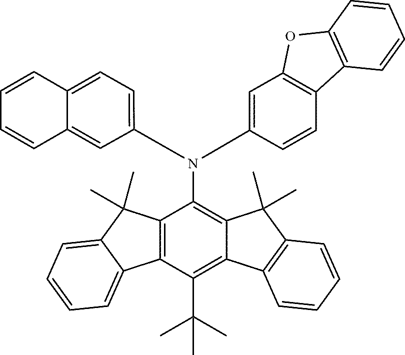 [SMILES: CC(C)(C)c1c2c(c(N(c3ccc4ccccc4c3)c3ccc4c(c3)oc3ccccc34)c3c1-c1ccccc1C3(C)C)C(C)(C)c1ccccc1-2]

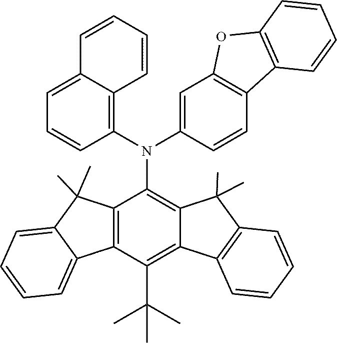 [SMILES: CC(C)(C)c1c2c(c(N(c3ccc4c(c3)oc3ccccc34)c3cccc4ccccc34)c3c1-c1ccccc1C3(C)C)C(C)(C)c1ccccc1-2]

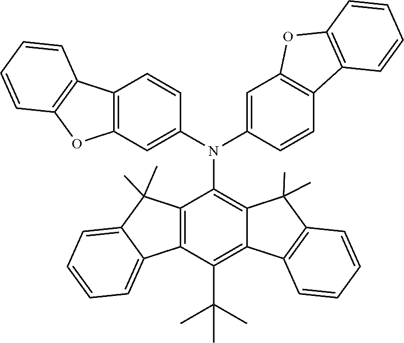 [SMILES: CC(C)(C)c1c2c(c(N(c3ccc4c(c3)oc3ccccc34)c3ccc4c(c3)oc3ccccc34)c3c1-c1ccccc1C3(C)C)C(C)(C)c1ccccc1-2]

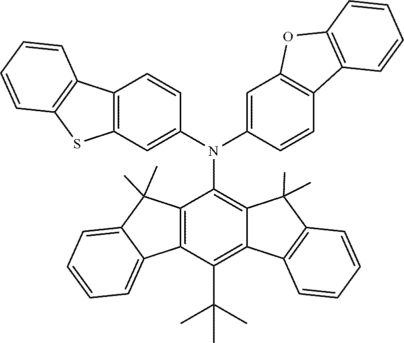 [SMILES: CC(C)(C)c1c2c(c(N(c3ccc4c(c3)oc3ccccc34)c3ccc4c(c3)sc3ccccc34)c3c1-c1ccccc1C3(C)C)C(C)(C)c1ccccc1-2]

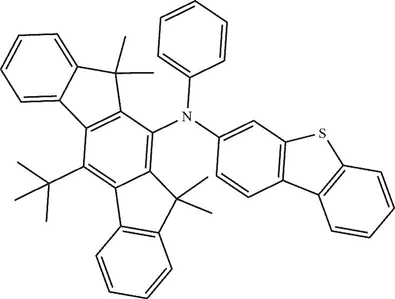 [SMILES: CC(C)(C)c1c2c(c(N(c3ccccc3)c3ccc4c(c3)sc3ccccc34)c3c1-c1ccccc1C3(C)C)C(C)(C)c1ccccc1-2]

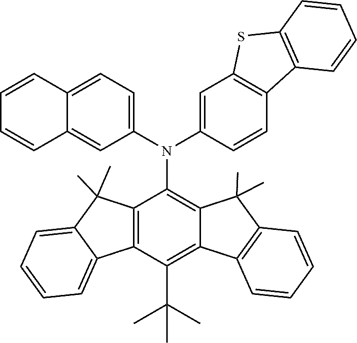 [SMILES: CC(C)(C)c1c2c(c(N(c3ccc4ccccc4c3)c3ccc4c(c3)sc3ccccc34)c3c1-c1ccccc1C3(C)C)C(C)(C)c1ccccc1-2]

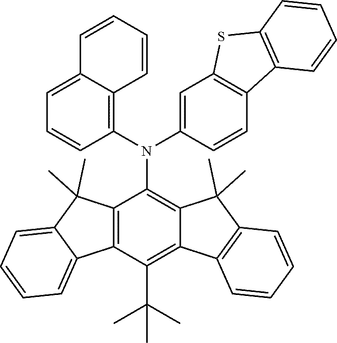 [SMILES: CC(C)(C)c1c2c(c(N(c3ccc4c(c3)sc3ccccc34)c3cccc4ccccc34)c3c1-c1ccccc1C3(C)C)C(C)(C)c1ccccc1-2]

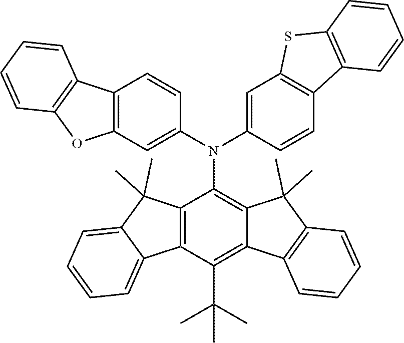 [SMILES: CC(C)(C)c1c2c(c(N(c3ccc4c(c3)oc3ccccc34)c3ccc4c(c3)sc3ccccc34)c3c1-c1ccccc1C3(C)C)C(C)(C)c1ccccc1-2]

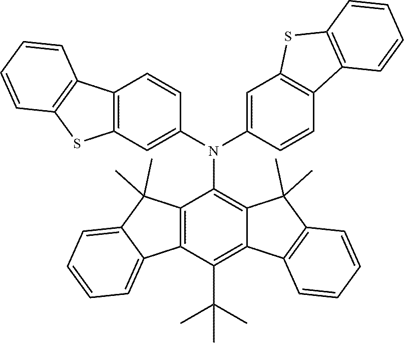 [SMILES: CC(C)(C)c1c2c(c(N(c3ccc4c(c3)sc3ccccc34)c3ccc4c(c3)sc3ccccc34)c3c1-c1ccccc1C3(C)C)C(C)(C)c1ccccc1-2]

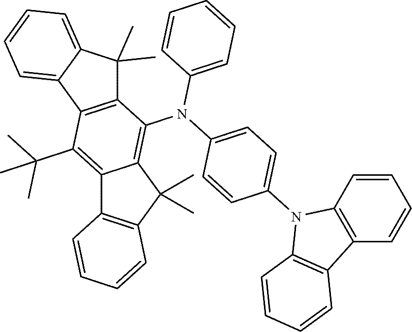 [SMILES: CC(C)(C)c1c2c(c(N(c3ccccc3)c3ccc(-n4c5ccccc5c5ccccc54)cc3)c3c1-c1ccccc1C3(C)C)C(C)(C)c1ccccc1-2]

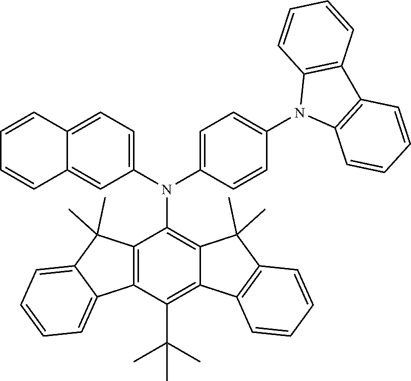 [SMILES: CC(C)(C)c1c2c(c(N(c3ccc(-n4c5ccccc5c5ccccc54)cc3)c3ccc4ccccc4c3)c3c1-c1ccccc1C3(C)C)C(C)(C)c1ccccc1-2]

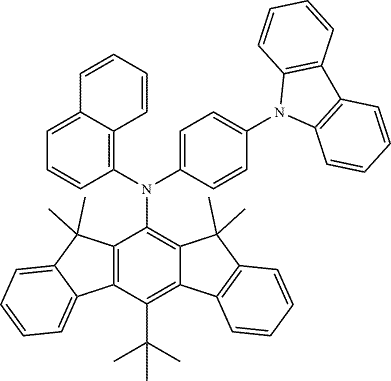 [SMILES: CC(C)(C)c1c2c(c(N(c3ccc(-n4c5ccccc5c5ccccc54)cc3)c3cccc4ccccc34)c3c1-c1ccccc1C3(C)C)C(C)(C)c1ccccc1-2]

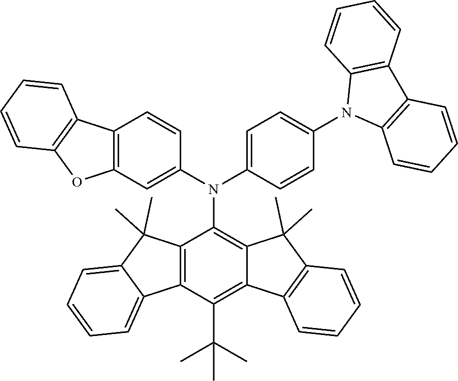 [SMILES: CC(C)(C)c1c2c(c(N(c3ccc(-n4c5ccccc5c5ccccc54)cc3)c3ccc4c(c3)oc3ccccc34)c3c1-c1ccccc1C3(C)C)C(C)(C)c1ccccc1-2]

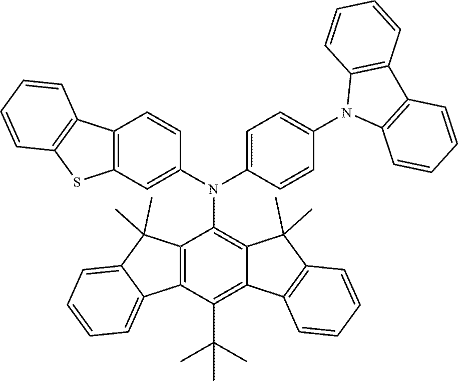 [SMILES: CC(C)(C)c1c2c(c(N(c3ccc(-n4c5ccccc5c5ccccc54)cc3)c3ccc4c(c3)sc3ccccc34)c3c1-c1ccccc1C3(C)C)C(C)(C)c1ccccc1-2]

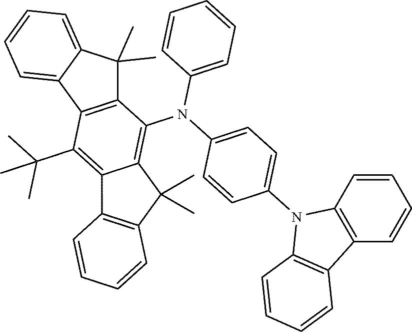 [SMILES: CC(C)(C)c1c2c(c(N(c3ccccc3)c3ccc(-n4c5ccccc5c5ccccc54)cc3)c3c1-c1ccccc1C3(C)C)C(C)(C)c1ccccc1-2]

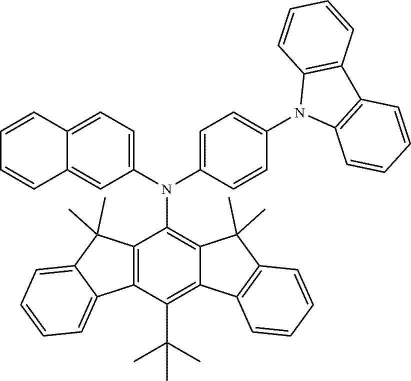 [SMILES: CC(C)(C)c1c2c(c(N(c3ccc(-n4c5ccccc5c5ccccc54)cc3)c3ccc4ccccc4c3)c3c1-c1ccccc1C3(C)C)C(C)(C)c1ccccc1-2]

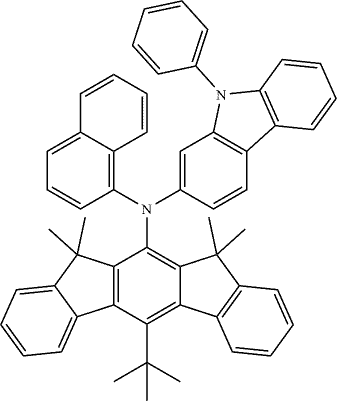 [SMILES: CC(C)(C)c1c2c(c(N(c3ccc4c5ccccc5n(-c5ccccc5)c4c3)c3cccc4ccccc34)c3c1-c1ccccc1C3(C)C)C(C)(C)c1ccccc1-2]

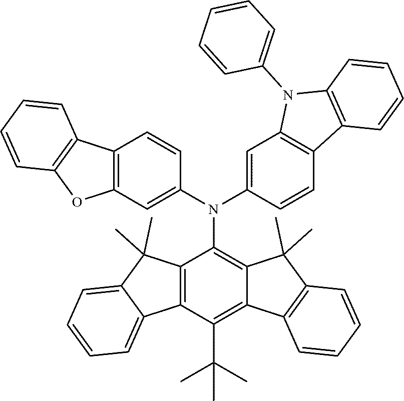 [SMILES: CC(C)(C)c1c2c(c(N(c3ccc4c(c3)oc3ccccc34)c3ccc4c5ccccc5n(-c5ccccc5)c4c3)c3c1-c1ccccc1C3(C)C)C(C)(C)c1ccccc1-2]

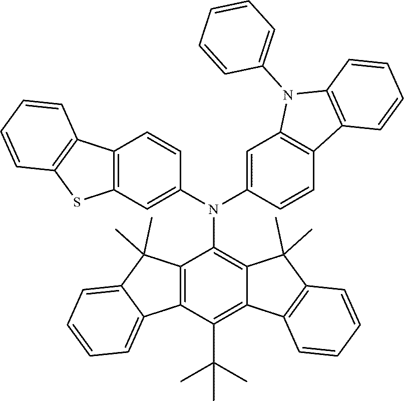 [SMILES: CC(C)(C)c1c2c(c(N(c3ccc4c(c3)sc3ccccc34)c3ccc4c5ccccc5n(-c5ccccc5)c4c3)c3c1-c1ccccc1C3(C)C)C(C)(C)c1ccccc1-2]

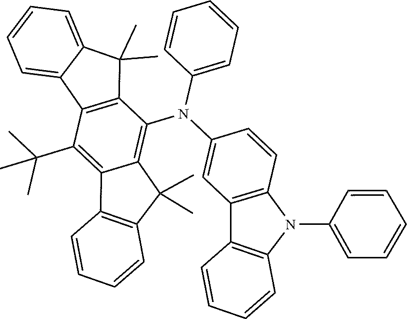 [SMILES: CC(C)(C)c1c2c(c(N(c3ccccc3)c3ccc4c(c3)c3ccccc3n4-c3ccccc3)c3c1-c1ccccc1C3(C)C)C(C)(C)c1ccccc1-2]

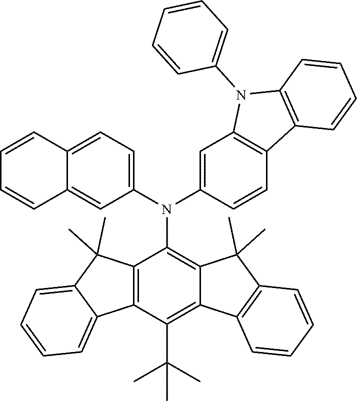 [SMILES: CC(C)(C)c1c2c(c(N(c3ccc4ccccc4c3)c3ccc4c5ccccc5n(-c5ccccc5)c4c3)c3c1-c1ccccc1C3(C)C)C(C)(C)c1ccccc1-2]

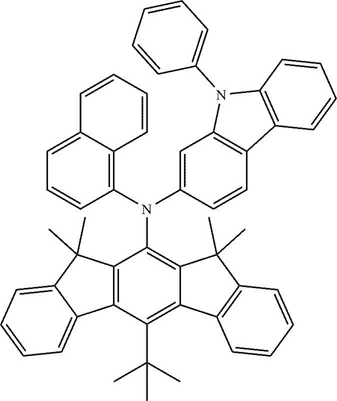 [SMILES: CC(C)(C)c1c2c(c(N(c3ccc4c5ccccc5n(-c5ccccc5)c4c3)c3cccc4ccccc34)c3c1-c1ccccc1C3(C)C)C(C)(C)c1ccccc1-2]

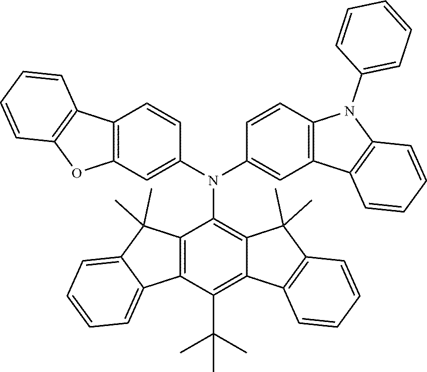 [SMILES: CC(C)(C)c1c2c(c(N(c3ccc4c(c3)oc3ccccc34)c3ccc4c(c3)c3ccccc3n4-c3ccccc3)c3c1-c1ccccc1C3(C)C)C(C)(C)c1ccccc1-2]

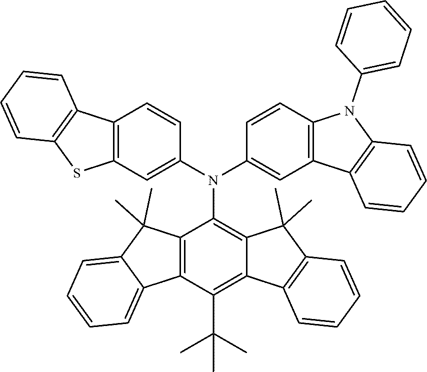 [SMILES: CC(C)(C)c1c2c(c(N(c3ccc4c(c3)sc3ccccc34)c3ccc4c(c3)c3ccccc3n4-c3ccccc3)c3c1-c1ccccc1C3(C)C)C(C)(C)c1ccccc1-2]

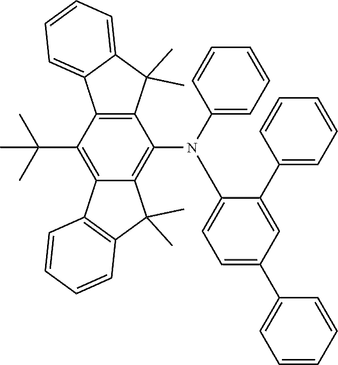 [SMILES: CC(C)(C)c1c2c(c(N(c3ccccc3)c3ccc(-c4ccccc4)cc3-c3ccccc3)c3c1-c1ccccc1C3(C)C)C(C)(C)c1ccccc1-2]

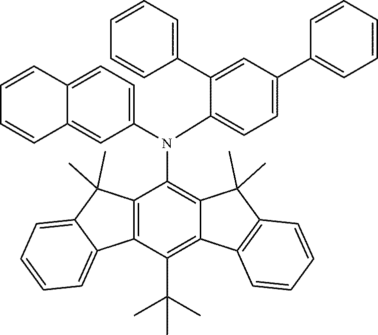 [SMILES: CC(C)(C)c1c2c(c(N(c3ccc4ccccc4c3)c3ccc(-c4ccccc4)cc3-c3ccccc3)c3c1-c1ccccc1C3(C)C)C(C)(C)c1ccccc1-2]

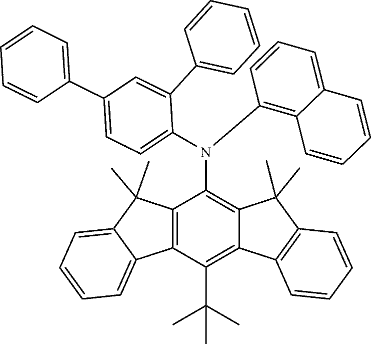 [SMILES: CC(C)(C)c1c2c(c(N(c3ccc(-c4ccccc4)cc3-c3ccccc3)c3cccc4ccccc34)c3c1-c1ccccc1C3(C)C)C(C)(C)c1ccccc1-2]

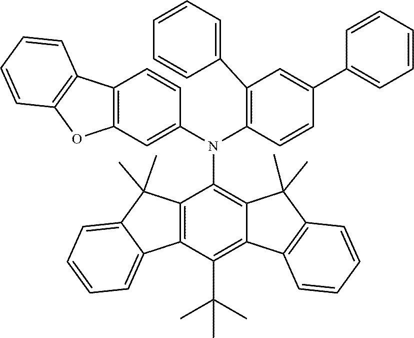 [SMILES: CC(C)(C)c1c2c(c(N(c3ccc4c(c3)oc3ccccc34)c3ccc(-c4ccccc4)cc3-c3ccccc3)c3c1-c1ccccc1C3(C)C)C(C)(C)c1ccccc1-2]

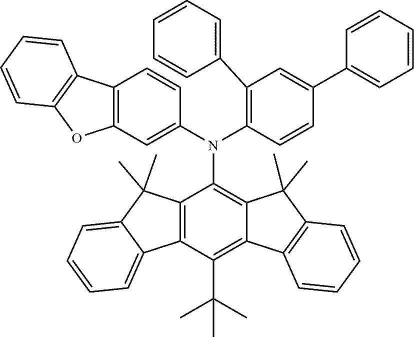 [SMILES: CC(C)(C)c1c2c(c(N(c3ccc4c(c3)oc3ccccc34)c3ccc(-c4ccccc4)cc3-c3ccccc3)c3c1-c1ccccc1C3(C)C)C(C)(C)c1ccccc1-2]

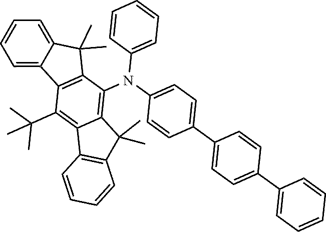 [SMILES: CC(C)(C)c1c2c(c(N(c3ccccc3)c3ccc(-c4ccc(-c5ccccc5)cc4)cc3)c3c1-c1ccccc1C3(C)C)C(C)(C)c1ccccc1-2]

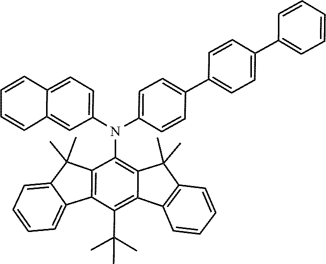 [SMILES: CC(C)(C)c1c2c(c(N(c3ccc(-c4ccc(-c5ccccc5)cc4)cc3)c3ccc4ccccc4c3)c3c1-c1ccccc1C3(C)C)C(C)(C)c1ccccc1-2]

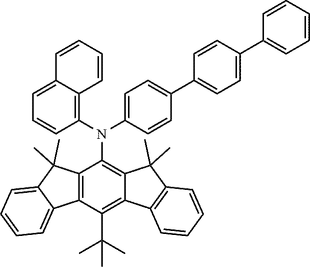 [SMILES: CC(C)(C)c1c2c(c(N(c3ccc(-c4ccc(-c5ccccc5)cc4)cc3)c3cccc4ccccc34)c3c1-c1ccccc1C3(C)C)C(C)(C)c1ccccc1-2]

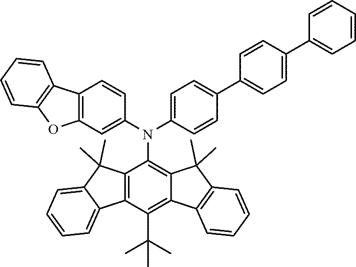 [SMILES: CC(C)(C)c1c2c(c(N(c3ccc(-c4ccc(-c5ccccc5)cc4)cc3)c3ccc4c(c3)oc3ccccc34)c3c1-c1ccccc1C3(C)C)C(C)(C)c1ccccc1-2]

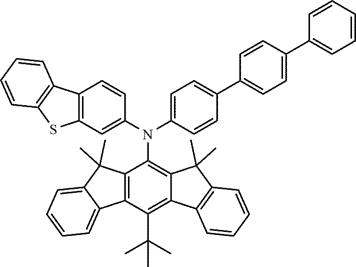 [SMILES: CC(C)(C)c1c2c(c(N(c3ccc(-c4ccc(-c5ccccc5)cc4)cc3)c3ccc4c(c3)sc3ccccc34)c3c1-c1ccccc1C3(C)C)C(C)(C)c1ccccc1-2]

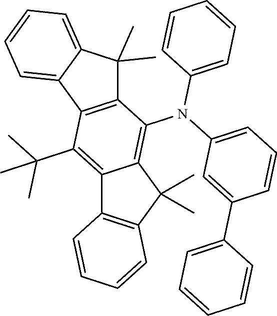 [SMILES: CC(C)(C)c1c2c(c(N(c3ccccc3)c3cccc(-c4ccccc4)c3)c3c1-c1ccccc1C3(C)C)C(C)(C)c1ccccc1-2]

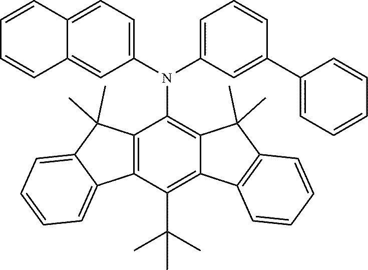 [SMILES: CC(C)(C)c1c2c(c(N(c3cccc(-c4ccccc4)c3)c3ccc4ccccc4c3)c3c1-c1ccccc1C3(C)C)C(C)(C)c1ccccc1-2]

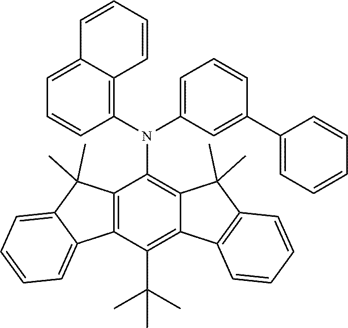 [SMILES: CC(C)(C)c1c2c(c(N(c3cccc(-c4ccccc4)c3)c3cccc4ccccc34)c3c1-c1ccccc1C3(C)C)C(C)(C)c1ccccc1-2]

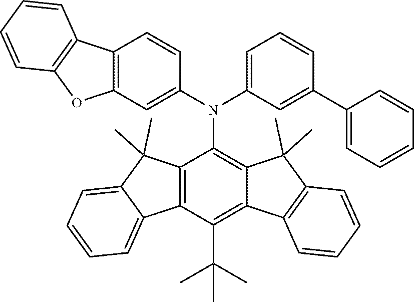 [SMILES: CC(C)(C)c1c2c(c(N(c3cccc(-c4ccccc4)c3)c3ccc4c(c3)oc3ccccc34)c3c1-c1ccccc1C3(C)C)C(C)(C)c1ccccc1-2]

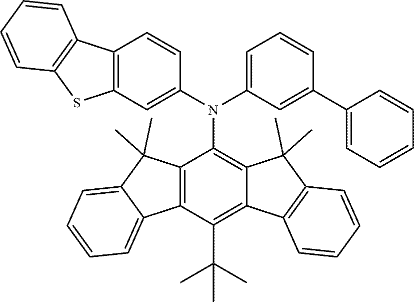 [SMILES: CC(C)(C)c1c2c(c(N(c3cccc(-c4ccccc4)c3)c3ccc4c(c3)sc3ccccc34)c3c1-c1ccccc1C3(C)C)C(C)(C)c1ccccc1-2]

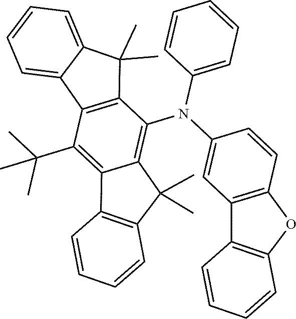 [SMILES: CC(C)(C)c1c2c(c(N(c3ccccc3)c3ccc4oc5ccccc5c4c3)c3c1-c1ccccc1C3(C)C)C(C)(C)c1ccccc1-2]

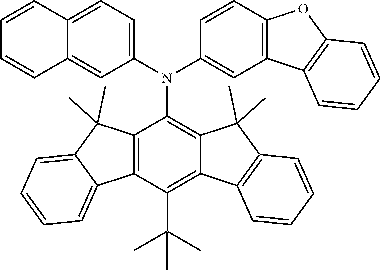 [SMILES: CC(C)(C)c1c2c(c(N(c3ccc4ccccc4c3)c3ccc4oc5ccccc5c4c3)c3c1-c1ccccc1C3(C)C)C(C)(C)c1ccccc1-2]

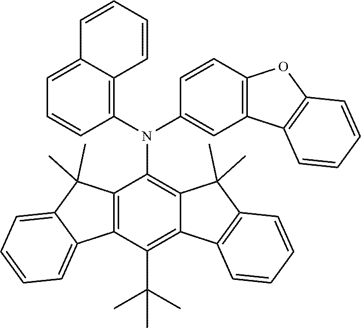 [SMILES: CC(C)(C)c1c2c(c(N(c3ccc4oc5ccccc5c4c3)c3cccc4ccccc34)c3c1-c1ccccc1C3(C)C)C(C)(C)c1ccccc1-2]

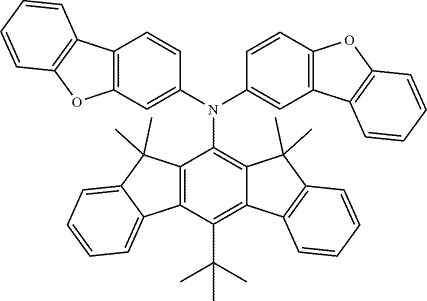 [SMILES: CC(C)(C)c1c2c(c(N(c3ccc4c(c3)oc3ccccc34)c3ccc4oc5ccccc5c4c3)c3c1-c1ccccc1C3(C)C)C(C)(C)c1ccccc1-2]

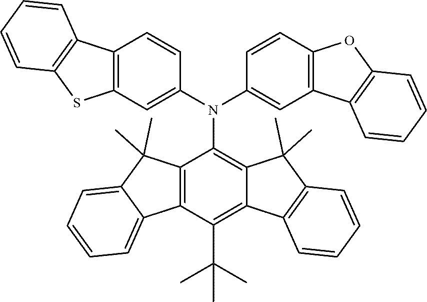 [SMILES: CC(C)(C)c1c2c(c(N(c3ccc4c(c3)sc3ccccc34)c3ccc4oc5ccccc5c4c3)c3c1-c1ccccc1C3(C)C)C(C)(C)c1ccccc1-2]

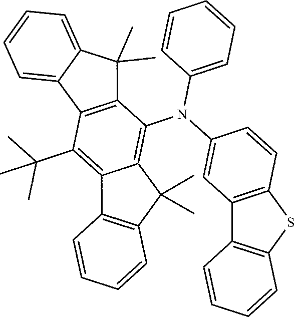 [SMILES: CC(C)(C)c1c2c(c(N(c3ccccc3)c3ccc4sc5ccccc5c4c3)c3c1-c1ccccc1C3(C)C)C(C)(C)c1ccccc1-2]

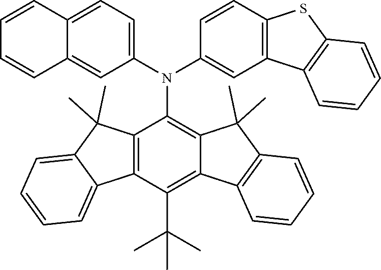 [SMILES: CC(C)(C)c1c2c(c(N(c3ccc4ccccc4c3)c3ccc4sc5ccccc5c4c3)c3c1-c1ccccc1C3(C)C)C(C)(C)c1ccccc1-2]

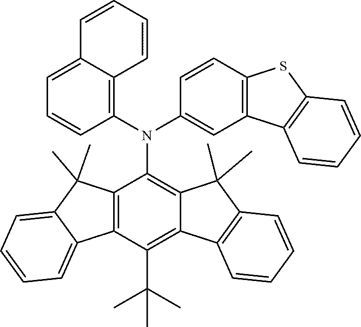 [SMILES: CC(C)(C)c1c2c(c(N(c3ccc4sc5ccccc5c4c3)c3cccc4ccccc34)c3c1-c1ccccc1C3(C)C)C(C)(C)c1ccccc1-2]

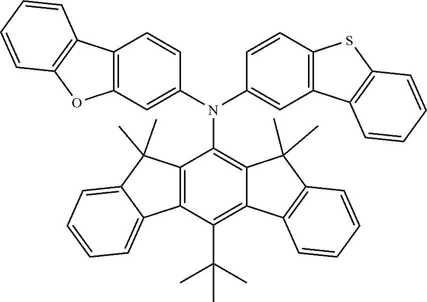 [SMILES: CC(C)(C)c1c2c(c(N(c3ccc4c(c3)oc3ccccc34)c3ccc4sc5ccccc5c4c3)c3c1-c1ccccc1C3(C)C)C(C)(C)c1ccccc1-2]

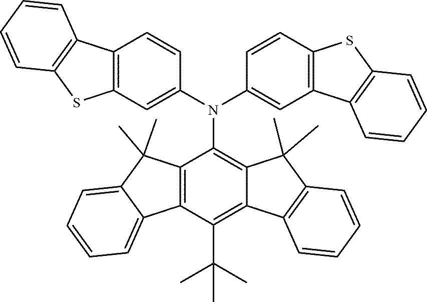 [SMILES: CC(C)(C)c1c2c(c(N(c3ccc4c(c3)sc3ccccc34)c3ccc4sc5ccccc5c4c3)c3c1-c1ccccc1C3(C)C)C(C)(C)c1ccccc1-2]

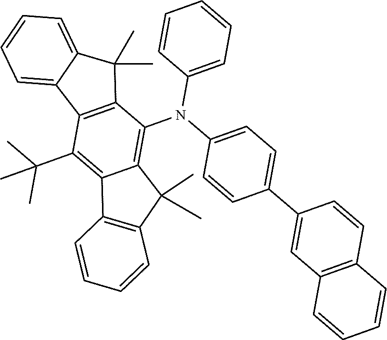 [SMILES: CC(C)(C)c1c2c(c(N(c3ccccc3)c3ccc(-c4ccc5ccccc5c4)cc3)c3c1-c1ccccc1C3(C)C)C(C)(C)c1ccccc1-2]

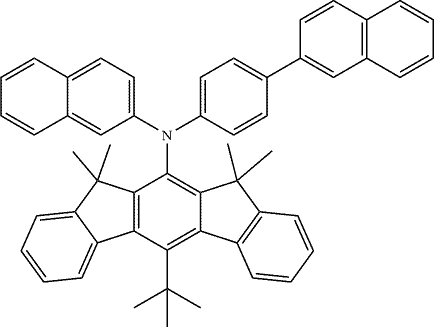 [SMILES: CC(C)(C)c1c2c(c(N(c3ccc(-c4ccc5ccccc5c4)cc3)c3ccc4ccccc4c3)c3c1-c1ccccc1C3(C)C)C(C)(C)c1ccccc1-2]

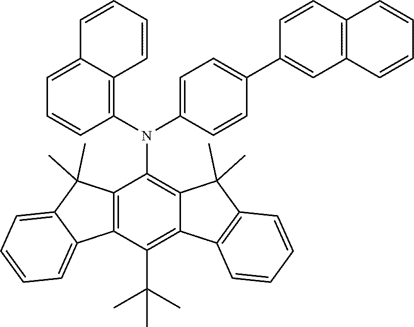 [SMILES: CC(C)(C)c1c2c(c(N(c3ccc(-c4ccc5ccccc5c4)cc3)c3cccc4ccccc34)c3c1-c1ccccc1C3(C)C)C(C)(C)c1ccccc1-2]

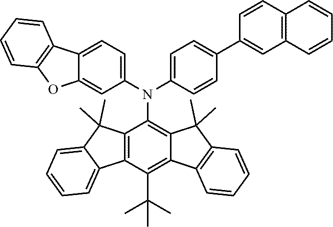 [SMILES: CC(C)(C)c1c2c(c(N(c3ccc(-c4ccc5ccccc5c4)cc3)c3ccc4c(c3)oc3ccccc34)c3c1-c1ccccc1C3(C)C)C(C)(C)c1ccccc1-2]

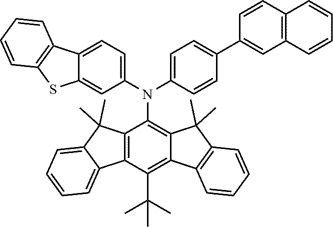 [SMILES: CC(C)(C)c1c2c(c(N(c3ccc(-c4ccc5ccccc5c4)cc3)c3ccc4c(c3)sc3ccccc34)c3c1-c1ccccc1C3(C)C)C(C)(C)c1ccccc1-2]

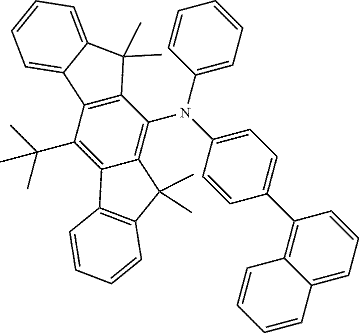 [SMILES: CC(C)(C)c1c2c(c(N(c3ccccc3)c3ccc(-c4cccc5ccccc45)cc3)c3c1-c1ccccc1C3(C)C)C(C)(C)c1ccccc1-2]

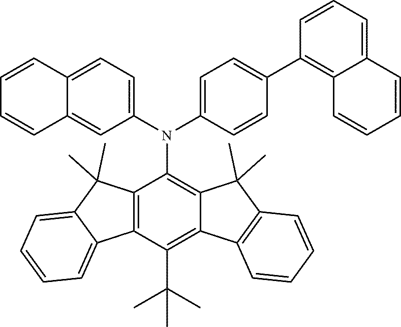 [SMILES: CC(C)(C)c1c2c(c(N(c3ccc(-c4cccc5ccccc45)cc3)c3ccc4ccccc4c3)c3c1-c1ccccc1C3(C)C)C(C)(C)c1ccccc1-2]

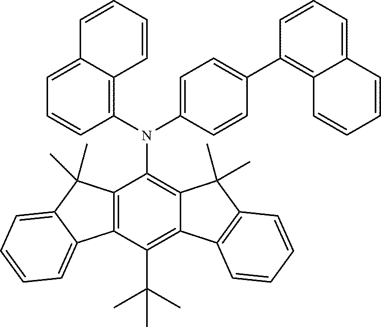 [SMILES: CC(C)(C)c1c2c(c(N(c3ccc(-c4cccc5ccccc45)cc3)c3cccc4ccccc34)c3c1-c1ccccc1C3(C)C)C(C)(C)c1ccccc1-2]

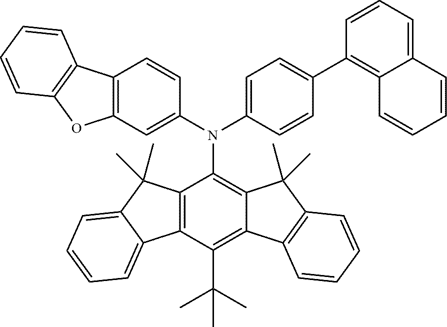 [SMILES: CC(C)(C)c1c2c(c(N(c3ccc(-c4cccc5ccccc45)cc3)c3ccc4c(c3)oc3ccccc34)c3c1-c1ccccc1C3(C)C)C(C)(C)c1ccccc1-2]

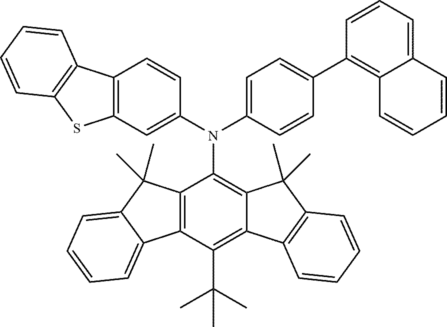 [SMILES: CC(C)(C)c1c2c(c(N(c3ccc(-c4cccc5ccccc45)cc3)c3ccc4c(c3)sc3ccccc34)c3c1-c1ccccc1C3(C)C)C(C)(C)c1ccccc1-2]

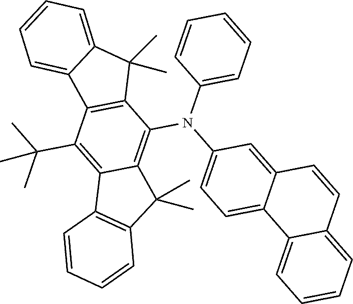 [SMILES: CC(C)(C)c1c2c(c(N(c3ccccc3)c3ccc4c(ccc5ccccc54)c3)c3c1-c1ccccc1C3(C)C)C(C)(C)c1ccccc1-2]

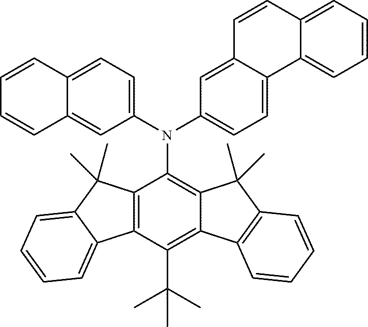 [SMILES: CC(C)(C)c1c2c(c(N(c3ccc4ccccc4c3)c3ccc4c(ccc5ccccc54)c3)c3c1-c1ccccc1C3(C)C)C(C)(C)c1ccccc1-2]

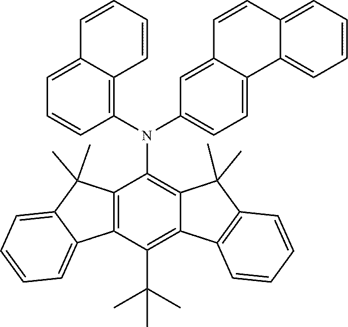 [SMILES: CC(C)(C)c1c2c(c(N(c3ccc4c(ccc5ccccc54)c3)c3cccc4ccccc34)c3c1-c1ccccc1C3(C)C)C(C)(C)c1ccccc1-2]

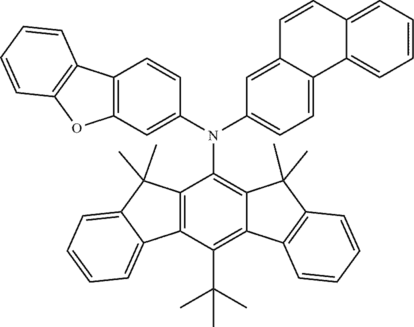 [SMILES: CC(C)(C)c1c2c(c(N(c3ccc4c(ccc5ccccc54)c3)c3ccc4c(c3)oc3ccccc34)c3c1-c1ccccc1C3(C)C)C(C)(C)c1ccccc1-2]

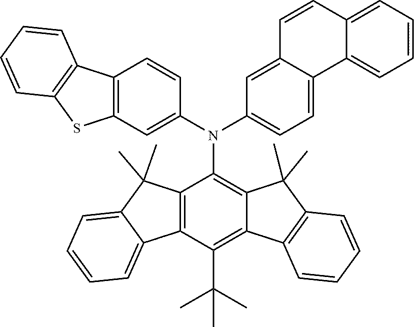 [SMILES: CC(C)(C)c1c2c(c(N(c3ccc4c(ccc5ccccc54)c3)c3ccc4c(c3)sc3ccccc34)c3c1-c1ccccc1C3(C)C)C(C)(C)c1ccccc1-2]

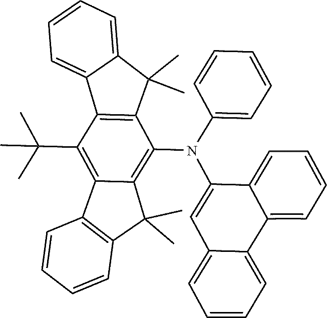 [SMILES: CC(C)(C)c1c2c(c(N(c3ccccc3)c3cc4ccccc4c4ccccc34)c3c1-c1ccccc1C3(C)C)C(C)(C)c1ccccc1-2]

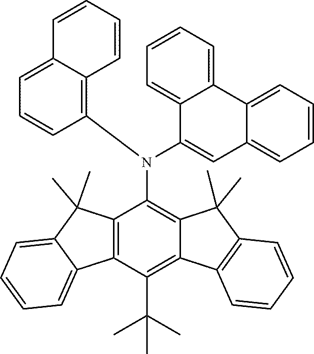 [SMILES: CC(C)(C)c1c2c(c(N(c3cccc4ccccc34)c3cc4ccccc4c4ccccc34)c3c1-c1ccccc1C3(C)C)C(C)(C)c1ccccc1-2]

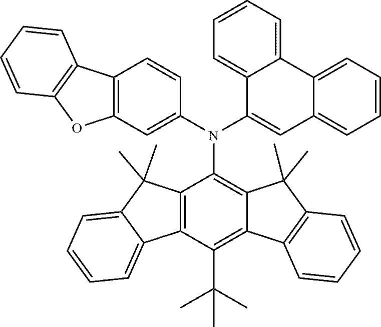 [SMILES: CC(C)(C)c1c2c(c(N(c3ccc4c(c3)oc3ccccc34)c3cc4ccccc4c4ccccc34)c3c1-c1ccccc1C3(C)C)C(C)(C)c1ccccc1-2]

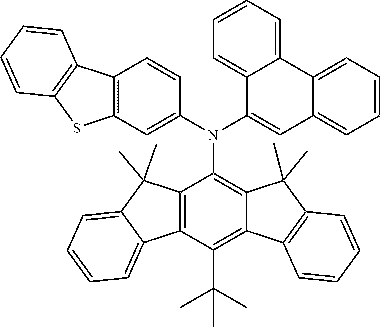 [SMILES: CC(C)(C)c1c2c(c(N(c3ccc4c(c3)sc3ccccc34)c3cc4ccccc4c4ccccc34)c3c1-c1ccccc1C3(C)C)C(C)(C)c1ccccc1-2]

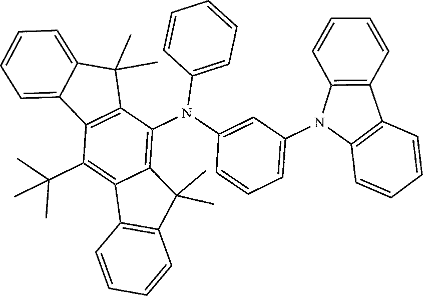

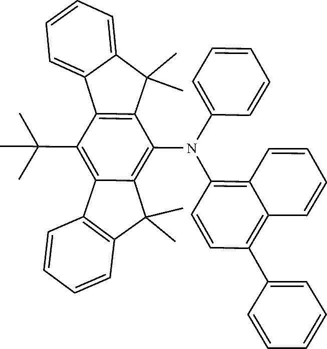 [SMILES: CC(C)(C)c1c2c(c(N(c3ccccc3)c3ccc(-c4ccccc4)c4ccccc34)c3c1-c1ccccc1C3(C)C)C(C)(C)c1ccccc1-2]

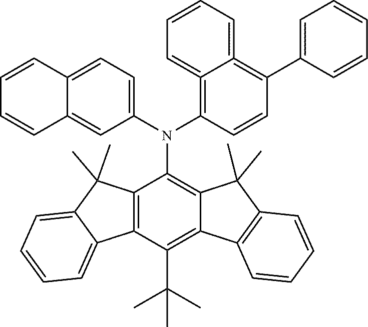

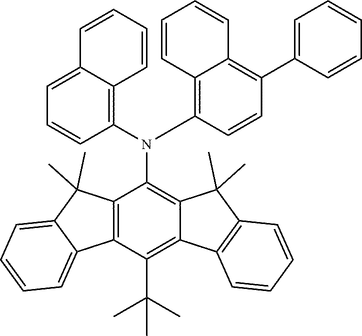 [SMILES: CC(C)(C)c1c2c(c(N(c3cccc4ccccc34)c3ccc(-c4ccccc4)c4ccccc34)c3c1-c1ccccc1C3(C)C)C(C)(C)c1ccccc1-2]

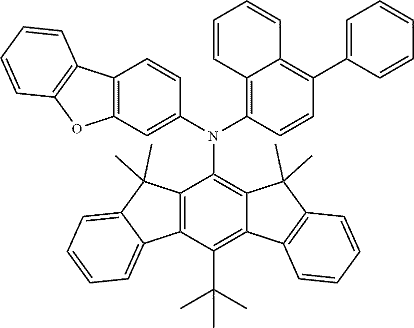 [SMILES: CC(C)(C)c1c2c(c(N(c3ccc4c(c3)oc3ccccc34)c3ccc(-c4ccccc4)c4ccccc34)c3c1-c1ccccc1C3(C)C)C(C)(C)c1ccccc1-2]

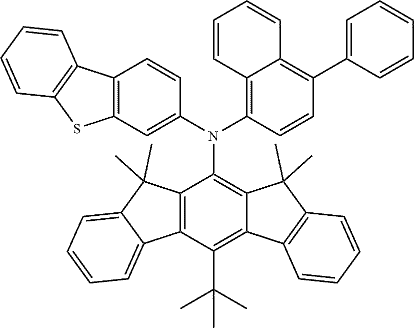 [SMILES: CC(C)(C)c1c2c(c(N(c3ccc4c(c3)sc3ccccc34)c3ccc(-c4ccccc4)c4ccccc34)c3c1-c1ccccc1C3(C)C)C(C)(C)c1ccccc1-2]

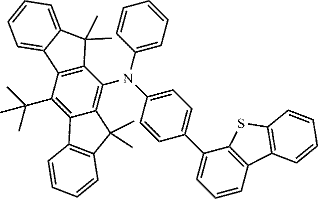 [SMILES: CC(C)(C)c1c2c(c(N(c3ccccc3)c3ccc(-c4cccc5c4sc4ccccc45)cc3)c3c1-c1ccccc1C3(C)C)C(C)(C)c1ccccc1-2]

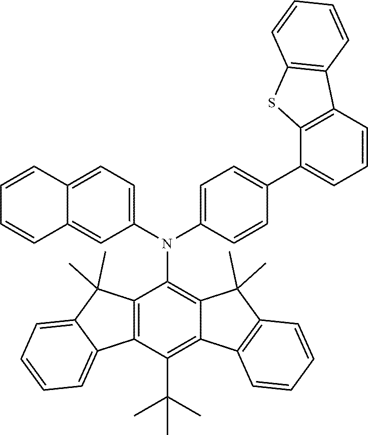 [SMILES: CC(C)(C)c1c2c(c(N(c3ccc(-c4cccc5c4sc4ccccc45)cc3)c3ccc4ccccc4c3)c3c1-c1ccccc1C3(C)C)C(C)(C)c1ccccc1-2]

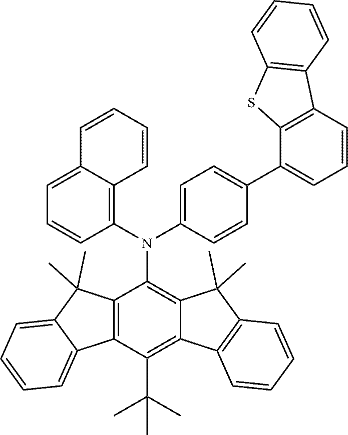 [SMILES: CC(C)(C)c1c2c(c(N(c3ccc(-c4cccc5c4sc4ccccc45)cc3)c3cccc4ccccc34)c3c1-c1ccccc1C3(C)C)C(C)(C)c1ccccc1-2]

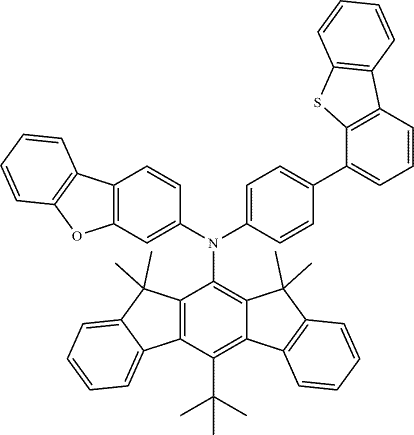 [SMILES: CC(C)(C)c1c2c(c(N(c3ccc(-c4cccc5c4sc4ccccc45)cc3)c3ccc4c(c3)oc3ccccc34)c3c1-c1ccccc1C3(C)C)C(C)(C)c1ccccc1-2]

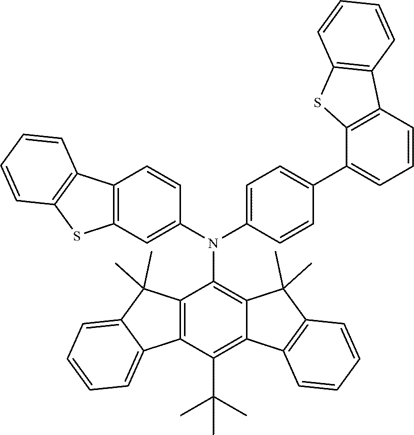 [SMILES: CC(C)(C)c1c2c(c(N(c3ccc(-c4cccc5c4sc4ccccc45)cc3)c3ccc4c(c3)sc3ccccc34)c3c1-c1ccccc1C3(C)C)C(C)(C)c1ccccc1-2]

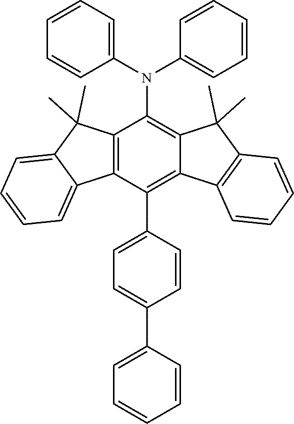 [SMILES: CC1(C)c2ccccc2-c2c(-c3ccc(-c4ccccc4)cc3)c3c(c(N(c4ccccc4)c4ccccc4)c21)C(C)(C)c1ccccc1-3]

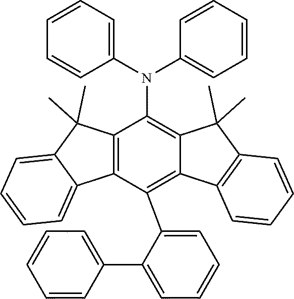 [SMILES: CC1(C)c2ccccc2-c2c(-c3ccccc3-c3ccccc3)c3c(c(N(c4ccccc4)c4ccccc4)c21)C(C)(C)c1ccccc1-3]

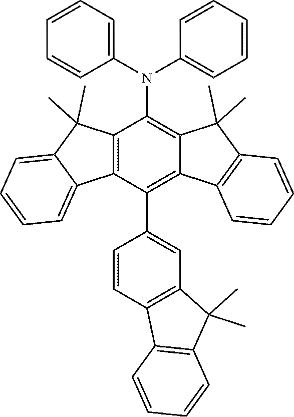 [SMILES: CC1(C)c2ccccc2-c2ccc(-c3c4c(c(N(c5ccccc5)c5ccccc5)c5c3-c3ccccc3C5(C)C)C(C)(C)c3ccccc3-4)cc21]

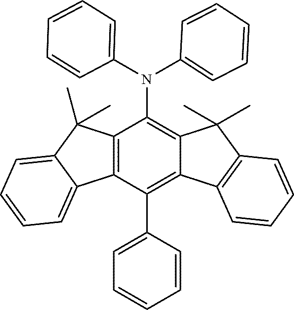 [SMILES: CC1(C)c2ccccc2-c2c(-c3ccccc3)c3c(c(N(c4ccccc4)c4ccccc4)c21)C(C)(C)c1ccccc1-3]

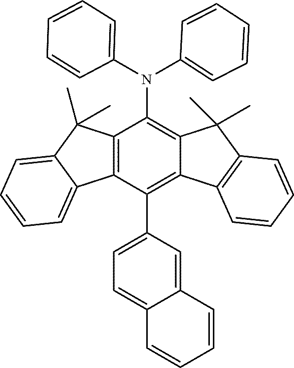 [SMILES: CC1(C)c2ccccc2-c2c(-c3ccc4ccccc4c3)c3c(c(N(c4ccccc4)c4ccccc4)c21)C(C)(C)c1ccccc1-3]

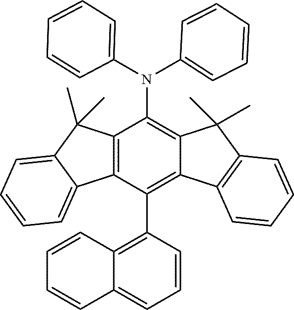 [SMILES: CC1(C)c2ccccc2-c2c(-c3cccc4ccccc34)c3c(c(N(c4ccccc4)c4ccccc4)c21)C(C)(C)c1ccccc1-3]

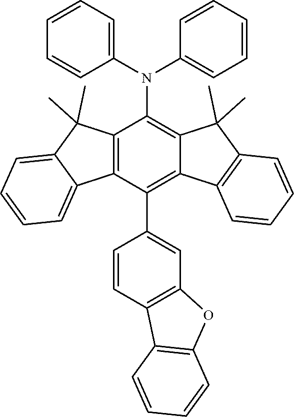 [SMILES: CC1(C)c2ccccc2-c2c(-c3ccc4c(c3)oc3ccccc34)c3c(c(N(c4ccccc4)c4ccccc4)c21)C(C)(C)c1ccccc1-3]

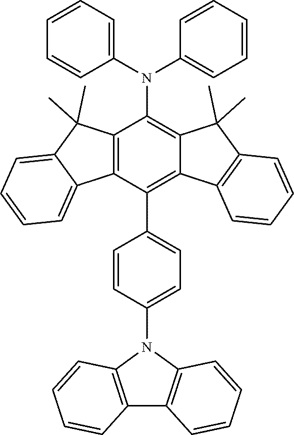 [SMILES: CC1(C)c2ccccc2-c2c(-c3ccc(-n4c5ccccc5c5ccccc54)cc3)c3c(c(N(c4ccccc4)c4ccccc4)c21)C(C)(C)c1ccccc1-3]

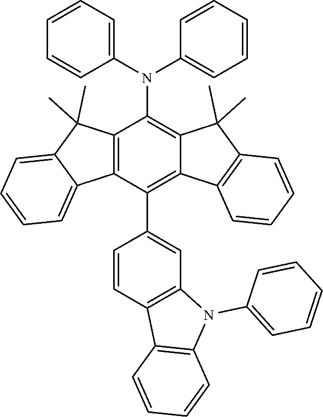 [SMILES: CC1(C)c2ccccc2-c2c(-c3ccc4c5ccccc5n(-c5ccccc5)c4c3)c3c(c(N(c4ccccc4)c4ccccc4)c21)C(C)(C)c1ccccc1-3]

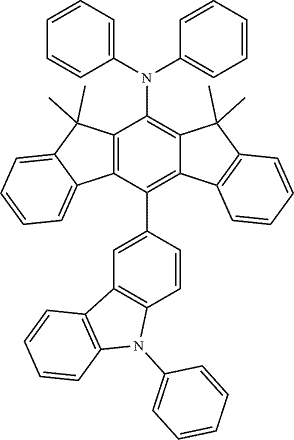 [SMILES: CC1(C)c2ccccc2-c2c(-c3ccc4c(c3)c3ccccc3n4-c3ccccc3)c3c(c(N(c4ccccc4)c4ccccc4)c21)C(C)(C)c1ccccc1-3]

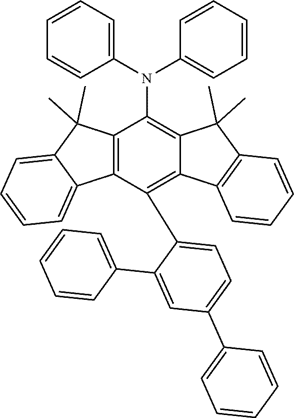 [SMILES: CC1(C)c2ccccc2-c2c(-c3ccc(-c4ccccc4)cc3-c3ccccc3)c3c(c(N(c4ccccc4)c4ccccc4)c21)C(C)(C)c1ccccc1-3]

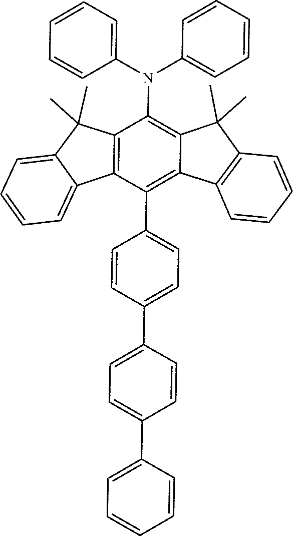 [SMILES: CC1(C)c2ccccc2-c2c(-c3ccc(-c4ccc(-c5ccccc5)cc4)cc3)c3c(c(N(c4ccccc4)c4ccccc4)c21)C(C)(C)c1ccccc1-3]

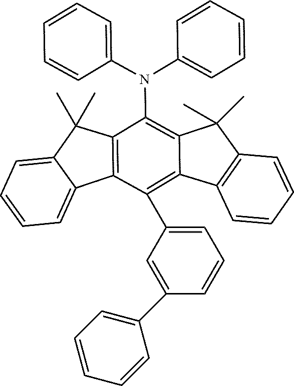 [SMILES: CC1(C)c2ccccc2-c2c(-c3cccc(-c4ccccc4)c3)c3c(c(N(c4ccccc4)c4ccccc4)c21)C(C)(C)c1ccccc1-3]

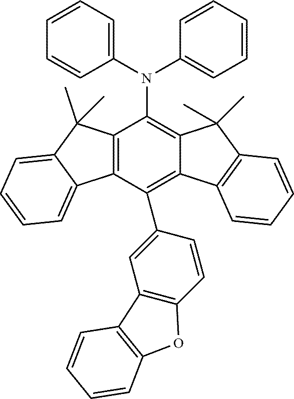 [SMILES: CC1(C)c2ccccc2-c2c(-c3ccc4oc5ccccc5c4c3)c3c(c(N(c4ccccc4)c4ccccc4)c21)C(C)(C)c1ccccc1-3]

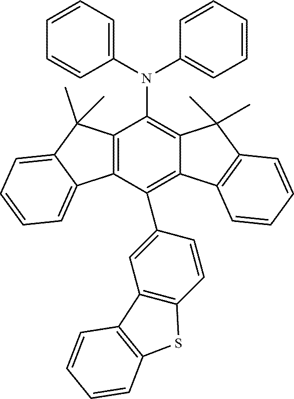 [SMILES: CC1(C)c2ccccc2-c2c(-c3ccc4sc5ccccc5c4c3)c3c(c(N(c4ccccc4)c4ccccc4)c21)C(C)(C)c1ccccc1-3]

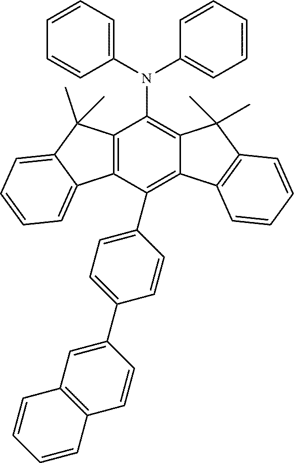 [SMILES: CC1(C)c2ccccc2-c2c(-c3ccc(-c4ccc5ccccc5c4)cc3)c3c(c(N(c4ccccc4)c4ccccc4)c21)C(C)(C)c1ccccc1-3]

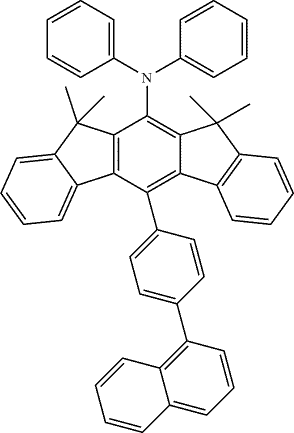 [SMILES: CC1(C)c2ccccc2-c2c(-c3ccc(-c4cccc5ccccc45)cc3)c3c(c(N(c4ccccc4)c4ccccc4)c21)C(C)(C)c1ccccc1-3]

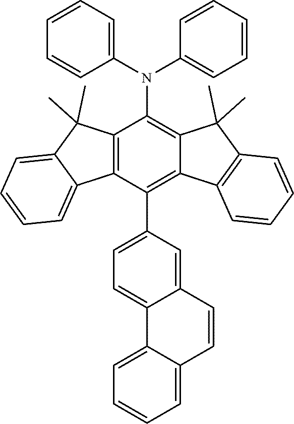 [SMILES: CC1(C)c2ccccc2-c2c(-c3ccc4c(ccc5ccccc54)c3)c3c(c(N(c4ccccc4)c4ccccc4)c21)C(C)(C)c1ccccc1-3]

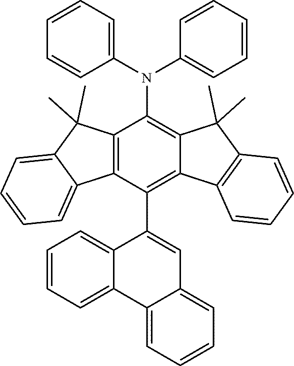 [SMILES: CC1(C)c2ccccc2-c2c(-c3cc4ccccc4c4ccccc34)c3c(c(N(c4ccccc4)c4ccccc4)c21)C(C)(C)c1ccccc1-3]

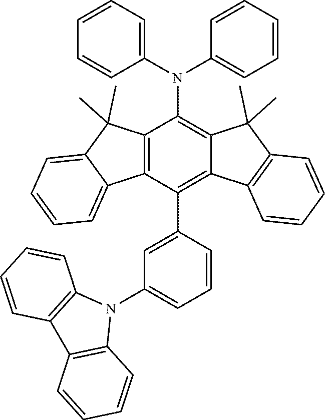 [SMILES: CC1(C)c2ccccc2-c2c(-c3cccc(-n4c5ccccc5c5ccccc54)c3)c3c(c(N(c4ccccc4)c4ccccc4)c21)C(C)(C)c1ccccc1-3]

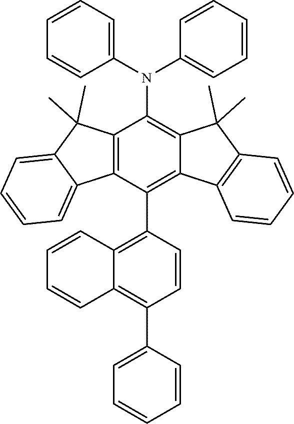 [SMILES: CC1(C)c2ccccc2-c2c(-c3ccc(-c4ccccc4)c4ccccc34)c3c(c(N(c4ccccc4)c4ccccc4)c21)C(C)(C)c1ccccc1-3]

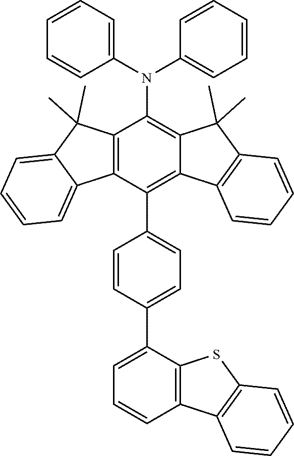 [SMILES: CC1(C)c2ccccc2-c2c(-c3ccc(-c4cccc5c4sc4ccccc45)cc3)c3c(c(N(c4ccccc4)c4ccccc4)c21)C(C)(C)c1ccccc1-3]

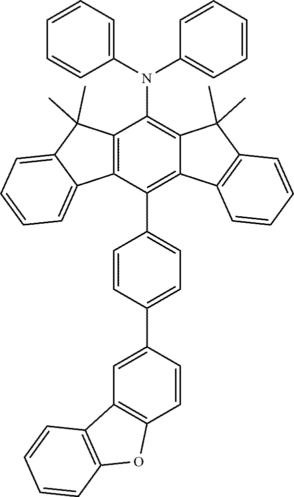 [SMILES: CC1(C)c2ccccc2-c2c(-c3ccc(-c4ccc5oc6ccccc6c5c4)cc3)c3c(c(N(c4ccccc4)c4ccccc4)c21)C(C)(C)c1ccccc1-3]

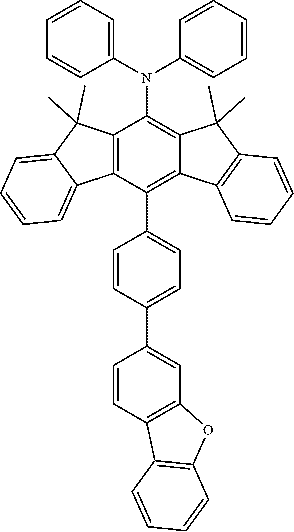 [SMILES: CC1(C)c2ccccc2-c2c(-c3ccc(-c4ccc5c(c4)oc4ccccc45)cc3)c3c(c(N(c4ccccc4)c4ccccc4)c21)C(C)(C)c1ccccc1-3]

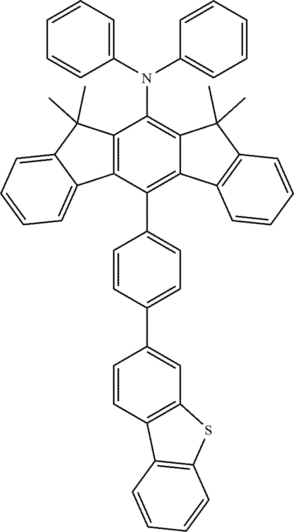 [SMILES: CC1(C)c2ccccc2-c2c(-c3ccc(-c4ccc5c(c4)sc4ccccc45)cc3)c3c(c(N(c4ccccc4)c4ccccc4)c21)C(C)(C)c1ccccc1-3]

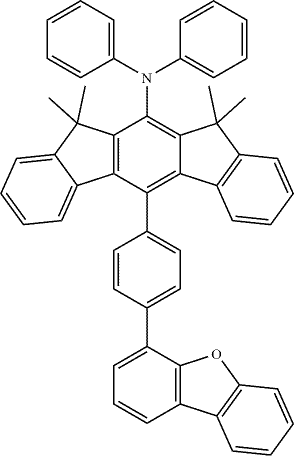 [SMILES: CC1(C)c2ccccc2-c2c(-c3ccc(-c4cccc5c4oc4ccccc45)cc3)c3c(c(N(c4ccccc4)c4ccccc4)c21)C(C)(C)c1ccccc1-3]

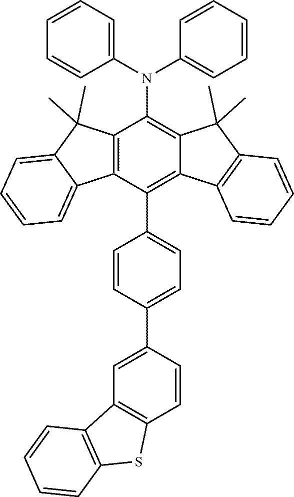 [SMILES: CC1(C)c2ccccc2-c2c(-c3ccc(-c4ccc5sc6ccccc6c5c4)cc3)c3c(c(N(c4ccccc4)c4ccccc4)c21)C(C)(C)c1ccccc1-3]

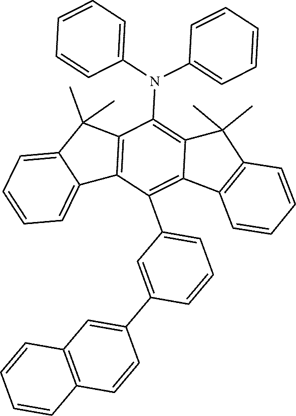 [SMILES: CC1(C)c2ccccc2-c2c(-c3cccc(-c4ccc5ccccc5c4)c3)c3c(c(N(c4ccccc4)c4ccccc4)c21)C(C)(C)c1ccccc1-3]

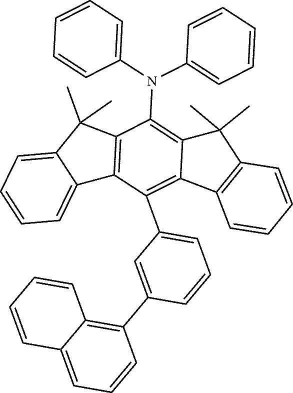 [SMILES: CC1(C)c2ccccc2-c2c(-c3cccc(-c4cccc5ccccc45)c3)c3c(c(N(c4ccccc4)c4ccccc4)c21)C(C)(C)c1ccccc1-3]

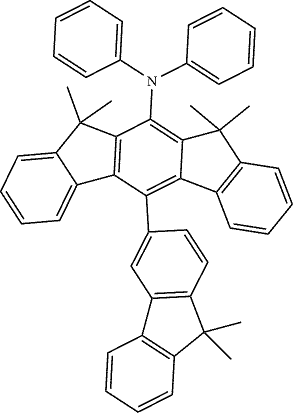 [SMILES: CC1(C)c2ccccc2-c2cc(-c3c4c(c(N(c5ccccc5)c5ccccc5)c5c3-c3ccccc3C5(C)C)C(C)(C)c3ccccc3-4)ccc21]

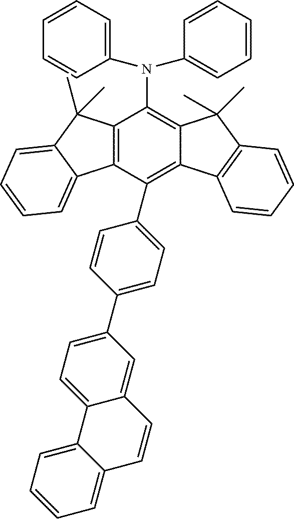 [SMILES: CC1(C)c2ccccc2-c2c(-c3ccc(-c4ccc5c(ccc6ccccc65)c4)cc3)c3c(c(N(c4ccccc4)c4ccccc4)c21)C(C)(C)c1ccccc1-3]

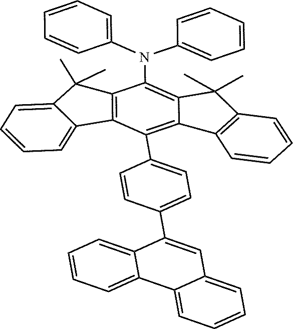 [SMILES: CC1(C)c2ccccc2-c2c(-c3ccc(-c4cc5ccccc5c5ccccc45)cc3)c3c(c(N(c4ccccc4)c4ccccc4)c21)C(C)(C)c1ccccc1-3]

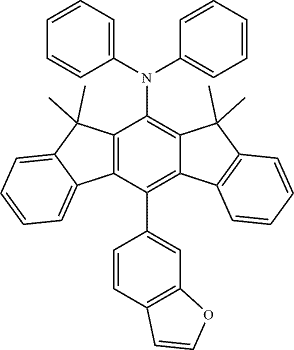 [SMILES: CC1(C)c2ccccc2-c2c(-c3ccc4ccoc4c3)c3c(c(N(c4ccccc4)c4ccccc4)c21)C(C)(C)c1ccccc1-3]

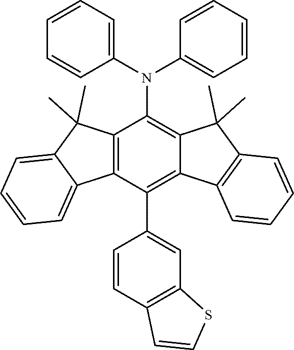 [SMILES: CC1(C)c2ccccc2-c2c(-c3ccc4ccsc4c3)c3c(c(N(c4ccccc4)c4ccccc4)c21)C(C)(C)c1ccccc1-3]

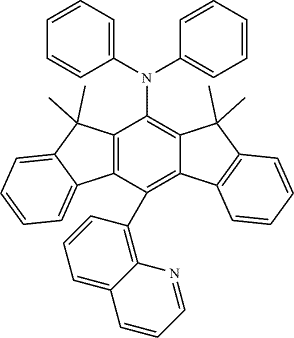 [SMILES: CC1(C)c2ccccc2-c2c(-c3cccc4cccnc34)c3c(c(N(c4ccccc4)c4ccccc4)c21)C(C)(C)c1ccccc1-3]

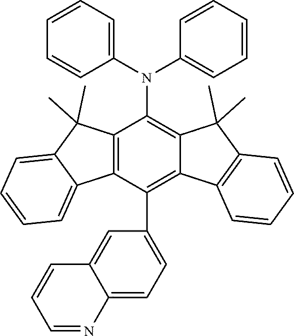 [SMILES: CC1(C)c2ccccc2-c2c(-c3ccc4ncccc4c3)c3c(c(N(c4ccccc4)c4ccccc4)c21)C(C)(C)c1ccccc1-3]

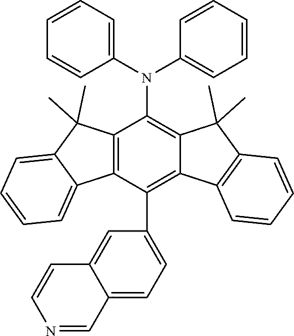 [SMILES: CC1(C)c2ccccc2-c2c(-c3ccc4cnccc4c3)c3c(c(N(c4ccccc4)c4ccccc4)c21)C(C)(C)c1ccccc1-3]

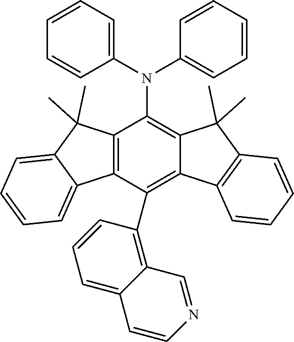 [SMILES: CC1(C)c2ccccc2-c2c(-c3cccc4ccncc34)c3c(c(N(c4ccccc4)c4ccccc4)c21)C(C)(C)c1ccccc1-3]

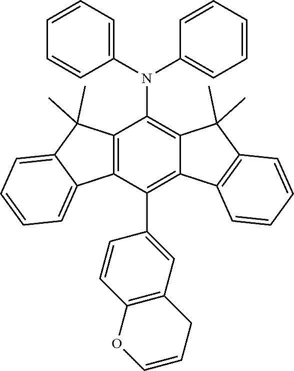 [SMILES: CC1(C)c2ccccc2-c2c(-c3ccc4c(c3)CC=CO4)c3c(c(N(c4ccccc4)c4ccccc4)c21)C(C)(C)c1ccccc1-3]

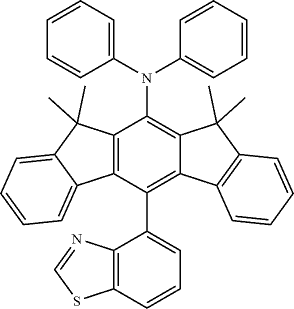 [SMILES: CC1(C)c2ccccc2-c2c(-c3cccc4scnc34)c3c(c(N(c4ccccc4)c4ccccc4)c21)C(C)(C)c1ccccc1-3]

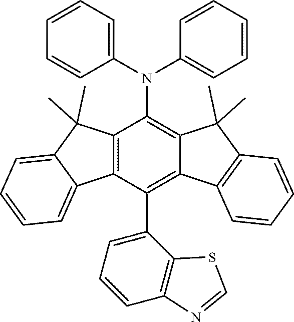 [SMILES: CC1(C)c2ccccc2-c2c(-c3cccc4ncsc34)c3c(c(N(c4ccccc4)c4ccccc4)c21)C(C)(C)c1ccccc1-3]

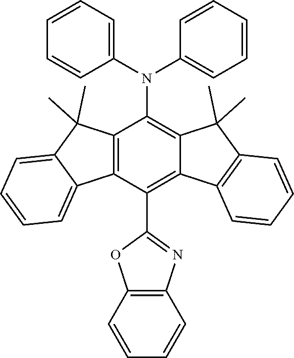 [SMILES: CC1(C)c2ccccc2-c2c(-c3nc4ccccc4o3)c3c(c(N(c4ccccc4)c4ccccc4)c21)C(C)(C)c1ccccc1-3]

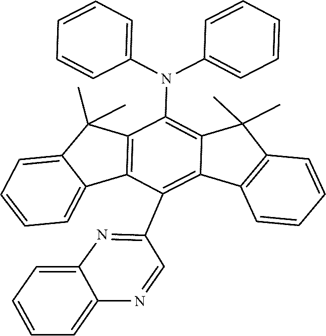 [SMILES: CC1(C)c2ccccc2-c2c(-c3cnc4ccccc4n3)c3c(c(N(c4ccccc4)c4ccccc4)c21)C(C)(C)c1ccccc1-3]

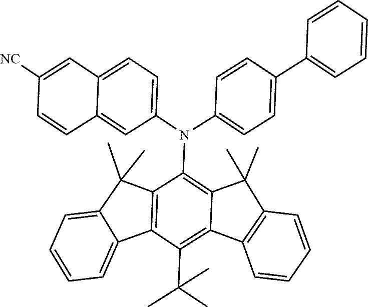 [SMILES: CC(C)(C)c1c2c(c(N(c3ccc(-c4ccccc4)cc3)c3ccc4cc(C#N)ccc4c3)c3c1-c1ccccc1C3(C)C)C(C)(C)c1ccccc1-2]

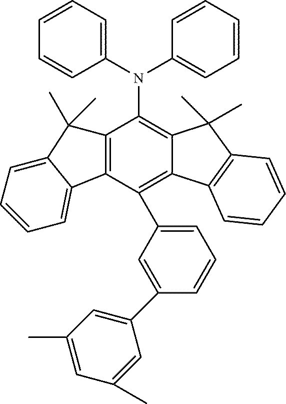 [SMILES: Cc1cc(C)cc(-c2cccc(-c3c4c(c(N(c5ccccc5)c5ccccc5)c5c3-c3ccccc3C5(C)C)C(C)(C)c3ccccc3-4)c2)c1]

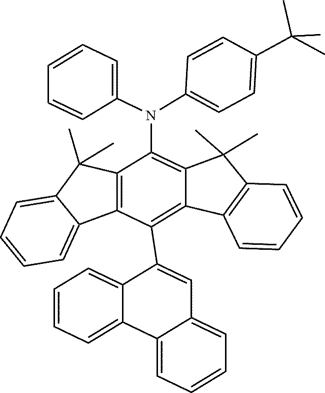 [SMILES: CC(C)(C)c1ccc(N(c2ccccc2)c2c3c(c(-c4cc5ccccc5c5ccccc45)c4c2C(C)(C)c2ccccc2-4)-c2ccccc2C3(C)C)cc1]

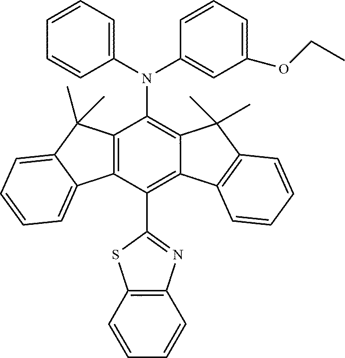 [SMILES: CCOc1cccc(N(c2ccccc2)c2c3c(c(-c4nc5ccccc5s4)c4c2C(C)(C)c2ccccc2-4)-c2ccccc2C3(C)C)c1]

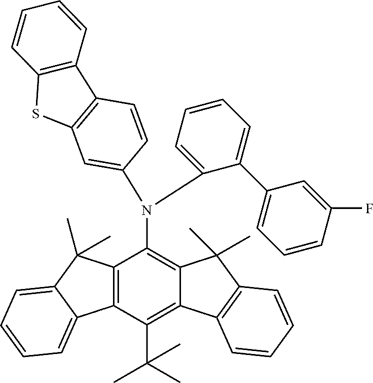 [SMILES: CC(C)(C)c1c2c(c(N(c3ccc4c(c3)sc3ccccc34)c3ccccc3-c3cccc(F)c3)c3c1-c1ccccc1C3(C)C)C(C)(C)c1ccccc1-2]

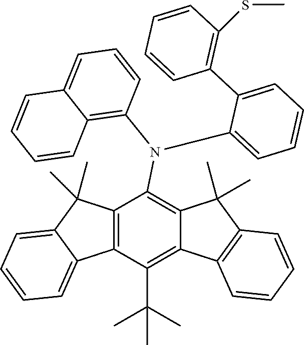 [SMILES: CSc1ccccc1-c1ccccc1N(c1c2c(c(C(C)(C)C)c3c1C(C)(C)c1ccccc1-3)-c1ccccc1C2(C)C)c1cccc2ccccc12]

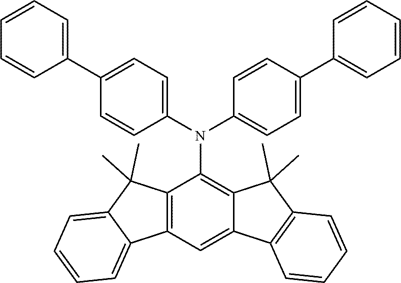 [SMILES: CC1(C)c2ccccc2-c2cc3c(c(N(c4ccc(-c5ccccc5)cc4)c4ccc(-c5ccccc5)cc4)c21)C(C)(C)c1ccccc1-3]

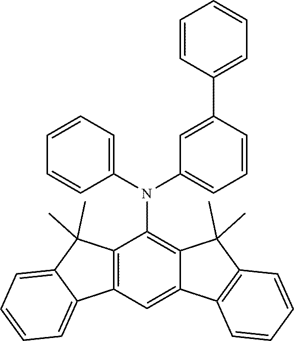 [SMILES: CC1(C)c2ccccc2-c2cc3c(c(N(c4ccccc4)c4cccc(-c5ccccc5)c4)c21)C(C)(C)c1ccccc1-3]

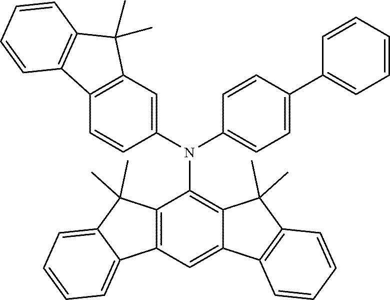 [SMILES: CC1(C)c2ccccc2-c2ccc(N(c3ccc(-c4ccccc4)cc3)c3c4c(cc5c3C(C)(C)c3ccccc3-5)-c3ccccc3C4(C)C)cc21]

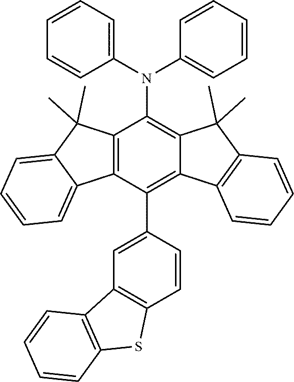 [SMILES: CC1(C)c2ccccc2-c2c(-c3ccc4sc5ccccc5c4c3)c3c(c(N(c4ccccc4)c4ccccc4)c21)C(C)(C)c1ccccc1-3]

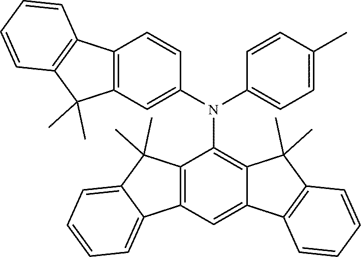 [SMILES: Cc1ccc(N(c2ccc3c(c2)C(C)(C)c2ccccc2-3)c2c3c(cc4c2C(C)(C)c2ccccc2-4)-c2ccccc2C3(C)C)cc1]

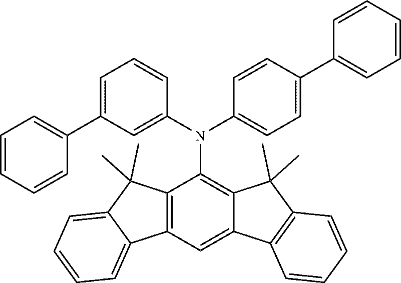 [SMILES: CC1(C)c2ccccc2-c2cc3c(c(N(c4ccc(-c5ccccc5)cc4)c4cccc(-c5ccccc5)c4)c21)C(C)(C)c1ccccc1-3]

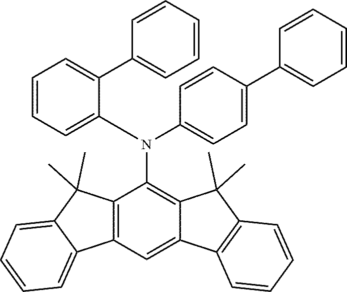 [SMILES: CC1(C)c2ccccc2-c2cc3c(c(N(c4ccc(-c5ccccc5)cc4)c4ccccc4-c4ccccc4)c21)C(C)(C)c1ccccc1-3]

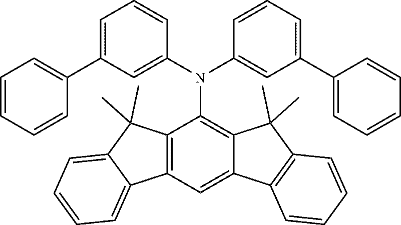 [SMILES: CC1(C)c2ccccc2-c2cc3c(c(N(c4cccc(-c5ccccc5)c4)c4cccc(-c5ccccc5)c4)c21)C(C)(C)c1ccccc1-3]

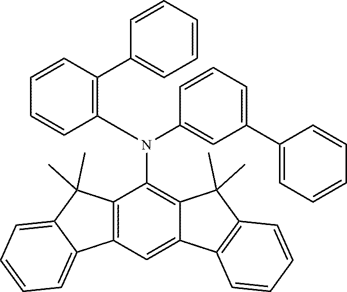 [SMILES: CC1(C)c2ccccc2-c2cc3c(c(N(c4cccc(-c5ccccc5)c4)c4ccccc4-c4ccccc4)c21)C(C)(C)c1ccccc1-3]

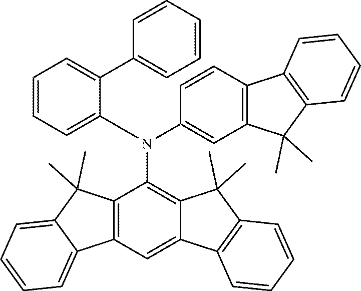 [SMILES: CC1(C)c2ccccc2-c2ccc(N(c3ccccc3-c3ccccc3)c3c4c(cc5c3C(C)(C)c3ccccc3-5)-c3ccccc3C4(C)C)cc21]

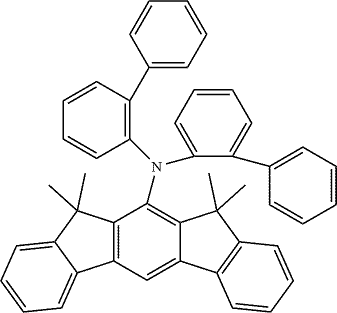 [SMILES: CC1(C)c2ccccc2-c2cc3c(c(N(c4ccccc4-c4ccccc4)c4ccccc4-c4ccccc4)c21)C(C)(C)c1ccccc1-3]

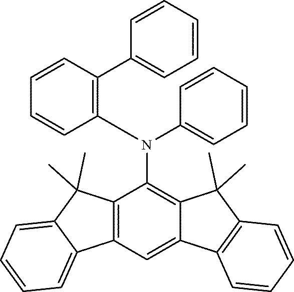 [SMILES: CC1(C)c2ccccc2-c2cc3c(c(N(c4ccccc4)c4ccccc4-c4ccccc4)c21)C(C)(C)c1ccccc1-3]

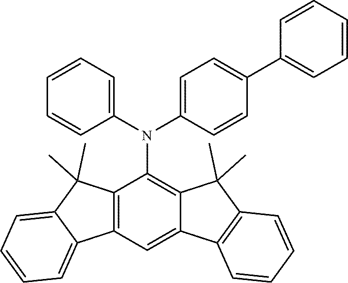 [SMILES: CC1(C)c2ccccc2-c2cc3c(c(N(c4ccccc4)c4ccc(-c5ccccc5)cc4)c21)C(C)(C)c1ccccc1-3]

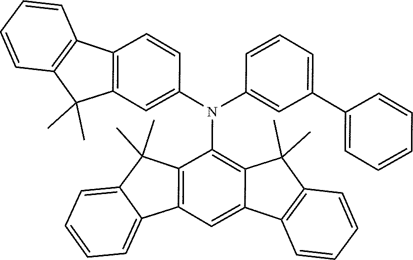 [SMILES: CC1(C)c2ccccc2-c2ccc(N(c3cccc(-c4ccccc4)c3)c3c4c(cc5c3C(C)(C)c3ccccc3-5)-c3ccccc3C4(C)C)cc21]

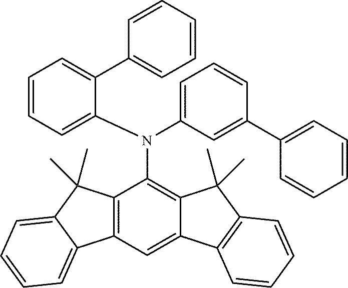 [SMILES: CC1(C)c2ccccc2-c2cc3c(c(N(c4cccc(-c5ccccc5)c4)c4ccccc4-c4ccccc4)c21)C(C)(C)c1ccccc1-3]

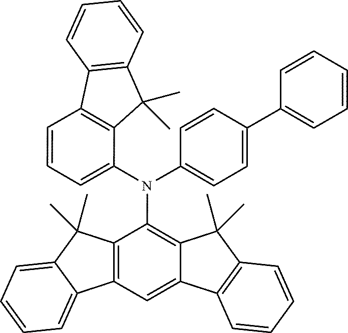 [SMILES: CC1(C)c2ccccc2-c2cccc(N(c3ccc(-c4ccccc4)cc3)c3c4c(cc5c3C(C)(C)c3ccccc3-5)-c3ccccc3C4(C)C)c21]

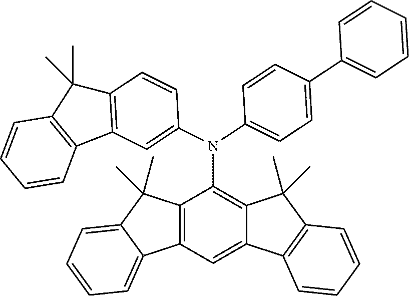 [SMILES: CC1(C)c2ccccc2-c2cc(N(c3ccc(-c4ccccc4)cc3)c3c4c(cc5c3C(C)(C)c3ccccc3-5)-c3ccccc3C4(C)C)ccc21]

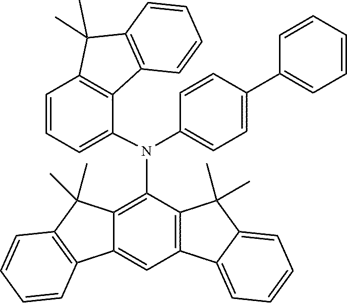 [SMILES: CC1(C)c2ccccc2-c2c(N(c3ccc(-c4ccccc4)cc3)c3c4c(cc5c3C(C)(C)c3ccccc3-5)-c3ccccc3C4(C)C)cccc21]

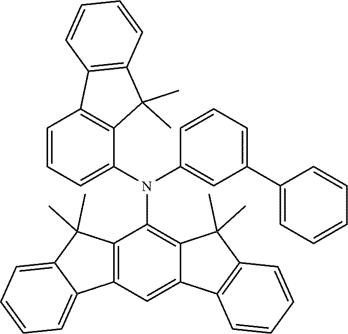 [SMILES: CC1(C)c2ccccc2-c2cccc(N(c3cccc(-c4ccccc4)c3)c3c4c(cc5c3C(C)(C)c3ccccc3-5)-c3ccccc3C4(C)C)c21]

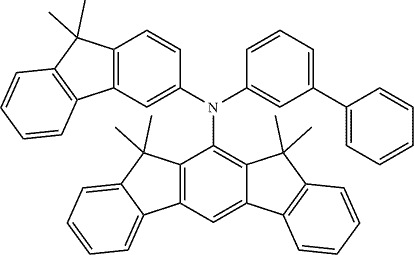 [SMILES: CC1(C)c2ccccc2-c2cc(N(c3cccc(-c4ccccc4)c3)c3c4c(cc5c3C(C)(C)c3ccccc3-5)-c3ccccc3C4(C)C)ccc21]

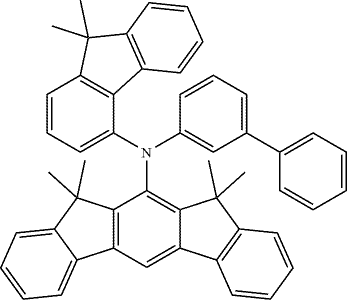 [SMILES: CC1(C)c2ccccc2-c2c(N(c3cccc(-c4ccccc4)c3)c3c4c(cc5c3C(C)(C)c3ccccc3-5)-c3ccccc3C4(C)C)cccc21]

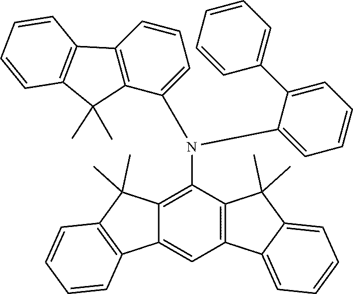 [SMILES: CC1(C)c2ccccc2-c2cccc(N(c3ccccc3-c3ccccc3)c3c4c(cc5c3C(C)(C)c3ccccc3-5)-c3ccccc3C4(C)C)c21]

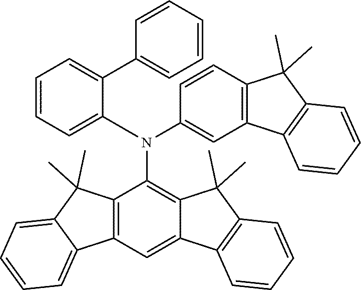 [SMILES: CC1(C)c2ccccc2-c2cc(N(c3ccccc3-c3ccccc3)c3c4c(cc5c3C(C)(C)c3ccccc3-5)-c3ccccc3C4(C)C)ccc21]

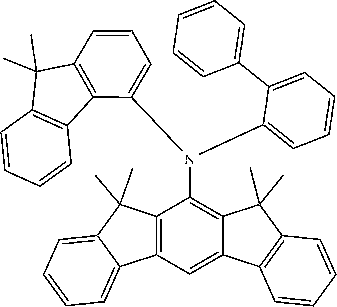 [SMILES: CC1(C)c2ccccc2-c2c(N(c3ccccc3-c3ccccc3)c3c4c(cc5c3C(C)(C)c3ccccc3-5)-c3ccccc3C4(C)C)cccc21]

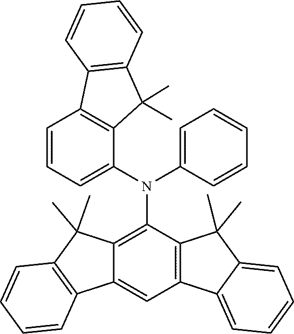 [SMILES: CC1(C)c2ccccc2-c2cccc(N(c3ccccc3)c3c4c(cc5c3C(C)(C)c3ccccc3-5)-c3ccccc3C4(C)C)c21]

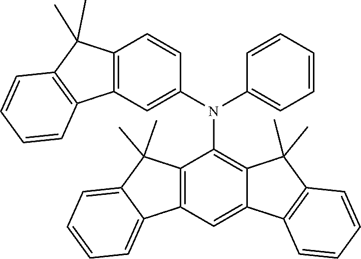 [SMILES: CC1(C)c2ccccc2-c2cc(N(c3ccccc3)c3c4c(cc5c3C(C)(C)c3ccccc3-5)-c3ccccc3C4(C)C)ccc21]

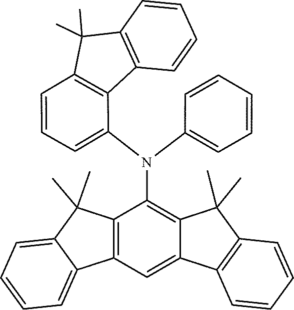 [SMILES: CC1(C)c2ccccc2-c2c(N(c3ccccc3)c3c4c(cc5c3C(C)(C)c3ccccc3-5)-c3ccccc3C4(C)C)cccc21]

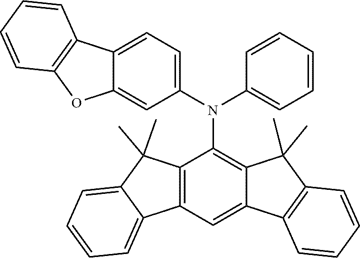 [SMILES: CC1(C)c2ccccc2-c2cc3c(c(N(c4ccccc4)c4ccc5c(c4)oc4ccccc45)c21)C(C)(C)c1ccccc1-3]

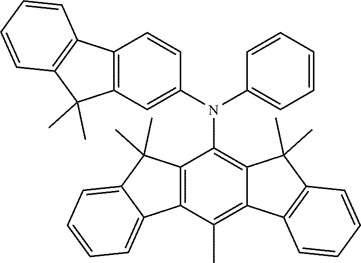 [SMILES: Cc1c2c(c(N(c3ccccc3)c3ccc4c(c3)C(C)(C)c3ccccc3-4)c3c1-c1ccccc1C3(C)C)C(C)(C)c1ccccc1-2]

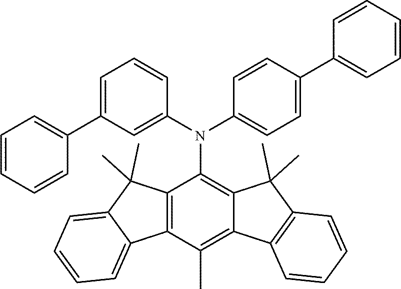 [SMILES: Cc1c2c(c(N(c3ccc(-c4ccccc4)cc3)c3cccc(-c4ccccc4)c3)c3c1-c1ccccc1C3(C)C)C(C)(C)c1ccccc1-2]

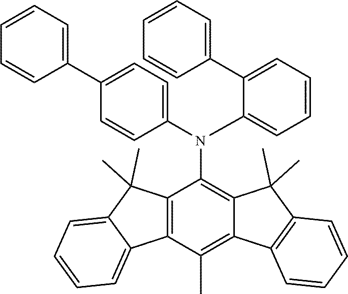 [SMILES: Cc1c2c(c(N(c3ccc(-c4ccccc4)cc3)c3ccccc3-c3ccccc3)c3c1-c1ccccc1C3(C)C)C(C)(C)c1ccccc1-2]

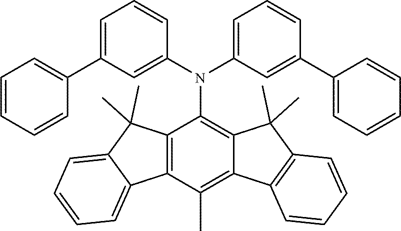 [SMILES: Cc1c2c(c(N(c3cccc(-c4ccccc4)c3)c3cccc(-c4ccccc4)c3)c3c1-c1ccccc1C3(C)C)C(C)(C)c1ccccc1-2]

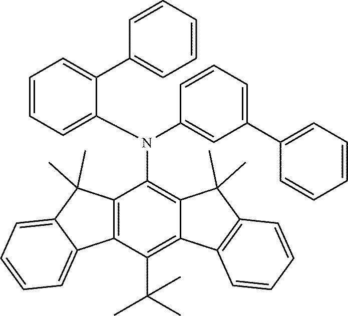 [SMILES: CC(C)(C)c1c2c(c(N(c3cccc(-c4ccccc4)c3)c3ccccc3-c3ccccc3)c3c1-c1ccccc1C3(C)C)C(C)(C)c1ccccc1-2]

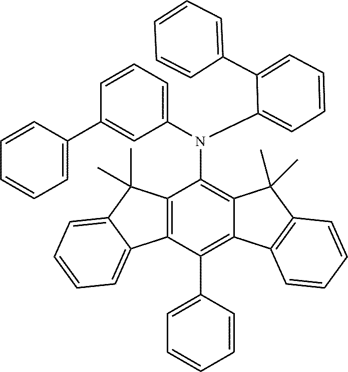 [SMILES: CC1(C)c2ccccc2-c2c(-c3ccccc3)c3c(c(N(c4cccc(-c5ccccc5)c4)c4ccccc4-c4ccccc4)c21)C(C)(C)c1ccccc1-3]

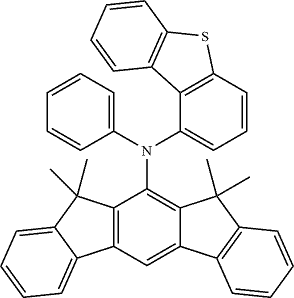 [SMILES: CC1(C)c2ccccc2-c2cc3c(c(N(c4ccccc4)c4cccc5sc6ccccc6c45)c21)C(C)(C)c1ccccc1-3]

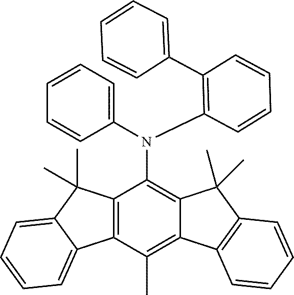 [SMILES: Cc1c2c(c(N(c3ccccc3)c3ccccc3-c3ccccc3)c3c1-c1ccccc1C3(C)C)C(C)(C)c1ccccc1-2]

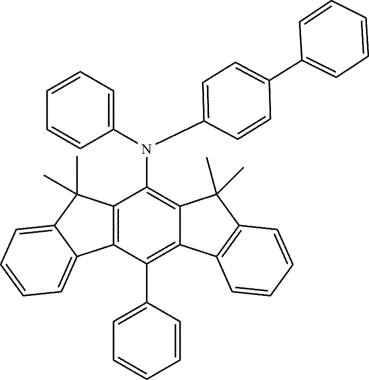 [SMILES: CC1(C)c2ccccc2-c2c(-c3ccccc3)c3c(c(N(c4ccccc4)c4ccc(-c5ccccc5)cc4)c21)C(C)(C)c1ccccc1-3]

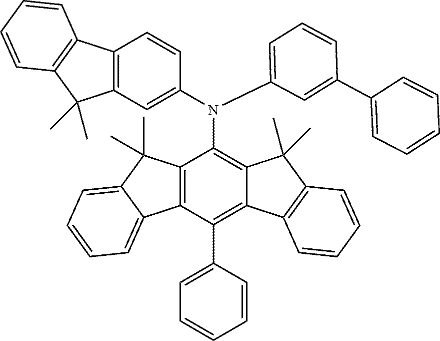 [SMILES: CC1(C)c2ccccc2-c2ccc(N(c3cccc(-c4ccccc4)c3)c3c4c(c(-c5ccccc5)c5c3C(C)(C)c3ccccc3-5)-c3ccccc3C4(C)C)cc21]

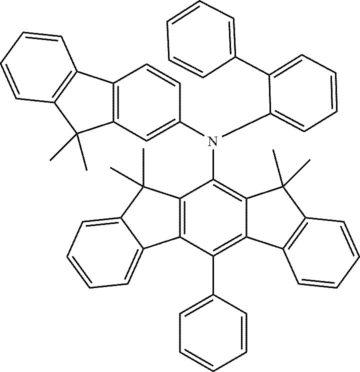 [SMILES: CC1(C)c2ccccc2-c2ccc(N(c3ccccc3-c3ccccc3)c3c4c(c(-c5ccccc5)c5c3C(C)(C)c3ccccc3-5)-c3ccccc3C4(C)C)cc21]

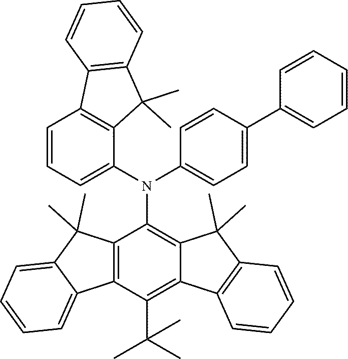 [SMILES: CC(C)(C)c1c2c(c(N(c3ccc(-c4ccccc4)cc3)c3cccc4c3C(C)(C)c3ccccc3-4)c3c1-c1ccccc1C3(C)C)C(C)(C)c1ccccc1-2]

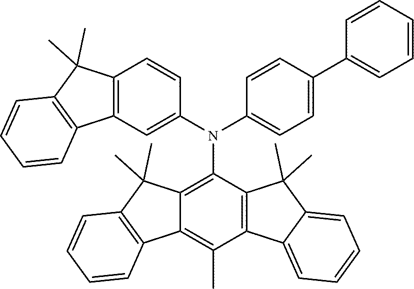 [SMILES: Cc1c2c(c(N(c3ccc(-c4ccccc4)cc3)c3ccc4c(c3)-c3ccccc3C4(C)C)c3c1-c1ccccc1C3(C)C)C(C)(C)c1ccccc1-2]

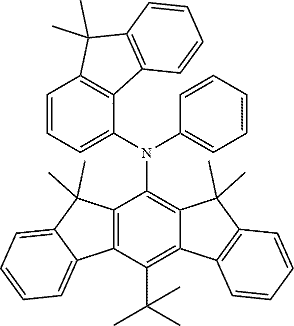 [SMILES: CC(C)(C)c1c2c(c(N(c3ccccc3)c3cccc4c3-c3ccccc3C4(C)C)c3c1-c1ccccc1C3(C)C)C(C)(C)c1ccccc1-2]

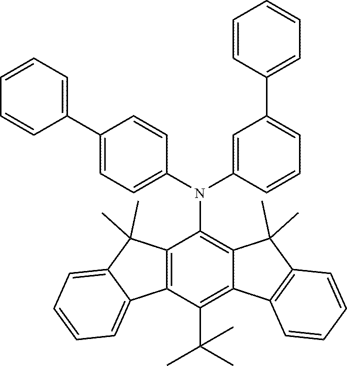 [SMILES: CC(C)(C)c1c2c(c(N(c3ccc(-c4ccccc4)cc3)c3cccc(-c4ccccc4)c3)c3c1-c1ccccc1C3(C)C)C(C)(C)c1ccccc1-2]

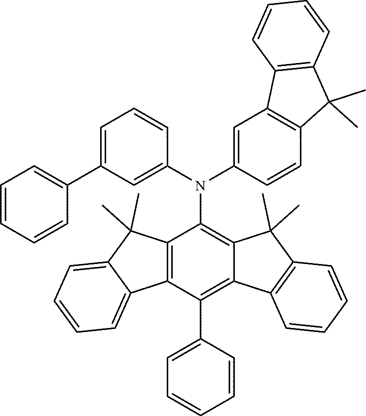 [SMILES: CC1(C)c2ccccc2-c2cc(N(c3cccc(-c4ccccc4)c3)c3c4c(c(-c5ccccc5)c5c3C(C)(C)c3ccccc3-5)-c3ccccc3C4(C)C)ccc21]

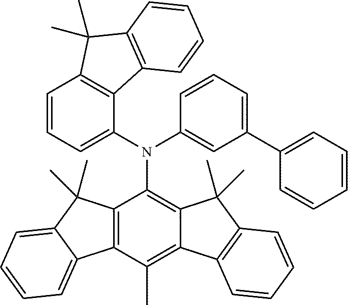 [SMILES: Cc1c2c(c(N(c3cccc(-c4ccccc4)c3)c3cccc4c3-c3ccccc3C4(C)C)c3c1-c1ccccc1C3(C)C)C(C)(C)c1ccccc1-2]

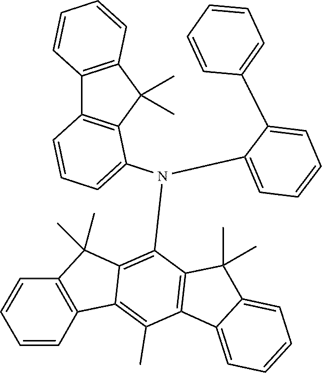 [SMILES: Cc1c2c(c(N(c3ccccc3-c3ccccc3)c3cccc4c3C(C)(C)c3ccccc3-4)c3c1-c1ccccc1C3(C)C)C(C)(C)c1ccccc1-2]

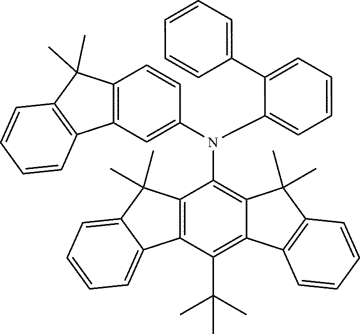 [SMILES: CC(C)(C)c1c2c(c(N(c3ccc4c(c3)-c3ccccc3C4(C)C)c3ccccc3-c3ccccc3)c3c1-c1ccccc1C3(C)C)C(C)(C)c1ccccc1-2]

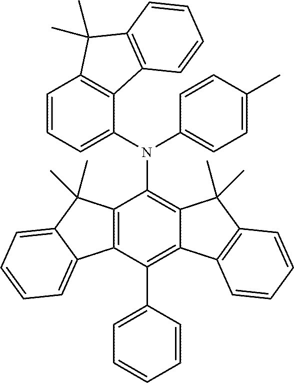 [SMILES: Cc1ccc(N(c2cccc3c2-c2ccccc2C3(C)C)c2c3c(c(-c4ccccc4)c4c2C(C)(C)c2ccccc2-4)-c2ccccc2C3(C)C)cc1]

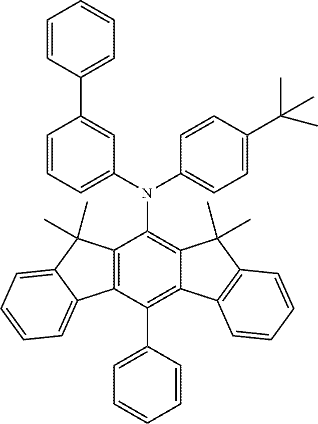 [SMILES: CC(C)(C)c1ccc(N(c2cccc(-c3ccccc3)c2)c2c3c(c(-c4ccccc4)c4c2C(C)(C)c2ccccc2-4)-c2ccccc2C3(C)C)cc1]

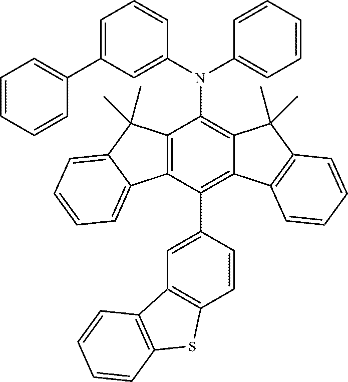 [SMILES: CC1(C)c2ccccc2-c2c(-c3ccc4sc5ccccc5c4c3)c3c(c(N(c4ccccc4)c4cccc(-c5ccccc5)c4)c21)C(C)(C)c1ccccc1-3]

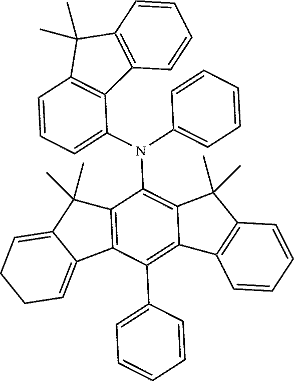 [SMILES: CC1(C)C2=CCCC=C2c2c(-c3ccccc3)c3c(c(N(c4ccccc4)c4cccc5c4-c4ccccc4C5(C)C)c21)C(C)(C)c1ccccc1-3]

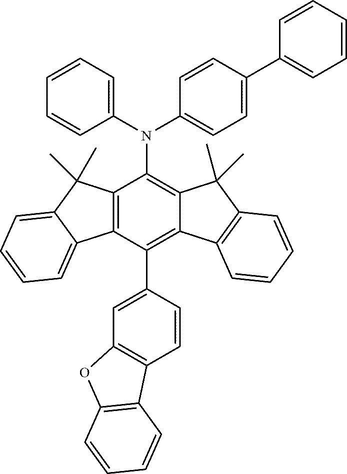 [SMILES: CC1(C)c2ccccc2-c2c(-c3ccc4c(c3)oc3ccccc34)c3c(c(N(c4ccccc4)c4ccc(-c5ccccc5)cc4)c21)C(C)(C)c1ccccc1-3]

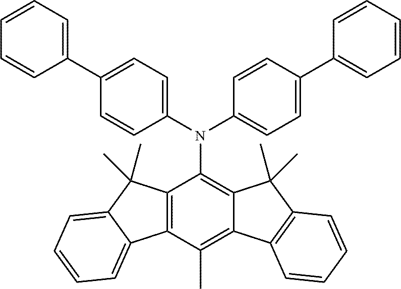 [SMILES: Cc1c2c(c(N(c3ccc(-c4ccccc4)cc3)c3ccc(-c4ccccc4)cc3)c3c1-c1ccccc1C3(C)C)C(C)(C)c1ccccc1-2]

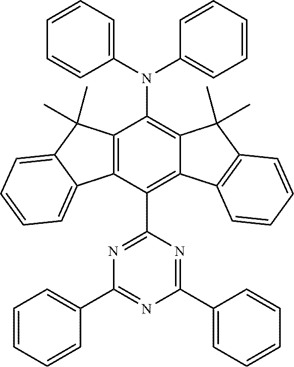 [SMILES: CC1(C)c2ccccc2-c2c(-c3nc(-c4ccccc4)nc(-c4ccccc4)n3)c3c(c(N(c4ccccc4)c4ccccc4)c21)C(C)(C)c1ccccc1-3]

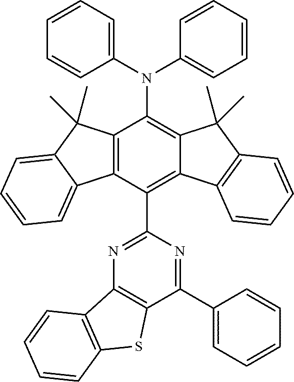 [SMILES: CC1(C)c2ccccc2-c2c(-c3nc(-c4ccccc4)c4sc5ccccc5c4n3)c3c(c(N(c4ccccc4)c4ccccc4)c21)C(C)(C)c1ccccc1-3]

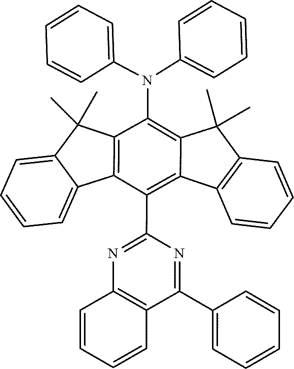 [SMILES: CC1(C)c2ccccc2-c2c(-c3nc(-c4ccccc4)c4ccccc4n3)c3c(c(N(c4ccccc4)c4ccccc4)c21)C(C)(C)c1ccccc1-3]

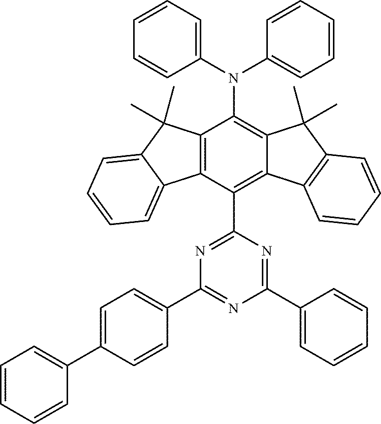 [SMILES: CC1(C)c2ccccc2-c2c(-c3nc(-c4ccccc4)nc(-c4ccc(-c5ccccc5)cc4)n3)c3c(c(N(c4ccccc4)c4ccccc4)c21)C(C)(C)c1ccccc1-3]

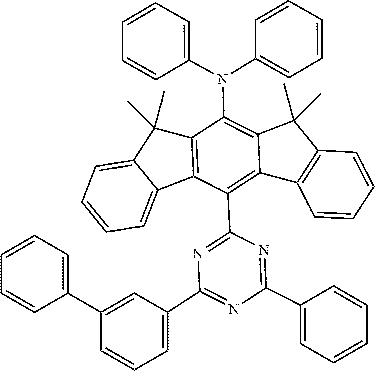 [SMILES: CC1(C)c2ccccc2-c2c(-c3nc(-c4ccccc4)nc(-c4cccc(-c5ccccc5)c4)n3)c3c(c(N(c4ccccc4)c4ccccc4)c21)C(C)(C)c1ccccc1-3]

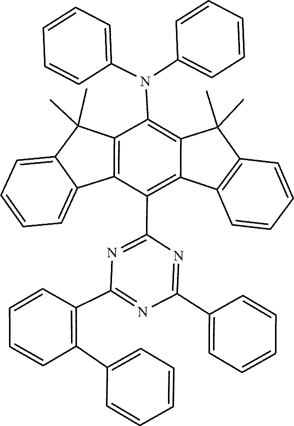 [SMILES: CC1(C)c2ccccc2-c2c(-c3nc(-c4ccccc4)nc(-c4ccccc4-c4ccccc4)n3)c3c(c(N(c4ccccc4)c4ccccc4)c21)C(C)(C)c1ccccc1-3]

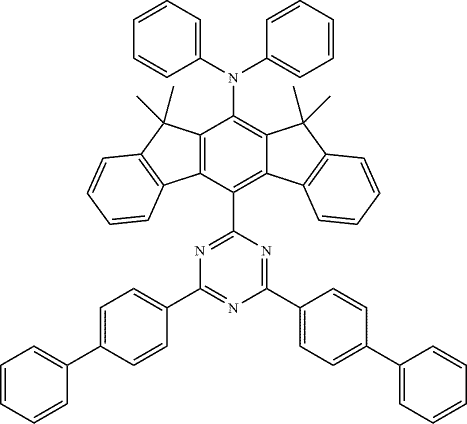 [SMILES: CC1(C)c2ccccc2-c2c(-c3nc(-c4ccc(-c5ccccc5)cc4)nc(-c4ccc(-c5ccccc5)cc4)n3)c3c(c(N(c4ccccc4)c4ccccc4)c21)C(C)(C)c1ccccc1-3]

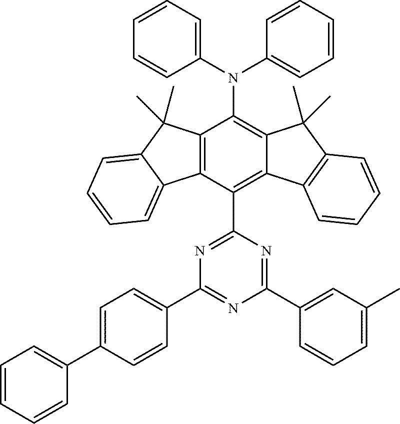 [SMILES: Cc1cccc(-c2nc(-c3ccc(-c4ccccc4)cc3)nc(-c3c4c(c(N(c5ccccc5)c5ccccc5)c5c3-c3ccccc3C5(C)C)C(C)(C)c3ccccc3-4)n2)c1]

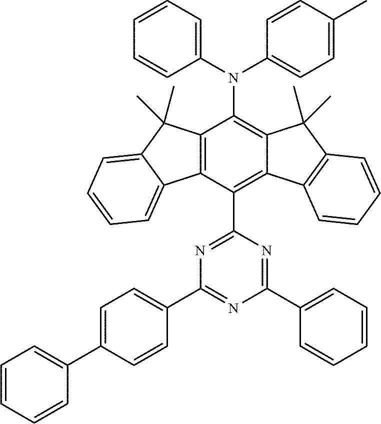 [SMILES: Cc1ccc(N(c2ccccc2)c2c3c(c(-c4nc(-c5ccccc5)nc(-c5ccc(-c6ccccc6)cc5)n4)c4c2C(C)(C)c2ccccc2-4)-c2ccccc2C3(C)C)cc1]

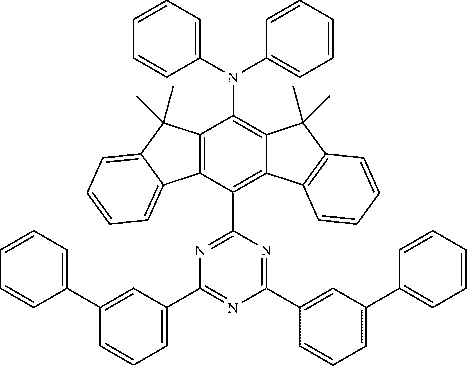 [SMILES: CC1(C)c2ccccc2-c2c(-c3nc(-c4cccc(-c5ccccc5)c4)nc(-c4cccc(-c5ccccc5)c4)n3)c3c(c(N(c4ccccc4)c4ccccc4)c21)C(C)(C)c1ccccc1-3]

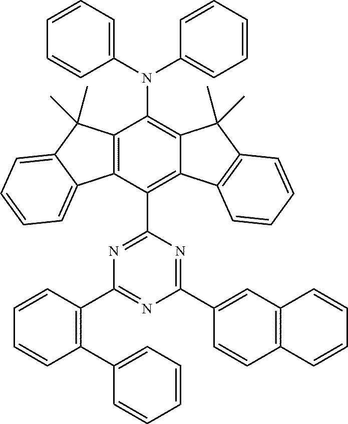 [SMILES: CC1(C)c2ccccc2-c2c(-c3nc(-c4ccc5ccccc5c4)nc(-c4ccccc4-c4ccccc4)n3)c3c(c(N(c4ccccc4)c4ccccc4)c21)C(C)(C)c1ccccc1-3]

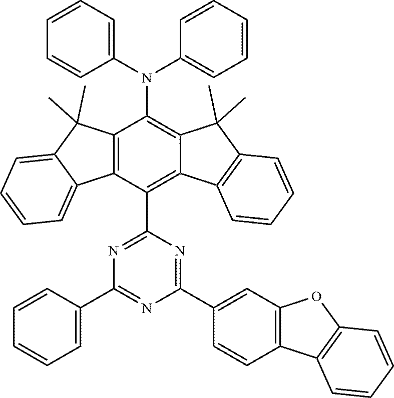 [SMILES: CC1(C)c2ccccc2-c2c(-c3nc(-c4ccccc4)nc(-c4ccc5c(c4)oc4ccccc45)n3)c3c(c(N(c4ccccc4)c4ccccc4)c21)C(C)(C)c1ccccc1-3]

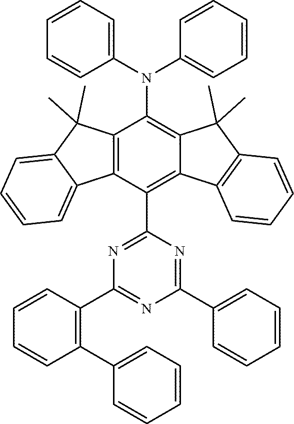 [SMILES: CC1(C)c2ccccc2-c2c(-c3nc(-c4ccccc4)nc(-c4ccccc4-c4ccccc4)n3)c3c(c(N(c4ccccc4)c4ccccc4)c21)C(C)(C)c1ccccc1-3]

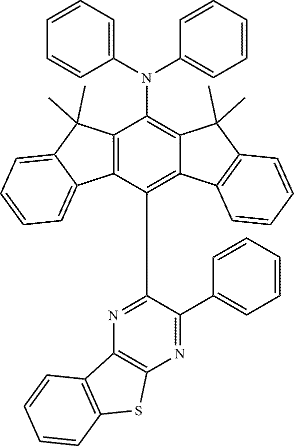 [SMILES: CC1(C)c2ccccc2-c2c(-c3nc4c(nc3-c3ccccc3)sc3ccccc34)c3c(c(N(c4ccccc4)c4ccccc4)c21)C(C)(C)c1ccccc1-3]

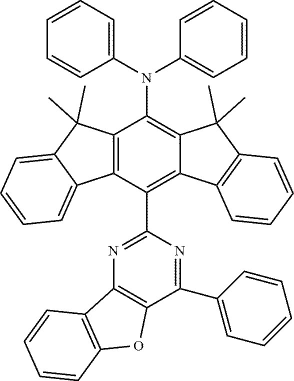 [SMILES: CC1(C)c2ccccc2-c2c(-c3nc(-c4ccccc4)c4oc5ccccc5c4n3)c3c(c(N(c4ccccc4)c4ccccc4)c21)C(C)(C)c1ccccc1-3]

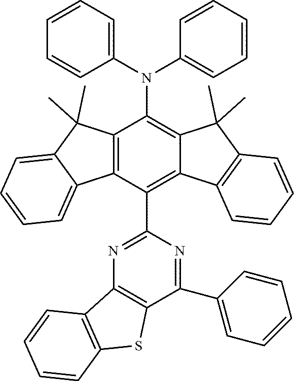 [SMILES: CC1(C)c2ccccc2-c2c(-c3nc(-c4ccccc4)c4sc5ccccc5c4n3)c3c(c(N(c4ccccc4)c4ccccc4)c21)C(C)(C)c1ccccc1-3]

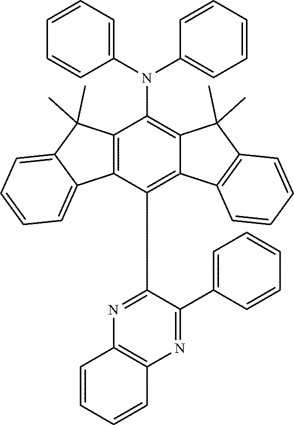 [SMILES: CC1(C)c2ccccc2-c2c(-c3nc4ccccc4nc3-c3ccccc3)c3c(c(N(c4ccccc4)c4ccccc4)c21)C(C)(C)c1ccccc1-3]

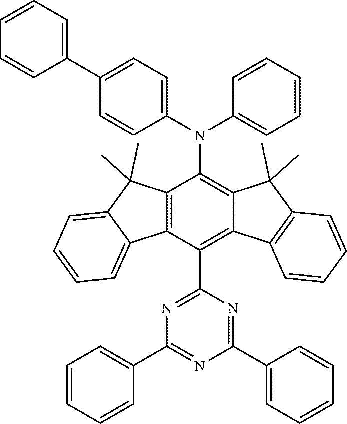 [SMILES: CC1(C)c2ccccc2-c2c(-c3nc(-c4ccccc4)nc(-c4ccccc4)n3)c3c(c(N(c4ccccc4)c4ccc(-c5ccccc5)cc4)c21)C(C)(C)c1ccccc1-3]

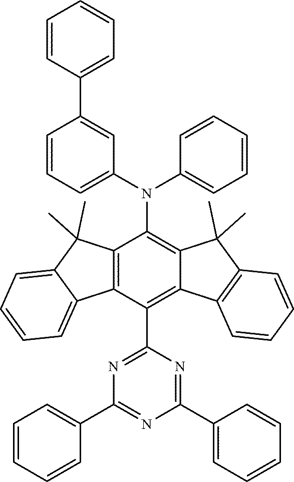 [SMILES: CC1(C)c2ccccc2-c2c(-c3nc(-c4ccccc4)nc(-c4ccccc4)n3)c3c(c(N(c4ccccc4)c4cccc(-c5ccccc5)c4)c21)C(C)(C)c1ccccc1-3]

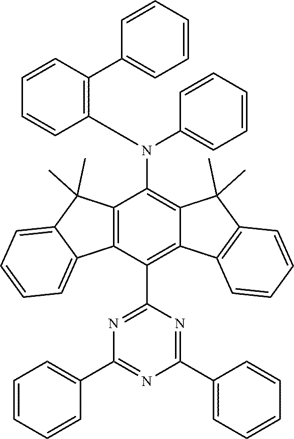 [SMILES: CC1(C)c2ccccc2-c2c(-c3nc(-c4ccccc4)nc(-c4ccccc4)n3)c3c(c(N(c4ccccc4)c4ccccc4-c4ccccc4)c21)C(C)(C)c1ccccc1-3]

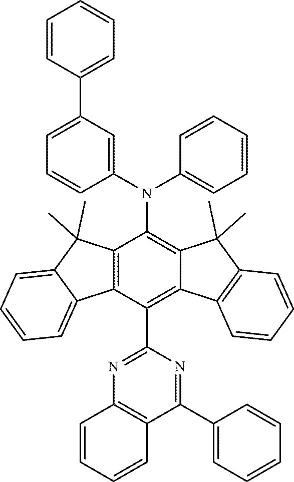 [SMILES: CC1(C)c2ccccc2-c2c(-c3nc(-c4ccccc4)c4ccccc4n3)c3c(c(N(c4ccccc4)c4cccc(-c5ccccc5)c4)c21)C(C)(C)c1ccccc1-3]

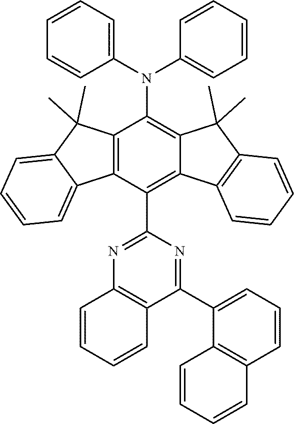 [SMILES: CC1(C)c2ccccc2-c2c(-c3nc(-c4cccc5ccccc45)c4ccccc4n3)c3c(c(N(c4ccccc4)c4ccccc4)c21)C(C)(C)c1ccccc1-3]

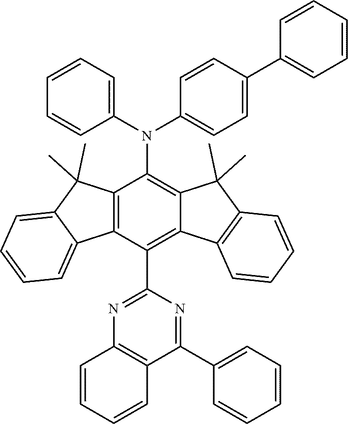 [SMILES: CC1(C)c2ccccc2-c2c(-c3nc(-c4ccccc4)c4ccccc4n3)c3c(c(N(c4ccccc4)c4ccc(-c5ccccc5)cc4)c21)C(C)(C)c1ccccc1-3]

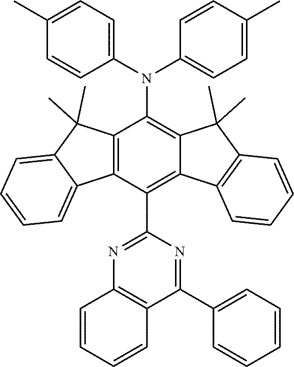 [SMILES: Cc1ccc(N(c2ccc(C)cc2)c2c3c(c(-c4nc(-c5ccccc5)c5ccccc5n4)c4c2C(C)(C)c2ccccc2-4)-c2ccccc2C3(C)C)cc1]

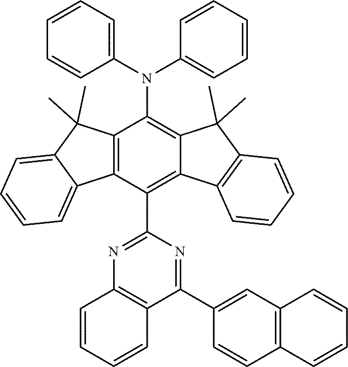 [SMILES: CC1(C)c2ccccc2-c2c(-c3nc(-c4ccc5ccccc5c4)c4ccccc4n3)c3c(c(N(c4ccccc4)c4ccccc4)c21)C(C)(C)c1ccccc1-3]

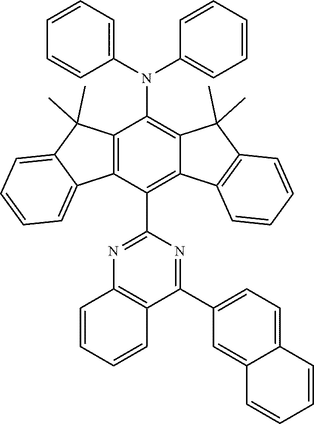 [SMILES: CC1(C)c2ccccc2-c2c(-c3nc(-c4ccc5ccccc5c4)c4ccccc4n3)c3c(c(N(c4ccccc4)c4ccccc4)c21)C(C)(C)c1ccccc1-3]

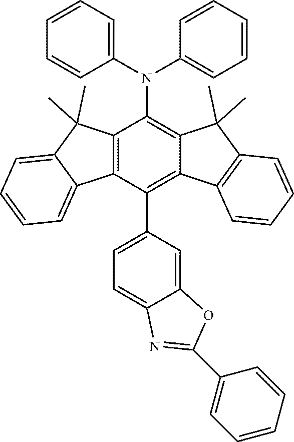 [SMILES: CC1(C)c2ccccc2-c2c(-c3ccc4nc(-c5ccccc5)oc4c3)c3c(c(N(c4ccccc4)c4ccccc4)c21)C(C)(C)c1ccccc1-3]

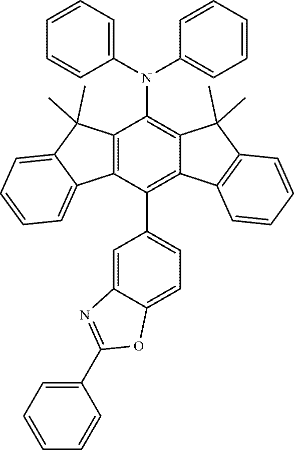 [SMILES: CC1(C)c2ccccc2-c2c(-c3ccc4oc(-c5ccccc5)nc4c3)c3c(c(N(c4ccccc4)c4ccccc4)c21)C(C)(C)c1ccccc1-3]

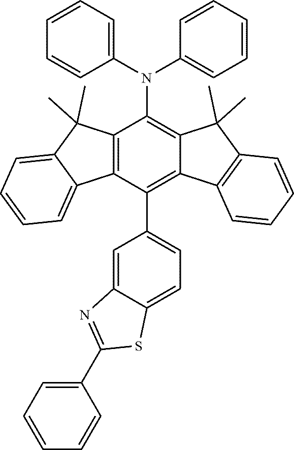 [SMILES: CC1(C)c2ccccc2-c2c(-c3ccc4sc(-c5ccccc5)nc4c3)c3c(c(N(c4ccccc4)c4ccccc4)c21)C(C)(C)c1ccccc1-3]

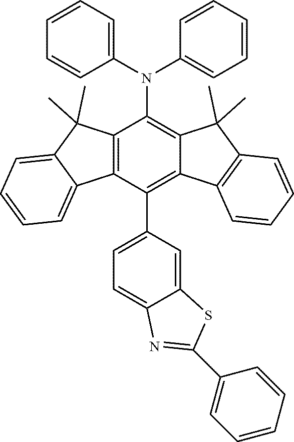 [SMILES: CC1(C)c2ccccc2-c2c(-c3ccc4nc(-c5ccccc5)sc4c3)c3c(c(N(c4ccccc4)c4ccccc4)c21)C(C)(C)c1ccccc1-3]

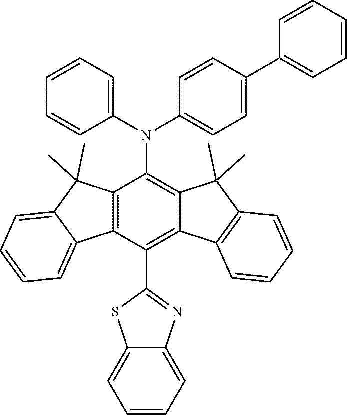 [SMILES: CC1(C)c2ccccc2-c2c(-c3nc4ccccc4s3)c3c(c(N(c4ccccc4)c4ccc(-c5ccccc5)cc4)c21)C(C)(C)c1ccccc1-3]

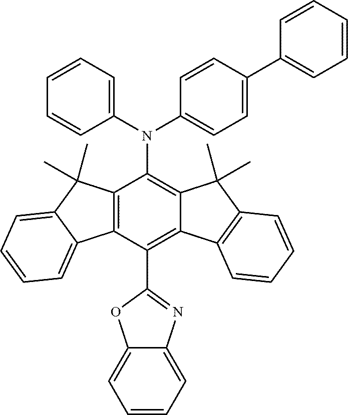 [SMILES: CC1(C)c2ccccc2-c2c(-c3nc4ccccc4o3)c3c(c(N(c4ccccc4)c4ccc(-c5ccccc5)cc4)c21)C(C)(C)c1ccccc1-3]

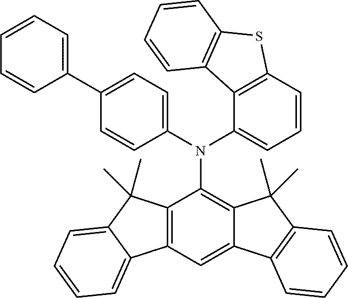 [SMILES: CC1(C)c2ccccc2-c2cc3c(c(N(c4ccc(-c5ccccc5)cc4)c4cccc5sc6ccccc6c45)c21)C(C)(C)c1ccccc1-3]

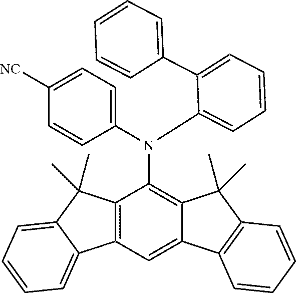

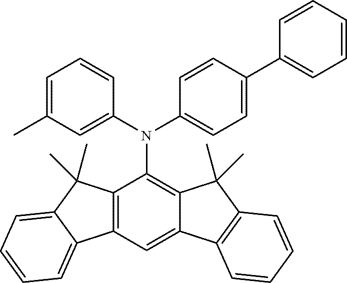 [SMILES: Cc1cccc(N(c2ccc(-c3ccccc3)cc2)c2c3c(cc4c2C(C)(C)c2ccccc2-4)-c2ccccc2C3(C)C)c1]

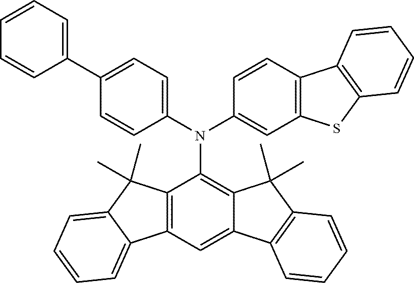 [SMILES: CC1(C)c2ccccc2-c2cc3c(c(N(c4ccc(-c5ccccc5)cc4)c4ccc5c(c4)sc4ccccc45)c21)C(C)(C)c1ccccc1-3]

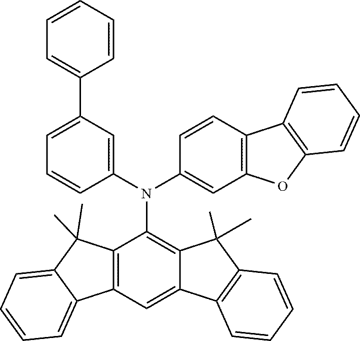 [SMILES: CC1(C)c2ccccc2-c2cc3c(c(N(c4cccc(-c5ccccc5)c4)c4ccc5c(c4)oc4ccccc45)c21)C(C)(C)c1ccccc1-3]

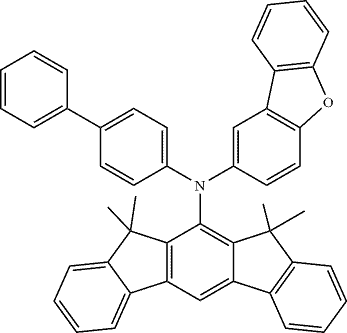 [SMILES: CC1(C)c2ccccc2-c2cc3c(c(N(c4ccc(-c5ccccc5)cc4)c4ccc5oc6ccccc6c5c4)c21)C(C)(C)c1ccccc1-3]

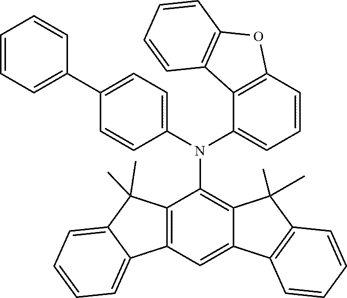 [SMILES: CC1(C)c2ccccc2-c2cc3c(c(N(c4ccc(-c5ccccc5)cc4)c4cccc5oc6ccccc6c45)c21)C(C)(C)c1ccccc1-3]

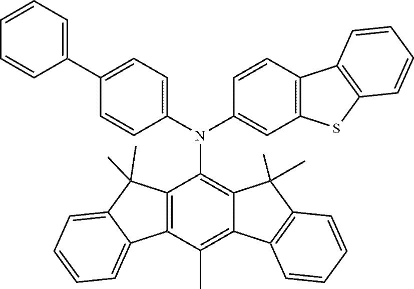 [SMILES: Cc1c2c(c(N(c3ccc(-c4ccccc4)cc3)c3ccc4c(c3)sc3ccccc34)c3c1-c1ccccc1C3(C)C)C(C)(C)c1ccccc1-2]

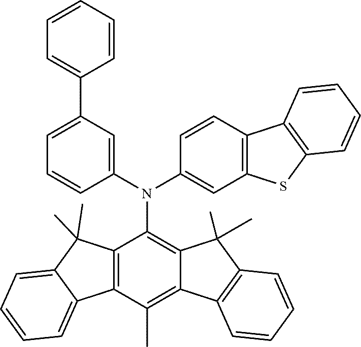 [SMILES: Cc1c2c(c(N(c3cccc(-c4ccccc4)c3)c3ccc4c(c3)sc3ccccc34)c3c1-c1ccccc1C3(C)C)C(C)(C)c1ccccc1-2]

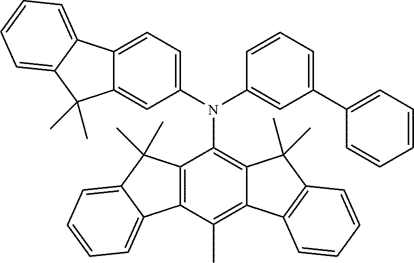 [SMILES: Cc1c2c(c(N(c3cccc(-c4ccccc4)c3)c3ccc4c(c3)C(C)(C)c3ccccc3-4)c3c1-c1ccccc1C3(C)C)C(C)(C)c1ccccc1-2]

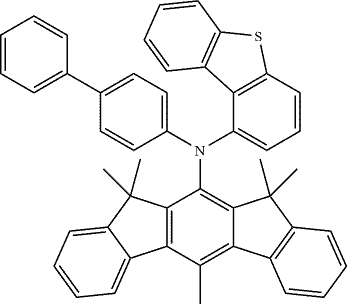 [SMILES: Cc1c2c(c(N(c3ccc(-c4ccccc4)cc3)c3cccc4sc5ccccc5c34)c3c1-c1ccccc1C3(C)C)C(C)(C)c1ccccc1-2]

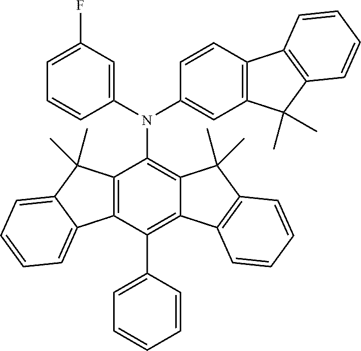 [SMILES: CC1(C)c2ccccc2-c2ccc(N(c3cccc(F)c3)c3c4c(c(-c5ccccc5)c5c3C(C)(C)c3ccccc3-5)-c3ccccc3C4(C)C)cc21]

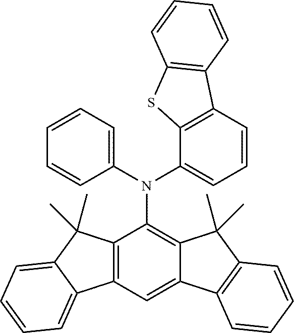 [SMILES: CC1(C)c2ccccc2-c2cc3c(c(N(c4ccccc4)c4cccc5c4sc4ccccc45)c21)C(C)(C)c1ccccc1-3]

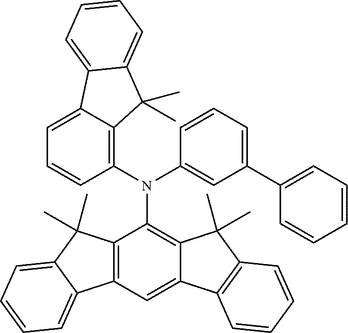 [SMILES: CC1(C)c2ccccc2-c2cccc(N(c3cccc(-c4ccccc4)c3)c3c4c(cc5c3C(C)(C)c3ccccc3-5)-c3ccccc3C4(C)C)c21]

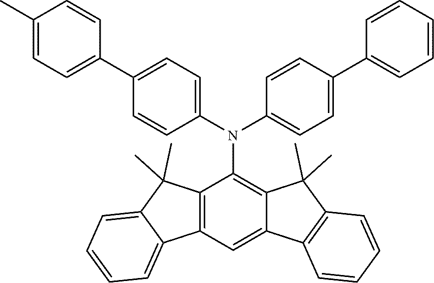 [SMILES: Cc1ccc(-c2ccc(N(c3ccc(-c4ccccc4)cc3)c3c4c(cc5c3C(C)(C)c3ccccc3-5)-c3ccccc3C4(C)C)cc2)cc1]

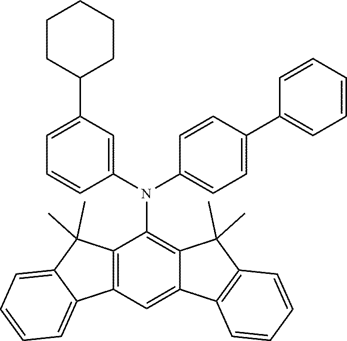 [SMILES: CC1(C)c2ccccc2-c2cc3c(c(N(c4ccc(-c5ccccc5)cc4)c4cccc(C5CCCCC5)c4)c21)C(C)(C)c1ccccc1-3]

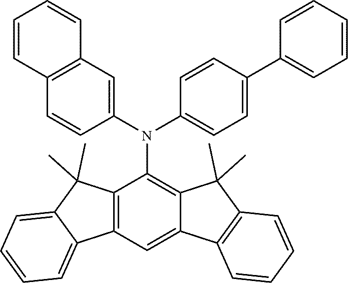 [SMILES: CC1(C)c2ccccc2-c2cc3c(c(N(c4ccc(-c5ccccc5)cc4)c4ccc5ccccc5c4)c21)C(C)(C)c1ccccc1-3]

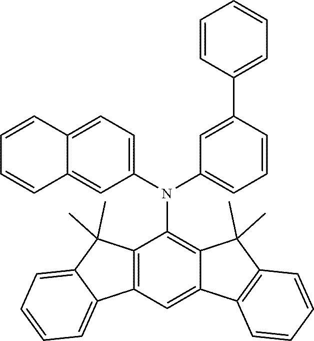 [SMILES: CC1(C)c2ccccc2-c2cc3c(c(N(c4cccc(-c5ccccc5)c4)c4ccc5ccccc5c4)c21)C(C)(C)c1ccccc1-3]

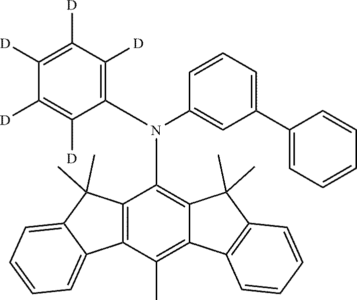 [SMILES: [2H]c1c([2H])c([2H])c(N(c2cccc(-c3ccccc3)c2)c2c3c(c(C)c4c2C(C)(C)c2ccccc2-4)-c2ccccc2C3(C)C)c([2H])c1[2H]]

288.

The synthesis method of the nitrogen-containing compound provided by the present disclosure is not specially limited, and those skilled in the art can determine a proper synthesis method according to the nitrogen-containing compound provided by the present disclosure in combination with the preparation method in the examples. In other words, the examples of the present disclosure exemplarily provide a preparation method of the nitrogen-containing compound, and the raw materials adopted can be obtained commercially or by a method well known in the art. All the nitrogen-containing compounds provided by the present disclosure can be obtained by those skilled in the art according to the preparation method in these exemplary examples, all specific preparation methods for preparing the nitrogen-containing compound are no longer detailed, and those skilled in the art should not understand it as limiting the disclosure.

In a second aspect, the present disclosure provides an organic electroluminescent device, including an anode, a cathode and a functional layer disposed between the anode and the cathode, and the functional layer includes the nitrogen-containing compound in the first aspect of the present disclosure.

The nitrogen-containing compound provided by the present disclosure can be used for forming at least one organic film layer in the functional layer so as to improve the characteristics such as the service life of the organic electroluminescent device. The organic electroluminescent device can be a blue light device, a green light device, or a red light device.

According to one embodiment, the functional layer includes a hole transporting layer, and the hole transporting layer contains the nitrogen-containing compound provided by the present disclosure. Wherein the hole transporting layer may be composed of the nitrogen-containing compound provided by the present disclosure, and may also be composed of the nitrogen-containing compound provided by the present disclosure and other materials together. The hole transporting layer may include one or two or more layers. Optionally, the hole transporting layer includes a first hole transporting layer and a second hole transporting layer (such as an electron blocking layer) which are stacked, and the first hole transporting layer is closer to the surface of the anode than the second hole transporting layer; and the first hole transporting layer and/or the second hole transporting layer contain/contains the nitrogen-containing compound provided by the present disclosure.

According to another embodiment, the functional layer includes a luminescent layer, wherein the luminescent layer includes a host material and a luminescent dopant, and the host material includes the nitrogen-containing compound.

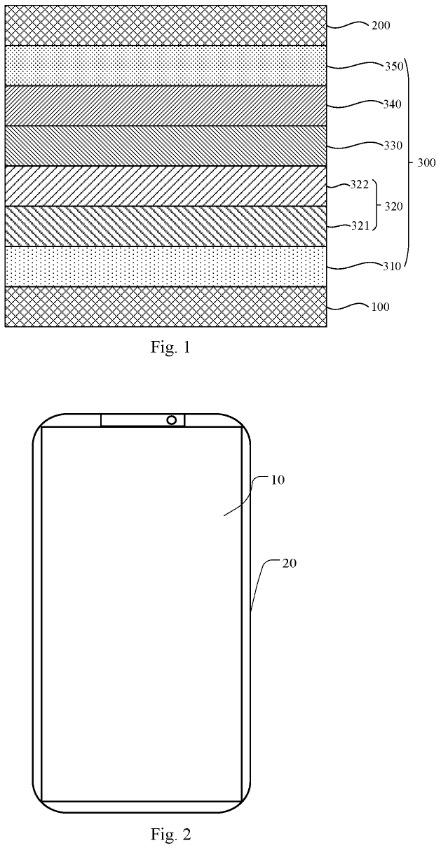

Optionally, as shown in FIG. 1, an organic electroluminescent device may include an anode 100, a first hole transporting layer 321, a second hole transporting layer 322, a luminescent layer 330 serving as an energy conversion layer, an electron transporting layer 340 and a cathode 200 which are sequentially stacked. The first hole transporting layer 321 and the second hole transporting layer 322 constitute a hole transporting layer 320.

According to one exemplary embodiment, the nitrogen-containing compound provided by the present disclosure may be applied to the first hole transporting layer 321 or the second hole transporting layer 322 of the organic electroluminescent device, so that the service life of the organic electroluminescent device is prolonged, and the luminous efficiency of the organic electroluminescent device is improved.

In the present disclosure, the anode 100 includes an anode material, which is preferably a material having a large work function that facilitates holes injecting into the functional layer. Specific examples of the anode material include, but not limited to, metals such as nickel, platinum, vanadium, chromium, copper, zinc, and aurum, or an alloy thereof; metal oxides such as zinc oxide, indium oxide, indium tin oxide (ITO) and indium zinc oxide (IZO); combination of metals and oxides, such as ZnO:Al or SnO₂:Sb; or a conductive polymer such as poly(3-methylthiophene), poly[3,4-(ethylene-1,2-dioxy)thiophene] (PEDT), polypyrrole, and polyaniline. A transparent electrode containing indium tin oxide (ITO) as the anode is preferably included.

Optionally, the luminescent layer 330 may consist of a single luminescent material, and may also include a host material and a luminescent dopant. In one specific embodiment, the luminescent layer 330 is composed of a host material and a luminescent dopant, the holes injected into the luminescent layer 330 and electrons injected into the luminescent layer 330 can be recombined in the luminescent layer 330 to form excitons, the excitons transmit energy to the host material, the host material transmits energy to a guest material, and then the guest material can emit light.

When the nitrogen-containing compound provided by the present disclosure is not selected as a host material, the host material of the luminescent layer 330 may be a metal chelated compound, a distyryl derivative, an aromatic amine derivative, a dibenzofuran derivative or other types of materials, which is not specially limited by the present disclosure.

In the present disclosure, the light-emitting dopant of the luminescent layer 330 may be a compound having a condensed aryl ring or a derivative thereof, a compound having a heteroaryl ring or a derivative thereof, an aromatic amine derivative, or other materials, which is not specially limited by the disclosure.

In the disclosure, the electron transporting layer 340 may be of a single-layer structure or a multi-layer structure and can include one or more electron transporting materials, and the electron transporting material may be selected from, but not limited to, a benzimidazole derivative, an oxadiazole derivative, a quinoxaline derivative, or other electron transporting materials. In one embodiment of the present disclosure, the electron transporting layer 340 may be composed of TPBi and LiQ.

In the present disclosure, the cathode 200 may include a cathode material, which is a material with small work function that contributes to electron injecting into the functional layer. Specific examples of the cathode material include, but not limited to, metals such as magnesium, calcium, sodium, potassium, titanium, indium, yttrium, lithium, gadolinium, aluminum, argentum, tin and plumbum, or an alloy thereof; or a plurality of layers of materials such as LiF/Al, Liq/Al, LiO₂/Al, LiF/Ca, LiF/Al, and BaF₂/Ca. A metal electrode containing aluminum as the cathode is preferably included.

Optionally, as shown in FIG. 1, a hole injection layer 310 can also be arranged between the anode 100 and the first hole transporting layer 321, so that the capability of injecting holes into the first hole transporting layer 321 is enhanced. The hole injection layer 310 can be made of a benzidine derivative, a starburst arylamine compound, a phthalocyanine derivative or other materials, which is not specially limited by the present disclosure. For example, the hole injection layer 310 may be composed of HAT-CN.

Optionally, as shown in FIG. 1, an electron injection layer 350 can also be arranged between the cathode 200 and the electron transporting layer 340 so as to enhance the capability of injecting electrons into the electron transporting layer 340. The electron injection layer 350 may include an inorganic material such as an alkali metal sulfide, an alkali metal halide and the like, or may include a complex compound of an alkali metal and an organic. For example, the electron injection layer 350 may include LiQ.

Optionally, as shown in FIG. 1, the hole injection layer 310, the first hole transporting layer 321, the second hole transporting layer 322, the luminescent layer 330, the electron transporting layer 340 and the electron injection layer 350 which are sequentially stacked form the functional layer 300.

In the third aspect, the present disclosure provides an electronic apparatus, including the organic electroluminescent device in the second aspect of the present disclosure.

The organic electroluminescent device provided by the present disclosure can be used in the electronic apparatus, and the electronic apparatus may be a mobile phone display screen, a computer display screen, a television display screen, an intelligent watch display screen, an intelligent automobile organic electroluminescent device, a VR or AR helmet display screen, a display screen of various intelligent devices or the like. According to one embodiment, the electronic apparatus is shown in FIG. 2, in FIG. 2,10 represents a mobile phone display panel including the organic electroluminescent device provided by the present disclosure, and 20 represents the electronic apparatus, particularly a mobile phone.

Several particular examples are exemplarily provided below to further interpret and describe the present disclosure.

In order to conveniently understand the present disclosure, the numbers between the following raw materials and the compounds prepared are corresponding. For example, “raw material 24E” refers to a raw material IE specifically selected when the compound 24 is prepared. Intermediate 1F refers to an intermediate IF selected when the compound 1 is prepared. Unless otherwise specified, the raw material IE can be commercially purchased, or is directly obtained from an aromatic amine and the corresponding halide by a Buchwald-Hartwisting reaction.

### Synthesis of Intermediate I-D

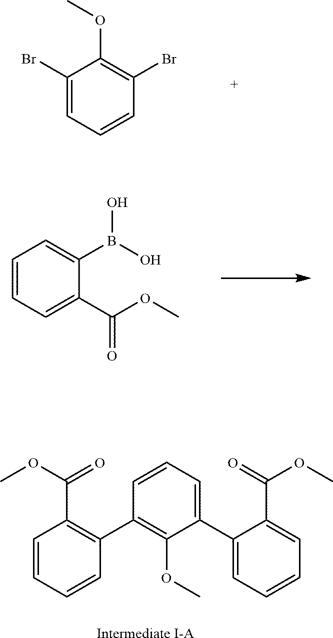

(1) 2,6-dibromoanisole (2.66 g, 10 mmol), 2-(methoxycarbonyl)phenylboronic acid (3.96 g, 22 mmol) and potassium carbonate (6.91 g, 50 mmol) were sequentially added into a three-necked flask equipped with a thermometer and a condenser tube, then 22 mL of toluene, 5 mL of ethanol and 2.5 mL of water were sequentially added, air in the reaction flask is replaced with nitrogen, tetrakis(triphenylphosphine)palladium (Pd(PPh₃)₄, 0.06 g, 0.05 mmol) was added under the protection of nitrogen, heating was started while magnetically stirring, and heated to reflux, the reaction was carried out for 8h, then a sample was taken for detecting, the reaction was stopped when the raw materials were completely reacted, then the system was cooled to room temperature for post-treatment. The reaction liquid was filtered, and allowed to stand for layering, then the obtained water phase layer was extracted with 30 mL of toluene, and organic phases were mixed, washed with water to be neutral, dried over anhydrous sodium sulfate for 1h, and filtered to remove the drying agent to obtain a filter cake, the filter cake was subjected to drip washing with a small amount of toluene, and the obtained filtrates were mixed, concentrated, and purified by passing through a column to obtain an off-white solid, namely intermediate I-A (3.46 g, yield: 92%).

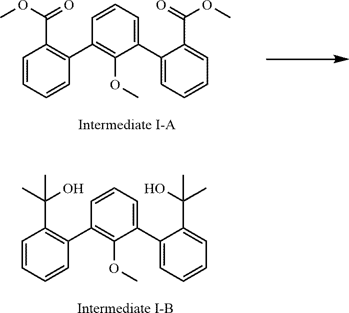

(2) 40 mL of THF was added into a three-necked flask, then the intermediate I-A (3.46 g, 9.19 mmol) was added, air in the reaction flask was replaced with nitrogen, the reaction system was cooled to 0° C., 18.4 mL of THF solution (3M) of MeMgBr was slowly added dropwise under the protection of nitrogen, the temperature was maintained for 1h, then heated to room temperature, and the reaction was carried out under stirring for 12h. When it is monitored that the raw materials were completely reacted, the reaction was stopped, saturated NH₄Cl aqueous solution was added to quench the reaction, the reaction liquid obtained was allowed to stand for layering, water phase layer was extracted with dichloromethane (DCM) twice, and with 60 mL in each time, organic phases were mixed, washed with saturated saline solution once, dried over anhydrous sodium sulfate, and filtered to remove the drying agent, and the filtrate obtained was concentrated, and allowed to pass through a column to obtain a white solid, namely intermediate I-B (2.59 g, yield: 75%).

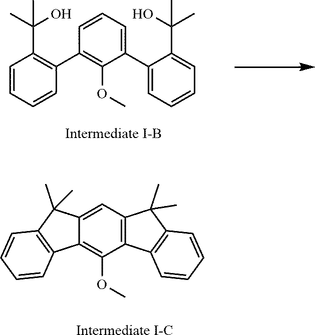

(3) 20 mL of acetic acid was added into a three-necked flask, then the intermediate I-B (2.60 g, 6.90 mmol) was added, the system was cooled to 0° C. and stirred for 10 min, then 15 mL of phosphoric acid (85%) was added, the reaction system was heated to room temperature and stirred for 3 h, after TLC monitored that the raw materials were completely reacted, a NaOH solution was added into the reaction system to adjust the pH to be neutral, then water phase was extracted with DCM for three times, with 40 mL in each time, the organic phases were mixed, washed with saturated saline solution once, dried over anhydrous sodium sulfate for 1h, and filtered, and the filtrate obtained was concentrated, and purified by passing through a silica gel column to obtain intermediate I-C (1.40 g, yield: 60%).

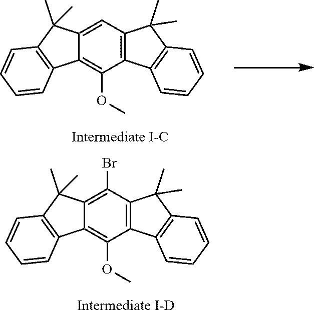

(4) The intermediate I-C (1.4 g, 4.14 mmol) and 10 mL of DMF (N,N-dimethylformamide) were added into a three-necked flask, stirring was started to allow complete dissolution of the raw material, then N-bromosuccinimide (NBS, 0.81 g, 4.55 mmol) was added in batches, obvious temperature rise was occured, the temperature was controlled to be 15 to 20° C., where the addition was completed within about 1.5 h, and the reaction was stopped after the raw materials were completely reacted. The reaction liquid was poured into water with stirring, stirring was performed for 5 min, the stirred material was allowed to stand for 30 min, suction filtration was performed, the obtained solid was boiled and washed for 1 hour (50° C.), suction filtration was performed while the material is hot, drying was performed to obtain a solid, and the solid was recrystallized with dichloroethane to obtain the intermediate I-D (1.56 g of off-white solid, yield: 90%).

Synthesis example 1: Synthesis of compound 1

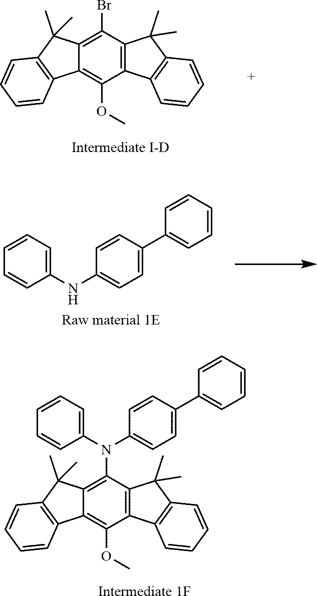

(1) The intermediate I-D (1.56 g, 3.73 mmol), raw material 1E (N-phenyl-4-benzidine, 1.10 g, 4.48 mmol), sodium tert-butoxide (0.90 g, 9.33 mmol) and 15 mL of toluene were sequentially added into a four-necked flask equipped with a mechanical stirrer, stirring was started, nitrogen was introduced, the system was heated to 110 to 115° C., refluxing and water segregation was performed for 1h, then tris(dibenzylideneacetone)dipalladium (Pd₂(dba)₃, 0.03 g, 0.03 mmol) and 2-dicyclohexylphosphino-2,4,6-triisopropylbiphenyl (X-phos, 0.02 g, 0.037 mmol) were added, and the reaction was carried out overnight while the reflux state was continuously kept. After the reaction liquid was cooled to room temperature, the reaction liquid was poured into water with stirring, liquid separation was performed, water phase was extracted twice with toluene, organic phases were mixed, washed twice with water, dried over anhydrous sodium sulfate, and concentrated to be dry to obtain a brown yellow oily substance, and the oily substance was subjected to column chromatography to obtain intermediate 1F (1.53 g of white solid, yield: 70%).

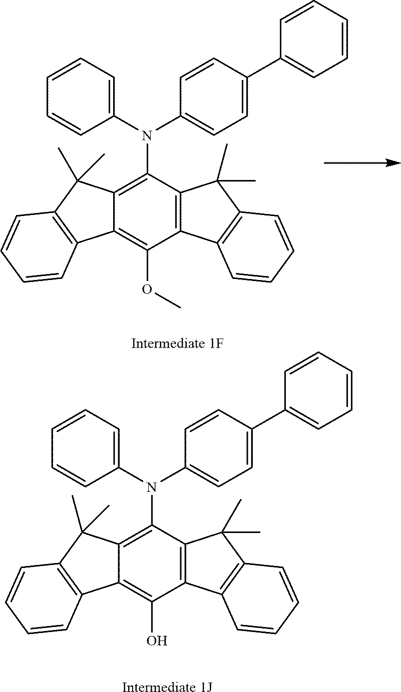

(2) The intermediate 1F (1.53 g, 2.61 mmol) was added into a three-necked flask equipped with 15 mL of DCM, air in the reaction flask was replaced with nitrogen, the flask was cooled to about -5° C., BBr₃ (0.78 g, 3.13 mmol) was dissolved into 10 mL of DCM, the obtained solution was slowly added into the reaction system under the protection of nitrogen, the reaction was continued to be carried out while maintaining the temperature after dropwise adding finished, and the reaction was stopped until TLC monitored that the raw materials were reacted completely. Water was slowly added for quenching the reaction under an ice bath condition, standing was performed for layering, water phase was extracted with DCM, organic phases were mixed, washed with saturated salt solution once, then dried over anhydrous sodium sulfate for 1h, and filtered to remove the drying agent, and the obtained filtrate was concentrated, and allowed to pass through a silica gel column to obtain a target product, namely intermediate 1J (1.34 g of off-white solid, yield: 90%).

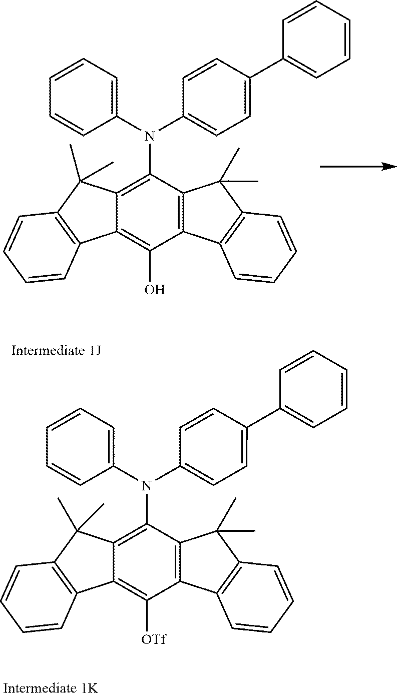

(3) The intermediate 1 J (1.34 g, 2.35 mmol) and 15 mL of dichloromethane were sequentially added into a three-necked flask, stirring was started, pyridine (0.37 g, 4.7 mmol) was added, the flask was cooled to 0° C. or below, trifluoromethanesulfonic anhydride (0.73 g, 2.59 mmol) was dropwise added while maintaining the temperature at about 0° C., dropwise adding was completed within about 1 hour, the reaction was carried out for 2.0h with temperature holding, then the system was heated naturally to room temperature. 2 M hydrochloric acid was added into the reaction liquid with stirring, stirring was performed for 10 min, then dichloromethane was added for extracting twice, and organic phase was washed with water twice, dried over anhydrous sodium sulfate for 0.5 h, and allowed to pass through a chromatographic column to obtain intermediate 1 K (1.25 g of white solid, yield: 76%).

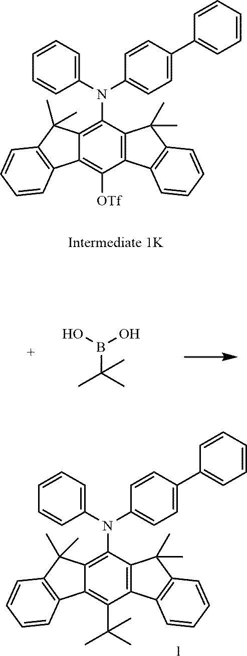

(4) The intermediate 1 K (1.25 g, 1.78 mmol), tert-butylboronic acid (0.20 g, 2.0 mmol) and potassium carbonate (0.69 g, 5 mmol) were sequentially added into a three-necked flask equipped with a thermometer and a condenser tube, then 10 mL of toluene, 5 mL of ethanol and 2.5 mL of water were sequentially added, air in the reaction flask was replaced with nitrogen, Pd(PPh₃)₄ (0.012 g, 0.01 mmol) was added under the protection of nitrogen, heating was started while magnetically stirring, the system was heated to reflux, the reaction was carried out for 8h, then a sample was taken for detecting, the reaction was stopped when the raw materials were completely reacted, then the system was cooled to room temperature, post-treatment was performed, the reaction liquid was filtered, and allowed to stand for layering, water phase layer was extracted with toluene, organic phases were mixed, washed with water to be neutral, dried over anhydrous sodium sulfate for 1h, and filtered to remove the drying agent to obtain a filter cake, the filter cake was subjected to drip washing with a small amount of toluene, and the filtrates obtained were mixed, concentrated, and separated by a chromatographic column to obtain off-white solid, namely compound 1 (0.98 g, yield: 90%), and m/z = 610.3 [M+H]⁺. NMR of the compound 1: ¹H NMR (CDCl₃, 300 MHz): δ (ppm) 8.26-8.21 (m, 2H), 8.09-8.03 (m, 2H), 7.78-7.74 (m, 2H), 7.86-7.81 (m, 5H), 7.72-7.67 (m, 4H), 7.53-7.49 (m, 2H), 7.45-7.41 (m, 2H), 7.32-7.29 (m, 2H), 7.24-7.19 (m, 1H), 2.65 (s, 12H), 2.21 (s, 9H).

### Synthesis Examples 2 to 3

Compound 4 and compound 6 were respectively synthesized according to the method in synthesis example 1, except that N-phenyl-4-benzidine in synthesis example 1 was adjusted to be a raw material IE in the following table. The main raw materials adopted, the structures of the compounds synthesized correspondingly, the total yield of the compounds and the mass spectrum characterization are shown in the following table.

TABLE 1

| Synthesis example No. | Compound No. | Intermediate I-D | Raw material IE | Tert-butylboronic acid | Compound structure | Yield, % | Mass spectrum (m/z), [M+H]⁺ |
|---|---|---|---|---|---|---|---|
| 2 | 4 | 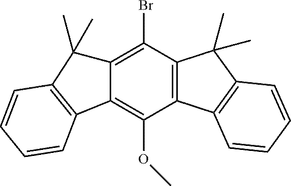 [SMILES: COc1c2c(c(Br)c3c1-c1ccccc1C3(C)C)C(C)(C)c1ccccc1-2] | 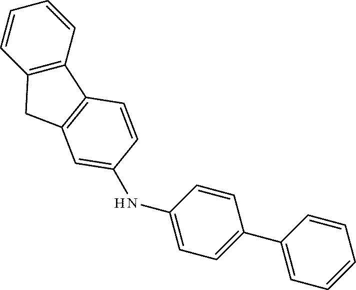 | 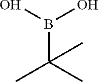 | 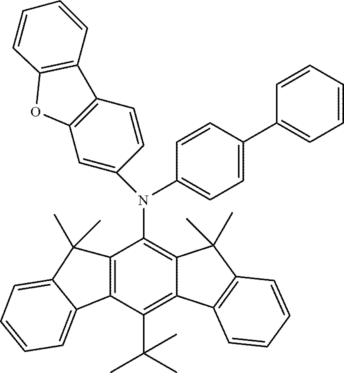 [SMILES: CC(C)(C)c1c2c(c(N(c3ccc(-c4ccccc4)cc3)c3ccc4c(c3)oc3ccccc34)c3c1-c1ccccc1C3(C)C)C(C)(C)c1ccccc1-2] | 28 | 700.8 |
| 3 | 6 | 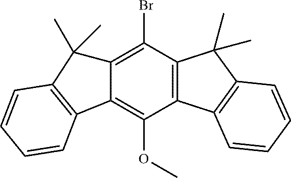 [SMILES: COc1c2c(c(Br)c3c1-c1ccccc1C3(C)C)C(C)(C)c1ccccc1-2] | 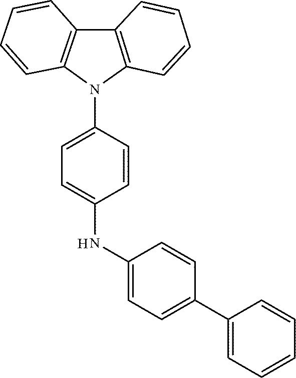 | 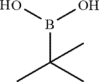 | 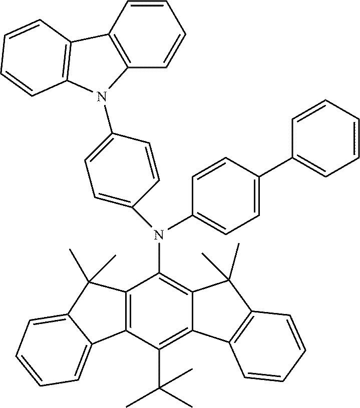 [SMILES: CC(C)(C)c1c2c(c(N(c3ccc(-c4ccccc4)cc3)c3ccc(-n4c5ccccc5c5ccccc54)cc3)c3c1-c1ccccc1C3(C)C)C(C)(C)c1ccccc1-2] | 32 | 774.9 |

Synthesis example 4: Synthesis of compound 8

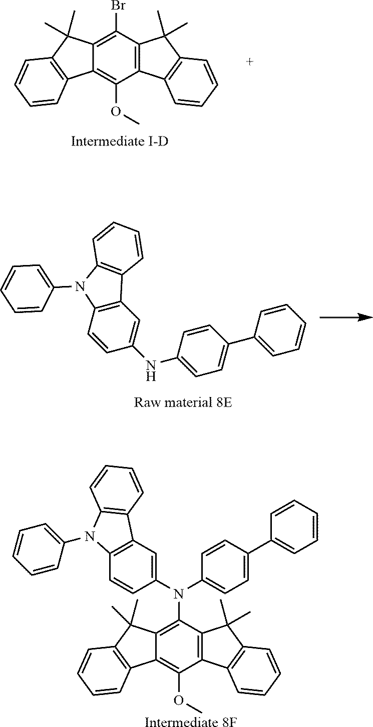

(1) The intermediate I-D (2.30 g, 5.5 mmol), raw material 8E (2.82 g, 6.60 mmol), sodium tert-butoxide (1.33 g, 13.75 mmol) and 25 mL of toluene were sequentially added into a four-necked flask equipped with a mechanical stirrer, stirring was started, nitrogen was introduced, the flask was heated to 110 to 115° C., refluxing and water segregation were performed for 1h, then Pd₂(dba)₃ (0.03 g, 0.03 mmol) and X-phos (0.02 g, 0.037 mmol) were added, and the reaction was carried out overnight while a reflux state was continuously kept. The reaction liquid was cooled to room temperature, poured into water with stirring, liquid separation was performed, water phase was extracted twice with toluene, organic phases were mixed, washed twice with water, dried over anhydrous sodium sulfate, and concentrated to be dry to obtain a brown yellow oily substance, and the oily substance was separated by column chromatography to obtain intermediate 8F 2.74 g of white solid yield: 65%.

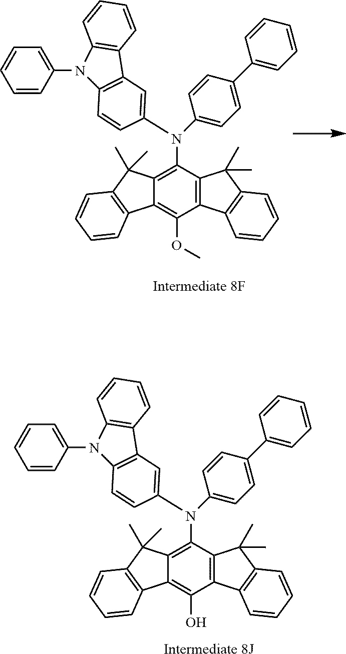

(2) The intermediate 8F (2.74 g, 3.58 mmol) was added into a three-necked flask containing 30 mL of DCM, air in the reaction flask was replaced with nitrogen, the flask was cooled to -5° C., BBr₃ (1.07 g, 4.30 mmol) was dissolved into 10mL of DCM, the solution obtained was slowly added into the reaction system under the protection of nitrogen, the reaction was continued to be carried out while maintaining the temperature after dropwise adding finished, and the reaction was stopped until TLC monitored that the raw materials were reacted completely. Water was slowly added for quenching the reaction under an ice bath condition, standing was performed for layering, water phase was extracted with DCM, organic phases were mixed, washed with saturated salt solution once, then dried over anhydrous sodium sulfate for 1h, and filtered to remove the drying agent, and the filtrate obtained was concentrated, and purified by passing through a silica gel column to obtain intermediate 8J (2.42 g of off-white solid, yield: 90%).

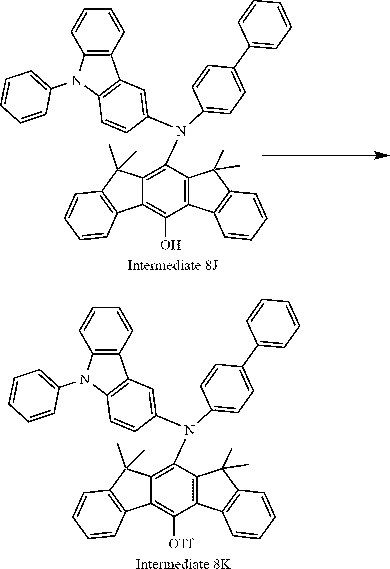

(3) The intermediate 8J (2.42 g, 3.22 mmol) and 25 mL of dichloromethane were sequentially added into a three-necked flask, stirring was started, pyridine (0.51 g, 6.44 mmol) was slowly added, the flask was cooled to about -3° C., trifluoromethanesulfonic anhydride (1.00 g, 3.54 mmol) was dropwise added while maintaining the temperature at about 0° C., where dropwise adding was completed within about 1h, the reaction was carried out for 2.0 h with temperature holding, then the system was heated naturally to room temperature. 2M hydrochloric acid was added into the reaction liquid with stirring, stirring was performed for 10 min, dichloromethane was added for extracting twice, and organic phase was washed with water twice, dried over anhydrous sodium sulfate for 0.5 h, and allowed to pass through a chromatographic column to obtain intermediate 8K (2.42 g of off-white solid, yield: 85%).

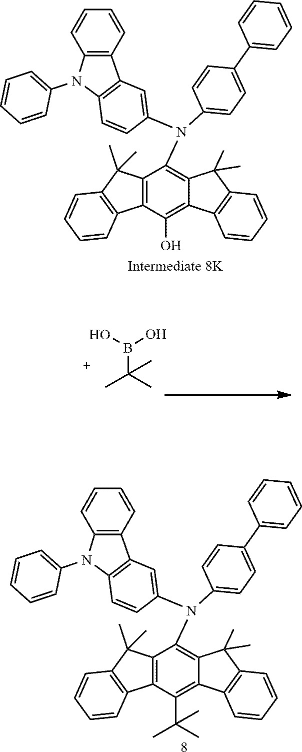

(4) The intermediate 8 K (2.42 g, 2.74 mmol), tert-butylboronic acid (0.28 g, 2.74 mmol) and potassium carbonate (0.95 g, 6.85 mmol) were sequentially added into a three-necked flask equipped with a thermometer and a condenser tube, then 20 mL of toluene, 5 mL of ethanol and 2.5 mL of water were sequentially added, air in the reaction flask was replaced with nitrogen, Pd(PPh₃)₄ (0.016 g, 0.014 mmol) was added under the protection of nitrogen, heating was started while magnetically stirring, the system was heated to reflux, the reaction was carried out for 8h, a sample was taken for detecting, the reaction was stopped when the raw materials were completely reacted, the system was cooled to room temperature, post-treatment was performed, the reaction liquid was filtered, and allowed to stand for layering, water phase layer was extracted with toluene, organic phases were mixed, washed with water to be neutral, dried over anhydrous sodium sulfate for 1h, and filtered to remove the drying agent to obtain a filter cake, the filter cake was subjected to drip washing with a small amount of toluene, and the filtrates obtained were mixed, concentrated, and separated by a chromatographic column to obtain an off-white solid, namely compound 8 (1.30 g, yield: 60%), and m/z = 775.6 [M+H]⁺.

### Synthesis Examples 5 to 22

According to the method in synthesis example 4, compounds listed in Table 2 were synthesized respectively, except that the raw material 8E was adjusted to be the raw material IE in the following table, tert-butylboronic acid was replaced by the IL in the following table, and the main raw materials adopted, the structures of the compounds synthesized correspondingly, the total yield of the compounds and the mass spectrum result are shown in Table 2.

TABLE 2

| Synthesis example No. | Compound No. | Raw material IE | Raw material IL | Compound structure | Yield, % | Mass spectru m (m/z), [M+H]⁺ |
|---|---|---|---|---|---|---|
| 5 | 24 | 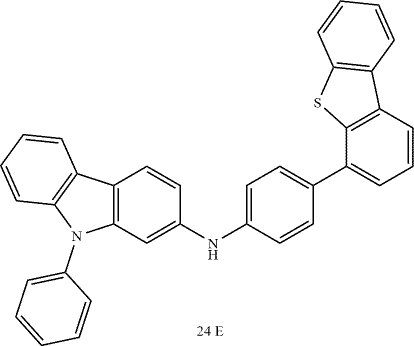 | 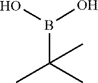 | 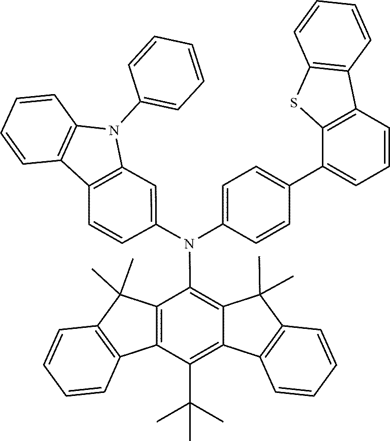 [SMILES: CC(C)(C)c1c2c(c(N(c3ccc(-c4cccc5c4sc4ccccc45)cc3)c3ccc4c5ccccc5n(-c5ccccc5)c4c3)c3c1-c1ccccc1C3(C)C)C(C)(C)c1ccccc1-2] | 31 | 880.6 |
| 6 | 27 | 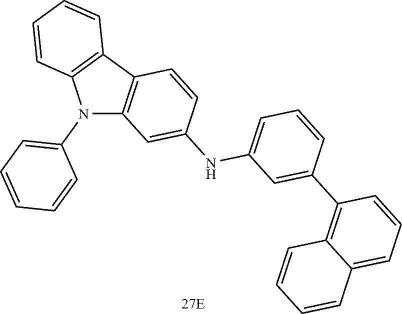 | 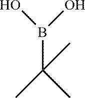 | 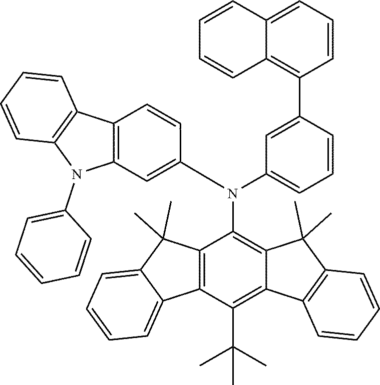 [SMILES: CC(C)(C)c1c2c(c(N(c3cccc(-c4cccc5ccccc45)c3)c3ccc4c5ccccc5n(-c5ccccc5)c4c3)c3c1-c1ccccc1C3(C)C)C(C)(C)c1ccccc1-2] | 30 | 825.1 |
| 7 | 33 | 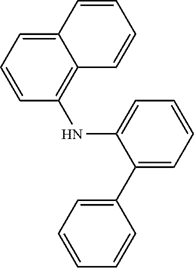 [SMILES: c1ccc(-c2ccccc2Nc2cccc3ccccc23)cc1] | 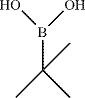 | 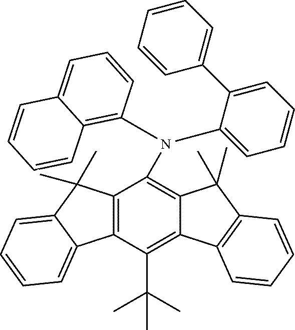 [SMILES: CC(C)(C)c1c2c(c(N(c3ccccc3-c3ccccc3)c3cccc4ccccc34)c3c1-c1ccccc1C3(C)C)C(C)(C)c1ccccc1-2] | 32 | 660.2 |
| 8 | 36 | 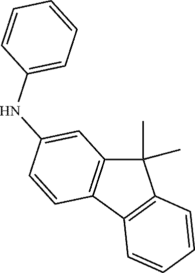 [SMILES: CC1(C)c2ccccc2-c2ccc(Nc3ccccc3)cc21] | 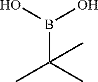 | 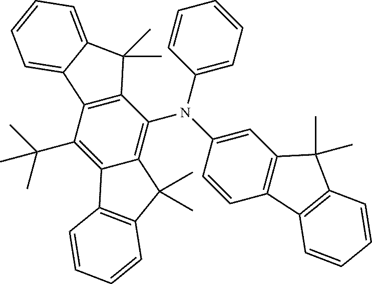 [SMILES: CC(C)(C)c1c2c(c(N(c3ccccc3)c3ccc4c(c3)C(C)(C)c3ccccc3-4)c3c1-c1ccccc1C3(C)C)C(C)(C)c1ccccc1-2] | 29 | 650.3 |
| 9 | 38 | 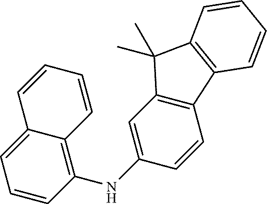 [SMILES: CC1(C)c2ccccc2-c2ccc(Nc3cccc4ccccc34)cc21] | 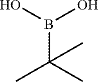 | 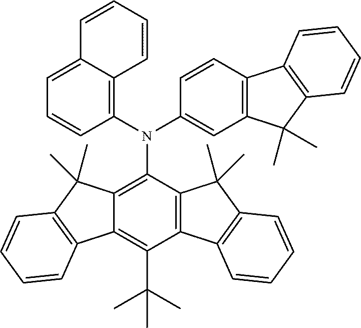 [SMILES: CC(C)(C)c1c2c(c(N(c3ccc4c(c3)C(C)(C)c3ccccc3-4)c3cccc4ccccc34)c3c1-c1ccccc1C3(C)C)C(C)(C)c1ccccc1-2] | 36 | 700.5 |
| 10 | 60 | 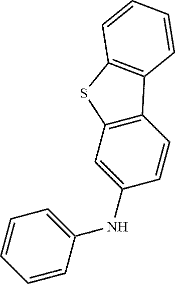 [SMILES: c1ccc(Nc2ccc3c(c2)sc2ccccc23)cc1] | 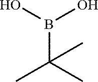 | 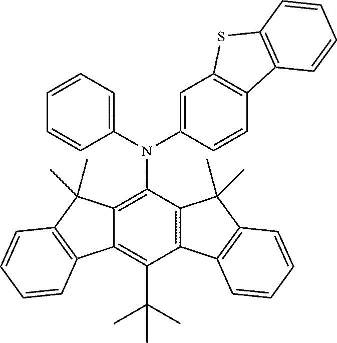 [SMILES: CC(C)(C)c1c2c(c(N(c3ccccc3)c3ccc4c(c3)sc3ccccc34)c3c1-c1ccccc1C3(C)C)C(C)(C)c1ccccc1-2] | 34 | 640.7 |
| 11 | 135 | 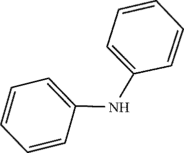 [SMILES: c1ccc(Nc2ccccc2)cc1] | 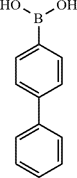 [SMILES: OB(O)c1ccc(-c2ccccc2)cc1] | 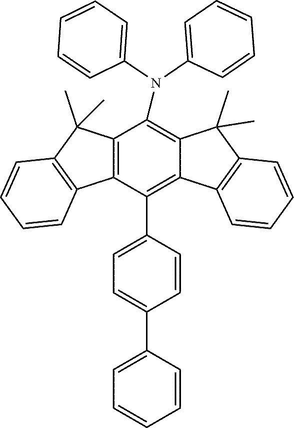 [SMILES: CC1(C)c2ccccc2-c2c(-c3ccc(-c4ccccc4)cc3)c3c(c(N(c4ccccc4)c4ccccc4)c21)C(C)(C)c1ccccc1-3] | 32 | 630.5 |
| 12 | 140 | 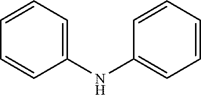 [SMILES: c1ccc(Nc2ccccc2)cc1] | 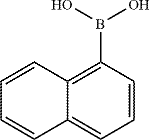 [SMILES: OB(O)c1cccc2ccccc12] | 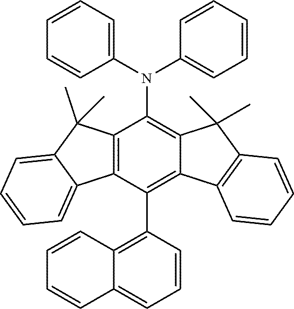 [SMILES: CC1(C)c2ccccc2-c2c(-c3cccc4ccccc34)c3c(c(N(c4ccccc4)c4ccccc4)c21)C(C)(C)c1ccccc1-3] | 28 | 604.0 |
| 13 | 144 | 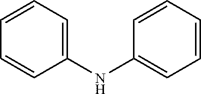 [SMILES: c1ccc(Nc2ccccc2)cc1] | 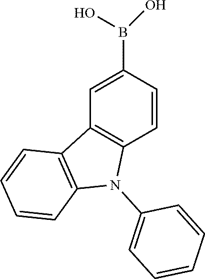 [SMILES: OB(O)c1ccc2c(c1)c1ccccc1n2-c1ccccc1] | 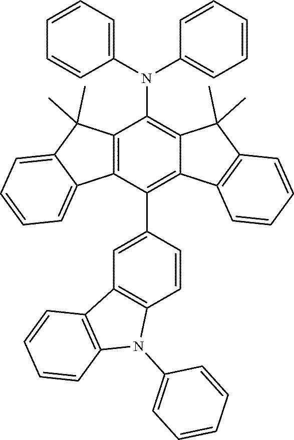 [SMILES: CC1(C)c2ccccc2-c2c(-c3ccc4c(c3)c3ccccc3n4-c3ccccc3)c3c(c(N(c4ccccc4)c4ccccc4)c21)C(C)(C)c1ccccc1-3] | 32 | 719.3 |
| 14 | 141 | 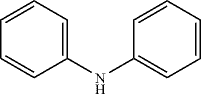 [SMILES: c1ccc(Nc2ccccc2)cc1] | 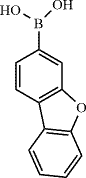 [SMILES: OB(O)c1ccc2c(c1)oc1ccccc12] | 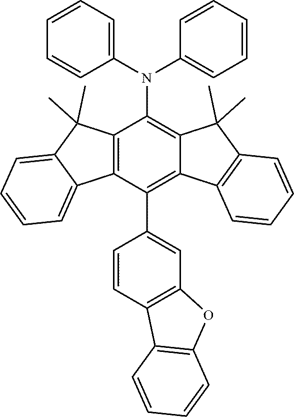 [SMILES: CC1(C)c2ccccc2-c2c(-c3ccc4c(c3)oc3ccccc34)c3c(c(N(c4ccccc4)c4ccccc4)c21)C(C)(C)c1ccccc1-3] | 35 | 644.6 |
| 15 | 136 | 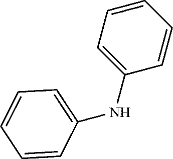 [SMILES: c1ccc(Nc2ccccc2)cc1] | 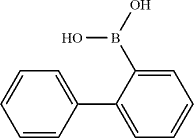 [SMILES: OB(O)c1ccccc1-c1ccccc1] | 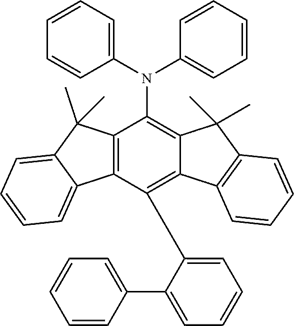 [SMILES: CC1(C)c2ccccc2-c2c(-c3ccccc3-c3ccccc3)c3c(c(N(c4ccccc4)c4ccccc4)c21)C(C)(C)c1ccccc1-3] | 35 | 630.3 |
| 16 | 159 | 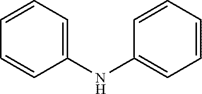 [SMILES: c1ccc(Nc2ccccc2)cc1] | 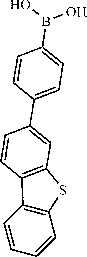 [SMILES: OB(O)c1ccc(-c2ccc3c(c2)sc2ccccc23)cc1] | 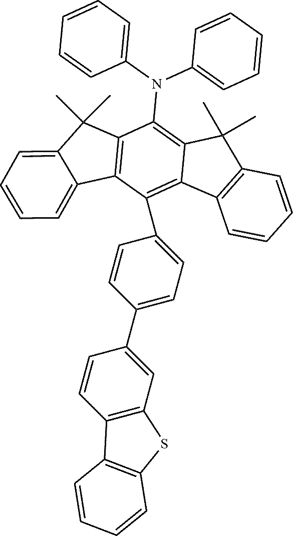 [SMILES: CC1(C)c2ccccc2-c2c(-c3ccc(-c4ccc5c(c4)sc4ccccc45)cc3)c3c(c(N(c4ccccc4)c4ccccc4)c21)C(C)(C)c1ccccc1-3] | 33 | 736.5 |
| 17 | 176 | 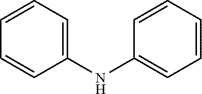 [SMILES: c1ccc(Nc2ccccc2)cc1] | 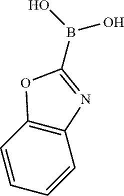 [SMILES: OB(O)c1nc2ccccc2o1] | 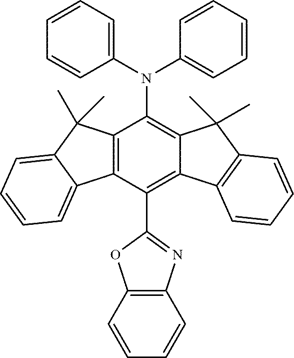 [SMILES: CC1(C)c2ccccc2-c2c(-c3nc4ccccc4o3)c3c(c(N(c4ccccc4)c4ccccc4)c21)C(C)(C)c1ccccc1-3] | 33 | 595.3 |
| 18 | 221 | 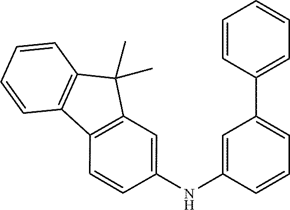 [SMILES: CC1(C)c2ccccc2-c2ccc(Nc3cccc(-c4ccccc4)c3)cc21] | 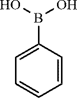 | 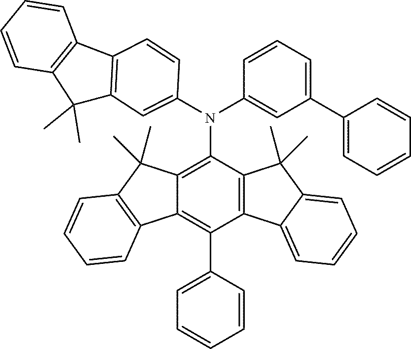 [SMILES: CC1(C)c2ccccc2-c2ccc(N(c3cccc(-c4ccccc4)c3)c3c4c(c(-c5ccccc5)c5c3C(C)(C)c3ccccc3-5)-c3ccccc3C4(C)C)cc21] | 36 | 746.4 |
| 19 | 231 | 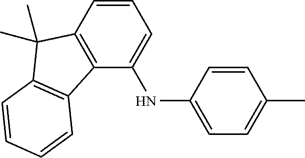 | 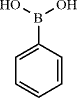 | 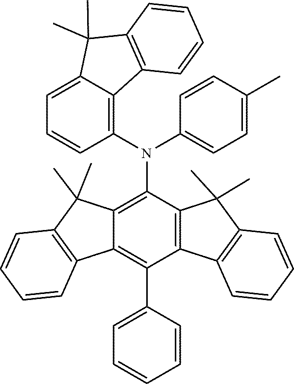 [SMILES: Cc1ccc(N(c2cccc3c2-c2ccccc2C3(C)C)c2c3c(c(-c4ccccc4)c4c2C(C)(C)c2ccccc2-4)-c2ccccc2C3(C)C)cc1] | 37 | 684.4 |
| 20 | 187 | 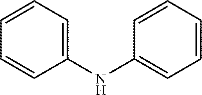 [SMILES: c1ccc(Nc2ccccc2)cc1] | 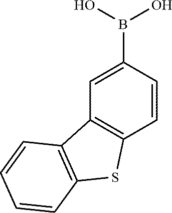 [SMILES: OB(O)c1ccc2sc3ccccc3c2c1] | 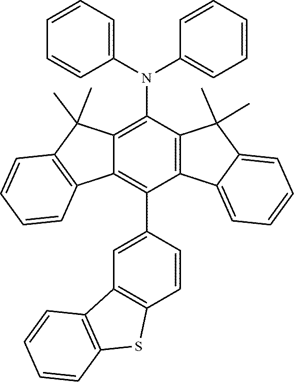 [SMILES: CC1(C)c2ccccc2-c2c(-c3ccc4sc5ccccc5c4c3)c3c(c(N(c4ccccc4)c4ccccc4)c21)C(C)(C)c1ccccc1-3] | 32 | 660.3 |
| 21 | 220 | 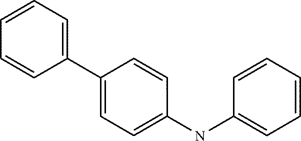 [SMILES: c1ccc(Nc2ccc(-c3ccccc3)cc2)cc1] | 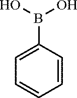 | 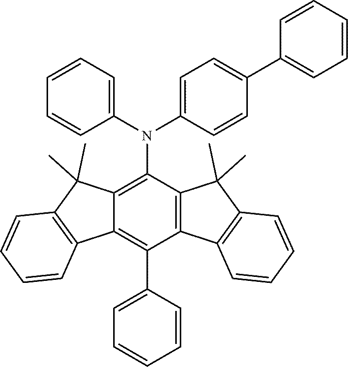 [SMILES: CC1(C)c2ccccc2-c2c(-c3ccccc3)c3c(c(N(c4ccccc4)c4ccc(-c5ccccc5)cc4)c21)C(C)(C)c1ccccc1-3] | 40 | 630.4 |
| 22 | 236 | 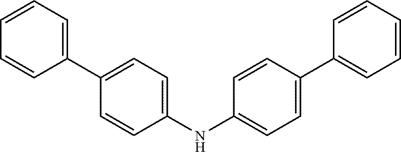 [SMILES: c1ccc(-c2ccc(Nc3ccc(-c4ccccc4)cc3)cc2)cc1] | 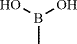 | 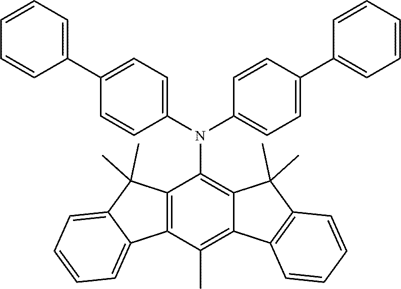 [SMILES: Cc1c2c(c(N(c3ccc(-c4ccccc4)cc3)c3ccc(-c4ccccc4)cc3)c3c1-c1ccccc1C3(C)C)C(C)(C)c1ccccc1-2] | 36 | 644.3 |

NMR of the compound 135: ¹H NMR (CDCl₃, 300 MHz): δ (ppm) 8.24-8.20 (m, 2H), 8.07-8.02 (m, 2H), 7.99-7.95 (m, 2H), 7.82-7.78 (m, 5H), 7.72-7.68 (m, 4H), 7.53-7.48 (m, 6H), 7.44-7.38 (m, 6H), 2.64 (s, 12H).

### Synthesis of Raw Materials 24E and 27E in Table 2

1) Synthesis of the raw material 24E:

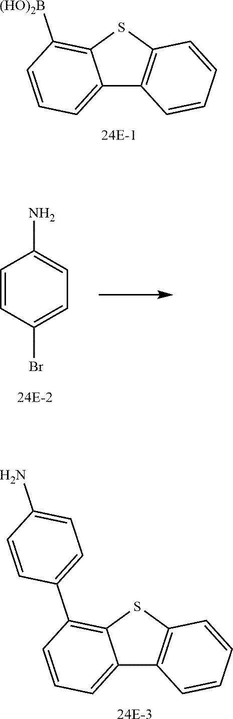

(1) 24E-1 (20 mmol), 24E-2 (21 mmol) and potassium carbonate (40 mmol) were sequentially added into a three-necked flask equipped with a thermometer and a condenser tube, then 50mL of toluene, 10mL of ethanol and 5mL of water were sequentially added, air in the reaction flask was replaced with nitrogen, Pd(PPh₃)₄ (0.05 mmol) was added under the protection of nitrogen, heating was started while magnetically stirring, the system was heated to 65 to 70° C. and reacted for 6h, then cooled to room temperature, the reaction liquid was allowed to stand for layering, water phase layer was extracted with 30mL of toluene, organic phases were mixed, washed with water to be neutral, dried over 5 g of anhydrous sodium sulfate, and filtered to remove the drying agent to obtain a filter cake, the filter cake was subjected to drip washing wih a small amount of toluene, and the filtrates obtained were mixed, allowed to pass through a column, and concentrated to be dry, 20 mL of ethanol was added, and filtering was performed to obtain intermediate 24E-3 (18 mmol, yield: 90%).

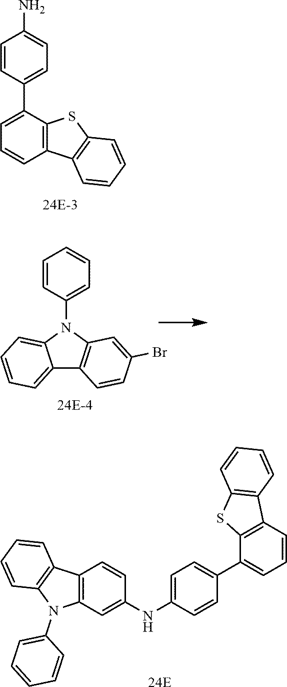

(2) The intermediate 24E-3 (15 mmol), raw material 24E-4 (14 mmol), sodium tert-butoxide (22.5 mmol) and 30 mL of toluene were sequentially added into a four-necked flask equipped with a mechanical stirrer, stirring was started, nitrogen was introduced, the system was heated to 110 to 115° C., refluxing and water segregation were performed for 1h, then Pd₂(dba)₃ (0.03 mmol) and X-phos (0.06 mmol) were added, and the reaction was carried out for 4h while the reflux state was continuously kept. The reaction liquid was cooled to the room temperature, then poured into water with stirring, liquid separation was performed, water phase was extracted twice with 20 mL of toluene, organic phases were mixed, washed twice with water, and dried over anhydrous sodium sulfate, the obtained filtrate was concentrated until the remaining volume was 10 mL, and cooled to room temperature, and filtering was performed to obtain 24E (12 mmol, yield: 86%).

2) Synthesis of raw material 27E The raw material 27E was prepared with reference to the step (2) in the synthetic method of the raw material 24E, except that 24E-3 was replaced by

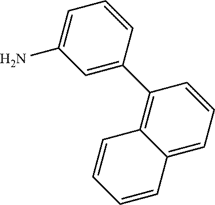 [SMILES: Nc1cccc(-c2cccc3ccccc23)c1]

to obtain the raw material 27E (9.9 mmol, yield: 71%).

Synthesis of intermediate I

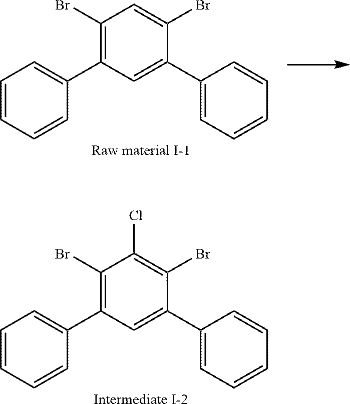

(1) Nitrogen (0.100 L/min) was introduced into a three-necked flask equipped with a mechanical stirrer and a thermometer for replacement for 15 min, raw material I-1 (38.6 g, 100 mmol) and 200 mL of tetrahydrofuran were sequentially added, stirring was started, the flask was cooled to -65° C. to -60° C., lithium diisopropylamide (11.8 g, 110 mmol) was dropwise added, the temperature was continuously kept for 1 hour after dropwise adding finished, a tetrahydrofuran (50 mL) solution of hexachloroethane (60 mmol) was dropwise added, the temperature was continuously kept for 1 hour after dropwise adding finished, 100 mL of water was added, the system was extracted with 200 mL of dichloromethane, water phase was extracted with 50 mL of dichloromethane, organic phases were mixed, washed with water twice, dried over 2 g of anhydrous sodium sulfate, filtered, and concentrated (40 to 45° C., -0.06 MPa to -0.05 MPa) until no liquid flowed out, and the obtained crude product was subjected to column chromatography with ethyl acetate and petroleum ether at a ratio of 1:12 (v/v) to obtain intermediate I-2 (16.8 g, yield: 40%).

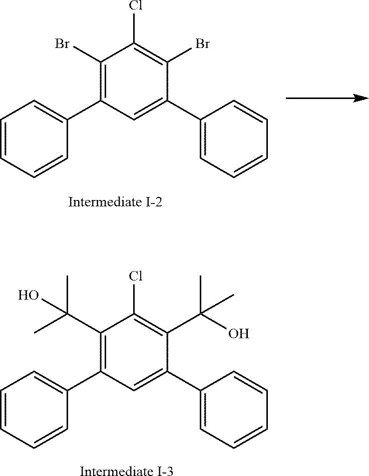

(2) Nitrogen (0.100 L/min) was introduced into a three-necked flask equipped with a mechanical stirrer and a thermometer for replacement for 15 min, the intermediate I-2 (16.8 g, 40 mmol) and 60 mL of tetrahydrofuran were sequentially added, stirring was started, the flask was cooled to -65° C. to -60° C., a n-hexane solution (with a concentration of 2 mol/L) of n-butyllith ium (90 mmol) was dropwise added, the temperature was continuously kept for 1 hour after dropwise adding finished, acetone (100 mmol) was dropwise added, the temperature was continuously kept for 1h after dropwise adding finished, 60 mL of water was added, the system was extracted with 100 mL of dichloromethane, water phase was extracted with 30 mL of dichloromethane, organic phases were mixed, washed with water twice, dried over 2 g of anhydrous sodium sulfate, filtered, and organic phase was concentrated (40 to 45° C., -0.06 MPa to -0.05 MPa) until no liquid flowed out, 20 mL of cyclohexane was added, and filtering was performed to obtain intermediate I-3 (9.8 g, yield: 64%).

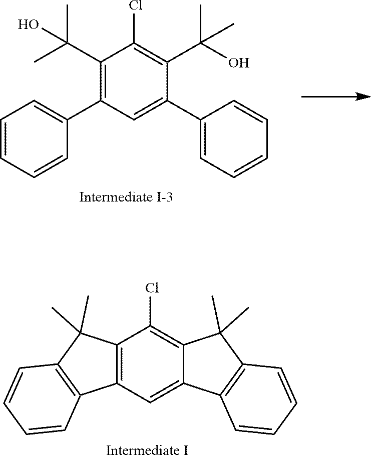

(3) Nitrogen (0.100 L/min) was introduced into a three-necked flask equipped with a mechanical stirrer, a thermometer and an Allihn condenser for replacement for 15 min, the intermediate I-3 (7.6 g, 20 mmol) and 40 mL of dichloroethane were sequentially added, stirring was started, the flask was cooled to 0 to 5° C., concentrated sulfuric acid (50 mmol) was dropwise added, the temperature was continuously kept for 1h after dropwise adding finished, 30 mL of water was added, liquid separation was performed, water phase was extracted with 20 mL of dichloroethane, organic phases were mixed, washed with water twice, dried over 2 g of anhydrous sodium sulfate, filtered, and organic phase was concentrated (40 to 45° C., -0.06 MPa to -0.05 MPa) until no liquid flowed out, 10mL of ethanol was added, and filtering was performed to obtain the intermediate I (3.8 g, yield: 55%).

Synthesis example 23: Synthesis of compound 184

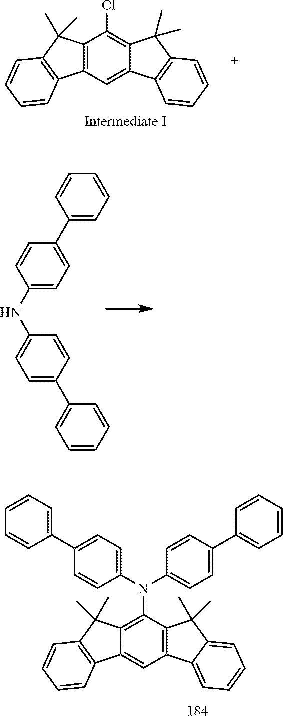

The intermediate I (1.29 g, 3.73 mmol), bis(4-biphenyl)amine (1.44 g, 4.48 mmol), sodium tert-butoxide (0.90 g, 9.33 mmol) and 20 mL of toluene were sequentially added into a four-necked flask equipped with a mechanical stirrer, stirring was started, nitrogen was introduced, the flask was heated to 110° C., refluxing and water segregation were performed for 1h, then Pd₂(dba)₃ (0.03 g, 0.03 mmol) and X-phos (0.037 mmol) were added, and the reaction was carried out overnight while the reflux state was continuously kept. The reaction liquid was cooled to room temperature then was poured into water with stirring, liquid separation was performed, water phase was extracted twice with toluene, organic phases were mixed, washed twice with water, dried over anhydrous sodium sulfate, and concentrated to be dry to obtain a brown yellow oily substance, and the oily substance was purified by column chromatography to obtain a white solid, namely the compound 184 (1.60 g, yield: 68%), and mass spectrum: m/z = 630.3 [M+H]⁺.

### Synthesis Examples 24 to 28

Compounds shown in Table 3 were synthesized by referring to the method in synthesis example 23, except that bis(4-biphenyl)amine was replaced by a raw material IE in Table 3, and the yield and mass spectrum characterization data of the synthesized compounds are shown in Table 3.

TABLE 3

| Synthesis example No. | Compound No. | Raw material IE | Compound structure | Yield, % | Mass spectrum (m/z), [M+H]⁺ |
|---|---|---|---|---|---|
| 24 | 185 | 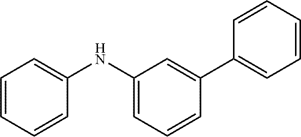 [SMILES: c1ccc(Nc2cccc(-c3ccccc3)c2)cc1] | 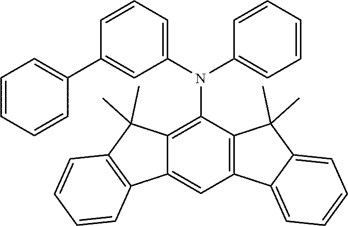 [SMILES: CC1(C)c2ccccc2-c2cc3c(c(N(c4ccccc4)c4cccc(-c5ccccc5)c4)c21)C(C)(C)c1ccccc1-3] | 72 | 554.3 |
| 25 | 186 | 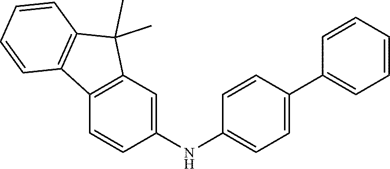 [SMILES: CC1(C)c2ccccc2-c2ccc(Nc3ccc(-c4ccccc4)cc3)cc21] | 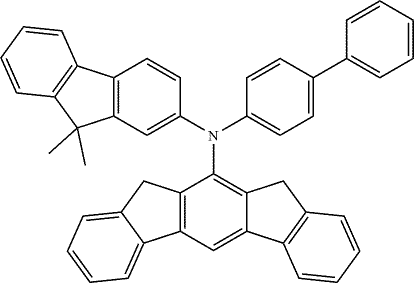 [SMILES: CC1(C)c2ccccc2-c2ccc(N(c3ccc(-c4ccccc4)cc3)c3c4c(cc5c3Cc3ccccc3-5)-c3ccccc3C4)cc21] | 65 | 670.4 |
| 26 | 188 | 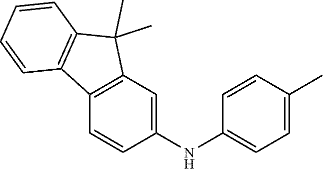 [SMILES: Cc1ccc(Nc2ccc3c(c2)C(C)(C)c2ccccc2-3)cc1] | 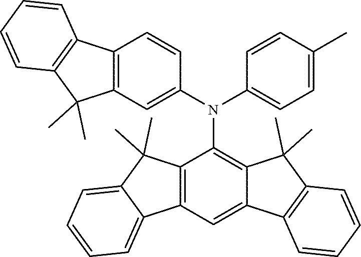 [SMILES: Cc1ccc(N(c2ccc3c(c2)C(C)(C)c2ccccc2-3)c2c3c(cc4c2C(C)(C)c2ccccc2-4)-c2ccccc2C3(C)C)cc1] | 60 | 608.3 |
| 27 | 209 | 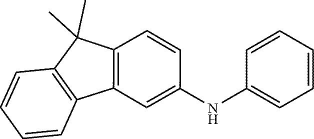 [SMILES: CC1(C)c2ccccc2-c2cc(Nc3ccccc3)ccc21] | 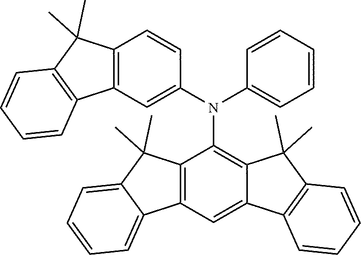 [SMILES: CC1(C)c2ccccc2-c2cc(N(c3ccccc3)c3c4c(cc5c3C(C)(C)c3ccccc3-5)-c3ccccc3C4(C)C)ccc21] | 75 | 594.3 |
| 28 | 189 | 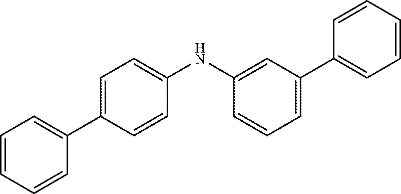 [SMILES: c1ccc(-c2ccc(Nc3cccc(-c4ccccc4)c3)cc2)cc1] | 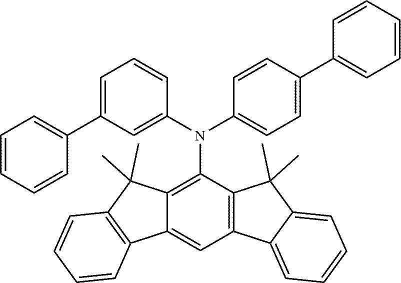 [SMILES: CC1(C)c2ccccc2-c2cc3c(c(N(c4ccc(-c5ccccc5)cc4)c4cccc(-c5ccccc5)c4)c21)C(C)(C)c1ccccc1-3] | 64 | 630.3 |

Synthesis example 29: Synthesis of compound 237

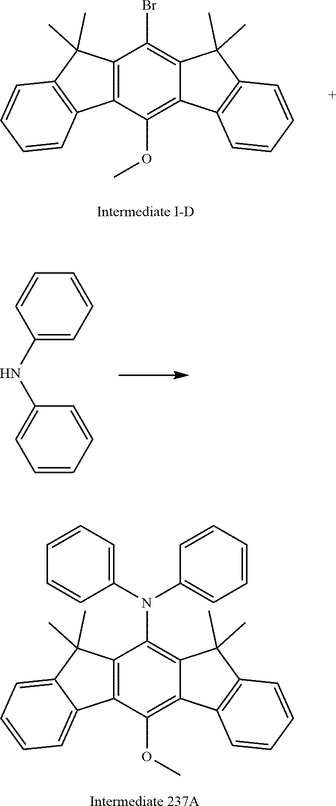

(1) The intermediate I-D (2.30 g, 5.5 mmol), diphenylamine (1.12 g, 6.60 mmol), sodium tert-butoxide (1.32 g, 13.75 mmol) and 25 mL of toluene were sequentially added into a four-necked flask equipped with a mechanical stirrer, stirring was started, nitrogen was introduced, the flask was heated to 110° C., refluxing and water segregation were performed for 1 h, then Pd₂(dba)₃ (0.03 g, 0.03 mmol) and X-phos (0.02 g, 0.037 mmol) were added, and the reaction was carried out overnight while the reflux state was continuously kept. The reaction liquid was cooled to room temperature, then poured into water with stirring, liquid separation was performed, water phase was extracted twice with toluene, organic phases were mixed, washed twice with water, dried over anhydrous sodium sulfate, and concentrated to be dry to obtain a brown yellow oily substance, and the oily substance was separated by column chromatography to obtain a white solid, namely intermediate 237A (2.04 g, yield: 73%).

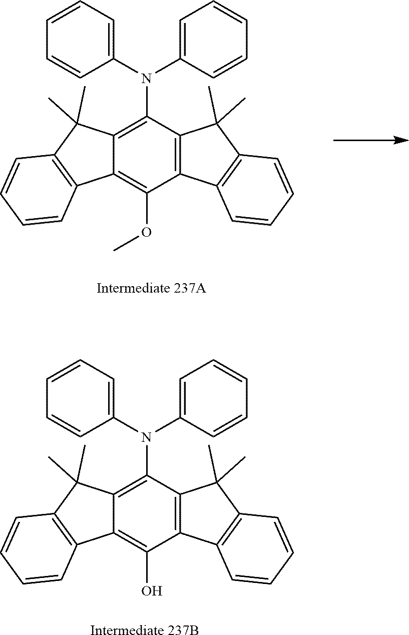

(2) The intermediate 237A (2.00 g, 3.96 mmol) was added into a three-necked flask containing 35 mL of DCM, air in the reaction flask was replaced with nitrogen, the flask was cooled to about -5° C., BBr₃ (1.18 g, 4.75 mmol) was dissolved into 20 mL of DCM, the obtained solution was slowly added into the reaction system under the protection of nitrogen, the reaction was continued to be carried out while maintaining the temperature after dropwise adding finished, and the reaction was stopped until TLC monitored that the raw materials were reacted completely. Water was slowly added for quenching the reaction under an ice bath condition, standing was performed for layering, water phase was extracted with DCM, organic phases were mixed, washed with saturated salt solution once, then dried over anhydrous sodium sulfate for 1.5h, and filtered to remove the drying agent, and the obtained filtrate was concentrated, and separated by passing through a silica gel column to obtain an off-white solid, namely intermediate 237B (1.76 g, yield: 90%).

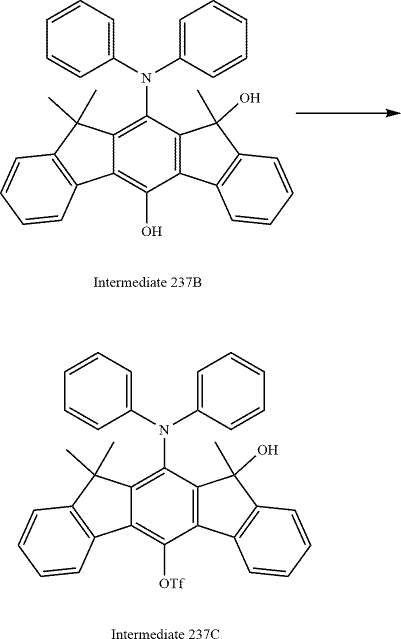

(3) The intermediate 237B (1.76 g, 3.56 mmol) and 20 mL of dichloromethane were sequentially added into a three-necked flask, stirring was started, pyridine (0.56 g, 7.12 mmol) was added, the flask was cooled to -3° C., trifluoromethanesulfonic anhydride (1.11 g, 3.92 mmol) was dropwise added while maintaining the temperature at about 0° C., where dropwise adding was completed within about 1h, the reaction was carried out for 2.0h with temperature holding, then the system was heated naturally to room temperature. 2M hydrochloric acid was added into the reaction liquid with stirring, stirring was performed for 10 min, dichloromethane was added for extracting twice, and organic phase was washed with water twice, dried over anhydrous sodium sulfate for 0.5h, and purified by passing through a chromatographic column to obtain an off-white solid, namely intermediate 237C (1.85 g, yield: 83%).

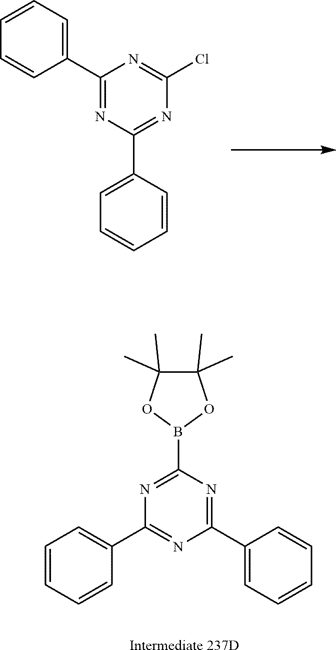

(4) Nitrogen (0.100 L/min) was introduced into a three-necked flask equipped with a mechanical stirrer and a thermometer for replacement for 15 min, 2-chloro-4,6-diphenyl-1,3,5-triazine (10.7 g, 40 mmol), bis(pinacolato)diboron (12.2 g, 48 mmol), potassium acetate (7.9 g, 80 mmol) and 80 mL of 1,4-dioxane were sequentially added, stirring was started, the system was heated to 60° C., bis(tricyclohexylphosphine)palladium dichloride (0.29 g, 0.4 mmol) was added, the obtained system was continuously heated to 85° C., the temperature was kept for 5 h, 60 mL of water was added, extraction was performed with 100 mL of dichloromethane, water phase was extracted with 30 mL of dichloromethane, organic phases were mixed, washed with water twice, dried over 2 g of anhydrous sodium sulfate, filtered, and organic phase was concentrated (40 to 45° C., -0.06 MPa to -0.05 MPa) until no liquid flowed out, 20 mL of cyclohexane was added, and filtering was performed to obtain intermediate 237D (10.77 g, yield: 75%).

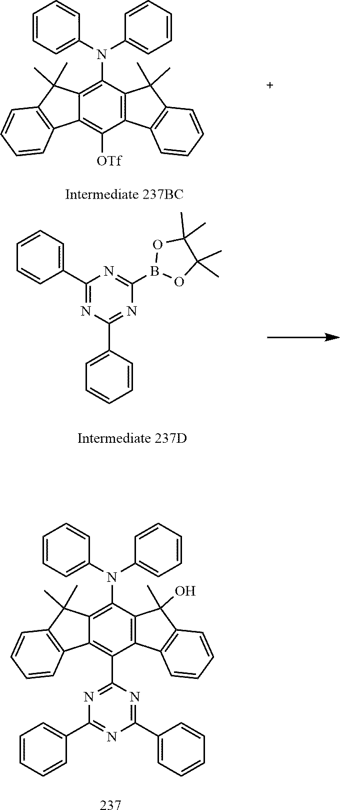

(5) The intermediate 237C (1.90 g, 3.03 mmol), the intermediate 237D (1.09 g, 3.03 mmol) and potassium carbonate (1.05 g, 7.58 mmol) were sequentially added into a three-necked flask equipped with a thermometer and a condenser tube, then 16 mL of toluene, 4 mL of ethanol and 2 mL of water were sequentially added, air in the reaction flask was replaced with nitrogen, Pd(PPh₃)₄ (0.02 g, 0.015 mmol) was added under the protection of nitrogen, heating was started while magnetically stirring, and the system was heated to reflux, the reaction was carried out for 8h, then a sample was taken for detecting, the reaction was stopped when the raw materials were completely reacted, the system was cooled to room temperature, post-treatment was performed, the resulting reaction solution was filtered, and allowed to stand for layering, water phase layer was extracted with toluene, organic phases were mixed, washed to be neutral, dried over anhydrous sodium sulfate for 1.5h, and filtered to remove the drying agent to obtain a filter cake, the filter cake was subj ected to drip washing with a small amount of toluene, and the obtained filtrates were mixed, concentrated, and purified by a chromatographic column to obtain an off-white solid, namely the compound 237 (1.54 g, yield: 72%), and m/z = 709.3 [M+H]⁺.

### Synthesis Examples 30 to 37

Compounds shown in Table 5 were synthesized by referring to the method in synthesis example 29, except that diphenylamine was replaced by the raw material IE in Table 5, 2-chloro-4,6-diphenyl-1,3,5-triazine was replaced by a raw material Ia in Table 5, and the yield (the yield of the last step) and mass spectrum characterization data of the synthesized compounds are shown in Table 5.

TABLE 5

| Synthesis example No. | Compound No. | Raw material IE | Raw material Ia | Compound structure | Yield, % | Mass spectrum, [M+H]⁺ |
|---|---|---|---|---|---|---|
| 30 | 241 | 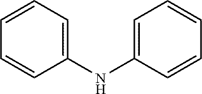 [SMILES: c1ccc(Nc2ccccc2)cc1] | 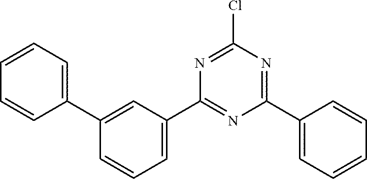 [SMILES: Clc1nc(-c2ccccc2)nc(-c2cccc(-c3ccccc3)c2)n1] | 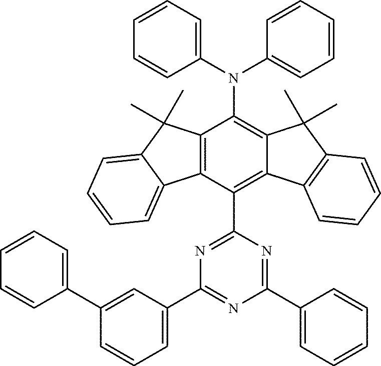 [SMILES: CC1(C)c2ccccc2-c2c(-c3nc(-c4ccccc4)nc(-c4cccc(-c5ccccc5)c4)n3)c3c(c(N(c4ccccc4)c4ccccc4)c21)C(C)(C)c1ccccc1-3] | 65 | 785.4 |
| 31 | 254 | 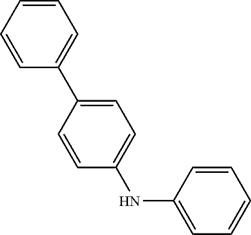 [SMILES: c1ccc(Nc2ccc(-c3ccccc3)cc2)cc1] | 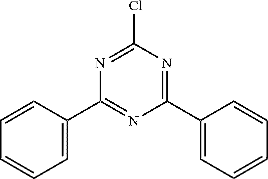 [SMILES: Clc1nc(-c2ccccc2)nc(-c2ccccc2)n1] | 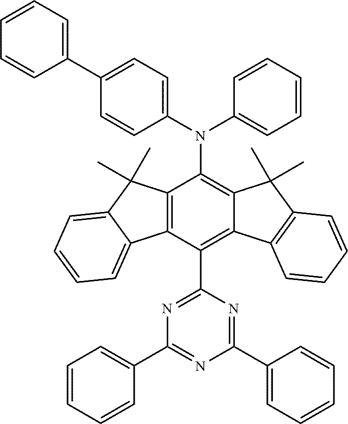 [SMILES: CC1(C)c2ccccc2-c2c(-c3nc(-c4ccccc4)nc(-c4ccccc4)n3)c3c(c(N(c4ccccc4)c4ccc(-c5ccccc5)cc4)c21)C(C)(C)c1ccccc1-3] | 67 | 785.4 |
| 32 | 255 | 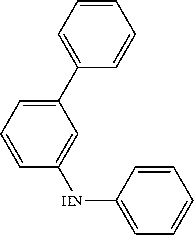 [SMILES: c1ccc(Nc2cccc(-c3ccccc3)c2)cc1] | 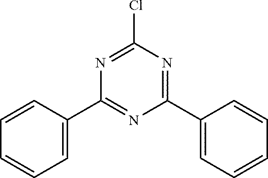 [SMILES: Clc1nc(-c2ccccc2)nc(-c2ccccc2)n1] | 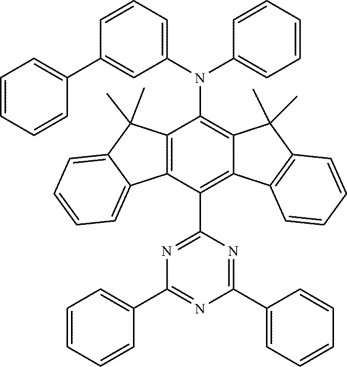 [SMILES: CC1(C)c2ccccc2-c2c(-c3nc(-c4ccccc4)nc(-c4ccccc4)n3)c3c(c(N(c4ccccc4)c4cccc(-c5ccccc5)c4)c21)C(C)(C)c1ccccc1-3] | 62 | 785.4 |
| 33 | 256 | 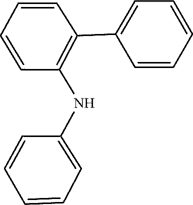 [SMILES: c1ccc(Nc2ccccc2-c2ccccc2)cc1] | 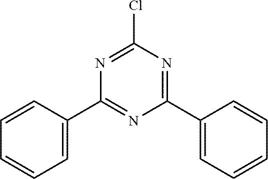 [SMILES: Clc1nc(-c2ccccc2)nc(-c2ccccc2)n1] | 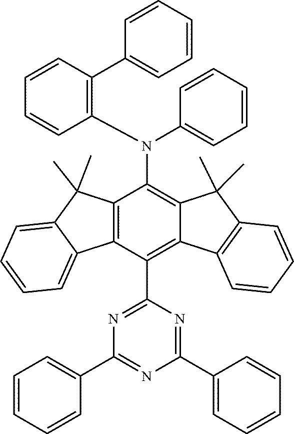 [SMILES: CC1(C)c2ccccc2-c2c(-c3nc(-c4ccccc4)nc(-c4ccccc4)n3)c3c(c(N(c4ccccc4)c4ccccc4-c4ccccc4)c21)C(C)(C)c1ccccc1-3] | 66 | 785.3 |
| 34 | 239 | 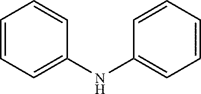 [SMILES: c1ccc(Nc2ccccc2)cc1] | 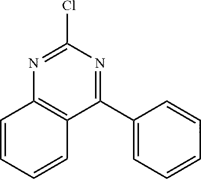 [SMILES: Clc1nc(-c2ccccc2)c2ccccc2n1] | 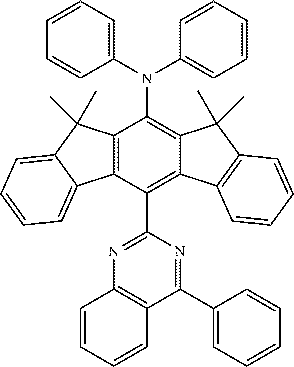 [SMILES: CC1(C)c2ccccc2-c2c(-c3nc(-c4ccccc4)c4ccccc4n3)c3c(c(N(c4ccccc4)c4ccccc4)c21)C(C)(C)c1ccccc1-3] | 53 | 682.4 |
| 35 | 258 | 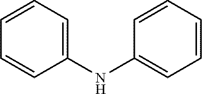 [SMILES: c1ccc(Nc2ccccc2)cc1] | 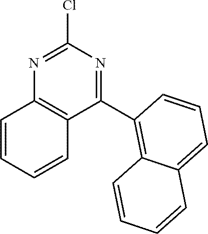 [SMILES: Clc1nc(-c2cccc3ccccc23)c2ccccc2n1] | 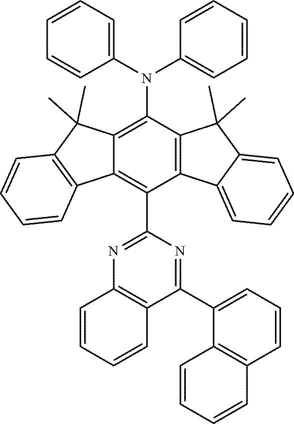 [SMILES: CC1(C)c2ccccc2-c2c(-c3nc(-c4cccc5ccccc45)c4ccccc4n3)c3c(c(N(c4ccccc4)c4ccccc4)c21)C(C)(C)c1ccccc1-3] | 58 | 732.5 |
| 36 | 259 | 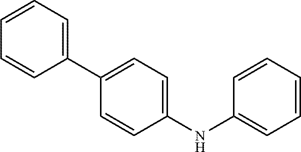 [SMILES: c1ccc(Nc2ccc(-c3ccccc3)cc2)cc1] | 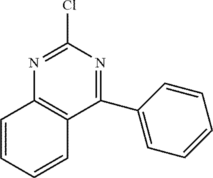 [SMILES: Clc1nc(-c2ccccc2)c2ccccc2n1] | 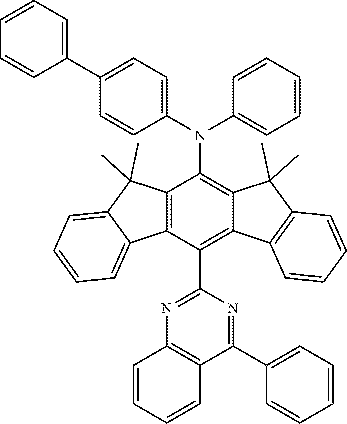 [SMILES: CC1(C)c2ccccc2-c2c(-c3nc(-c4ccccc4)c4ccccc4n3)c3c(c(N(c4ccccc4)c4ccc(-c5ccccc5)cc4)c21)C(C)(C)c1ccccc1-3] | 55 | 758.4 |
| 37 | 260 | | | | 52 | 710.5 |

### Manufacturing of an Organic Electroluminescent Device

### Example 1

A glass bottom plate with an indium tin oxide (ITO) electrode of 1500Å was ultrasonically cleaned by using distilled water and methanol sequentially, dried, and was cleaned for 5 min by using oxygen plasma, then the cleaned anode bottom plate was loaded into vacuum deposition equipment; and

a compound 2T-NATA (CAS: 185690-41-9) was vacuum deposited on the ITO electrode to form a hole injection layer with a thickness of 500Å, the compound 1 was vacuum evaporated on the hole injection layer to form a hole transporting layer with a thickness of 600Å, and TQTPA (CAS: 1142945-07-0) was evaporated on the hole transporting layer to form an electron blocking layer with a thickness of 200Å.

Then a host luminescent material CBP (CAS: 58328-31-7) and a dopant BCzVB (CAS: 62608-15-5) were codeposited on a hole transporting region (namely the electron blocking layer) at a mass ratio of 96:4 to form a luminescent layer with a thickness of 300Å;

TPBi (CAS: 192198-85-9) was vacuum deposited on the luminescent layer to form a hole blocking layer with a thickness of 200Å; DBimiBphen and LiQ were mixed at a weight ratio of 1:1, the mixture was vacuum deposited on the hole blocking layer to form an electron transporting layer with a thickness of 300Å, and LiQ is evaporated on the electron transporting layer to form an electron injection layer with a thickness of 15Å;

Then, magnesium (Mg) and argentum (Ag) were mixed at an evaporation rate of 1:9, and the mixture was vacuum evaporated on the electron injection layer to form a cathode with a thickness of 120Å.

In addition, CP-1 was evaporated on the cathode to form a capping layer (CPL) with a thickness of 630Å, thereby completing manufacturing of the organic luminescent device. The structures of DBimiBphen, LiQ and CP-1 are as follows:

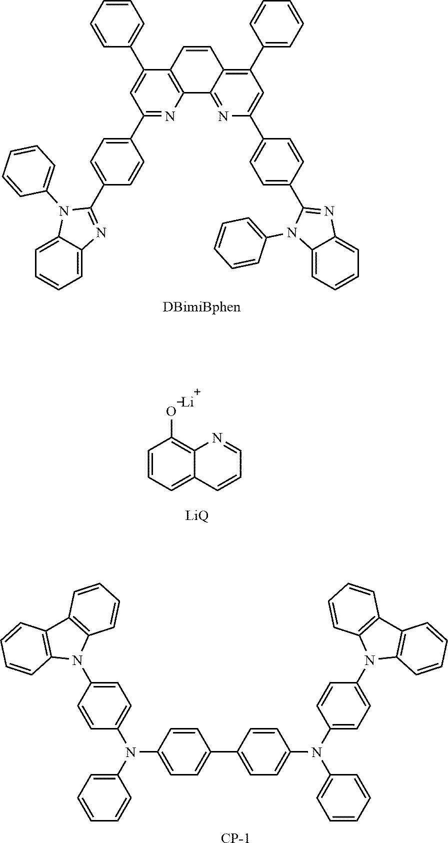

### Examples 2 to 10

The organic electroluminescent device was manufactured according to the same method in example 1, except that when the hole transporting layer was formed, compounds shown in Table 6 were used for replacing the compound 1, so that the organic electroluminescent devices were manufactured respectively.

### Comparative Examples 1 to 3

The organic electroluminescent device was manufactured according to the same method in example 1, except that NPB, a compound A and a compound B were respectively used for replacing the compound 1 when the hole transporting layer was formed, so that the organic electroluminescent devices were respectively manufactured. The structures of NPB, the compound A and the compound B are as follows:

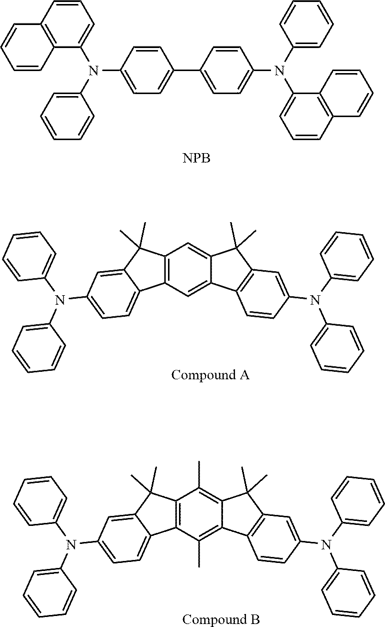

The performance of the devices prepared in the examples and the comparative examples was analyzed, the result is shown in Table 6, wherein the driving voltage, the efficiency and the chromaticity coordinate were tested under a constant current density of 10 mA/cm², and the T95 service life of the device was tested under a constant current density of 15 mA/cm².

TABLE 6

| No. | Hole transporting material | Driving voltage (V) | Current efficiency Cd/A | External quantum efficiency EQE (%) | Chromaticity coordinate (CIEy) | T95 service life (h) |
|---|---|---|---|---|---|---|
| Example 1 | Compound 1 | 3.81 | 6.29 | 13.01 | 0.051 | 289 |
| Example 2 | Compound 4 | 3.89 | 6.46 | 13.32 | 0.053 | 294 |
| Example 3 | Compound 6 | 3.85 | 6.46 | 13.39 | 0.052 | 262 |
| Example 4 | Compound 8 | 3.89 | 6.69 | 13.84 | 0.051 | 245 |
| Example 5 | Compound 24 | 3.88 | 6.46 | 13.36 | 0.052 | 261 |
| Example 6 | Compound 27 | 3.88 | 6.81 | 14.08 | 0.052 | 265 |
| Example 7 | Compound 33 | 3.88 | 6.34 | 13.06 | 0.055 | 249 |
| Example 8 | Compound 36 | 3.93 | 6.35 | 13.12 | 0.054 | 296 |
| Example 9 | Compound 38 | 3.81 | 6.31 | 13.16 | 0.051 | 245 |
| Example 10 | Compound 60 | 3.84 | 6.46 | 13.40 | 0.051 | 265 |
| Comparative example 1 | NPB | 4.60 | 5.50 | 11.08 | 0.052 | 165 |
| Comparative example 2 | Compound A | 4.45 | 6.00 | 12.42 | 0.051 | 229 |
| Comparative example 3 | Compound B | 4.23 | 6.10 | 12.60 | 0.050 | 217 |

According to the above results, it can be seen that the driving voltage of the organic electroluminescent devices prepared in examples 1 to 10 is at least 0.3 V lower than that of the organic electroluminescent devices prepared in comparative examples to 3, and the service life of the devices is at least prolonged by 7.0%. In addition, the organic electroluminescent devices in examples 1 to 10 also have relatively high luminous efficiency. Therefore, compared with the comparative examples, the organic electroluminescent devices prepared in examples 1 to 10 have lower driving voltage and longer service life, and also have higher photoelectric efficiency.

### Example 11

An anode was prepared by the following processes: a substrate (manufactured by Corning) with an ITO thickness of 1200 Å was cut into a size of 40 mm × 40 mm × 0.7 mm to be prepared into an experimental substrate with a cathode lap joint area, an anode and an insulating layer pattern by adopting a photoetching process, and surface treatment was performed by utilizing ultraviolet ozone and O₂:N₂ plasma to increase the work function of the anode (the experiment substrate), and to remove scum.

m-MTDATA was vacuum evaporated on the experiment substrate (the anode) to form a hole injection layer with a thickness of 100Å, and TPD was vacuum evaporated on the hole injection layer to form a hole transporting layer with a thickness of 800Å. A compound 135 was evaporated on the hole transporting layer to form an electron blocking layer with a thickness of 260Å.

Then α,β-ADN as a host was doped with N-BDAVBi at a film thickness ratio of 100:3 to form a luminescent layer with the thickness of 310Å.

3TPYMB and LiQ were mixed at a weight ratio of 1:1, and the mixture was was allowed to form an electron transporting layer with the thickness of 280Å by an evaporation process. Then, LiQ was evaporated on the electron transporting layer to form an electron injection layer with a thickness of 14 Å.

Then, magnesium (Mg) and argentum (Ag) were mixed at an evaporation rate of 1:9, and the mixture was vacuum evaporated on the electron injection layer to form a cathode with a thickness of 120 Å.

In addition, CP-1 was evaporated on the cathode to form a capping layer (CPL) with a thickness of 650Å, thereby completing manufacturing of the organic luminescent device. The chemical structures of main materials used for preparing the device are as follows.

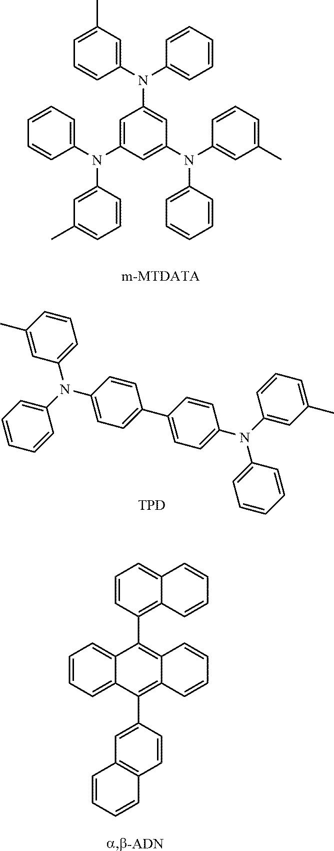

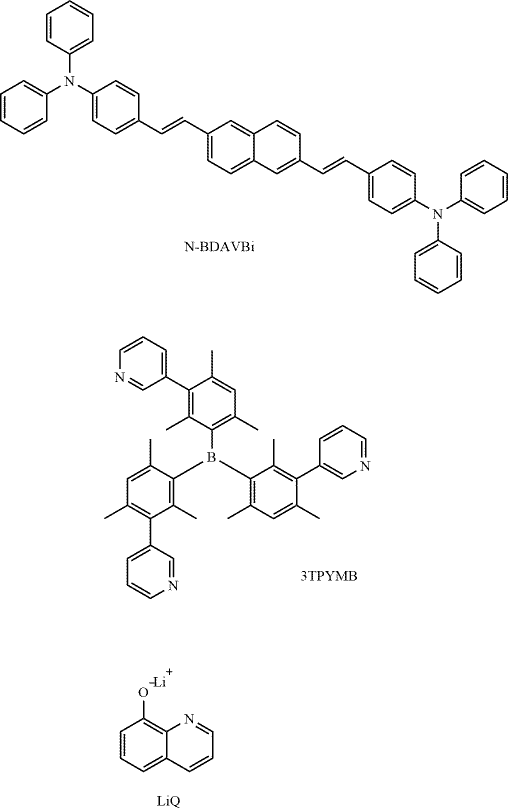

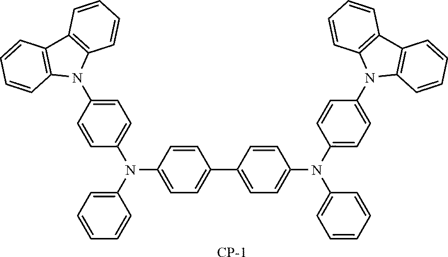 [SMILES: c1ccc(N(c2ccc(-c3ccc(N(c4ccccc4)c4ccc(-n5c6ccccc6c6ccccc65)cc4)cc3)cc2)c2ccc(-n3c4ccccc4c4ccccc43)cc2)cc1 ; "CP-1"]

### Examples 12 to 27

The organic electroluminescent device was manufactured by the same method as in example 11, except that the compound 135 was replaced by compounds shown in Table 7 when the electron blocking layer was formed.

### Comparative Examples 4 to 5

The organic electroluminescent device was manufactured by the same method in example 11, except that the compound 135 was replaced by a compound C and a compound D, respectively when the electron blocking layer was formed. The chemical structures of the compound C and the compound D are as follows:

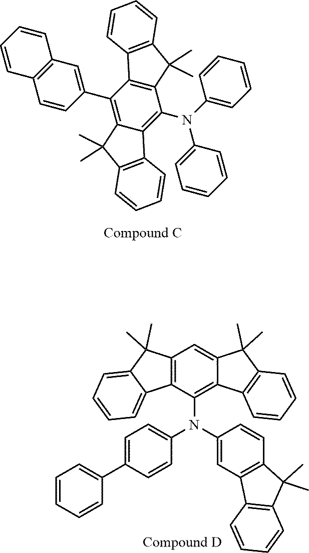

The T95 service life of the prepared organic electroluminescent device was tested under the condition of 15 mA/cm², and the driving voltage, the efficiency and the chromaticity coordinate of the prepared organic electroluminescent device were tested under a constant current density of 10 mA/cm², and the test results are shown in Table 7.

TABLE 7

| No. | Electron blocking layer | Driving voltage (V) | Current efficiency Cd/A | Chromaticity coordinate CIEy | External quantum efficiency EQE (%) | T95 service life (h) |
|---|---|---|---|---|---|---|
| Example 11 | Compound 135 | 3.93 | 6.40 | 0.051 | 13.18 | 227 |
| Example 12 | Compound 136 | 3.91 | 6.51 | 0.050 | 13.28 | 215 |
| Example 13 | Compound 140 | 3.85 | 6.40 | 0.050 | 13.16 | 217 |
| Example 14 | Compound 141 | 3.89 | 6.46 | 0.049 | 13.24 | 214 |
| Example 15 | Compound 144 | 3.92 | 6.38 | 0.050 | 13.14 | 198 |
| Example 16 | Compound 159 | 3.94 | 6.43 | 0.050 | 13.20 | 184 |
| Example 17 | Compound 184 | 3.95 | 6.44 | 0.051 | 13.22 | 232 |
| Example 18 | Compound 185 | 3.93 | 6.55 | 0.050 | 13.49 | 226 |
| Example 19 | Compound 186 | 3.90 | 6.38 | 0.052 | 13.16 | 234 |
| Example 20 | Compound 187 | 3.92 | 6.53 | 0.049 | 13.31 | 211 |
| Example 21 | Compound 188 | 3.94 | 6.41 | 0.051 | 13.18 | 229 |
| Example 22 | Compound 189 | 3.92 | 6.39 | 0.050 | 13.15 | 230 |
| Example 23 | Compound 209 | 3.88 | 6.45 | 0.049 | 13.22 | 235 |
| Example 24 | Compound 220 | 3.95 | 6.35 | 0.051 | 13.09 | 232 |
| Example 25 | Compound 221 | 3.90 | 6.54 | 0.053 | 13.39 | 225 |
| Example 26 | Compound 231 | 3.96 | 6.36 | 0.052 | 13.04 | 212 |
| Example 27 | Compound 236 | 3.91 | 6.37 | 0.050 | 13.12 | 223 |
| Comparative example 4 | Compound C | 4.22 | 5.91 | 0.051 | 12.13 | 162 |
| Comparative example 5 | Compound D | 4.17 | 6.02 | 0.049 | 12.37 | 157 |

In combination with the result in Table 7, the driving voltage of the organic electroluminescent devices prepared in examples 11 to 27 is at least 0.21 V lower than that of the organic electroluminescent devices prepared in comparative examples 4 to 5; the T95 service life of the devices in examples 11 to 27 is at least improved by 13.6% compared with the T95 service life of the devices in comparative examples 4 to 5, and meanwhile, the devices in examples 11 to 27 also have relatively high luminous efficiency. Therefore, compared with the comparative examples, the organic electroluminescent devices prepared in examples 11 to 27 can further reduce the driving voltage of the device and prolong the service life of the device while ensuring that the device has higher luminous efficiency.

### Example 28

An anode was prepared by the following processes: a substrate (manufactured by Corning) with an ITO thickness of 1100 Å was cut into a size of 40 mm × 40 mm × 0.7 mm to be prepared into an experimental substrate with a cathode lap joint area, an anode and an insulating layer pattern by adopting a photoetching process, and surface treatment was performed by utilizing ultraviolet ozone and O₂:N₂ plasma to increase the work function of the anode (the experiment substrate), and to remove scum.

NATA was vacuum-evaporated on the experiment substrate (the anode) to form a hole injection layer with a thickness of 100Å, and DPFL-NPB was evaporated on the hole injection layer to form a hole transporting layer with a thickness of 800Å. EB-1 was vacuum evaporated on the hole transporting layer to form an electron blocking layer with a thickness of 850Å.

The compound 237 as a host material was doped with DCJT as a guest material, and evaporated at a film thickness ratio of 100:3 on the electron blocking layer to form a luminescent layer with a thickness of 310Å.

TPBi and LiQ were mixed at a weight ratio of 1:1, and the mixture was evaporated to form an electron transporting layer with a thickness of 220Å, LiQ was evaporated on the electron transporting layer to form an electron injection layer with a thickness of 16Å, then magnesium (Mg) and argentum (Ag) were mixed at an evaporation rate of 1:9, and the mixture was vacuum evaporated on the electron injection layer to form a cathode with a thickness of 105Å.

In addition, CP-1 was evaporated on the cathode to form an organic capping layer (CPL) with a thickness of 700Å, thereby completing manufacturing of the organic luminescent device. The structures of main materials used for preparing the device are as follows.

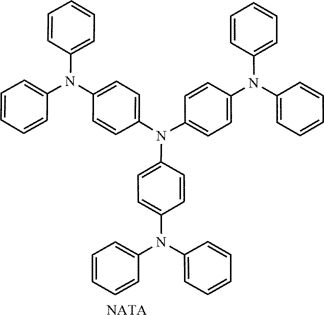

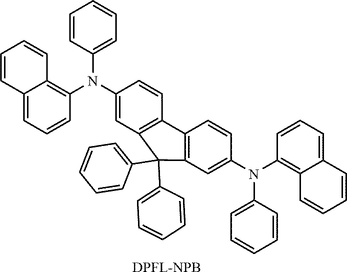

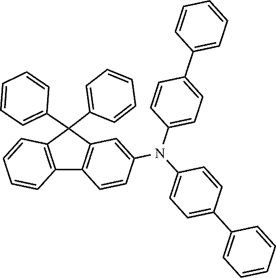 [SMILES: c1ccc(-c2ccc(N(c3ccc(-c4ccccc4)cc3)c3ccc4c(c3)C(c3ccccc3)(c3ccccc3)c3ccccc3-4)cc2)cc1]

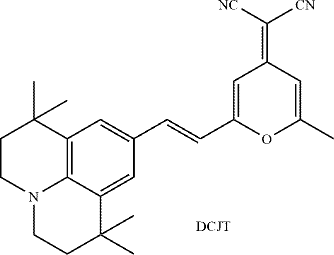

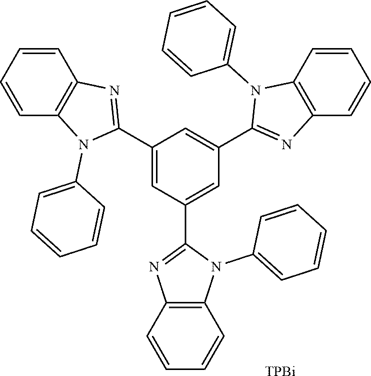

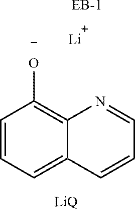

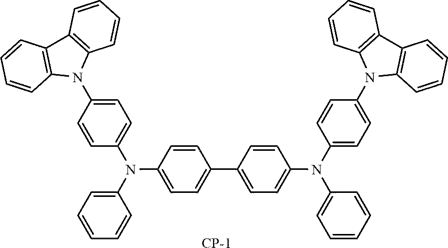 [SMILES: c1ccc(N(c2ccc(-c3ccc(N(c4ccccc4)c4ccc(-n5c6ccccc6c6ccccc65)cc4)cc3)cc2)c2ccc(-n3c4ccccc4c4ccccc43)cc2)cc1 ; "CP-1"]

### Examples 29 to 37

The organic electroluminescent device was manufactured by the same method as in example 28, except that the compound 237 was replaced by the compounds shown in Table 8 as a luminescent host when the luminescent layer was formed.

### Comparative Example 6

The organic electroluminescent device was manufactured by the same method as in example 28, except that the compound 237 was replaced by Compound E as a luminescent host when the luminescent layer was formed, wherein structural formula of Compound E is shown below.

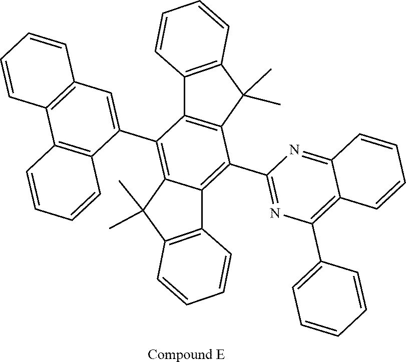

The T95 service life of the prepared organic electroluminescent device was tested under the condition of 15 mA/cm², and the driving voltage, the efficiency and the chromaticity coordinate of the prepared organic electroluminescent device were tested under a constant current density of 10 mA/cm², and the result is shown in Table 8.

TABLE 8

| No. | Light-emitting host material | Driving voltage (V) | Current efficiency Cd/A | Chromaticity coordinate CIEx | External quantum efficiency EQE (%) | T95 service life (h) |
|---|---|---|---|---|---|---|
| Example 28 | Compound 237 | 4.05 | 33.52 | 0.678 | 24.75 | 317 |
| Example 29 | Compound 241 | 4.02 | 34.16 | 0.677 | 26.67 | 312 |
| Example 30 | Compound 254 | 4.06 | 33.81 | 0.680 | 25.58 | 313 |
| Example 31 | Compound 255 | 4.02 | 33.61 | 0.678 | 25.12 | 325 |
| Example 32 | Compound 256 | 4.07 | 33.12 | 0.679 | 24.58 | 313 |
| Example 33 | Compound 239 | 4.08 | 34.45 | 0.678 | 27.15 | 338 |
| Example 34 | Compound 258 | 4.09 | 33.85 | 0.677 | 25.89 | 341 |
| Example 35 | Compound 259 | 4.02 | 33.67 | 0.680 | 25.31 | 316 |
| Example 36 | Compound 260 | 4.01 | 32.94 | 0.681 | 23.86 | 325 |
| Example 37 | Compound 176 | 4.04 | 32.68 | 0.680 | 25.72 | 309 |
| Comparative example 6 | Compound E | 4.14 | 28.36 | 0.679 | 21.57 | 244 |

In combination with the result shown in Table 8, the current efficiency of the organic electroluminescent devices prepared in examples 28 to 37 is at least improved by 15.2% compared with that of comparative example 6, the external quantum efficiency is at least improved by 10.6%, and the T95 service life of the organic electroluminescent devices in examples 28 to 37 is at least improved by 26.6% compared with that of comparative example 6. In addition, the organic electroluminescent devices prepared in examples 28 to 37 also have relatively low driving voltage. Therefore, the nitrogen-containing compound provided by the present disclosure used as a host material, can further improve the service life and the photoelectric efficiency of the device under the condition of ensuring that the device has relatively low driving voltage.

Although the present disclosure is disclosed as above in examples, it is not intended to limit the claims. Those skilled in the art can make several possible changes and modifications without departing from the concept of the present disclosure. Therefore, the protection scope of the present disclosure should be subject to the scope defined in the claims of the present disclosure.

1. A nitrogen-containing compound, having a structure as shown in chemical formula 1: 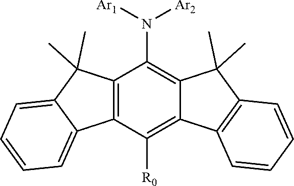
wherein Ar₁ and Ar₂ are the same or different, and are each independently selected from a substituted or unsubstituted C6-C30 aryl, or a substituted or unsubstituted C3-C30 heteroaryl;_ and Ar₁ and Ar₂ are each independently selected from substituted or unsubstituted phenyl, substituted or unsubstituted naphthyl, substituted or unsubstituted biphenyl, substituted or unsubstituted fluorenyl, substituted or unsubstituted anthryl, substituted or unsubstituted phenanthryl, substituted or unsubstituted dibenzofuranyl, substituted or unsubstituted dibenzothienyl, or N-phenylcarbazolyl; or a new group formed by connecting any two above groups via a single bond; the substituents in Ar₁ and Ar₂ are each independently selected from deuterium, methyl, ethyl, n-propyl, isopropyl, tert-butyl, cyano, or fluorine, the number of the substituents is one or two or more, and when the number of the substituents is two or more, each substituent is the same or different; and
R₀ is selected from hydrogen, a C1-C6 alkyl, a substituted or unsubstituted C6-C30 aryl, or a substituted or unsubstituted C3-C30 heteroaryl; and the substituted or unsubstituted C6-C30 aryl, and the substituted or unsubstituted C3-C30 heteroaryl are selected from the following groups: 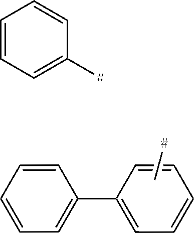 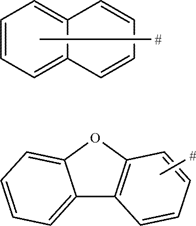 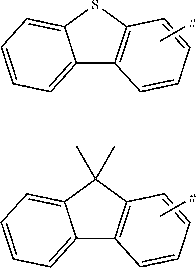 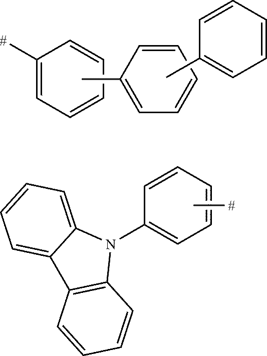 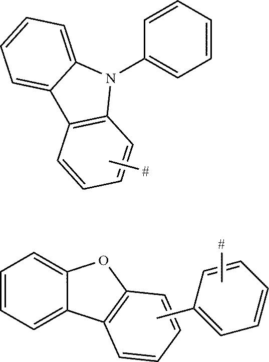 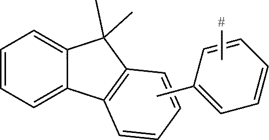 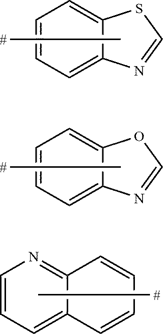 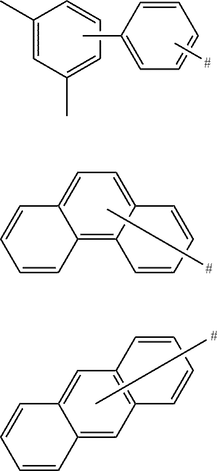 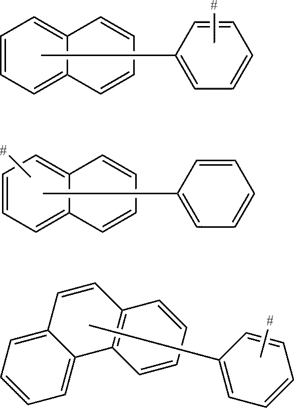 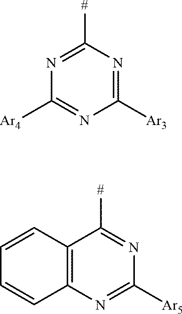 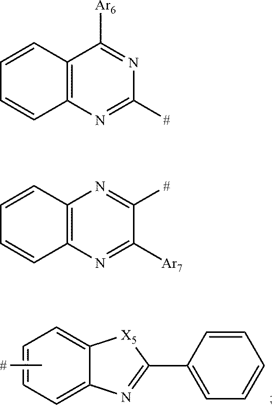
wherein X₅ is selected from O atom or S atom; and Ar₃ to Ar₇ are each independently selected from substituted or unsubstituted phenyl, substituted or unsubstituted naphthyl, substituted or unsubstituted biphenyl, substituted or unsubstituted dibenzofuranyl, substituted or unsubstituted dibenzothien; the substituents of Ar₃ to Ar₇ are selected from deuterium, fluorine, methyl, or tert-butyl.

2. The nitrogen-containing compound of claim 1, wherein
R₀ is selected from a C1-C6 alkyl .

3-4. (canceled)

5. The nitrogen-containing compound of claim 1, wherein
R₀ is hydrogen, or a C1-C4 alkyl .

6. (canceled)

7. The nitrogen-containing compound of claim 1, wherein Ar₁, Ar₂ and R₀ are each independently selected from any one of the following groups: 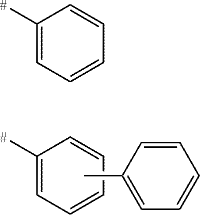 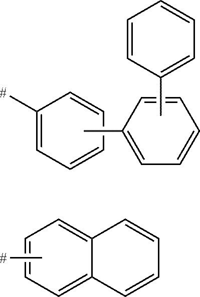 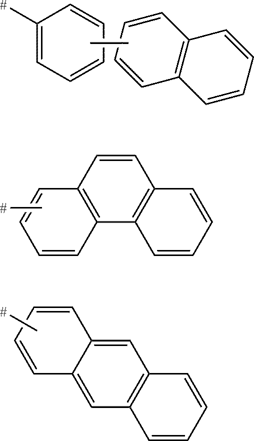 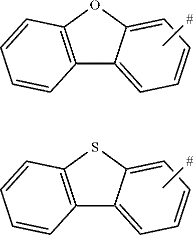 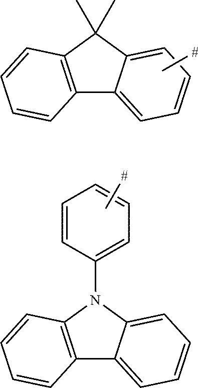 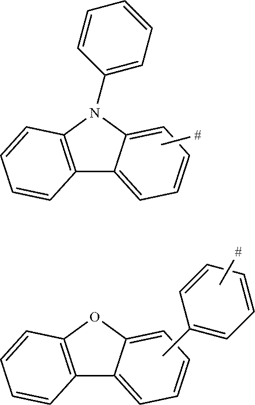 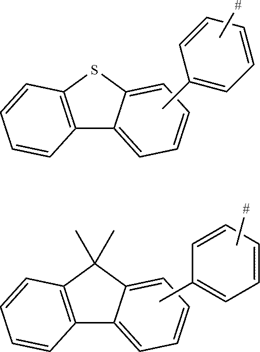 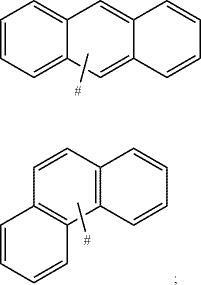
# represents a connection position.

8. The nitrogen-containing compound of claim 1, wherein R₀ is selected from the following group: 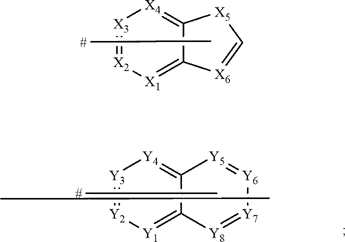
wherein X₁, X₂, X₃ and X₄ are CH, X₅ is selected from O atom or S atom, and X₆ is N.

9. The nitrogen-containing compound of claim 1, wherein R₀ is selected from hydrogen, methyl, ethyl, isopropyl, or tert-butyl.

10-11. (canceled)

12. The nitrogen-containing compound of claim 1, wherein the nitrogen-containing compound is selected from the following compounds: 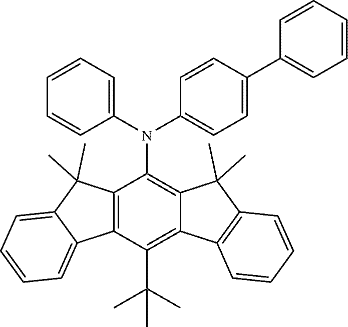 [SMILES: CC(C)(C)c1c2c(c(N(c3ccccc3)c3ccc(-c4ccccc4)cc3)c3c1-c1ccccc1C3(C)C)C(C)(C)c1ccccc1-2] 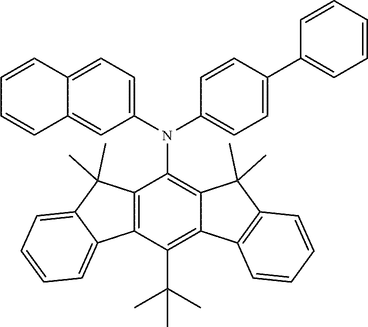 [SMILES: CC(C)(C)c1c2c(c(N(c3ccc(-c4ccccc4)cc3)c3ccc4ccccc4c3)c3c1-c1ccccc1C3(C)C)C(C)(C)c1ccccc1-2] 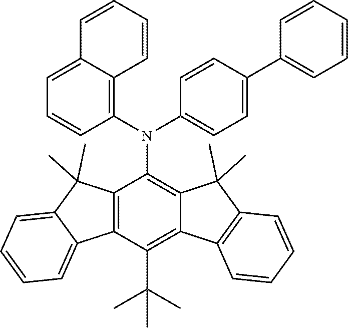 [SMILES: CC(C)(C)c1c2c(c(N(c3ccc(-c4ccccc4)cc3)c3cccc4ccccc34)c3c1-c1ccccc1C3(C)C)C(C)(C)c1ccccc1-2] 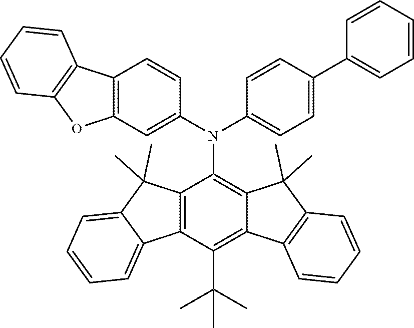 [SMILES: CC(C)(C)c1c2c(c(N(c3ccc(-c4ccccc4)cc3)c3ccc4c(c3)oc3ccccc34)c3c1-c1ccccc1C3(C)C)C(C)(C)c1ccccc1-2] 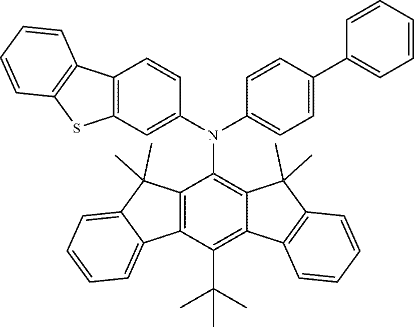 [SMILES: CC(C)(C)c1c2c(c(N(c3ccc(-c4ccccc4)cc3)c3ccc4c(c3)sc3ccccc34)c3c1-c1ccccc1C3(C)C)C(C)(C)c1ccccc1-2] 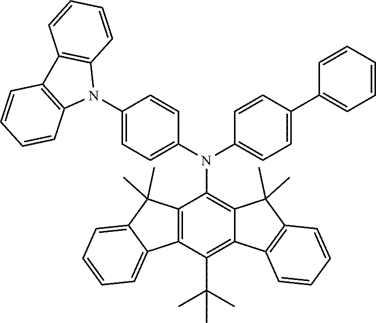 [SMILES: CC(C)(C)c1c2c(c(N(c3ccc(-c4ccccc4)cc3)c3ccc(-n4c5ccccc5c5ccccc54)cc3)c3c1-c1ccccc1C3(C)C)C(C)(C)c1ccccc1-2] 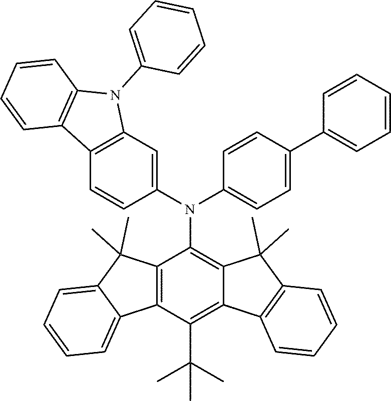 [SMILES: CC(C)(C)c1c2c(c(N(c3ccc(-c4ccccc4)cc3)c3ccc4c5ccccc5n(-c5ccccc5)c4c3)c3c1-c1ccccc1C3(C)C)C(C)(C)c1ccccc1-2] 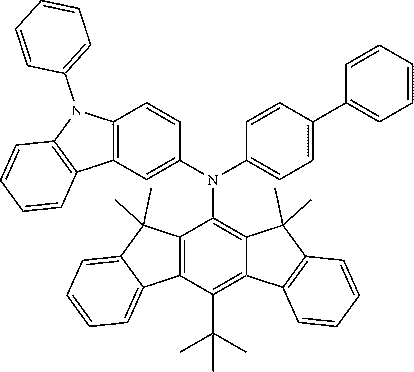 [SMILES: CC(C)(C)c1c2c(c(N(c3ccc(-c4ccccc4)cc3)c3ccc4c(c3)c3ccccc3n4-c3ccccc3)c3c1-c1ccccc1C3(C)C)C(C)(C)c1ccccc1-2] 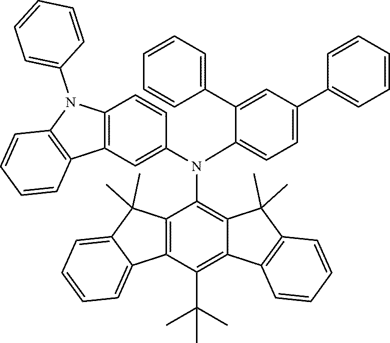 [SMILES: CC(C)(C)c1c2c(c(N(c3ccc4c(c3)c3ccccc3n4-c3ccccc3)c3ccc(-c4ccccc4)cc3-c3ccccc3)c3c1-c1ccccc1C3(C)C)C(C)(C)c1ccccc1-2] 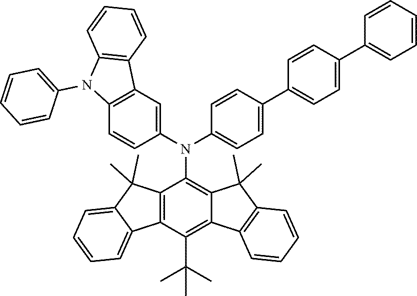 [SMILES: CC(C)(C)c1c2c(c(N(c3ccc(-c4ccc(-c5ccccc5)cc4)cc3)c3ccc4c(c3)c3ccccc3n4-c3ccccc3)c3c1-c1ccccc1C3(C)C)C(C)(C)c1ccccc1-2] 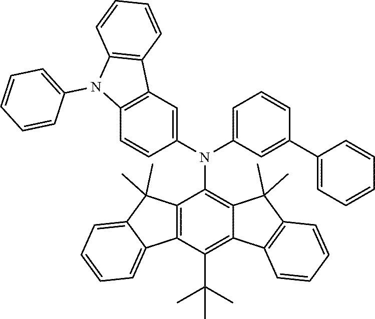 [SMILES: CC(C)(C)c1c2c(c(N(c3cccc(-c4ccccc4)c3)c3ccc4c(c3)c3ccccc3n4-c3ccccc3)c3c1-c1ccccc1C3(C)C)C(C)(C)c1ccccc1-2] 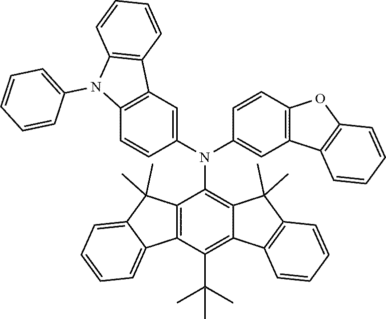 [SMILES: CC(C)(C)c1c2c(c(N(c3ccc4oc5ccccc5c4c3)c3ccc4c(c3)c3ccccc3n4-c3ccccc3)c3c1-c1ccccc1C3(C)C)C(C)(C)c1ccccc1-2] 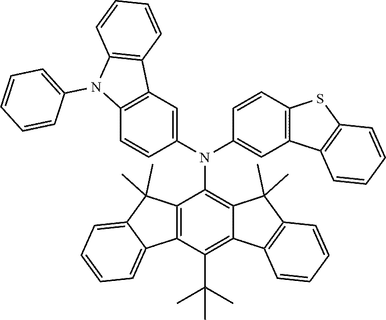 [SMILES: CC(C)(C)c1c2c(c(N(c3ccc4sc5ccccc5c4c3)c3ccc4c(c3)c3ccccc3n4-c3ccccc3)c3c1-c1ccccc1C3(C)C)C(C)(C)c1ccccc1-2] 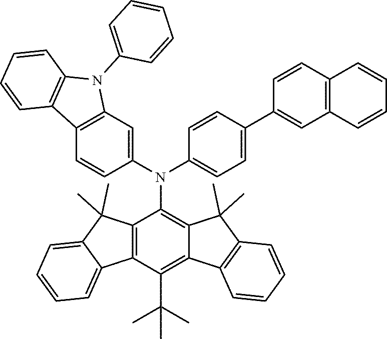 [SMILES: CC(C)(C)c1c2c(c(N(c3ccc(-c4ccc5ccccc5c4)cc3)c3ccc4c5ccccc5n(-c5ccccc5)c4c3)c3c1-c1ccccc1C3(C)C)C(C)(C)c1ccccc1-2] 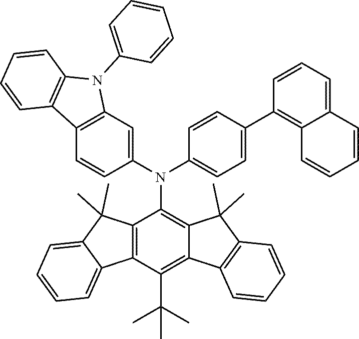 [SMILES: CC(C)(C)c1c2c(c(N(c3ccc(-c4cccc5ccccc45)cc3)c3ccc4c5ccccc5n(-c5ccccc5)c4c3)c3c1-c1ccccc1C3(C)C)C(C)(C)c1ccccc1-2] 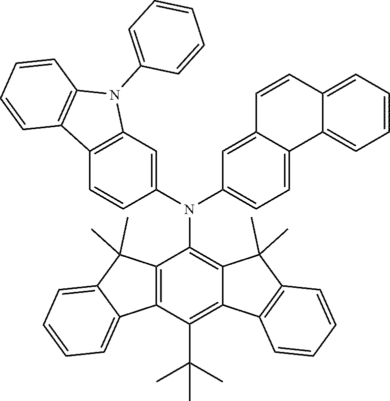 [SMILES: CC(C)(C)c1c2c(c(N(c3ccc4c(ccc5ccccc54)c3)c3ccc4c5ccccc5n(-c5ccccc5)c4c3)c3c1-c1ccccc1C3(C)C)C(C)(C)c1ccccc1-2] 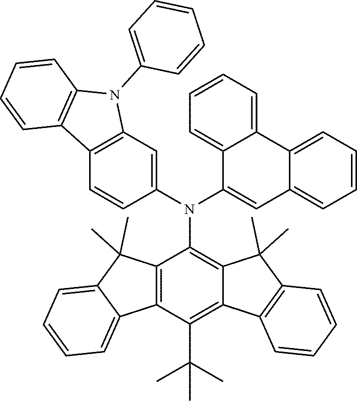 [SMILES: CC(C)(C)c1c2c(c(N(c3ccc4c5ccccc5n(-c5ccccc5)c4c3)c3cc4ccccc4c4ccccc34)c3c1-c1ccccc1C3(C)C)C(C)(C)c1ccccc1-2] 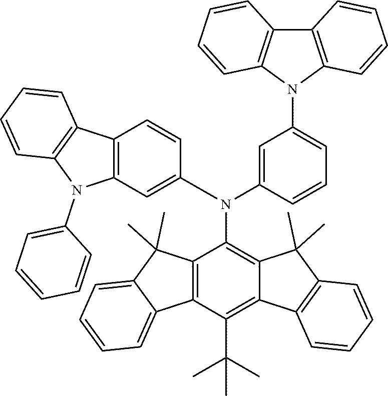 [SMILES: CC(C)(C)c1c2c(c(N(c3cccc(-n4c5ccccc5c5ccccc54)c3)c3ccc4c5ccccc5n(-c5ccccc5)c4c3)c3c1-c1ccccc1C3(C)C)C(C)(C)c1ccccc1-2] 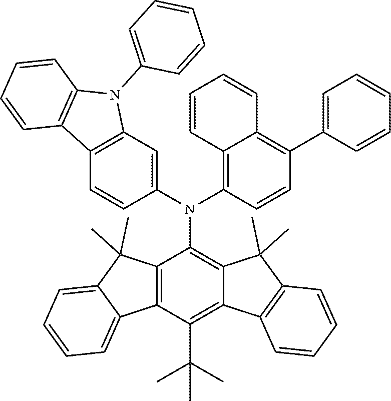 [SMILES: CC(C)(C)c1c2c(c(N(c3ccc4c5ccccc5n(-c5ccccc5)c4c3)c3ccc(-c4ccccc4)c4ccccc34)c3c1-c1ccccc1C3(C)C)C(C)(C)c1ccccc1-2] 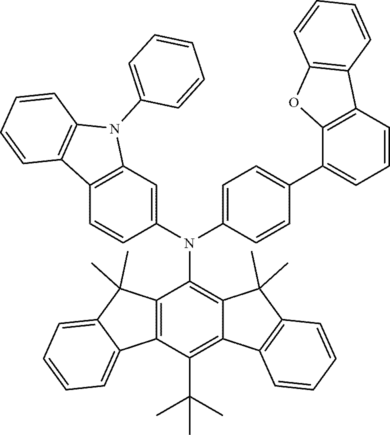 [SMILES: CC(C)(C)c1c2c(c(N(c3ccc(-c4cccc5c4oc4ccccc45)cc3)c3ccc4c5ccccc5n(-c5ccccc5)c4c3)c3c1-c1ccccc1C3(C)C)C(C)(C)c1ccccc1-2] 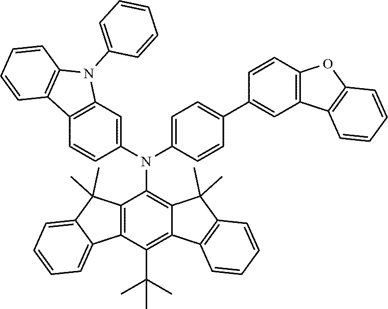 [SMILES: CC(C)(C)c1c2c(c(N(c3ccc(-c4ccc5oc6ccccc6c5c4)cc3)c3ccc4c5ccccc5n(-c5ccccc5)c4c3)c3c1-c1ccccc1C3(C)C)C(C)(C)c1ccccc1-2] 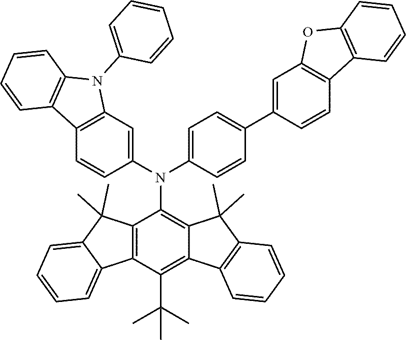 [SMILES: CC(C)(C)c1c2c(c(N(c3ccc(-c4ccc5c(c4)oc4ccccc45)cc3)c3ccc4c5ccccc5n(-c5ccccc5)c4c3)c3c1-c1ccccc1C3(C)C)C(C)(C)c1ccccc1-2] 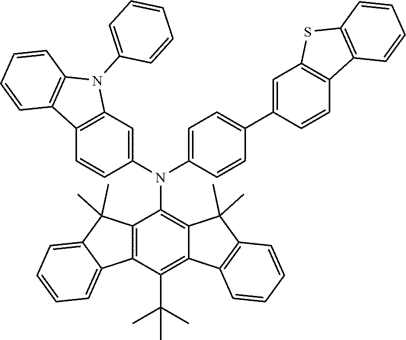 [SMILES: CC(C)(C)c1c2c(c(N(c3ccc(-c4ccc5c(c4)sc4ccccc45)cc3)c3ccc4c5ccccc5n(-c5ccccc5)c4c3)c3c1-c1ccccc1C3(C)C)C(C)(C)c1ccccc1-2] 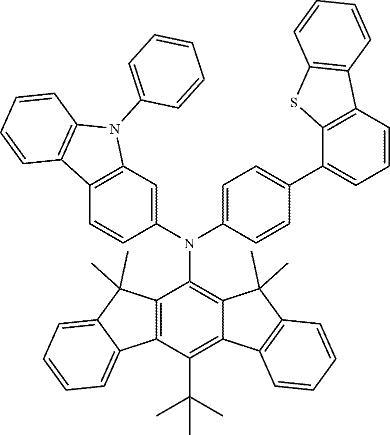 [SMILES: CC(C)(C)c1c2c(c(N(c3ccc(-c4cccc5c4sc4ccccc45)cc3)c3ccc4c5ccccc5n(-c5ccccc5)c4c3)c3c1-c1ccccc1C3(C)C)C(C)(C)c1ccccc1-2] 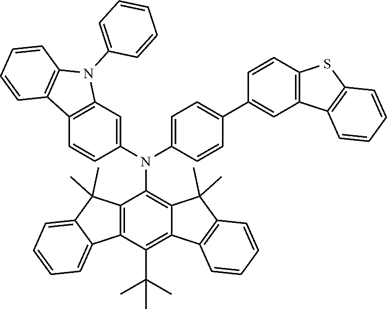 [SMILES: CC(C)(C)c1c2c(c(N(c3ccc(-c4ccc5sc6ccccc6c5c4)cc3)c3ccc4c5ccccc5n(-c5ccccc5)c4c3)c3c1-c1ccccc1C3(C)C)C(C)(C)c1ccccc1-2] 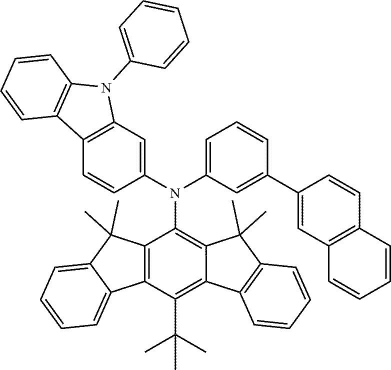 [SMILES: CC(C)(C)c1c2c(c(N(c3cccc(-c4ccc5ccccc5c4)c3)c3ccc4c5ccccc5n(-c5ccccc5)c4c3)c3c1-c1ccccc1C3(C)C)C(C)(C)c1ccccc1-2] 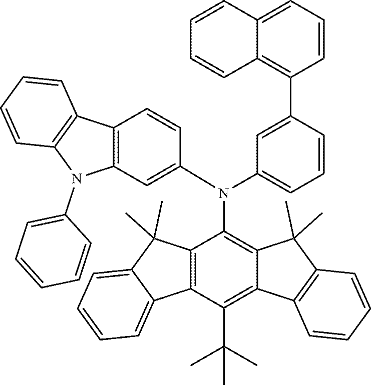 [SMILES: CC(C)(C)c1c2c(c(N(c3cccc(-c4cccc5ccccc45)c3)c3ccc4c5ccccc5n(-c5ccccc5)c4c3)c3c1-c1ccccc1C3(C)C)C(C)(C)c1ccccc1-2] 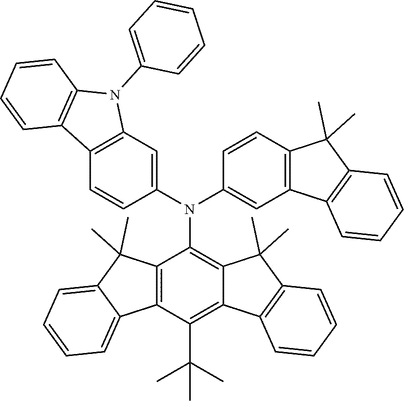 [SMILES: CC(C)(C)c1c2c(c(N(c3ccc4c(c3)-c3ccccc3C4(C)C)c3ccc4c5ccccc5n(-c5ccccc5)c4c3)c3c1-c1ccccc1C3(C)C)C(C)(C)c1ccccc1-2] 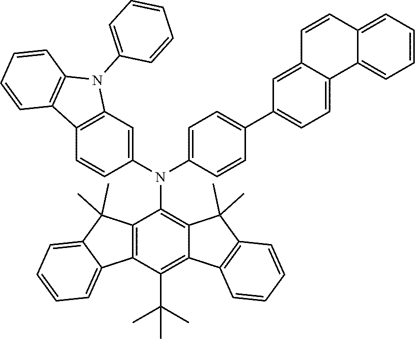 [SMILES: CC(C)(C)c1c2c(c(N(c3ccc(-c4ccc5c(ccc6ccccc65)c4)cc3)c3ccc4c5ccccc5n(-c5ccccc5)c4c3)c3c1-c1ccccc1C3(C)C)C(C)(C)c1ccccc1-2] 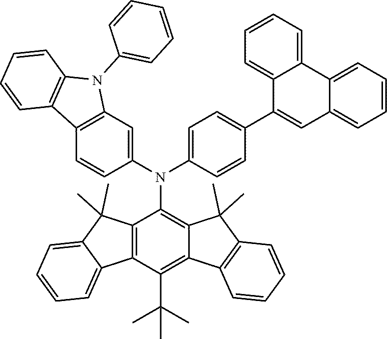 [SMILES: CC(C)(C)c1c2c(c(N(c3ccc(-c4cc5ccccc5c5ccccc45)cc3)c3ccc4c5ccccc5n(-c5ccccc5)c4c3)c3c1-c1ccccc1C3(C)C)C(C)(C)c1ccccc1-2] 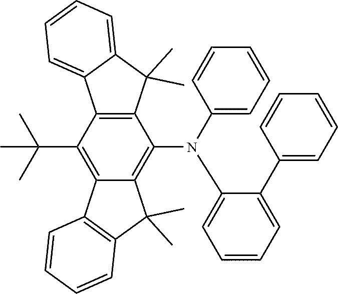 [SMILES: CC(C)(C)c1c2c(c(N(c3ccccc3)c3ccccc3-c3ccccc3)c3c1-c1ccccc1C3(C)C)C(C)(C)c1ccccc1-2] 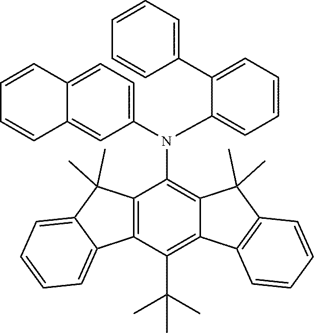 [SMILES: CC(C)(C)c1c2c(c(N(c3ccc4ccccc4c3)c3ccccc3-c3ccccc3)c3c1-c1ccccc1C3(C)C)C(C)(C)c1ccccc1-2] 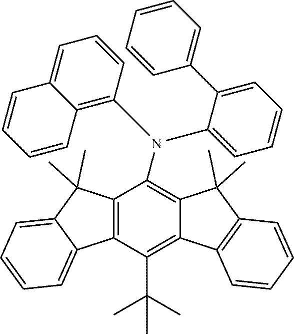 [SMILES: CC(C)(C)c1c2c(c(N(c3ccccc3-c3ccccc3)c3cccc4ccccc34)c3c1-c1ccccc1C3(C)C)C(C)(C)c1ccccc1-2] 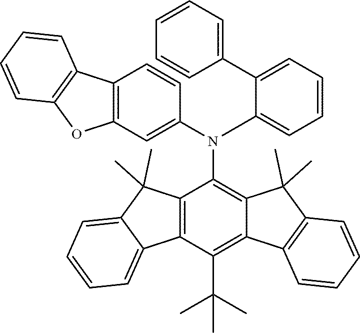 [SMILES: CC(C)(C)c1c2c(c(N(c3ccc4c(c3)oc3ccccc34)c3ccccc3-c3ccccc3)c3c1-c1ccccc1C3(C)C)C(C)(C)c1ccccc1-2] 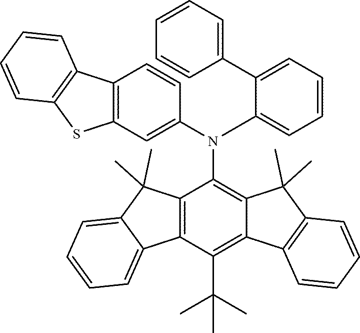 [SMILES: CC(C)(C)c1c2c(c(N(c3ccc4c(c3)sc3ccccc34)c3ccccc3-c3ccccc3)c3c1-c1ccccc1C3(C)C)C(C)(C)c1ccccc1-2] 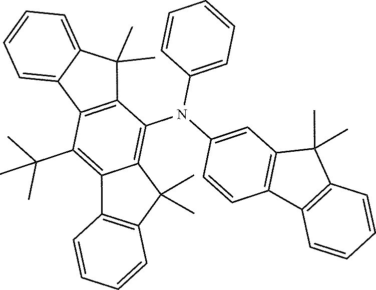 [SMILES: CC(C)(C)c1c2c(c(N(c3ccccc3)c3ccc4c(c3)C(C)(C)c3ccccc3-4)c3c1-c1ccccc1C3(C)C)C(C)(C)c1ccccc1-2] 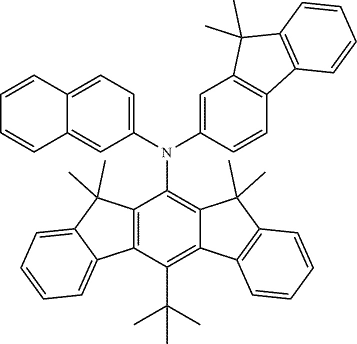 [SMILES: CC(C)(C)c1c2c(c(N(c3ccc4c(c3)C(C)(C)c3ccccc3-4)c3ccc4ccccc4c3)c3c1-c1ccccc1C3(C)C)C(C)(C)c1ccccc1-2] 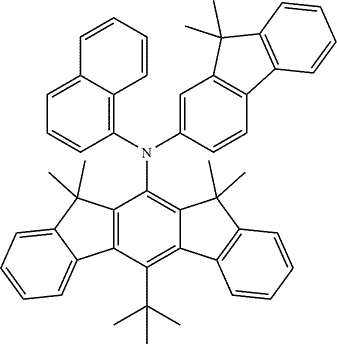 [SMILES: CC(C)(C)c1c2c(c(N(c3ccc4c(c3)C(C)(C)c3ccccc3-4)c3cccc4ccccc34)c3c1-c1ccccc1C3(C)C)C(C)(C)c1ccccc1-2] 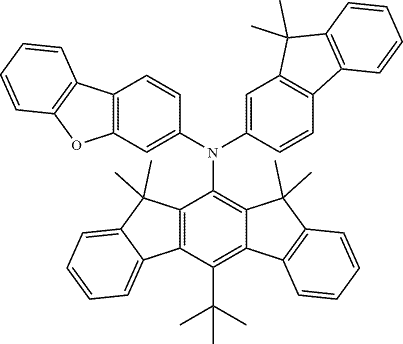 [SMILES: CC(C)(C)c1c2c(c(N(c3ccc4c(c3)C(C)(C)c3ccccc3-4)c3ccc4c(c3)oc3ccccc34)c3c1-c1ccccc1C3(C)C)C(C)(C)c1ccccc1-2] 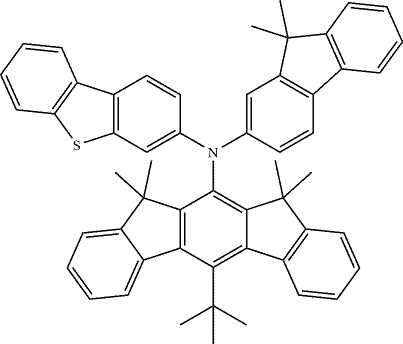 [SMILES: CC(C)(C)c1c2c(c(N(c3ccc4c(c3)C(C)(C)c3ccccc3-4)c3ccc4c(c3)sc3ccccc34)c3c1-c1ccccc1C3(C)C)C(C)(C)c1ccccc1-2] 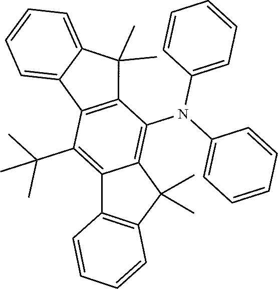 [SMILES: CC(C)(C)c1c2c(c(N(c3ccccc3)c3ccccc3)c3c1-c1ccccc1C3(C)C)C(C)(C)c1ccccc1-2] 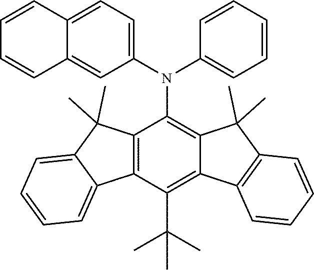 [SMILES: CC(C)(C)c1c2c(c(N(c3ccccc3)c3ccc4ccccc4c3)c3c1-c1ccccc1C3(C)C)C(C)(C)c1ccccc1-2] 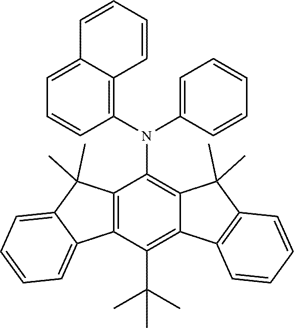 [SMILES: CC(C)(C)c1c2c(c(N(c3ccccc3)c3cccc4ccccc34)c3c1-c1ccccc1C3(C)C)C(C)(C)c1ccccc1-2] 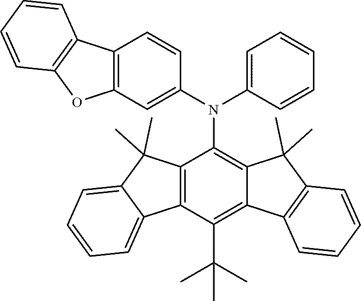 [SMILES: CC(C)(C)c1c2c(c(N(c3ccccc3)c3ccc4c(c3)oc3ccccc34)c3c1-c1ccccc1C3(C)C)C(C)(C)c1ccccc1-2] 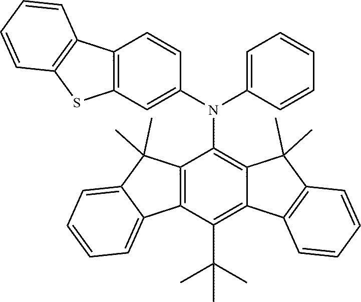 [SMILES: CC(C)(C)c1c2c(c(N(c3ccccc3)c3ccc4c(c3)sc3ccccc34)c3c1-c1ccccc1C3(C)C)C(C)(C)c1ccccc1-2] 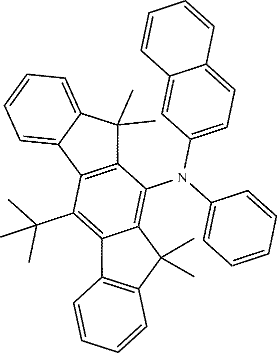 [SMILES: CC(C)(C)c1c2c(c(N(c3ccccc3)c3ccc4ccccc4c3)c3c1-c1ccccc1C3(C)C)C(C)(C)c1ccccc1-2] 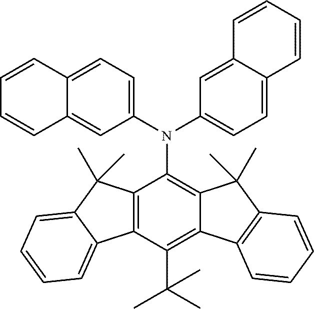 [SMILES: CC(C)(C)c1c2c(c(N(c3ccc4ccccc4c3)c3ccc4ccccc4c3)c3c1-c1ccccc1C3(C)C)C(C)(C)c1ccccc1-2] 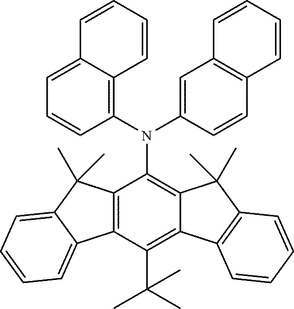 [SMILES: CC(C)(C)c1c2c(c(N(c3ccc4ccccc4c3)c3cccc4ccccc34)c3c1-c1ccccc1C3(C)C)C(C)(C)c1ccccc1-2] 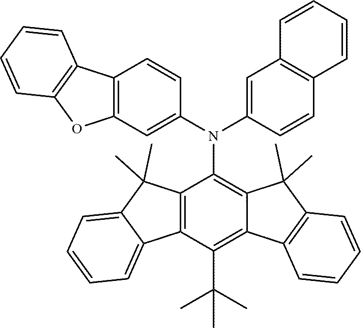 [SMILES: CC(C)(C)c1c2c(c(N(c3ccc4ccccc4c3)c3ccc4c(c3)oc3ccccc34)c3c1-c1ccccc1C3(C)C)C(C)(C)c1ccccc1-2] 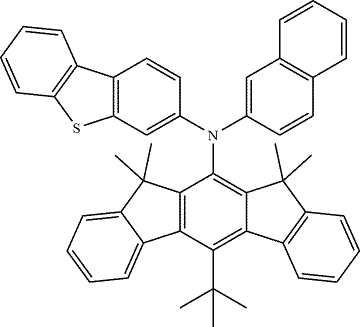 [SMILES: CC(C)(C)c1c2c(c(N(c3ccc4ccccc4c3)c3ccc4c(c3)sc3ccccc34)c3c1-c1ccccc1C3(C)C)C(C)(C)c1ccccc1-2] 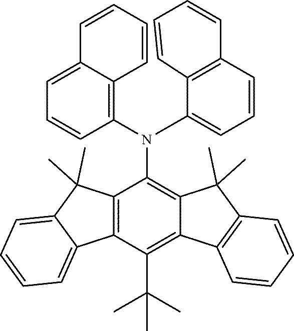 [SMILES: CC(C)(C)c1c2c(c(N(c3cccc4ccccc34)c3cccc4ccccc34)c3c1-c1ccccc1C3(C)C)C(C)(C)c1ccccc1-2] 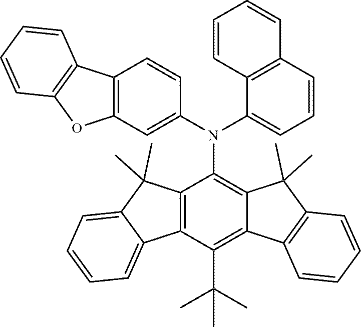 [SMILES: CC(C)(C)c1c2c(c(N(c3ccc4c(c3)oc3ccccc34)c3cccc4ccccc34)c3c1-c1ccccc1C3(C)C)C(C)(C)c1ccccc1-2] 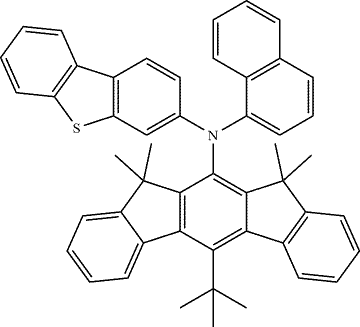 [SMILES: CC(C)(C)c1c2c(c(N(c3ccc4c(c3)sc3ccccc34)c3cccc4ccccc34)c3c1-c1ccccc1C3(C)C)C(C)(C)c1ccccc1-2] 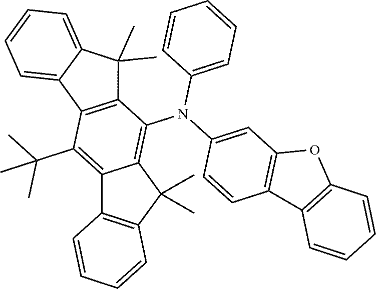 [SMILES: CC(C)(C)c1c2c(c(N(c3ccccc3)c3ccc4c(c3)oc3ccccc34)c3c1-c1ccccc1C3(C)C)C(C)(C)c1ccccc1-2] 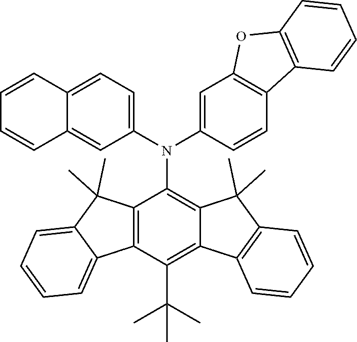 [SMILES: CC(C)(C)c1c2c(c(N(c3ccc4ccccc4c3)c3ccc4c(c3)oc3ccccc34)c3c1-c1ccccc1C3(C)C)C(C)(C)c1ccccc1-2] 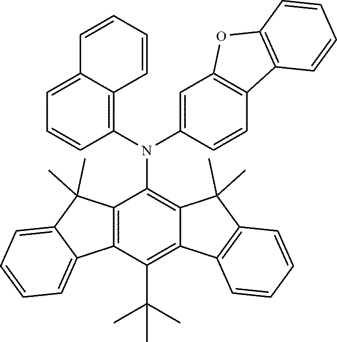 [SMILES: CC(C)(C)c1c2c(c(N(c3ccc4c(c3)oc3ccccc34)c3cccc4ccccc34)c3c1-c1ccccc1C3(C)C)C(C)(C)c1ccccc1-2] 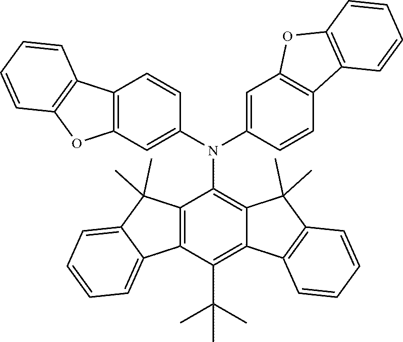 [SMILES: CC(C)(C)c1c2c(c(N(c3ccc4c(c3)oc3ccccc34)c3ccc4c(c3)oc3ccccc34)c3c1-c1ccccc1C3(C)C)C(C)(C)c1ccccc1-2] 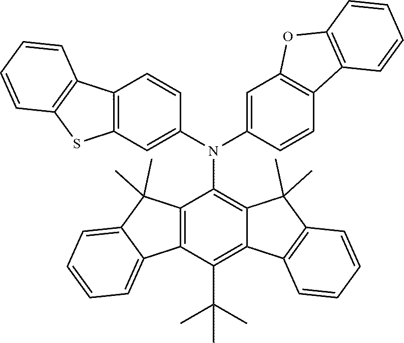 [SMILES: CC(C)(C)c1c2c(c(N(c3ccc4c(c3)oc3ccccc34)c3ccc4c(c3)sc3ccccc34)c3c1-c1ccccc1C3(C)C)C(C)(C)c1ccccc1-2] 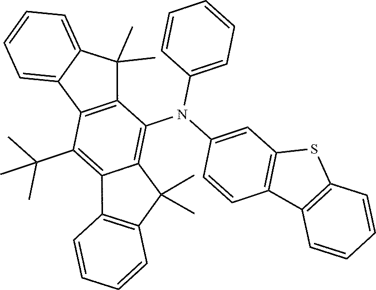 [SMILES: CC(C)(C)c1c2c(c(N(c3ccccc3)c3ccc4c(c3)sc3ccccc34)c3c1-c1ccccc1C3(C)C)C(C)(C)c1ccccc1-2] 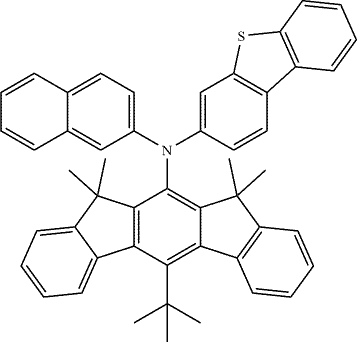 [SMILES: CC(C)(C)c1c2c(c(N(c3ccc4ccccc4c3)c3ccc4c(c3)sc3ccccc34)c3c1-c1ccccc1C3(C)C)C(C)(C)c1ccccc1-2] 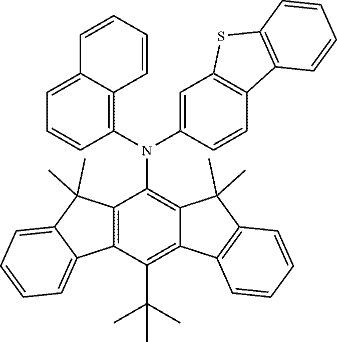 [SMILES: CC(C)(C)c1c2c(c(N(c3ccc4c(c3)sc3ccccc34)c3cccc4ccccc34)c3c1-c1ccccc1C3(C)C)C(C)(C)c1ccccc1-2] 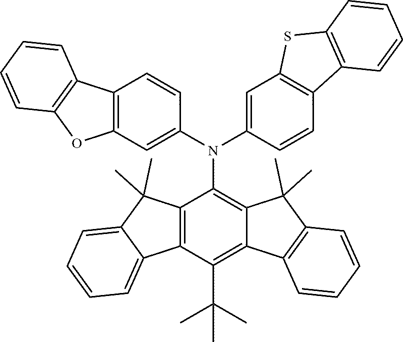 [SMILES: CC(C)(C)c1c2c(c(N(c3ccc4c(c3)oc3ccccc34)c3ccc4c(c3)sc3ccccc34)c3c1-c1ccccc1C3(C)C)C(C)(C)c1ccccc1-2] 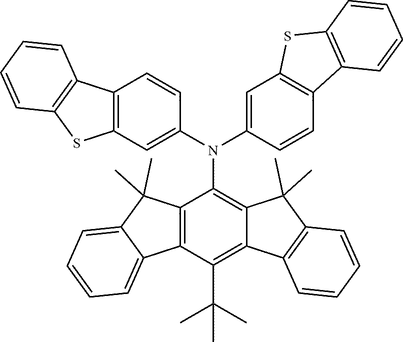 [SMILES: CC(C)(C)c1c2c(c(N(c3ccc4c(c3)sc3ccccc34)c3ccc4c(c3)sc3ccccc34)c3c1-c1ccccc1C3(C)C)C(C)(C)c1ccccc1-2] 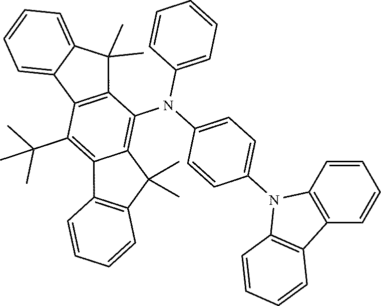 [SMILES: CC(C)(C)c1c2c(c(N(c3ccccc3)c3ccc(-n4c5ccccc5c5ccccc54)cc3)c3c1-c1ccccc1C3(C)C)C(C)(C)c1ccccc1-2] 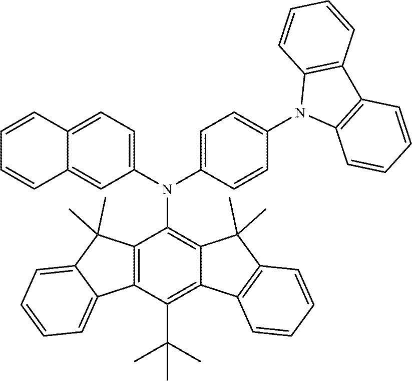 [SMILES: CC(C)(C)c1c2c(c(N(c3ccc(-n4c5ccccc5c5ccccc54)cc3)c3ccc4ccccc4c3)c3c1-c1ccccc1C3(C)C)C(C)(C)c1ccccc1-2] 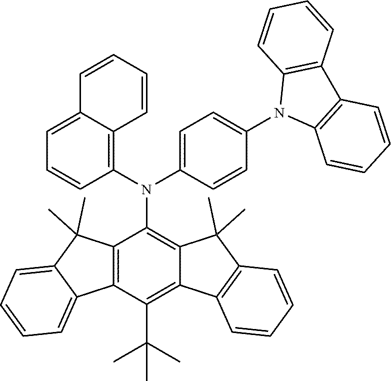 [SMILES: CC(C)(C)c1c2c(c(N(c3ccc(-n4c5ccccc5c5ccccc54)cc3)c3cccc4ccccc34)c3c1-c1ccccc1C3(C)C)C(C)(C)c1ccccc1-2] 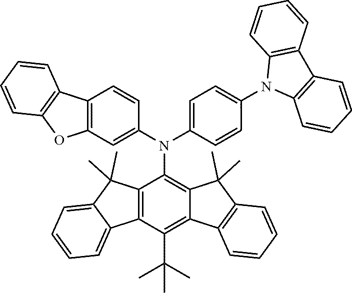 [SMILES: CC(C)(C)c1c2c(c(N(c3ccc(-n4c5ccccc5c5ccccc54)cc3)c3ccc4c(c3)oc3ccccc34)c3c1-c1ccccc1C3(C)C)C(C)(C)c1ccccc1-2] 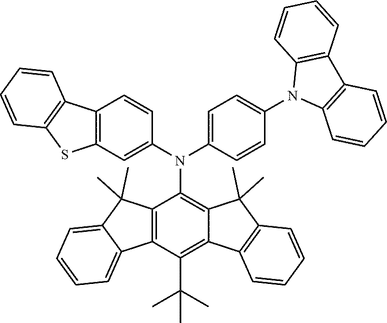 [SMILES: CC(C)(C)c1c2c(c(N(c3ccc(-n4c5ccccc5c5ccccc54)cc3)c3ccc4c(c3)sc3ccccc34)c3c1-c1ccccc1C3(C)C)C(C)(C)c1ccccc1-2] 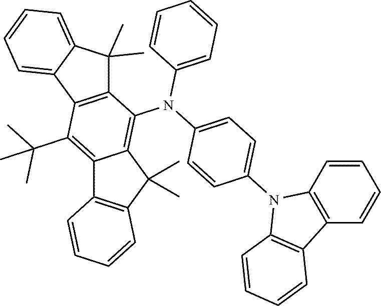 [SMILES: CC(C)(C)c1c2c(c(N(c3ccccc3)c3ccc(-n4c5ccccc5c5ccccc54)cc3)c3c1-c1ccccc1C3(C)C)C(C)(C)c1ccccc1-2] 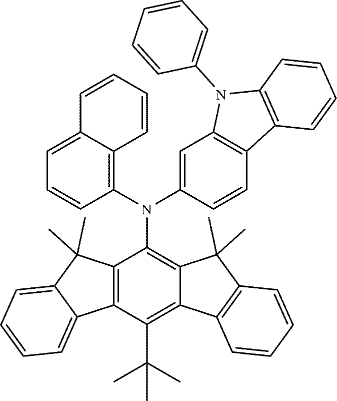 [SMILES: CC(C)(C)c1c2c(c(N(c3ccc4c5ccccc5n(-c5ccccc5)c4c3)c3cccc4ccccc34)c3c1-c1ccccc1C3(C)C)C(C)(C)c1ccccc1-2] 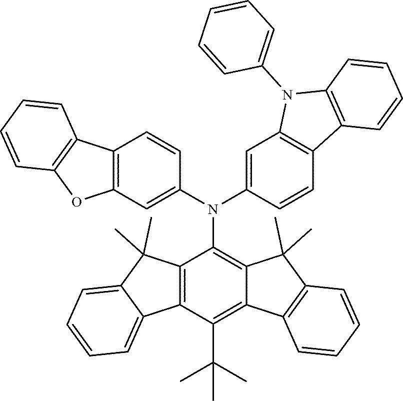 [SMILES: CC(C)(C)c1c2c(c(N(c3ccc4c(c3)oc3ccccc34)c3ccc4c5ccccc5n(-c5ccccc5)c4c3)c3c1-c1ccccc1C3(C)C)C(C)(C)c1ccccc1-2] 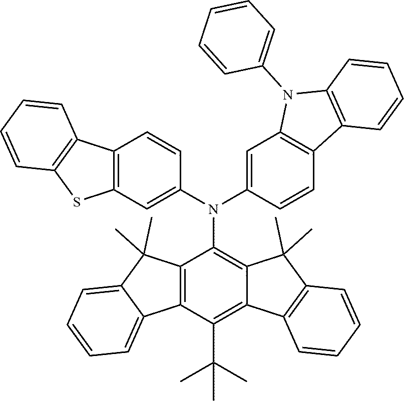 [SMILES: CC(C)(C)c1c2c(c(N(c3ccc4c(c3)sc3ccccc34)c3ccc4c5ccccc5n(-c5ccccc5)c4c3)c3c1-c1ccccc1C3(C)C)C(C)(C)c1ccccc1-2] 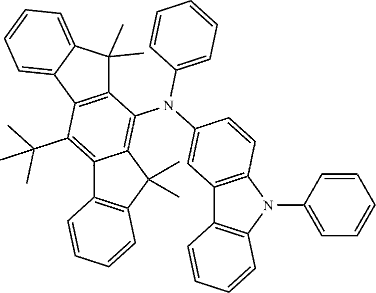 [SMILES: CC(C)(C)c1c2c(c(N(c3ccccc3)c3ccc4c(c3)c3ccccc3n4-c3ccccc3)c3c1-c1ccccc1C3(C)C)C(C)(C)c1ccccc1-2] 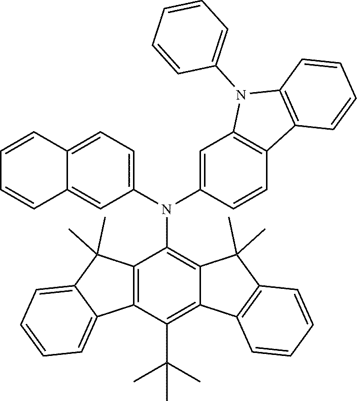 [SMILES: CC(C)(C)c1c2c(c(N(c3ccc4ccccc4c3)c3ccc4c5ccccc5n(-c5ccccc5)c4c3)c3c1-c1ccccc1C3(C)C)C(C)(C)c1ccccc1-2] 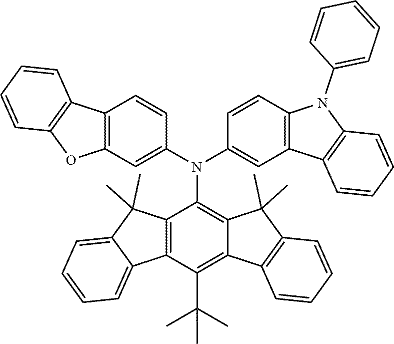 [SMILES: CC(C)(C)c1c2c(c(N(c3ccc4c(c3)oc3ccccc34)c3ccc4c(c3)c3ccccc3n4-c3ccccc3)c3c1-c1ccccc1C3(C)C)C(C)(C)c1ccccc1-2] 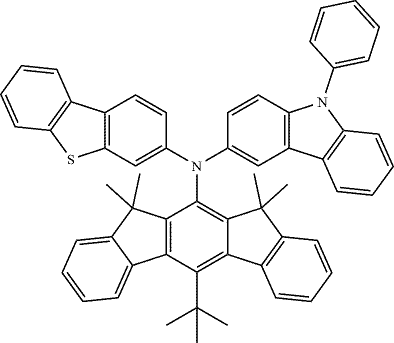 [SMILES: CC(C)(C)c1c2c(c(N(c3ccc4c(c3)sc3ccccc34)c3ccc4c(c3)c3ccccc3n4-c3ccccc3)c3c1-c1ccccc1C3(C)C)C(C)(C)c1ccccc1-2] 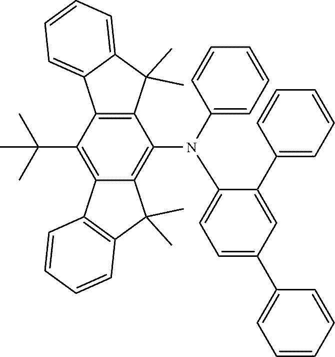 [SMILES: CC(C)(C)c1c2c(c(N(c3ccccc3)c3ccc(-c4ccccc4)cc3-c3ccccc3)c3c1-c1ccccc1C3(C)C)C(C)(C)c1ccccc1-2] 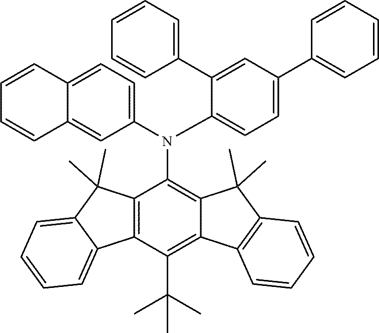 [SMILES: CC(C)(C)c1c2c(c(N(c3ccc4ccccc4c3)c3ccc(-c4ccccc4)cc3-c3ccccc3)c3c1-c1ccccc1C3(C)C)C(C)(C)c1ccccc1-2] 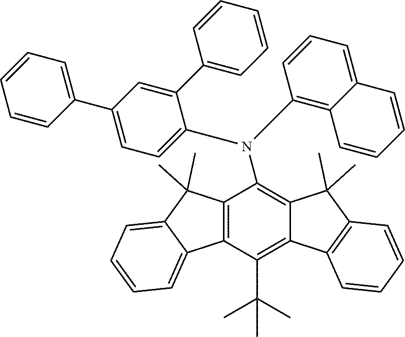 [SMILES: CC(C)(C)c1c2c(c(N(c3ccc(-c4ccccc4)cc3-c3ccccc3)c3cccc4ccccc34)c3c1-c1ccccc1C3(C)C)C(C)(C)c1ccccc1-2] 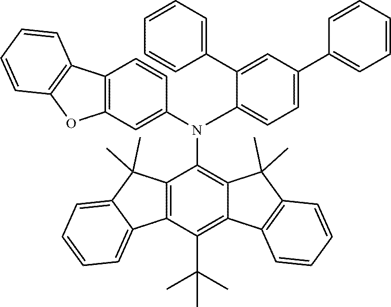 [SMILES: CC(C)(C)c1c2c(c(N(c3ccc4c(c3)oc3ccccc34)c3ccc(-c4ccccc4)cc3-c3ccccc3)c3c1-c1ccccc1C3(C)C)C(C)(C)c1ccccc1-2] 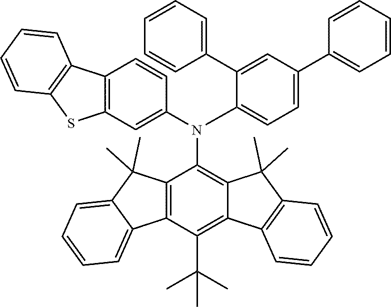 [SMILES: CC(C)(C)c1c2c(c(N(c3ccc4c(c3)sc3ccccc34)c3ccc(-c4ccccc4)cc3-c3ccccc3)c3c1-c1ccccc1C3(C)C)C(C)(C)c1ccccc1-2] 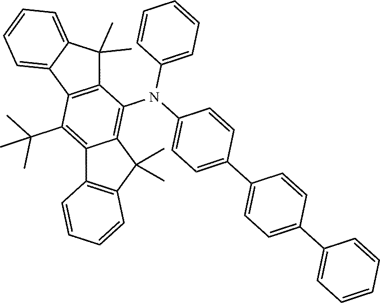 [SMILES: CC(C)(C)c1c2c(c(N(c3ccccc3)c3ccc(-c4ccc(-c5ccccc5)cc4)cc3)c3c1-c1ccccc1C3(C)C)C(C)(C)c1ccccc1-2] 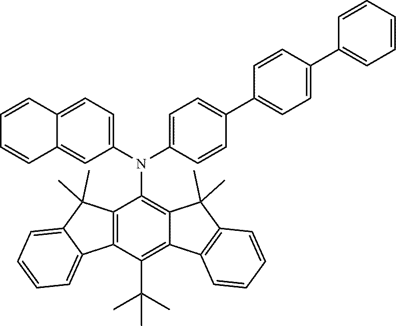 [SMILES: CC(C)(C)c1c2c(c(N(c3ccc(-c4ccc(-c5ccccc5)cc4)cc3)c3ccc4ccccc4c3)c3c1-c1ccccc1C3(C)C)C(C)(C)c1ccccc1-2] 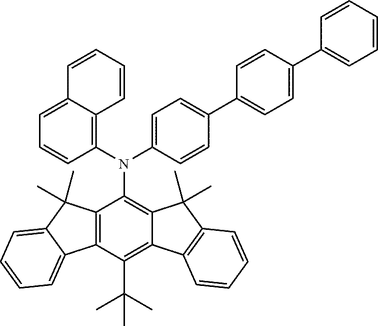 [SMILES: CC(C)(C)c1c2c(c(N(c3ccc(-c4ccc(-c5ccccc5)cc4)cc3)c3cccc4ccccc34)c3c1-c1ccccc1C3(C)C)C(C)(C)c1ccccc1-2] 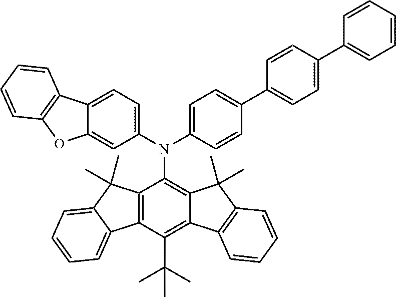 [SMILES: CC(C)(C)c1c2c(c(N(c3ccc(-c4ccc(-c5ccccc5)cc4)cc3)c3ccc4c(c3)oc3ccccc34)c3c1-c1ccccc1C3(C)C)C(C)(C)c1ccccc1-2] 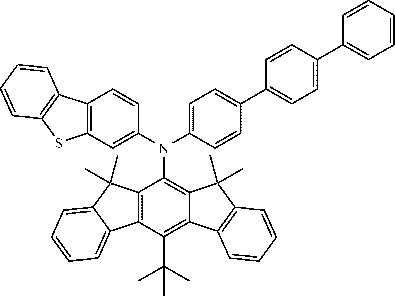 [SMILES: CC(C)(C)c1c2c(c(N(c3ccc(-c4ccc(-c5ccccc5)cc4)cc3)c3ccc4c(c3)sc3ccccc34)c3c1-c1ccccc1C3(C)C)C(C)(C)c1ccccc1-2] 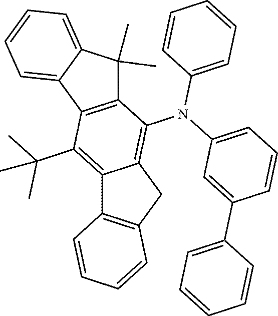 [SMILES: CC(C)(C)c1c2c(c(N(c3ccccc3)c3cccc(-c4ccccc4)c3)c3c1-c1ccccc1C3(C)C)Cc1ccccc1-2] 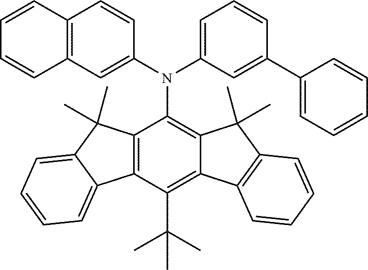 [SMILES: CC(C)(C)c1c2c(c(N(c3cccc(-c4ccccc4)c3)c3ccc4ccccc4c3)c3c1-c1ccccc1C3(C)C)C(C)(C)c1ccccc1-2] 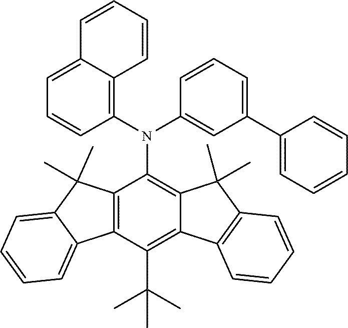 [SMILES: CC(C)(C)c1c2c(c(N(c3cccc(-c4ccccc4)c3)c3cccc4ccccc34)c3c1-c1ccccc1C3(C)C)C(C)(C)c1ccccc1-2] 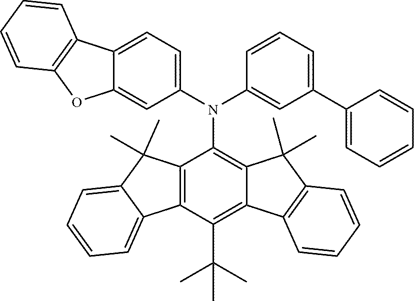 [SMILES: CC(C)(C)c1c2c(c(N(c3cccc(-c4ccccc4)c3)c3ccc4c(c3)oc3ccccc34)c3c1-c1ccccc1C3(C)C)C(C)(C)c1ccccc1-2] 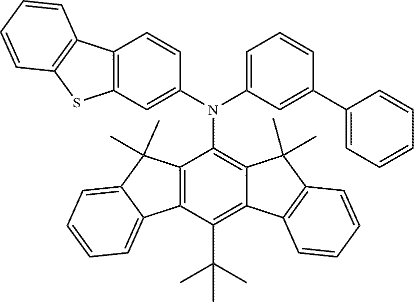 [SMILES: CC(C)(C)c1c2c(c(N(c3cccc(-c4ccccc4)c3)c3ccc4c(c3)sc3ccccc34)c3c1-c1ccccc1C3(C)C)C(C)(C)c1ccccc1-2] 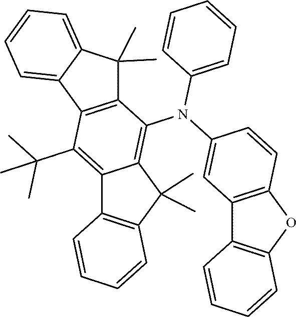 [SMILES: CC(C)(C)c1c2c(c(N(c3ccccc3)c3ccc4oc5ccccc5c4c3)c3c1-c1ccccc1C3(C)C)C(C)(C)c1ccccc1-2] 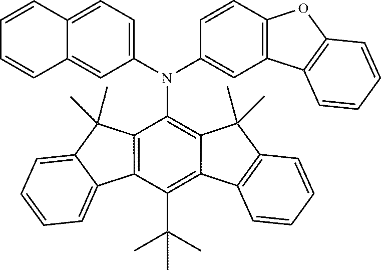 [SMILES: CC(C)(C)c1c2c(c(N(c3ccc4ccccc4c3)c3ccc4oc5ccccc5c4c3)c3c1-c1ccccc1C3(C)C)C(C)(C)c1ccccc1-2] 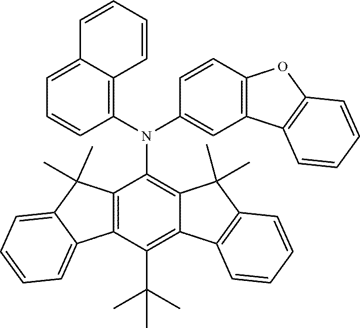 [SMILES: CC(C)(C)c1c2c(c(N(c3ccc4oc5ccccc5c4c3)c3cccc4ccccc34)c3c1-c1ccccc1C3(C)C)C(C)(C)c1ccccc1-2] 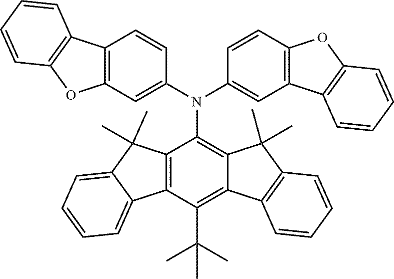 [SMILES: CC(C)(C)c1c2c(c(N(c3ccc4c(c3)oc3ccccc34)c3ccc4oc5ccccc5c4c3)c3c1-c1ccccc1C3(C)C)C(C)(C)c1ccccc1-2] 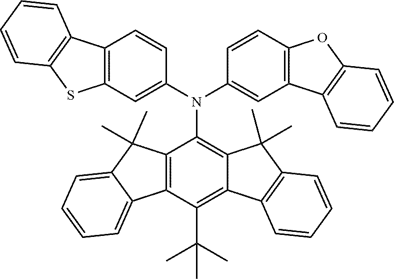 [SMILES: CC(C)(C)c1c2c(c(N(c3ccc4c(c3)sc3ccccc34)c3ccc4oc5ccccc5c4c3)c3c1-c1ccccc1C3(C)C)C(C)(C)c1ccccc1-2] 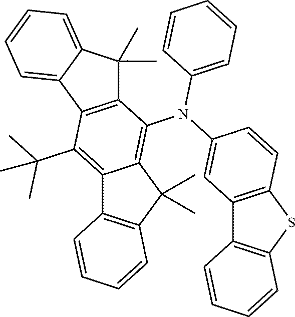 [SMILES: CC(C)(C)c1c2c(c(N(c3ccccc3)c3ccc4sc5ccccc5c4c3)c3c1-c1ccccc1C3(C)C)C(C)(C)c1ccccc1-2] 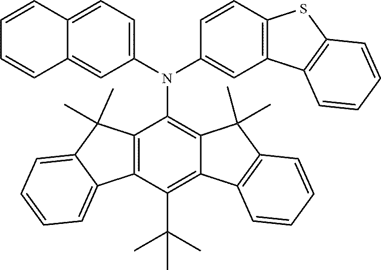 [SMILES: CC(C)(C)c1c2c(c(N(c3ccc4ccccc4c3)c3ccc4sc5ccccc5c4c3)c3c1-c1ccccc1C3(C)C)C(C)(C)c1ccccc1-2] 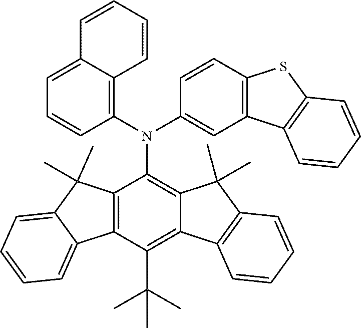 [SMILES: CC(C)(C)c1c2c(c(N(c3ccc4sc5ccccc5c4c3)c3cccc4ccccc34)c3c1-c1ccccc1C3(C)C)C(C)(C)c1ccccc1-2] 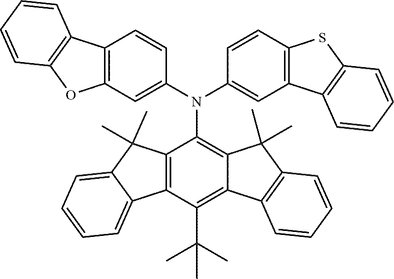 [SMILES: CC(C)(C)c1c2c(c(N(c3ccc4c(c3)oc3ccccc34)c3ccc4sc5ccccc5c4c3)c3c1-c1ccccc1C3(C)C)C(C)(C)c1ccccc1-2] 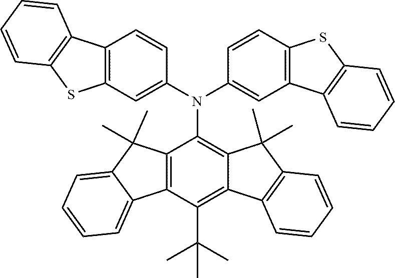 [SMILES: CC(C)(C)c1c2c(c(N(c3ccc4c(c3)sc3ccccc34)c3ccc4sc5ccccc5c4c3)c3c1-c1ccccc1C3(C)C)C(C)(C)c1ccccc1-2] 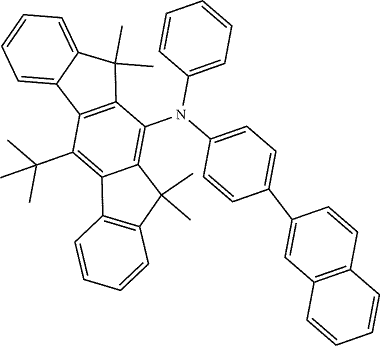 [SMILES: CC(C)(C)c1c2c(c(N(c3ccccc3)c3ccc(-c4ccc5ccccc5c4)cc3)c3c1-c1ccccc1C3(C)C)C(C)(C)c1ccccc1-2] 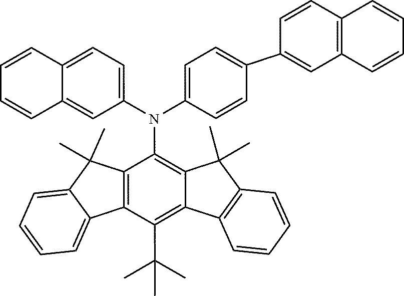 [SMILES: CC(C)(C)c1c2c(c(N(c3ccc(-c4ccc5ccccc5c4)cc3)c3ccc4ccccc4c3)c3c1-c1ccccc1C3(C)C)C(C)(C)c1ccccc1-2] 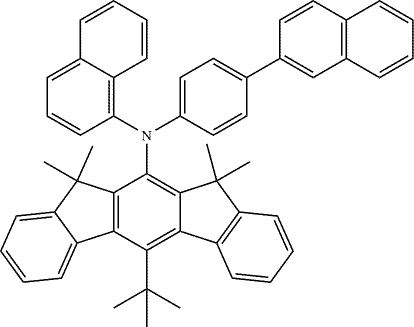 [SMILES: CC(C)(C)c1c2c(c(N(c3ccc(-c4ccc5ccccc5c4)cc3)c3cccc4ccccc34)c3c1-c1ccccc1C3(C)C)C(C)(C)c1ccccc1-2] 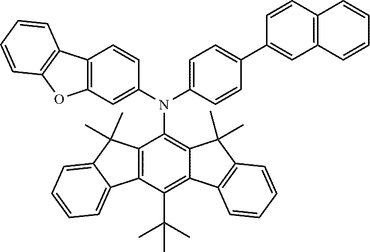 [SMILES: CC(C)(C)c1c2c(c(N(c3ccc(-c4ccc5ccccc5c4)cc3)c3ccc4c(c3)oc3ccccc34)c3c1-c1ccccc1C3(C)C)C(C)(C)c1ccccc1-2] 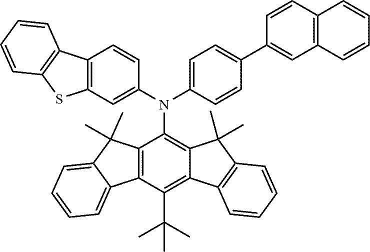 [SMILES: CC(C)(C)c1c2c(c(N(c3ccc(-c4ccc5ccccc5c4)cc3)c3ccc4c(c3)sc3ccccc34)c3c1-c1ccccc1C3(C)C)C(C)(C)c1ccccc1-2] 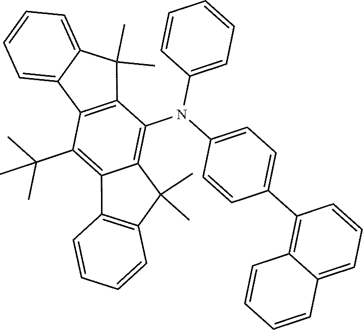 [SMILES: CC(C)(C)c1c2c(c(N(c3ccccc3)c3ccc(-c4cccc5ccccc45)cc3)c3c1-c1ccccc1C3(C)C)C(C)(C)c1ccccc1-2] 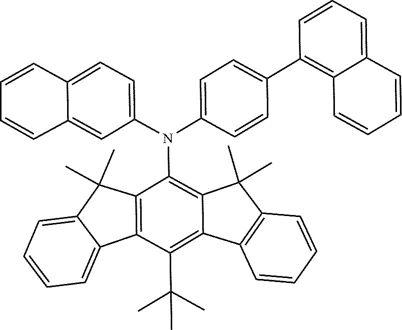 [SMILES: CC(C)(C)c1c2c(c(N(c3ccc(-c4cccc5ccccc45)cc3)c3ccc4ccccc4c3)c3c1-c1ccccc1C3(C)C)C(C)(C)c1ccccc1-2] 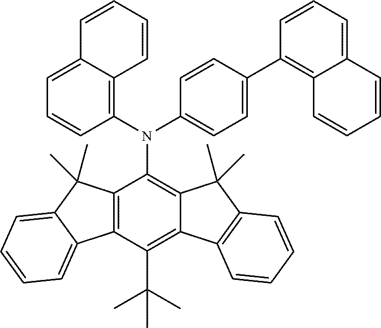 [SMILES: CC(C)(C)c1c2c(c(N(c3ccc(-c4cccc5ccccc45)cc3)c3cccc4ccccc34)c3c1-c1ccccc1C3(C)C)C(C)(C)c1ccccc1-2] 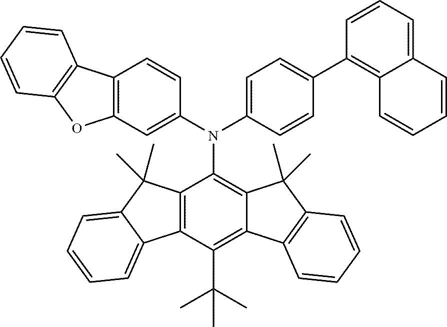 [SMILES: CC(C)(C)c1c2c(c(N(c3ccc(-c4cccc5ccccc45)cc3)c3ccc4c(c3)oc3ccccc34)c3c1-c1ccccc1C3(C)C)C(C)(C)c1ccccc1-2] 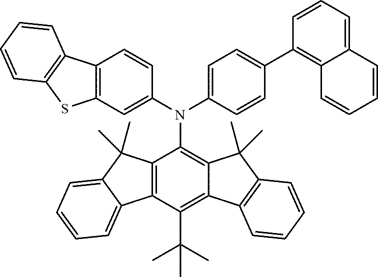 [SMILES: CC(C)(C)c1c2c(c(N(c3ccc(-c4cccc5ccccc45)cc3)c3ccc4c(c3)sc3ccccc34)c3c1-c1ccccc1C3(C)C)C(C)(C)c1ccccc1-2] 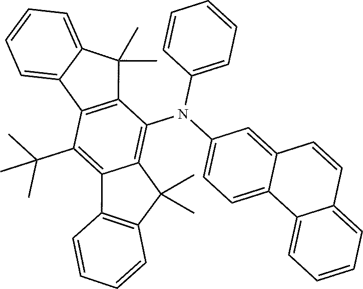 [SMILES: CC(C)(C)c1c2c(c(N(c3ccccc3)c3ccc4c(ccc5ccccc54)c3)c3c1-c1ccccc1C3(C)C)C(C)(C)c1ccccc1-2] 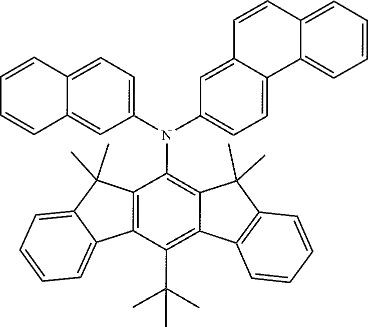 [SMILES: CC(C)(C)c1c2c(c(N(c3ccc4ccccc4c3)c3ccc4c(ccc5ccccc54)c3)c3c1-c1ccccc1C3(C)C)C(C)(C)c1ccccc1-2] 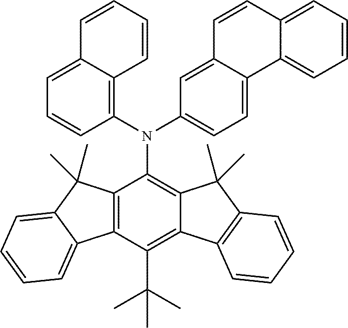 [SMILES: CC(C)(C)c1c2c(c(N(c3ccc4c(ccc5ccccc54)c3)c3cccc4ccccc34)c3c1-c1ccccc1C3(C)C)C(C)(C)c1ccccc1-2] 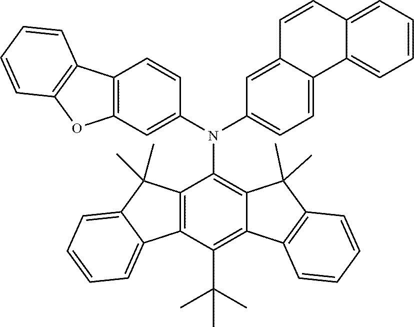 [SMILES: CC(C)(C)c1c2c(c(N(c3ccc4c(ccc5ccccc54)c3)c3ccc4c(c3)oc3ccccc34)c3c1-c1ccccc1C3(C)C)C(C)(C)c1ccccc1-2] 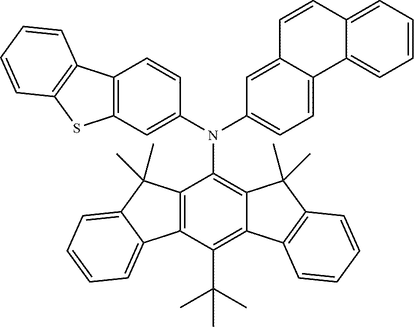 [SMILES: CC(C)(C)c1c2c(c(N(c3ccc4c(ccc5ccccc54)c3)c3ccc4c(c3)sc3ccccc34)c3c1-c1ccccc1C3(C)C)C(C)(C)c1ccccc1-2] 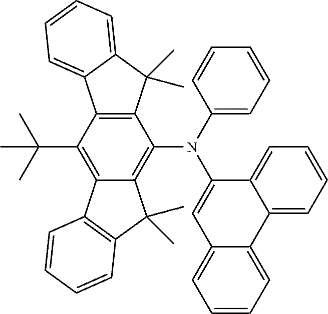 [SMILES: CC(C)(C)c1c2c(c(N(c3ccccc3)c3cc4ccccc4c4ccccc34)c3c1-c1ccccc1C3(C)C)C(C)(C)c1ccccc1-2] 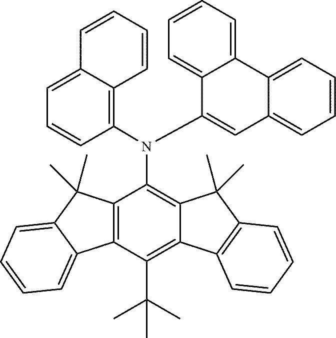 [SMILES: CC(C)(C)c1c2c(c(N(c3cccc4ccccc34)c3cc4ccccc4c4ccccc34)c3c1-c1ccccc1C3(C)C)C(C)(C)c1ccccc1-2] 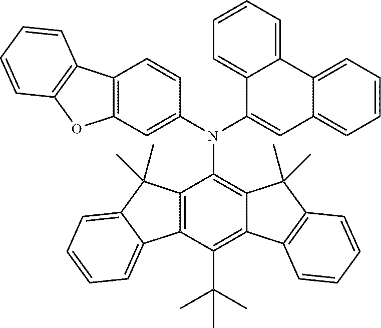 [SMILES: CC(C)(C)c1c2c(c(N(c3ccc4c(c3)oc3ccccc34)c3cc4ccccc4c4ccccc34)c3c1-c1ccccc1C3(C)C)C(C)(C)c1ccccc1-2] 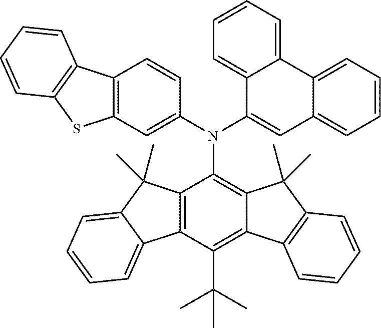 [SMILES: CC(C)(C)c1c2c(c(N(c3ccc4c(c3)sc3ccccc34)c3cc4ccccc4c4ccccc34)c3c1-c1ccccc1C3(C)C)C(C)(C)c1ccccc1-2] 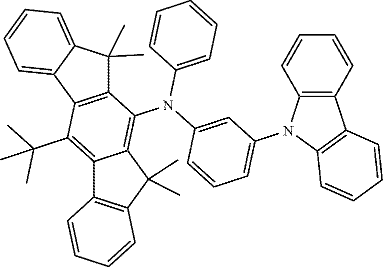 [SMILES: CC(C)(C)c1c2c(c(N(c3ccccc3)c3cccc(-n4c5ccccc5c5ccccc54)c3)c3c1-c1ccccc1C3(C)C)C(C)(C)c1ccccc1-2] 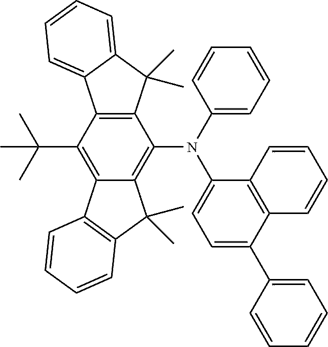 [SMILES: CC(C)(C)c1c2c(c(N(c3ccccc3)c3ccc(-c4ccccc4)c4ccccc34)c3c1-c1ccccc1C3(C)C)C(C)(C)c1ccccc1-2] 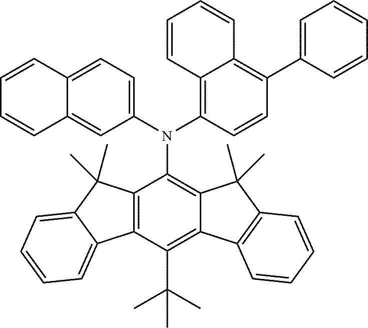 [SMILES: CC(C)(C)c1c2c(c(N(c3ccc4ccccc4c3)c3ccc(-c4ccccc4)c4ccccc34)c3c1-c1ccccc1C3(C)C)C(C)(C)c1ccccc1-2] 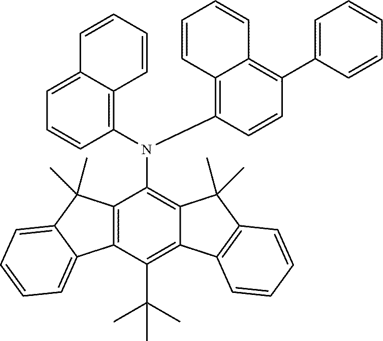 [SMILES: CC(C)(C)c1c2c(c(N(c3cccc4ccccc34)c3ccc(-c4ccccc4)c4ccccc34)c3c1-c1ccccc1C3(C)C)C(C)(C)c1ccccc1-2] 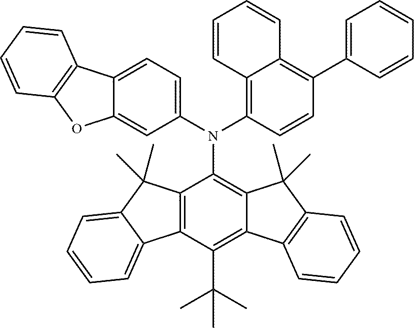 [SMILES: CC(C)(C)c1c2c(c(N(c3ccc4c(c3)oc3ccccc34)c3ccc(-c4ccccc4)c4ccccc34)c3c1-c1ccccc1C3(C)C)C(C)(C)c1ccccc1-2] 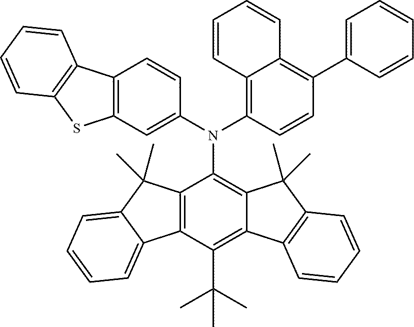 [SMILES: CC(C)(C)c1c2c(c(N(c3ccc4c(c3)sc3ccccc34)c3ccc(-c4ccccc4)c4ccccc34)c3c1-c1ccccc1C3(C)C)C(C)(C)c1ccccc1-2] 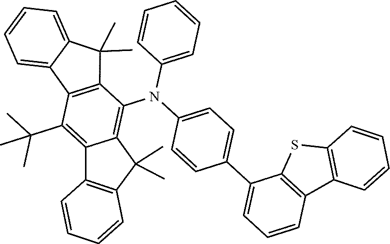 [SMILES: CC(C)(C)c1c2c(c(N(c3ccccc3)c3ccc(-c4cccc5c4sc4ccccc45)cc3)c3c1-c1ccccc1C3(C)C)C(C)(C)c1ccccc1-2] 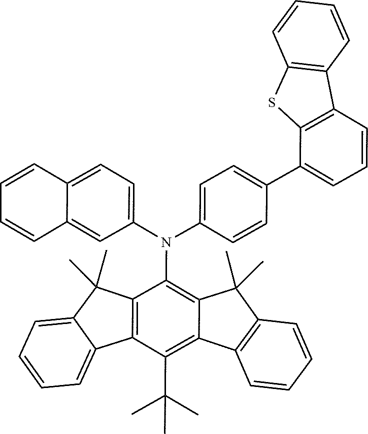 [SMILES: CC(C)(C)c1c2c(c(N(c3ccc(-c4cccc5c4sc4ccccc45)cc3)c3ccc4ccccc4c3)c3c1-c1ccccc1C3(C)C)C(C)(C)c1ccccc1-2] 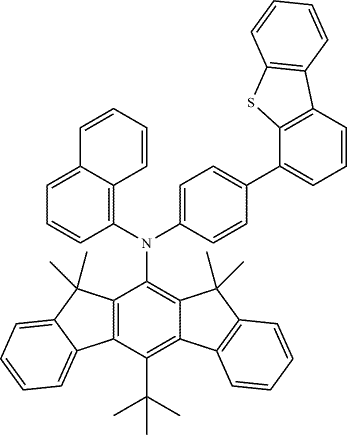 [SMILES: CC(C)(C)c1c2c(c(N(c3ccc(-c4cccc5c4sc4ccccc45)cc3)c3cccc4ccccc34)c3c1-c1ccccc1C3(C)C)C(C)(C)c1ccccc1-2] 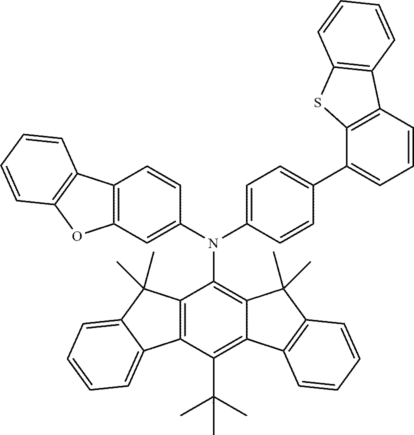 [SMILES: CC(C)(C)c1c2c(c(N(c3ccc(-c4cccc5c4sc4ccccc45)cc3)c3ccc4c(c3)oc3ccccc34)c3c1-c1ccccc1C3(C)C)C(C)(C)c1ccccc1-2] 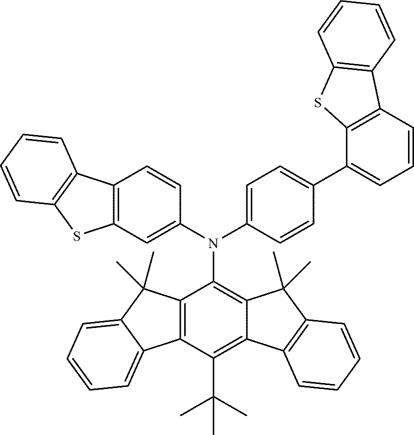 [SMILES: CC(C)(C)c1c2c(c(N(c3ccc(-c4cccc5c4sc4ccccc45)cc3)c3ccc4c(c3)sc3ccccc34)c3c1-c1ccccc1C3(C)C)C(C)(C)c1ccccc1-2] 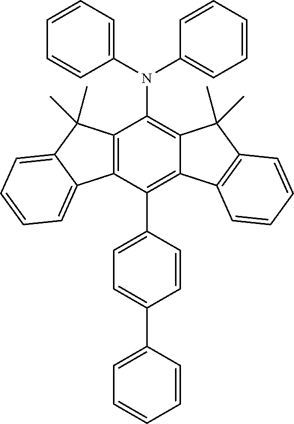 [SMILES: CC1(C)c2ccccc2-c2c(-c3ccc(-c4ccccc4)cc3)c3c(c(N(c4ccccc4)c4ccccc4)c21)C(C)(C)c1ccccc1-3] 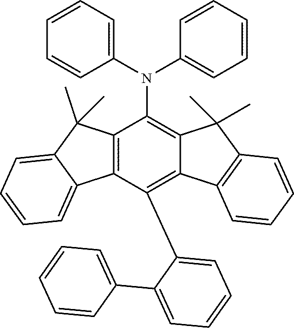 [SMILES: CC1(C)c2ccccc2-c2c(-c3ccccc3-c3ccccc3)c3c(c(N(c4ccccc4)c4ccccc4)c21)C(C)(C)c1ccccc1-3] 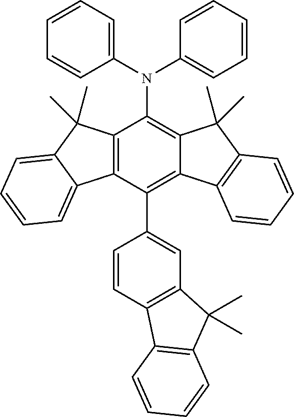 [SMILES: CC1(C)c2ccccc2-c2ccc(-c3c4c(c(N(c5ccccc5)c5ccccc5)c5c3-c3ccccc3C5(C)C)C(C)(C)c3ccccc3-4)cc21] 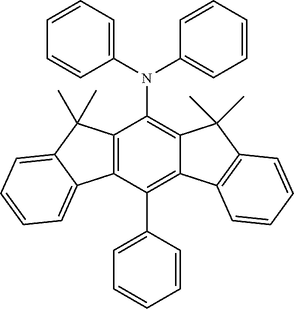 [SMILES: CC1(C)c2ccccc2-c2c(-c3ccccc3)c3c(c(N(c4ccccc4)c4ccccc4)c21)C(C)(C)c1ccccc1-3] 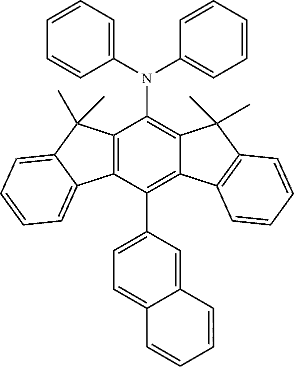 [SMILES: CC1(C)c2ccccc2-c2c(-c3ccc4ccccc4c3)c3c(c(N(c4ccccc4)c4ccccc4)c21)C(C)(C)c1ccccc1-3] 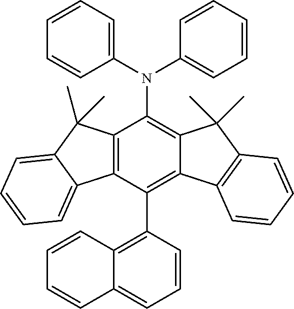 [SMILES: CC1(C)c2ccccc2-c2c(-c3cccc4ccccc34)c3c(c(N(c4ccccc4)c4ccccc4)c21)C(C)(C)c1ccccc1-3] 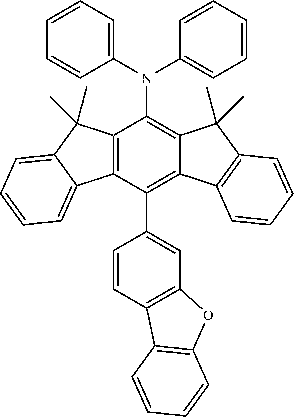 [SMILES: CC1(C)c2ccccc2-c2c(-c3ccc4c(c3)oc3ccccc34)c3c(c(N(c4ccccc4)c4ccccc4)c21)C(C)(C)c1ccccc1-3] 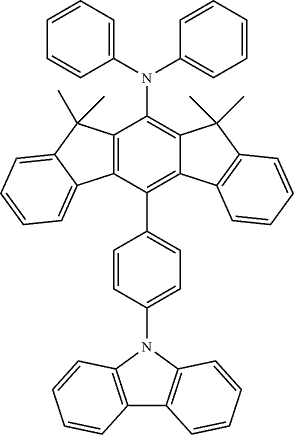 [SMILES: CC1(C)c2ccccc2-c2c(-c3ccc(-n4c5ccccc5c5ccccc54)cc3)c3c(c(N(c4ccccc4)c4ccccc4)c21)C(C)(C)c1ccccc1-3] 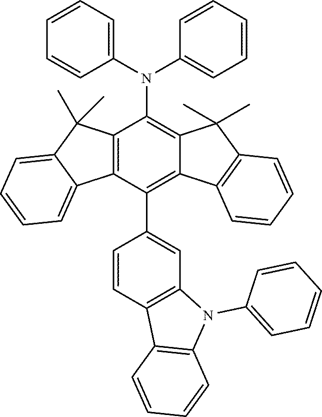 [SMILES: CC1(C)c2ccccc2-c2c(-c3ccc4c5ccccc5n(-c5ccccc5)c4c3)c3c(c(N(c4ccccc4)c4ccccc4)c21)C(C)(C)c1ccccc1-3] 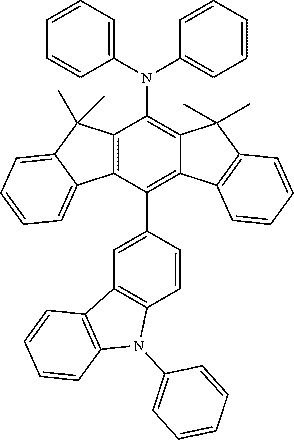 [SMILES: CC1(C)c2ccccc2-c2c(-c3ccc4c(c3)c3ccccc3n4-c3ccccc3)c3c(c(N(c4ccccc4)c4ccccc4)c21)C(C)(C)c1ccccc1-3] 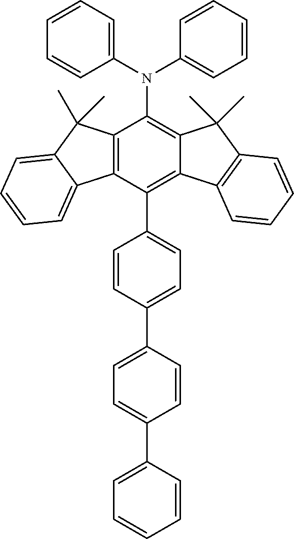 [SMILES: CC1(C)c2ccccc2-c2c(-c3ccc(-c4ccc(-c5ccccc5)cc4)cc3)c3c(c(N(c4ccccc4)c4ccccc4)c21)C(C)(C)c1ccccc1-3] 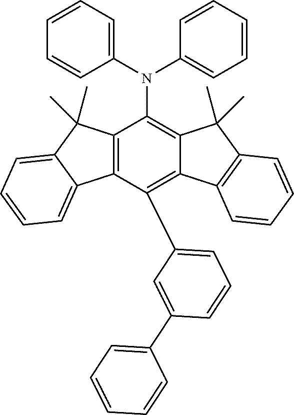 [SMILES: CC1(C)c2ccccc2-c2c(-c3cccc(-c4ccccc4)c3)c3c(c(N(c4ccccc4)c4ccccc4)c21)C(C)(C)c1ccccc1-3] 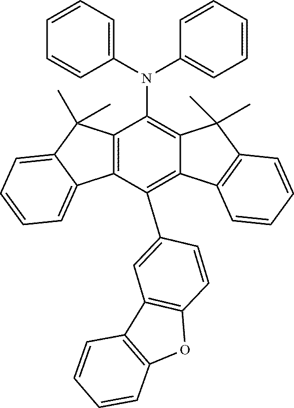 [SMILES: CC1(C)c2ccccc2-c2c(-c3ccc4oc5ccccc5c4c3)c3c(c(N(c4ccccc4)c4ccccc4)c21)C(C)(C)c1ccccc1-3] 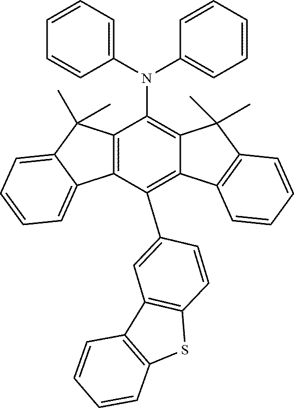 [SMILES: CC1(C)c2ccccc2-c2c(-c3ccc4sc5ccccc5c4c3)c3c(c(N(c4ccccc4)c4ccccc4)c21)C(C)(C)c1ccccc1-3] 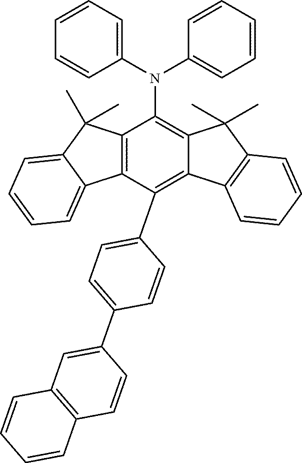 [SMILES: CC1(C)c2ccccc2-c2c(-c3ccc(-c4ccc5ccccc5c4)cc3)c3c(c(N(c4ccccc4)c4ccccc4)c21)C(C)(C)c1ccccc1-3] 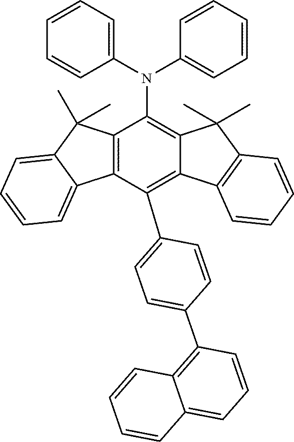 [SMILES: CC1(C)c2ccccc2-c2c(-c3ccc(-c4cccc5ccccc45)cc3)c3c(c(N(c4ccccc4)c4ccccc4)c21)C(C)(C)c1ccccc1-3] 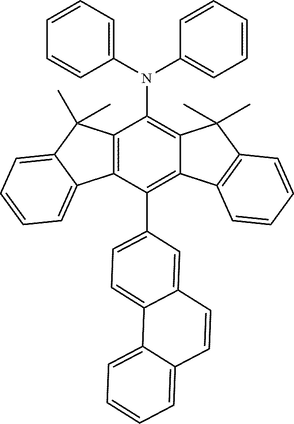 [SMILES: CC1(C)c2ccccc2-c2c(-c3ccc4c(ccc5ccccc54)c3)c3c(c(N(c4ccccc4)c4ccccc4)c21)C(C)(C)c1ccccc1-3] 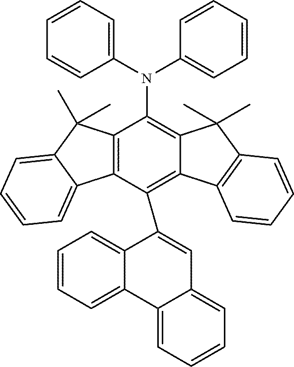 [SMILES: CC1(C)c2ccccc2-c2c(-c3cc4ccccc4c4ccccc34)c3c(c(N(c4ccccc4)c4ccccc4)c21)C(C)(C)c1ccccc1-3] 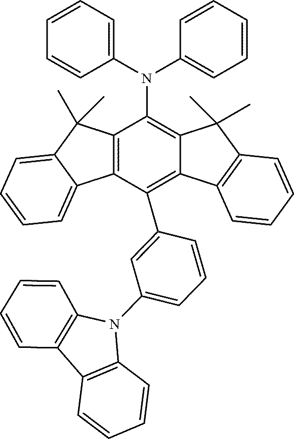 [SMILES: CC1(C)c2ccccc2-c2c(-c3cccc(-n4c5ccccc5c5ccccc54)c3)c3c(c(N(c4ccccc4)c4ccccc4)c21)C(C)(C)c1ccccc1-3] 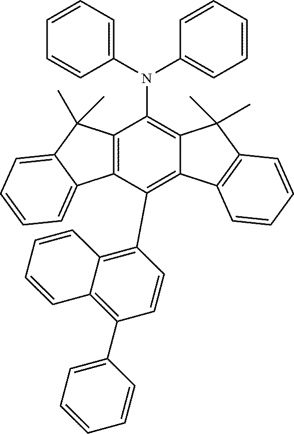 [SMILES: CC1(C)c2ccccc2-c2c(-c3ccc(-c4ccccc4)c4ccccc34)c3c(c(N(c4ccccc4)c4ccccc4)c21)C(C)(C)c1ccccc1-3] 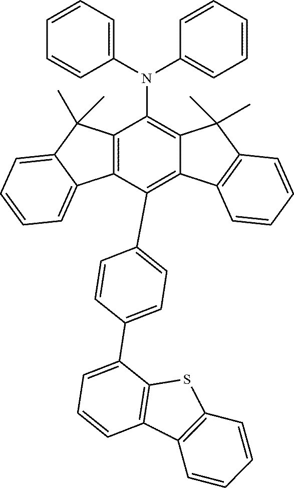 [SMILES: CC1(C)c2ccccc2-c2c(-c3ccc(-c4cccc5c4sc4ccccc45)cc3)c3c(c(N(c4ccccc4)c4ccccc4)c21)C(C)(C)c1ccccc1-3] 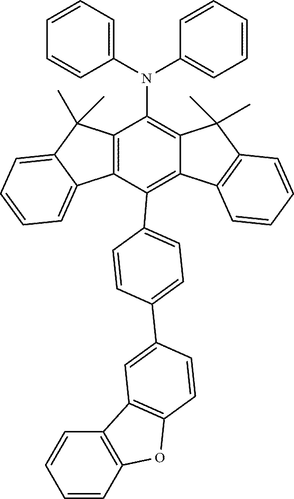 [SMILES: CC1(C)c2ccccc2-c2c(-c3ccc(-c4ccc5oc6ccccc6c5c4)cc3)c3c(c(N(c4ccccc4)c4ccccc4)c21)C(C)(C)c1ccccc1-3] 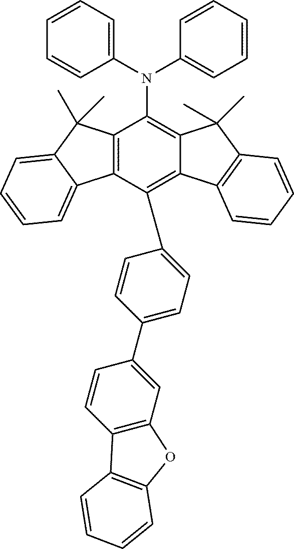 [SMILES: CC1(C)c2ccccc2-c2c(-c3ccc(-c4ccc5c(c4)oc4ccccc45)cc3)c3c(c(N(c4ccccc4)c4ccccc4)c21)C(C)(C)c1ccccc1-3] 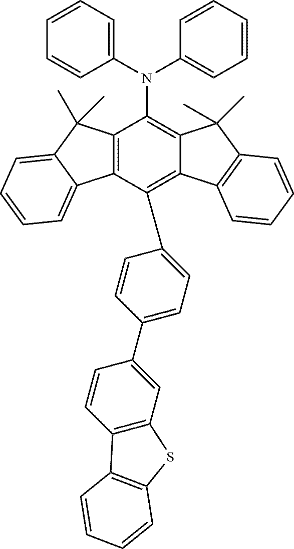 [SMILES: CC1(C)c2ccccc2-c2c(-c3ccc(-c4ccc5c(c4)sc4ccccc45)cc3)c3c(c(N(c4ccccc4)c4ccccc4)c21)C(C)(C)c1ccccc1-3] 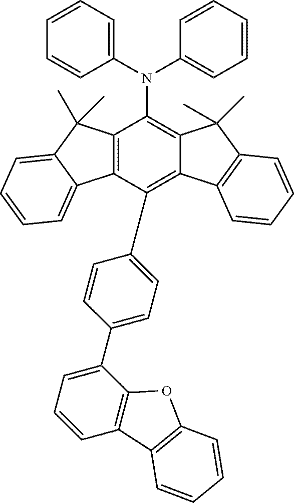 [SMILES: CC1(C)c2ccccc2-c2c(-c3ccc(-c4cccc5c4oc4ccccc45)cc3)c3c(c(N(c4ccccc4)c4ccccc4)c21)C(C)(C)c1ccccc1-3] 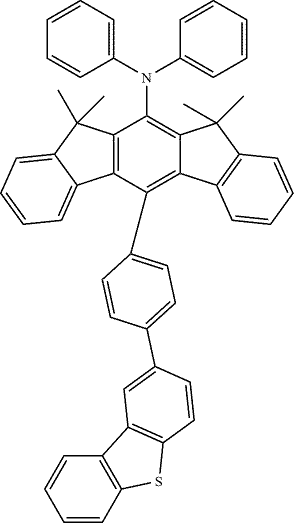 [SMILES: CC1(C)c2ccccc2-c2c(-c3ccc(-c4ccc5sc6ccccc6c5c4)cc3)c3c(c(N(c4ccccc4)c4ccccc4)c21)C(C)(C)c1ccccc1-3] 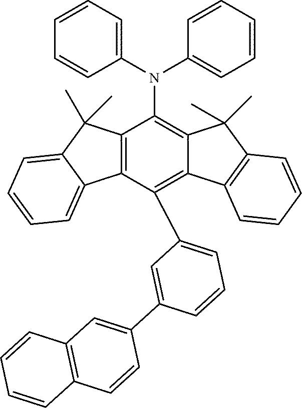 [SMILES: CC1(C)c2ccccc2-c2c(-c3cccc(-c4ccc5ccccc5c4)c3)c3c(c(N(c4ccccc4)c4ccccc4)c21)C(C)(C)c1ccccc1-3] 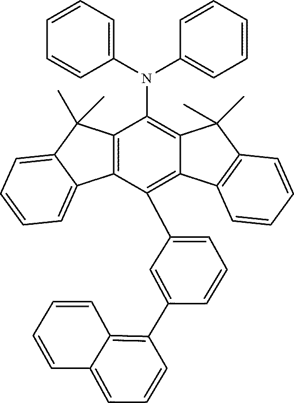 [SMILES: CC1(C)c2ccccc2-c2c(-c3cccc(-c4cccc5ccccc45)c3)c3c(c(N(c4ccccc4)c4ccccc4)c21)C(C)(C)c1ccccc1-3] 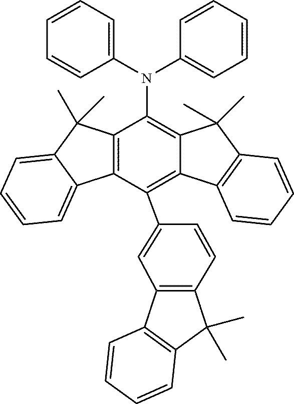 [SMILES: CC1(C)c2ccccc2-c2cc(-c3c4c(c(N(c5ccccc5)c5ccccc5)c5c3-c3ccccc3C5(C)C)C(C)(C)c3ccccc3-4)ccc21] 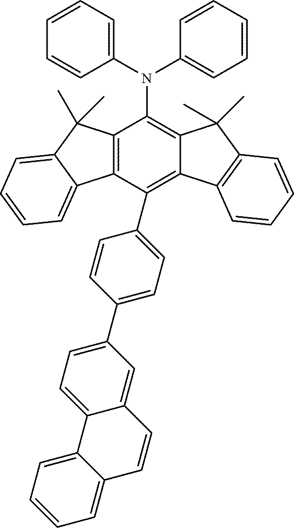 [SMILES: CC1(C)c2ccccc2-c2c(-c3ccc(-c4ccc5c(ccc6ccccc65)c4)cc3)c3c(c(N(c4ccccc4)c4ccccc4)c21)C(C)(C)c1ccccc1-3] 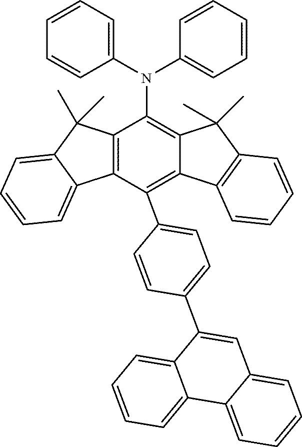 [SMILES: CC1(C)c2ccccc2-c2c(-c3ccc(-c4cc5ccccc5c5ccccc45)cc3)c3c(c(N(c4ccccc4)c4ccccc4)c21)C(C)(C)c1ccccc1-3] 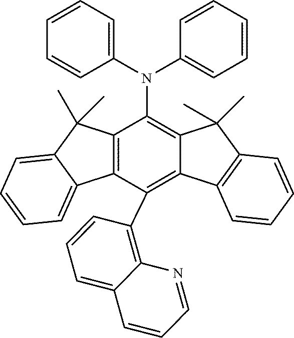 [SMILES: CC1(C)c2ccccc2-c2c(-c3cccc4cccnc34)c3c(c(N(c4ccccc4)c4ccccc4)c21)C(C)(C)c1ccccc1-3] 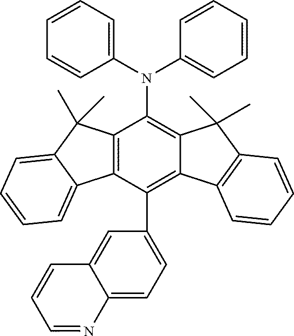 [SMILES: CC1(C)c2ccccc2-c2c(-c3ccc4ncccc4c3)c3c(c(N(c4ccccc4)c4ccccc4)c21)C(C)(C)c1ccccc1-3] 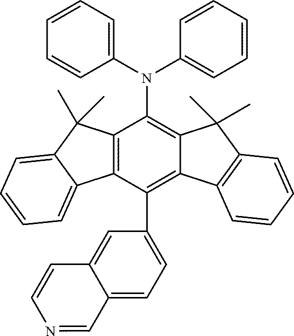 [SMILES: CC1(C)c2ccccc2-c2c(-c3ccc4cnccc4c3)c3c(c(N(c4ccccc4)c4ccccc4)c21)C(C)(C)c1ccccc1-3] 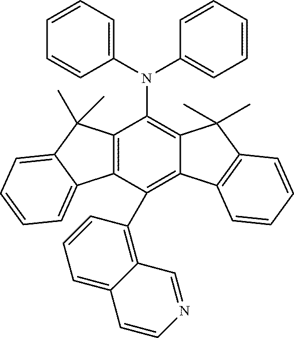 [SMILES: CC1(C)c2ccccc2-c2c(-c3cccc4ccncc34)c3c(c(N(c4ccccc4)c4ccccc4)c21)C(C)(C)c1ccccc1-3] 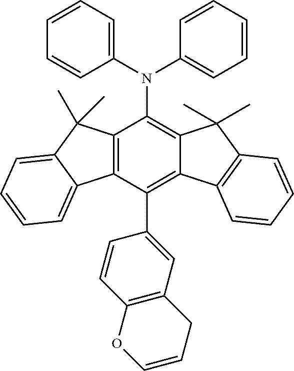 [SMILES: CC1(C)c2ccccc2-c2c(-c3ccc4c(c3)CC=CO4)c3c(c(N(c4ccccc4)c4ccccc4)c21)C(C)(C)c1ccccc1-3] 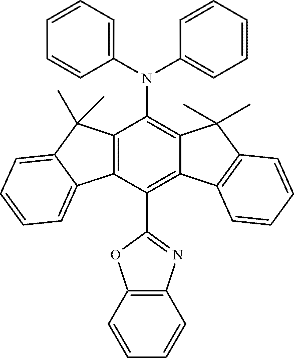 [SMILES: CC1(C)c2ccccc2-c2c(-c3nc4ccccc4o3)c3c(c(N(c4ccccc4)c4ccccc4)c21)C(C)(C)c1ccccc1-3] 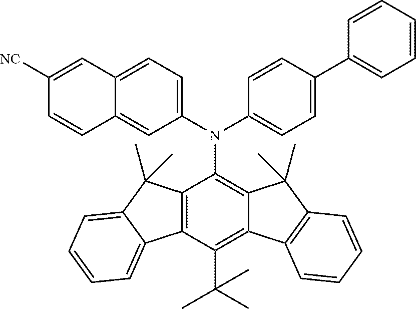 [SMILES: CC(C)(C)c1c2c(c(N(c3ccc(-c4ccccc4)cc3)c3ccc4cc(C#N)ccc4c3)c3c1-c1ccccc1C3(C)C)C(C)(C)c1ccccc1-2] 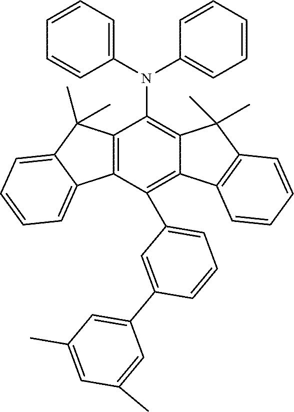 [SMILES: Cc1cc(C)cc(-c2cccc(-c3c4c(c(N(c5ccccc5)c5ccccc5)c5c3-c3ccccc3C5(C)C)C(C)(C)c3ccccc3-4)c2)c1] 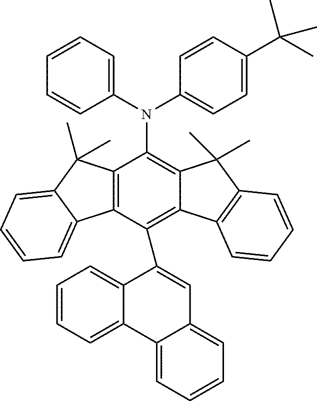 [SMILES: CC(C)(C)c1ccc(N(c2ccccc2)c2c3c(c(-c4cc5ccccc5c5ccccc45)c4c2C(C)(C)c2ccccc2-4)-c2ccccc2C3(C)C)cc1] 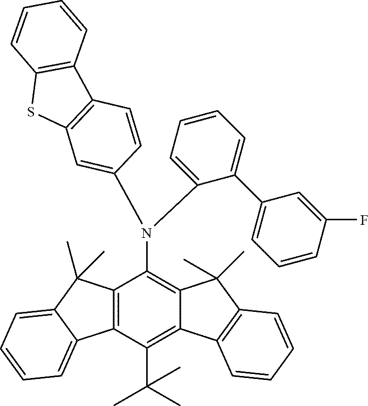 [SMILES: CC(C)(C)c1c2c(c(N(c3ccc4c(c3)sc3ccccc34)c3ccccc3-c3cccc(F)c3)c3c1-c1ccccc1C3(C)C)C(C)(C)c1ccccc1-2] 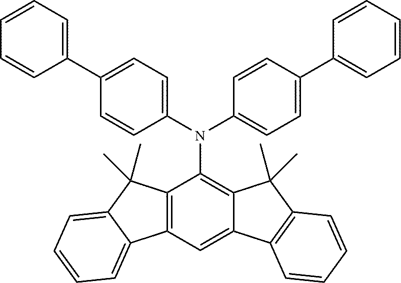 [SMILES: CC1(C)c2ccccc2-c2cc3c(c(N(c4ccc(-c5ccccc5)cc4)c4ccc(-c5ccccc5)cc4)c21)C(C)(C)c1ccccc1-3] 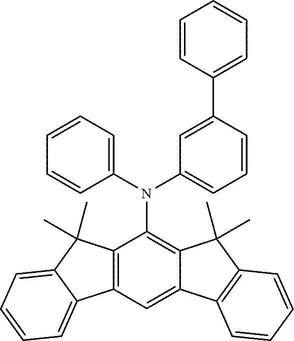 [SMILES: CC1(C)c2ccccc2-c2cc3c(c(N(c4ccccc4)c4cccc(-c5ccccc5)c4)c21)C(C)(C)c1ccccc1-3] 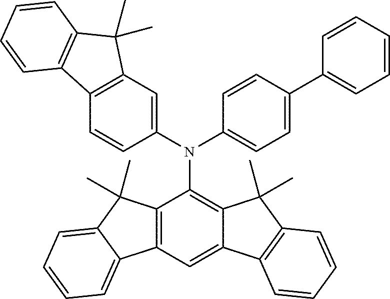 [SMILES: CC1(C)c2ccccc2-c2ccc(N(c3ccc(-c4ccccc4)cc3)c3c4c(cc5c3C(C)(C)c3ccccc3-5)-c3ccccc3C4(C)C)cc21] 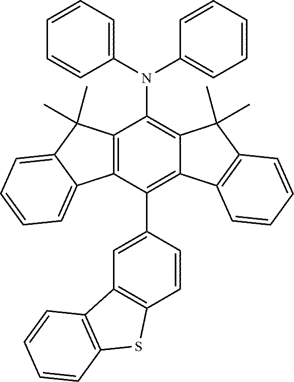 [SMILES: CC1(C)c2ccccc2-c2c(-c3ccc4sc5ccccc5c4c3)c3c(c(N(c4ccccc4)c4ccccc4)c21)C(C)(C)c1ccccc1-3] 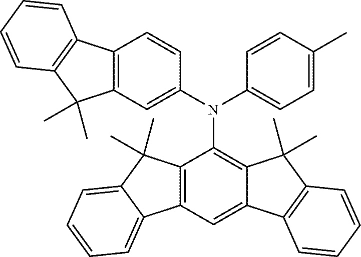 [SMILES: Cc1ccc(N(c2ccc3c(c2)C(C)(C)c2ccccc2-3)c2c3c(cc4c2C(C)(C)c2ccccc2-4)-c2ccccc2C3(C)C)cc1] 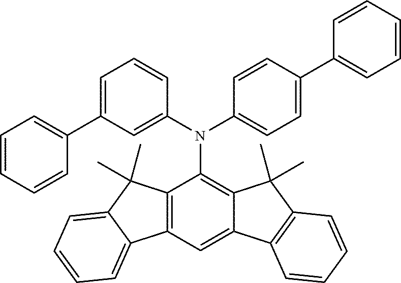 [SMILES: CC1(C)c2ccccc2-c2cc3c(c(N(c4ccc(-c5ccccc5)cc4)c4cccc(-c5ccccc5)c4)c21)C(C)(C)c1ccccc1-3] 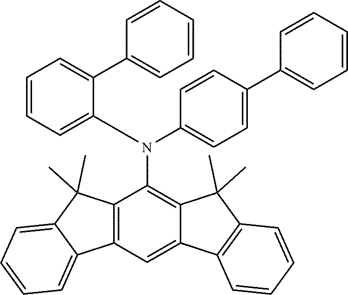 [SMILES: CC1(C)c2ccccc2-c2cc3c(c(N(c4ccc(-c5ccccc5)cc4)c4ccccc4-c4ccccc4)c21)C(C)(C)c1ccccc1-3] 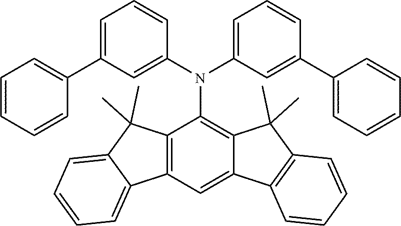 [SMILES: CC1(C)c2ccccc2-c2cc3c(c(N(c4cccc(-c5ccccc5)c4)c4cccc(-c5ccccc5)c4)c21)C(C)(C)c1ccccc1-3] 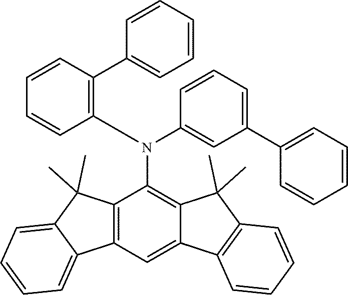 [SMILES: CC1(C)c2ccccc2-c2cc3c(c(N(c4cccc(-c5ccccc5)c4)c4ccccc4-c4ccccc4)c21)C(C)(C)c1ccccc1-3] 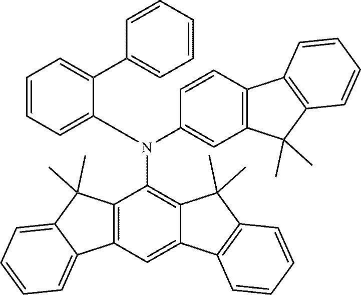 [SMILES: CC1(C)c2ccccc2-c2ccc(N(c3ccccc3-c3ccccc3)c3c4c(cc5c3C(C)(C)c3ccccc3-5)-c3ccccc3C4(C)C)cc21] 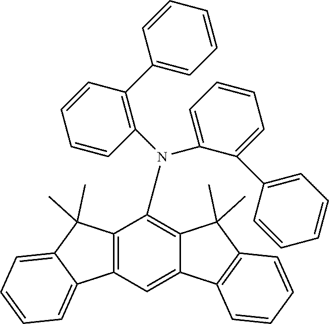 [SMILES: CC1(C)c2ccccc2-c2cc3c(c(N(c4ccccc4-c4ccccc4)c4ccccc4-c4ccccc4)c21)C(C)(C)c1ccccc1-3] 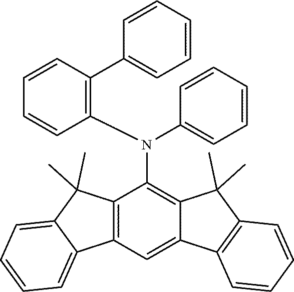 [SMILES: CC1(C)c2ccccc2-c2cc3c(c(N(c4ccccc4)c4ccccc4-c4ccccc4)c21)C(C)(C)c1ccccc1-3] 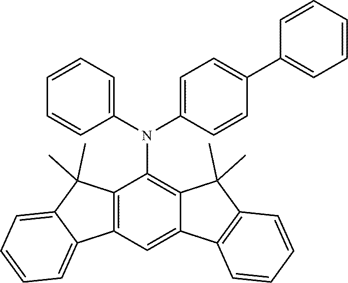 [SMILES: CC1(C)c2ccccc2-c2cc3c(c(N(c4ccccc4)c4ccc(-c5ccccc5)cc4)c21)C(C)(C)c1ccccc1-3] 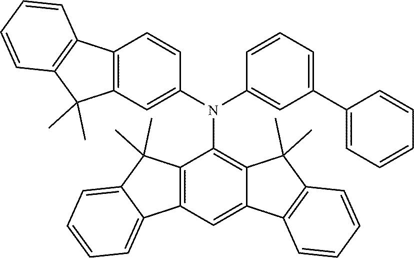 [SMILES: CC1(C)c2ccccc2-c2ccc(N(c3cccc(-c4ccccc4)c3)c3c4c(cc5c3C(C)(C)c3ccccc3-5)-c3ccccc3C4(C)C)cc21] 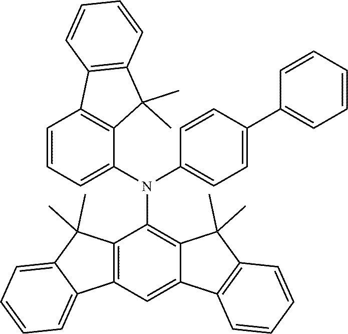 [SMILES: CC1(C)c2ccccc2-c2cccc(N(c3ccc(-c4ccccc4)cc3)c3c4c(cc5c3C(C)(C)c3ccccc3-5)-c3ccccc3C4(C)C)c21] 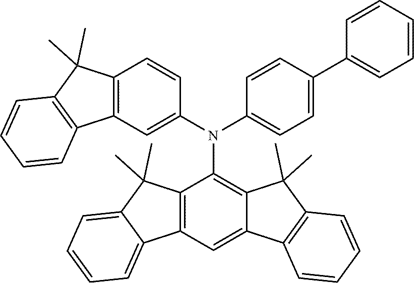 [SMILES: CC1(C)c2ccccc2-c2cc(N(c3ccc(-c4ccccc4)cc3)c3c4c(cc5c3C(C)(C)c3ccccc3-5)-c3ccccc3C4(C)C)ccc21] 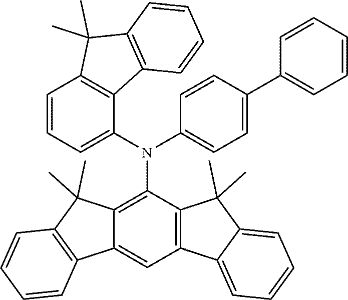 [SMILES: CC1(C)c2ccccc2-c2c(N(c3ccc(-c4ccccc4)cc3)c3c4c(cc5c3C(C)(C)c3ccccc3-5)-c3ccccc3C4(C)C)cccc21] 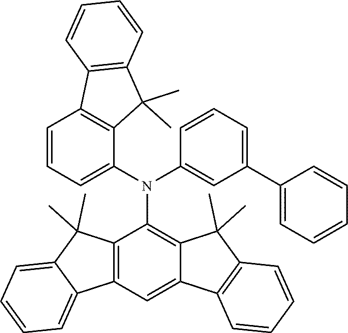 [SMILES: CC1(C)c2ccccc2-c2cccc(N(c3cccc(-c4ccccc4)c3)c3c4c(cc5c3C(C)(C)c3ccccc3-5)-c3ccccc3C4(C)C)c21] 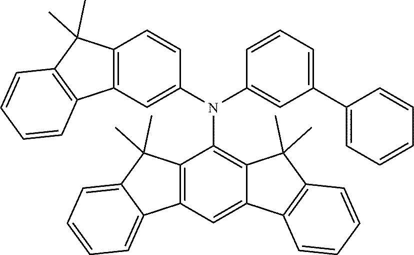 [SMILES: CC1(C)c2ccccc2-c2cc(N(c3cccc(-c4ccccc4)c3)c3c4c(cc5c3C(C)(C)c3ccccc3-5)-c3ccccc3C4(C)C)ccc21] 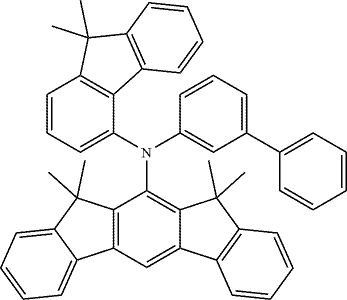 [SMILES: CC1(C)c2ccccc2-c2c(N(c3cccc(-c4ccccc4)c3)c3c4c(cc5c3C(C)(C)c3ccccc3-5)-c3ccccc3C4(C)C)cccc21] 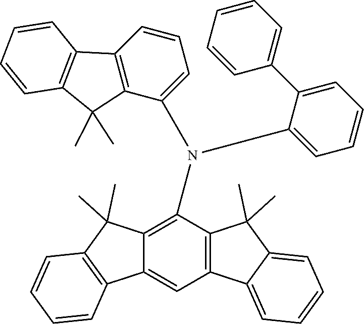 [SMILES: CC1(C)c2ccccc2-c2cccc(N(c3ccccc3-c3ccccc3)c3c4c(cc5c3C(C)(C)c3ccccc3-5)-c3ccccc3C4(C)C)c21] 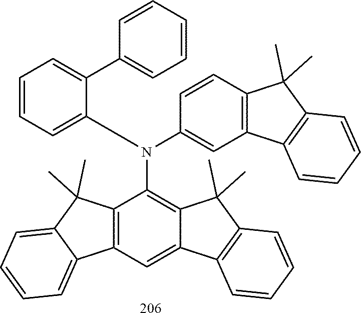 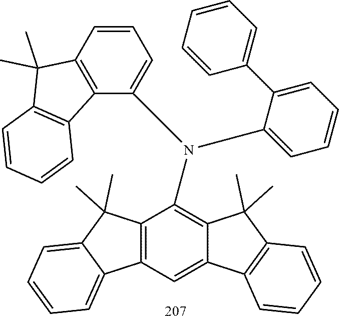 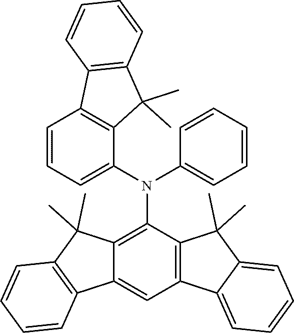 [SMILES: CC1(C)c2ccccc2-c2cccc(N(c3ccccc3)c3c4c(cc5c3C(C)(C)c3ccccc3-5)-c3ccccc3C4(C)C)c21] 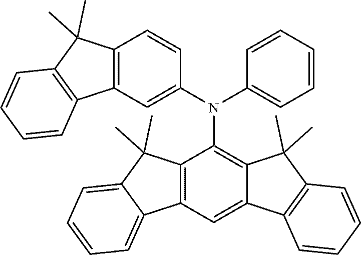 [SMILES: CC1(C)c2ccccc2-c2cc(N(c3ccccc3)c3c4c(cc5c3C(C)(C)c3ccccc3-5)-c3ccccc3C4(C)C)ccc21] 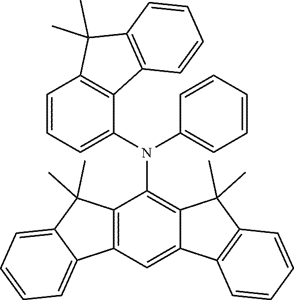 [SMILES: CC1(C)c2ccccc2-c2c(N(c3ccccc3)c3c4c(cc5c3C(C)(C)c3ccccc3-5)-c3ccccc3C4(C)C)cccc21] 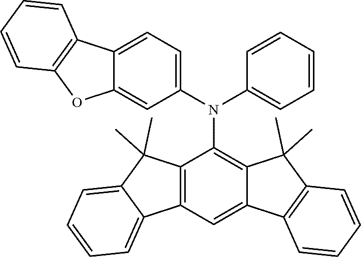 [SMILES: CC1(C)c2ccccc2-c2cc3c(c(N(c4ccccc4)c4ccc5c(c4)oc4ccccc45)c21)C(C)(C)c1ccccc1-3] 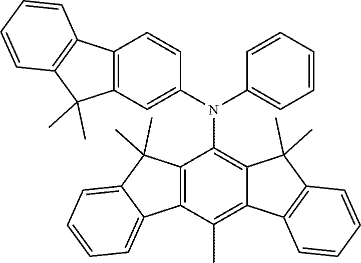 [SMILES: Cc1c2c(c(N(c3ccccc3)c3ccc4c(c3)C(C)(C)c3ccccc3-4)c3c1-c1ccccc1C3(C)C)C(C)(C)c1ccccc1-2] 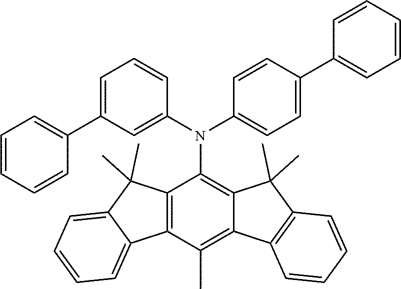 [SMILES: Cc1c2c(c(N(c3ccc(-c4ccccc4)cc3)c3cccc(-c4ccccc4)c3)c3c1-c1ccccc1C3(C)C)C(C)(C)c1ccccc1-2] 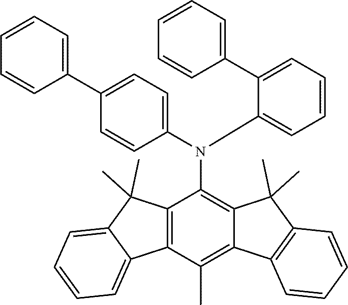 [SMILES: Cc1c2c(c(N(c3ccc(-c4ccccc4)cc3)c3ccccc3-c3ccccc3)c3c1-c1ccccc1C3(C)C)C(C)(C)c1ccccc1-2] 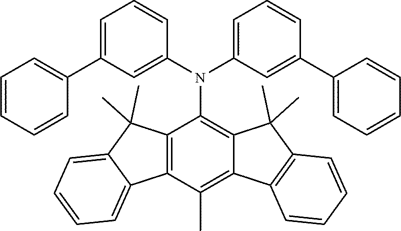 [SMILES: Cc1c2c(c(N(c3cccc(-c4ccccc4)c3)c3cccc(-c4ccccc4)c3)c3c1-c1ccccc1C3(C)C)C(C)(C)c1ccccc1-2] 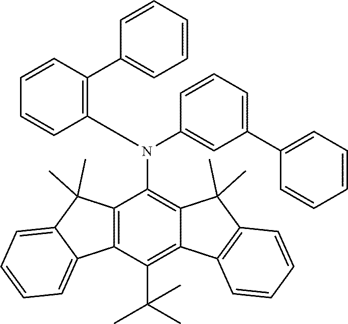 [SMILES: CC(C)(C)c1c2c(c(N(c3cccc(-c4ccccc4)c3)c3ccccc3-c3ccccc3)c3c1-c1ccccc1C3(C)C)C(C)(C)c1ccccc1-2] 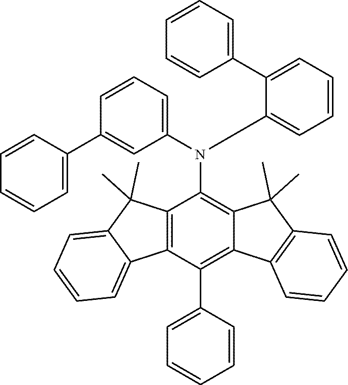 [SMILES: CC1(C)c2ccccc2-c2c(-c3ccccc3)c3c(c(N(c4cccc(-c5ccccc5)c4)c4ccccc4-c4ccccc4)c21)C(C)(C)c1ccccc1-3] 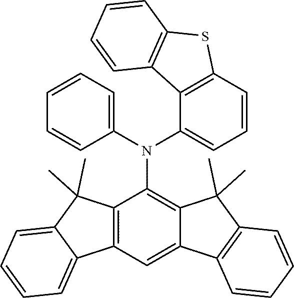 [SMILES: CC1(C)c2ccccc2-c2cc3c(c(N(c4ccccc4)c4cccc5sc6ccccc6c45)c21)C(C)(C)c1ccccc1-3] 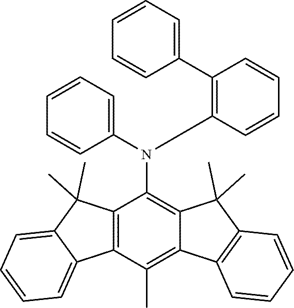 [SMILES: Cc1c2c(c(N(c3ccccc3)c3ccccc3-c3ccccc3)c3c1-c1ccccc1C3(C)C)C(C)(C)c1ccccc1-2] 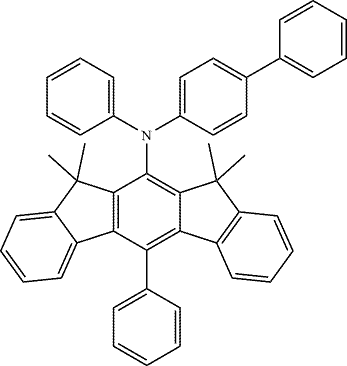 [SMILES: CC1(C)c2ccccc2-c2c(-c3ccccc3)c3c(c(N(c4ccccc4)c4ccc(-c5ccccc5)cc4)c21)C(C)(C)c1ccccc1-3] 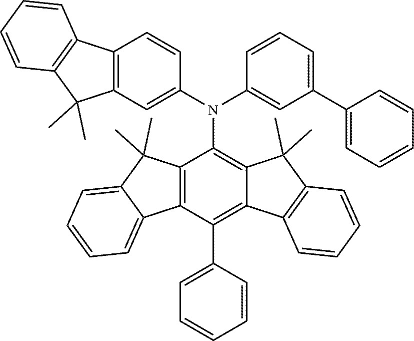 [SMILES: CC1(C)c2ccccc2-c2ccc(N(c3cccc(-c4ccccc4)c3)c3c4c(c(-c5ccccc5)c5c3C(C)(C)c3ccccc3-5)-c3ccccc3C4(C)C)cc21] 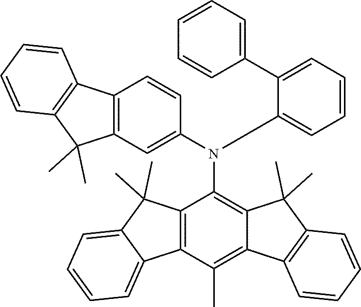 [SMILES: Cc1c2c(c(N(c3ccc4c(c3)C(C)(C)c3ccccc3-4)c3ccccc3-c3ccccc3)c3c1-c1ccccc1C3(C)C)C(C)(C)c1ccccc1-2] 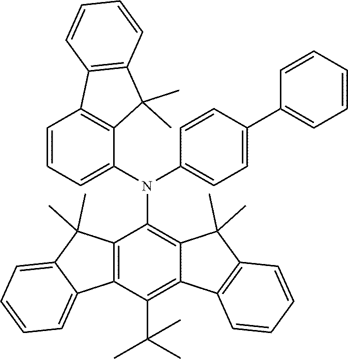 [SMILES: CC(C)(C)c1c2c(c(N(c3ccc(-c4ccccc4)cc3)c3cccc4c3C(C)(C)c3ccccc3-4)c3c1-c1ccccc1C3(C)C)C(C)(C)c1ccccc1-2] 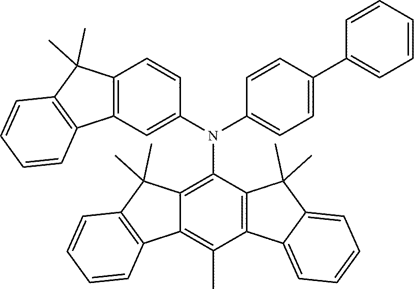 [SMILES: Cc1c2c(c(N(c3ccc(-c4ccccc4)cc3)c3ccc4c(c3)-c3ccccc3C4(C)C)c3c1-c1ccccc1C3(C)C)C(C)(C)c1ccccc1-2] 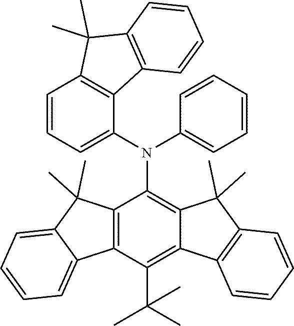 [SMILES: CC(C)(C)c1c2c(c(N(c3ccccc3)c3cccc4c3-c3ccccc3C4(C)C)c3c1-c1ccccc1C3(C)C)C(C)(C)c1ccccc1-2] 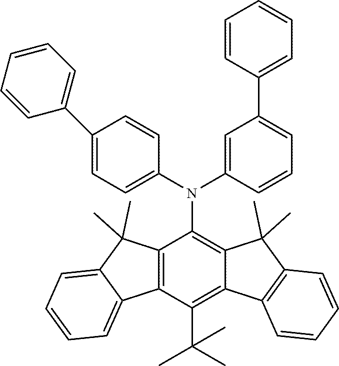 [SMILES: CC(C)(C)c1c2c(c(N(c3ccc(-c4ccccc4)cc3)c3cccc(-c4ccccc4)c3)c3c1-c1ccccc1C3(C)C)C(C)(C)c1ccccc1-2] 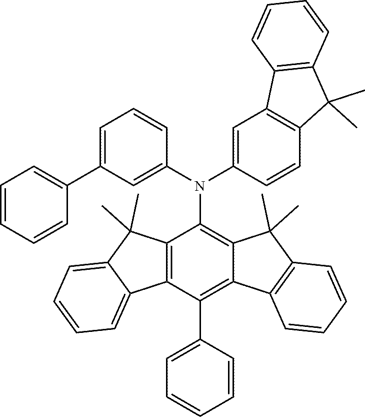 [SMILES: CC1(C)c2ccccc2-c2cc(N(c3cccc(-c4ccccc4)c3)c3c4c(c(-c5ccccc5)c5c3C(C)(C)c3ccccc3-5)-c3ccccc3C4(C)C)ccc21] 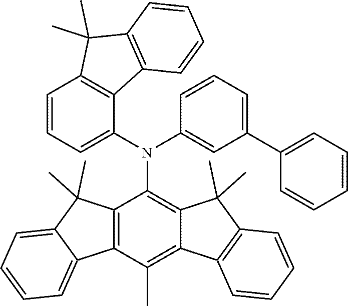 [SMILES: Cc1c2c(c(N(c3cccc(-c4ccccc4)c3)c3cccc4c3-c3ccccc3C4(C)C)c3c1-c1ccccc1C3(C)C)C(C)(C)c1ccccc1-2] 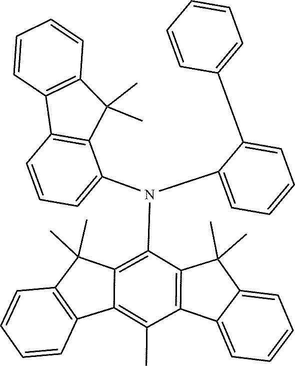 [SMILES: Cc1c2c(c(N(c3ccccc3-c3ccccc3)c3cccc4c3C(C)(C)c3ccccc3-4)c3c1-c1ccccc1C3(C)C)C(C)(C)c1ccccc1-2] 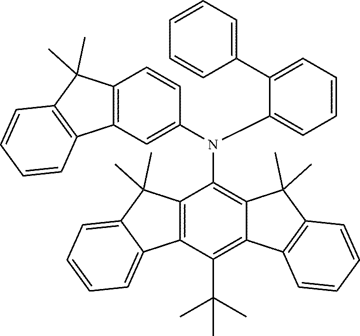 [SMILES: CC(C)(C)c1c2c(c(N(c3ccc4c(c3)-c3ccccc3C4(C)C)c3ccccc3-c3ccccc3)c3c1-c1ccccc1C3(C)C)C(C)(C)c1ccccc1-2] 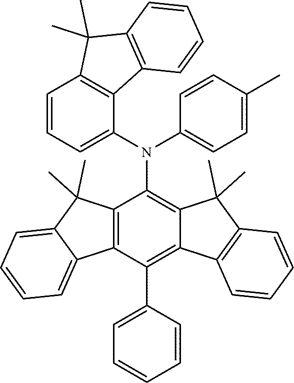 [SMILES: Cc1ccc(N(c2cccc3c2-c2ccccc2C3(C)C)c2c3c(c(-c4ccccc4)c4c2C(C)(C)c2ccccc2-4)-c2ccccc2C3(C)C)cc1] 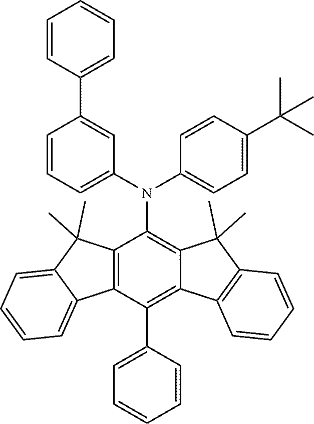 [SMILES: CC(C)(C)c1ccc(N(c2cccc(-c3ccccc3)c2)c2c3c(c(-c4ccccc4)c4c2C(C)(C)c2ccccc2-4)-c2ccccc2C3(C)C)cc1] 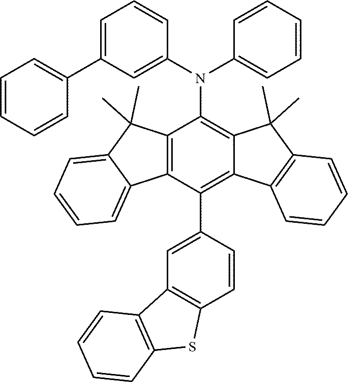 [SMILES: CC1(C)c2ccccc2-c2c(-c3ccc4sc5ccccc5c4c3)c3c(c(N(c4ccccc4)c4cccc(-c5ccccc5)c4)c21)C(C)(C)c1ccccc1-3] 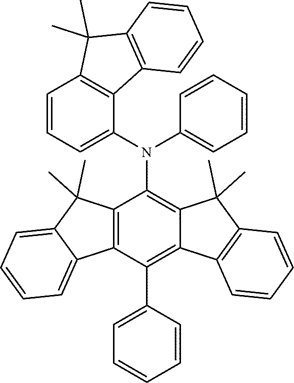 [SMILES: CC1(C)c2ccccc2-c2c(N(c3ccccc3)c3c4c(c(-c5ccccc5)c5c3C(C)(C)c3ccccc3-5)-c3ccccc3C4(C)C)cccc21] 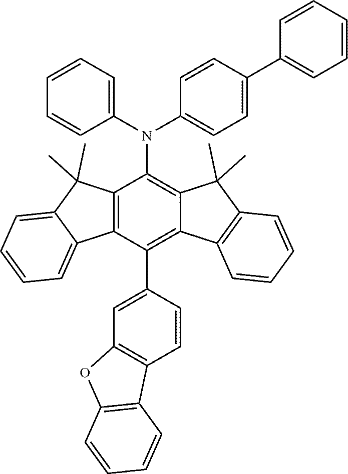 [SMILES: CC1(C)c2ccccc2-c2c(-c3ccc4c(c3)oc3ccccc34)c3c(c(N(c4ccccc4)c4ccc(-c5ccccc5)cc4)c21)C(C)(C)c1ccccc1-3] 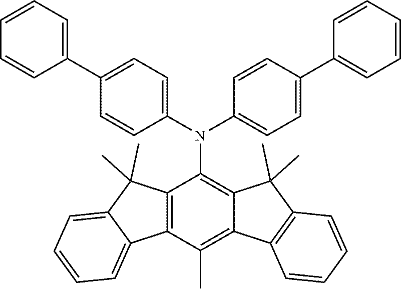 [SMILES: Cc1c2c(c(N(c3ccc(-c4ccccc4)cc3)c3ccc(-c4ccccc4)cc3)c3c1-c1ccccc1C3(C)C)C(C)(C)c1ccccc1-2] 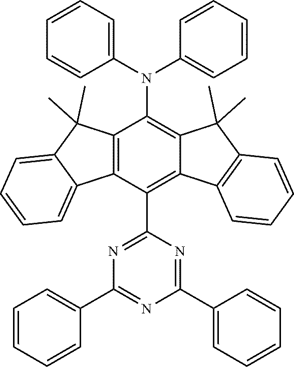 [SMILES: CC1(C)c2ccccc2-c2c(-c3nc(-c4ccccc4)nc(-c4ccccc4)n3)c3c(c(N(c4ccccc4)c4ccccc4)c21)C(C)(C)c1ccccc1-3] 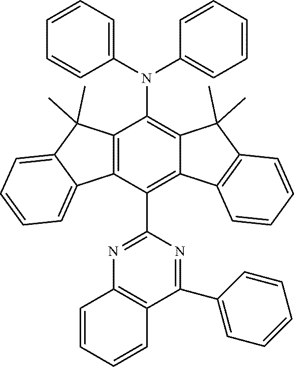 [SMILES: CC1(C)c2ccccc2-c2c(-c3nc(-c4ccccc4)c4ccccc4n3)c3c(c(N(c4ccccc4)c4ccccc4)c21)C(C)(C)c1ccccc1-3] 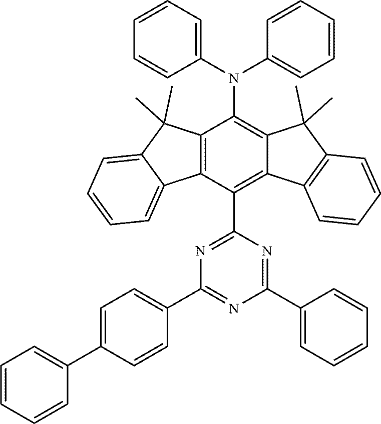 [SMILES: CC1(C)c2ccccc2-c2c(-c3nc(-c4ccccc4)nc(-c4ccc(-c5ccccc5)cc4)n3)c3c(c(N(c4ccccc4)c4ccccc4)c21)C(C)(C)c1ccccc1-3] 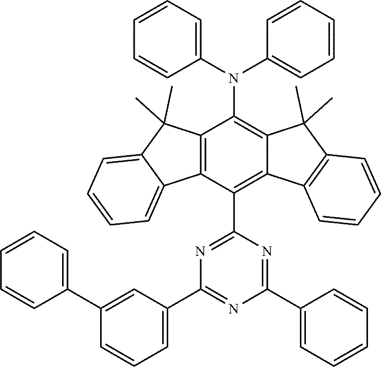 [SMILES: CC1(C)c2ccccc2-c2c(-c3nc(-c4ccccc4)nc(-c4cccc(-c5ccccc5)c4)n3)c3c(c(N(c4ccccc4)c4ccccc4)c21)C(C)(C)c1ccccc1-3] 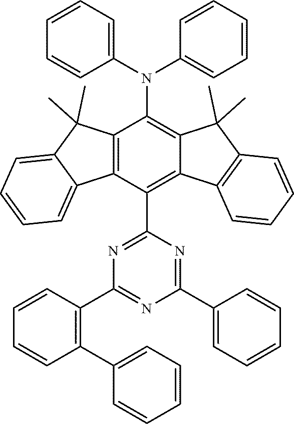 [SMILES: CC1(C)c2ccccc2-c2c(-c3nc(-c4ccccc4)nc(-c4ccccc4-c4ccccc4)n3)c3c(c(N(c4ccccc4)c4ccccc4)c21)C(C)(C)c1ccccc1-3] 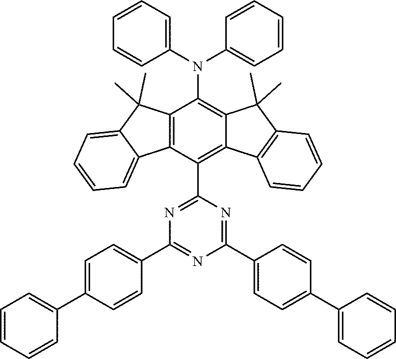 [SMILES: CC1(C)c2ccccc2-c2c(-c3nc(-c4ccc(-c5ccccc5)cc4)nc(-c4ccc(-c5ccccc5)cc4)n3)c3c(c(N(c4ccccc4)c4ccccc4)c21)C(C)(C)c1ccccc1-3] 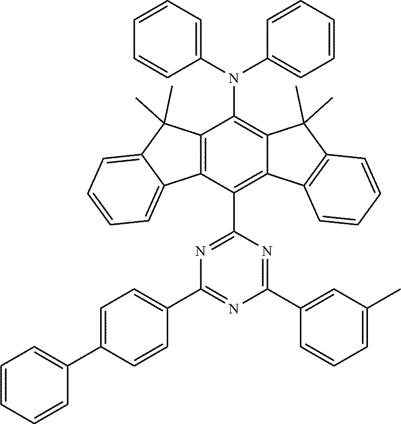 [SMILES: Cc1cccc(-c2nc(-c3ccc(-c4ccccc4)cc3)nc(-c3c4c(c(N(c5ccccc5)c5ccccc5)c5c3-c3ccccc3C5(C)C)C(C)(C)c3ccccc3-4)n2)c1] 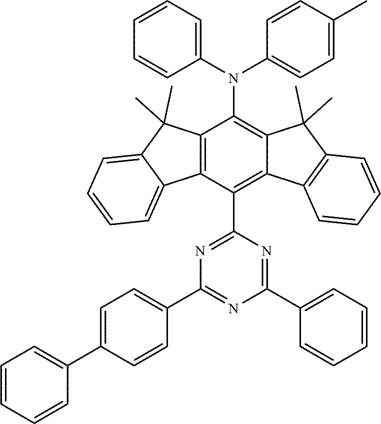 [SMILES: Cc1ccc(N(c2ccccc2)c2c3c(c(-c4nc(-c5ccccc5)nc(-c5ccc(-c6ccccc6)cc5)n4)c4c2C(C)(C)c2ccccc2-4)-c2ccccc2C3(C)C)cc1] 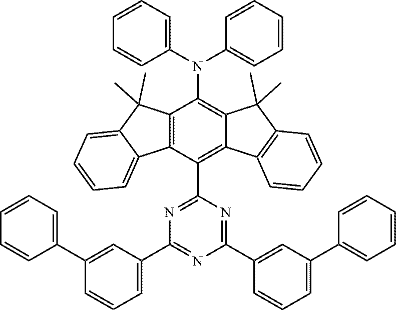 [SMILES: CC1(C)c2ccccc2-c2c(-c3nc(-c4cccc(-c5ccccc5)c4)nc(-c4cccc(-c5ccccc5)c4)n3)c3c(c(N(c4ccccc4)c4ccccc4)c21)C(C)(C)c1ccccc1-3] 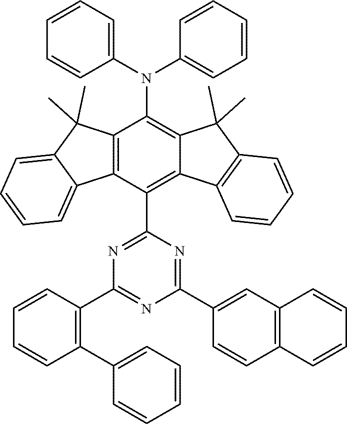 [SMILES: CC1(C)c2ccccc2-c2c(-c3nc(-c4ccc5ccccc5c4)nc(-c4ccccc4-c4ccccc4)n3)c3c(c(N(c4ccccc4)c4ccccc4)c21)C(C)(C)c1ccccc1-3] 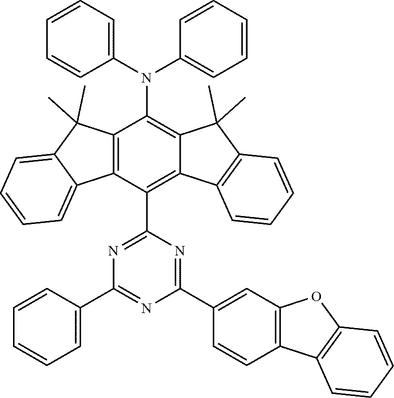 [SMILES: CC1(C)c2ccccc2-c2c(-c3nc(-c4ccccc4)nc(-c4ccc5c(c4)oc4ccccc45)n3)c3c(c(N(c4ccccc4)c4ccccc4)c21)C(C)(C)c1ccccc1-3] 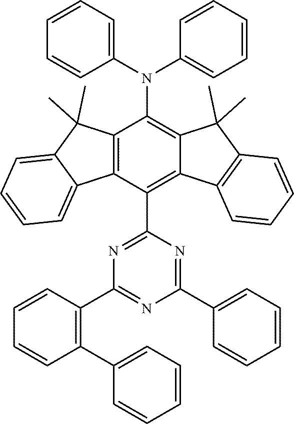 [SMILES: CC1(C)c2ccccc2-c2c(-c3nc(-c4ccccc4)nc(-c4ccccc4-c4ccccc4)n3)c3c(c(N(c4ccccc4)c4ccccc4)c21)C(C)(C)c1ccccc1-3] 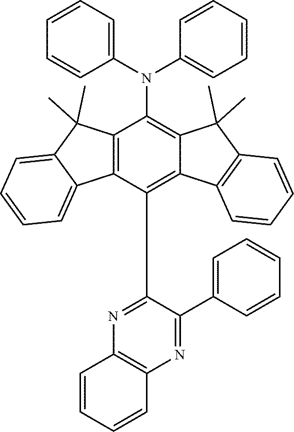 [SMILES: CC1(C)c2ccccc2-c2c(-c3nc4ccccc4nc3-c3ccccc3)c3c(c(N(c4ccccc4)c4ccccc4)c21)C(C)(C)c1ccccc1-3] 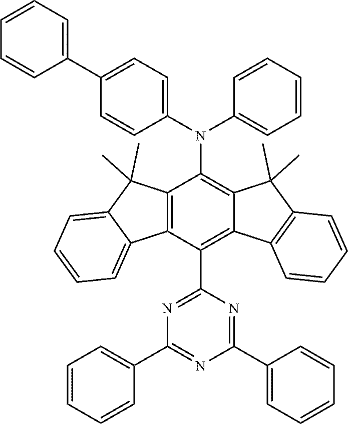 [SMILES: CC1(C)c2ccccc2-c2c(-c3nc(-c4ccccc4)nc(-c4ccccc4)n3)c3c(c(N(c4ccccc4)c4ccc(-c5ccccc5)cc4)c21)C(C)(C)c1ccccc1-3] 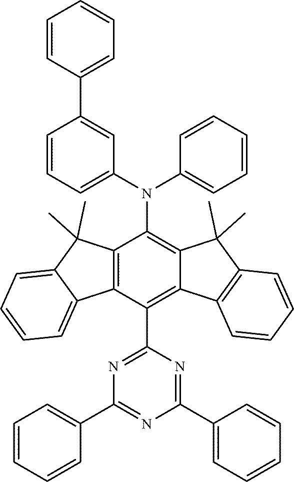 [SMILES: CC1(C)c2ccccc2-c2c(-c3nc(-c4ccccc4)nc(-c4ccccc4)n3)c3c(c(N(c4ccccc4)c4cccc(-c5ccccc5)c4)c21)C(C)(C)c1ccccc1-3] 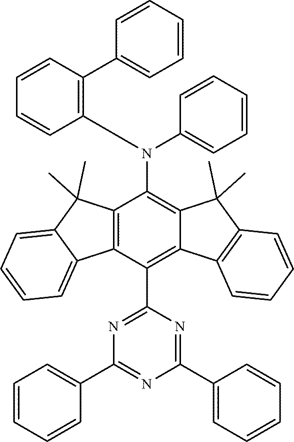 [SMILES: CC1(C)c2ccccc2-c2c(-c3nc(-c4ccccc4)nc(-c4ccccc4)n3)c3c(c(N(c4ccccc4)c4ccccc4-c4ccccc4)c21)C(C)(C)c1ccccc1-3] 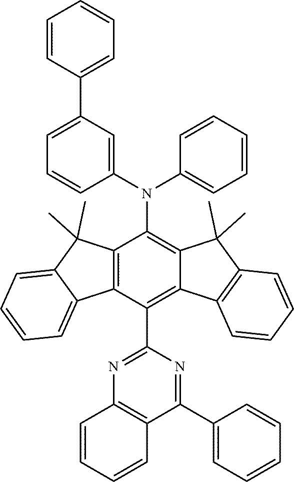 [SMILES: CC1(C)c2ccccc2-c2c(-c3nc(-c4ccccc4)c4ccccc4n3)c3c(c(N(c4ccccc4)c4cccc(-c5ccccc5)c4)c21)C(C)(C)c1ccccc1-3] 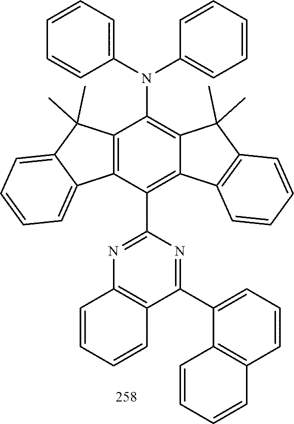 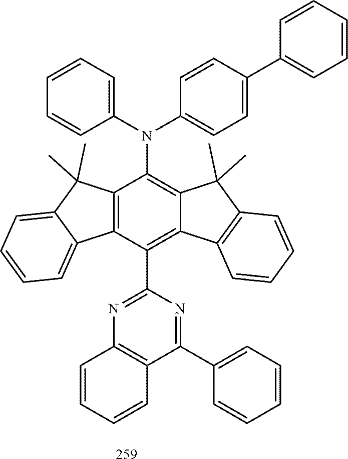 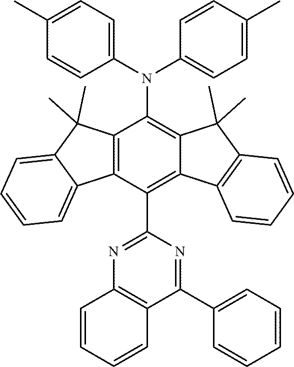 [SMILES: Cc1ccc(N(c2ccc(C)cc2)c2c3c(c(-c4nc(-c5ccccc5)c5ccccc5n4)c4c2C(C)(C)c2ccccc2-4)-c2ccccc2C3(C)C)cc1] 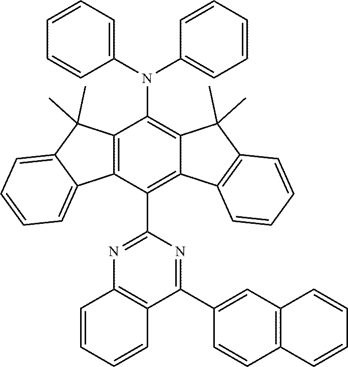 [SMILES: CC1(C)c2ccccc2-c2c(-c3nc(-c4ccc5ccccc5c4)c4ccccc4n3)c3c(c(N(c4ccccc4)c4ccccc4)c21)C(C)(C)c1ccccc1-3] 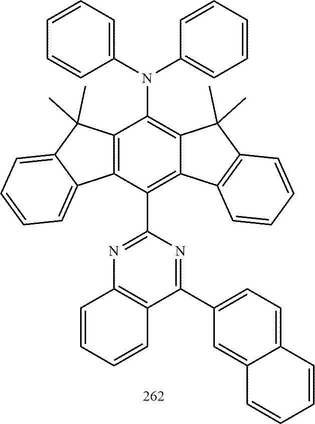 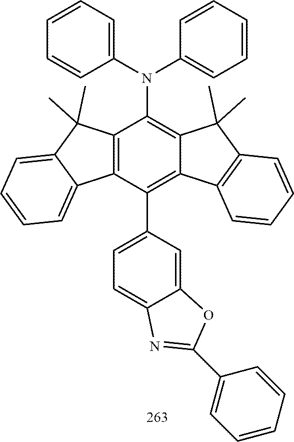 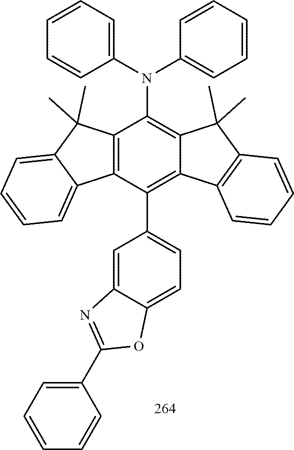 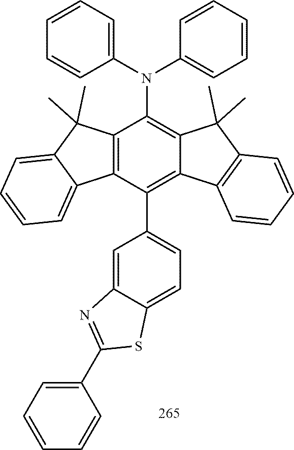 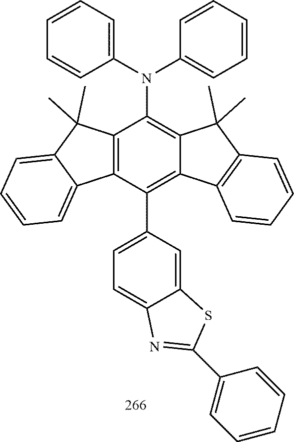 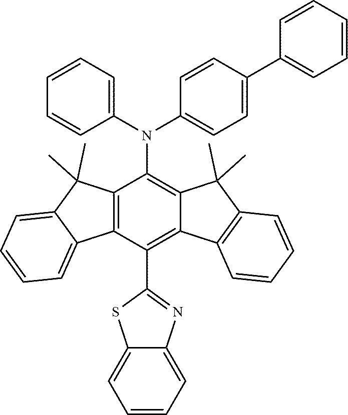 [SMILES: CC1(C)c2ccccc2-c2c(-c3nc4ccccc4s3)c3c(c(N(c4ccccc4)c4ccc(-c5ccccc5)cc4)c21)C(C)(C)c1ccccc1-3] 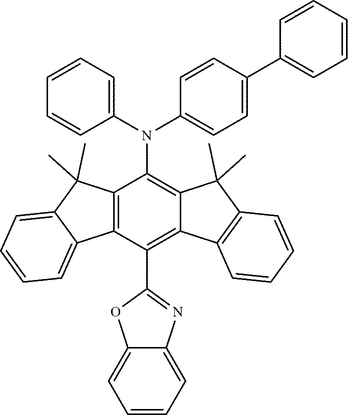 [SMILES: CC1(C)c2ccccc2-c2c(-c3nc4ccccc4o3)c3c(c(N(c4ccccc4)c4ccc(-c5ccccc5)cc4)c21)C(C)(C)c1ccccc1-3] 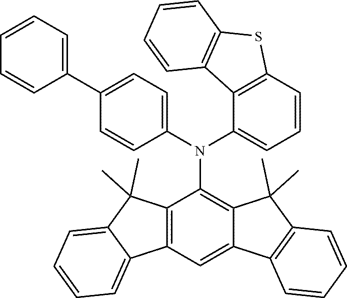 [SMILES: CC1(C)c2ccccc2-c2cc3c(c(N(c4ccc(-c5ccccc5)cc4)c4cccc5sc6ccccc6c45)c21)C(C)(C)c1ccccc1-3] 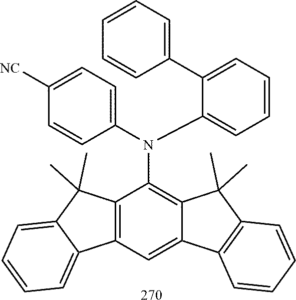 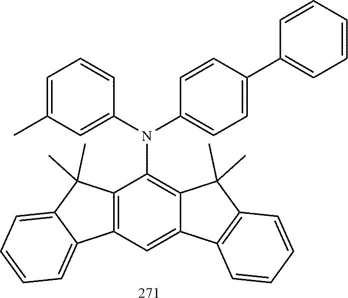 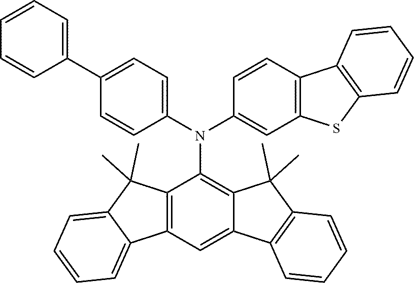 [SMILES: CC1(C)c2ccccc2-c2cc3c(c(N(c4ccc(-c5ccccc5)cc4)c4ccc5c(c4)sc4ccccc45)c21)C(C)(C)c1ccccc1-3] 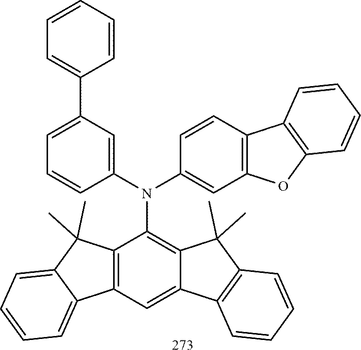 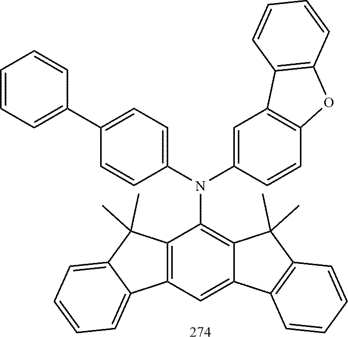 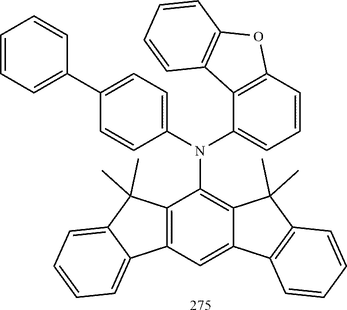 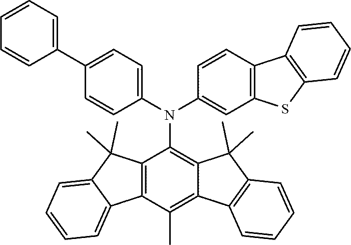 [SMILES: Cc1c2c(c(N(c3ccc(-c4ccccc4)cc3)c3ccc4c(c3)sc3ccccc34)c3c1-c1ccccc1C3(C)C)C(C)(C)c1ccccc1-2] 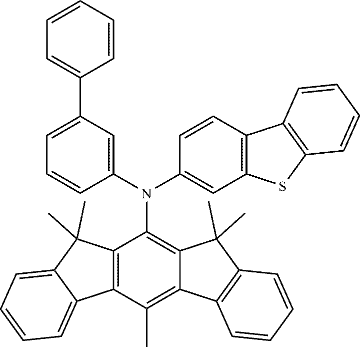 [SMILES: Cc1c2c(c(N(c3cccc(-c4ccccc4)c3)c3ccc4c(c3)sc3ccccc34)c3c1-c1ccccc1C3(C)C)C(C)(C)c1ccccc1-2] 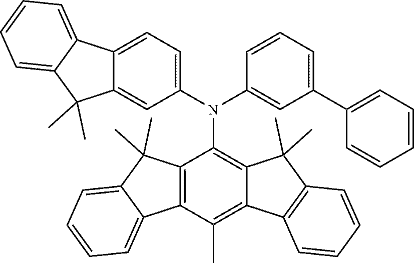 [SMILES: Cc1c2c(c(N(c3cccc(-c4ccccc4)c3)c3ccc4c(c3)C(C)(C)c3ccccc3-4)c3c1-c1ccccc1C3(C)C)C(C)(C)c1ccccc1-2] 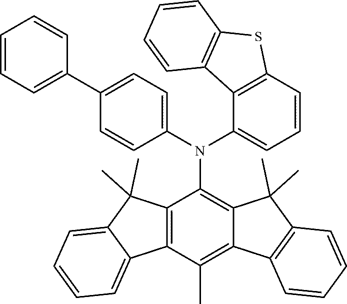 [SMILES: Cc1c2c(c(N(c3ccc(-c4ccccc4)cc3)c3cccc4sc5ccccc5c34)c3c1-c1ccccc1C3(C)C)C(C)(C)c1ccccc1-2] 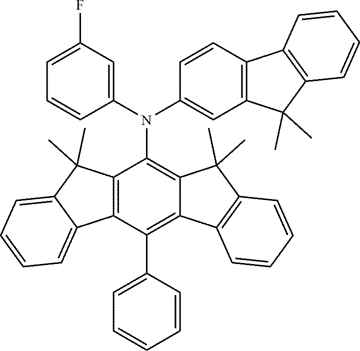 [SMILES: CC1(C)c2ccccc2-c2ccc(N(c3cccc(F)c3)c3c4c(c(-c5ccccc5)c5c3C(C)(C)c3ccccc3-5)-c3ccccc3C4(C)C)cc21] 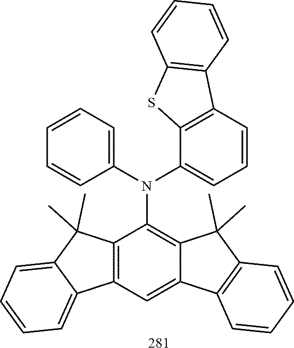 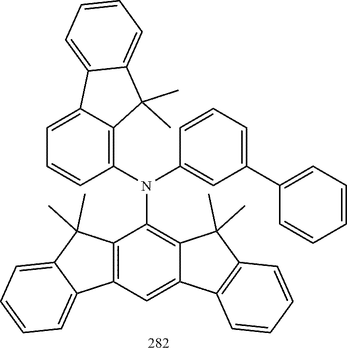 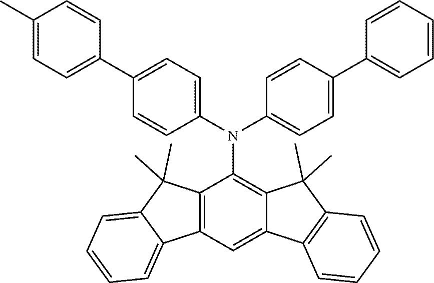 [SMILES: Cc1ccc(-c2ccc(N(c3ccc(-c4ccccc4)cc3)c3c4c(cc5c3C(C)(C)c3ccccc3-5)-c3ccccc3C4(C)C)cc2)cc1] 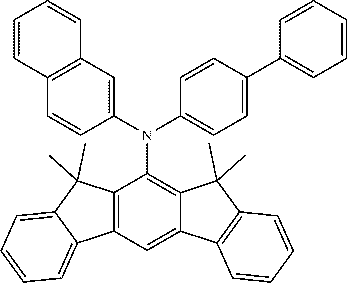 [SMILES: CC1(C)c2ccccc2-c2cc3c(c(N(c4ccc(-c5ccccc5)cc4)c4ccc5ccccc5c4)c21)C(C)(C)c1ccccc1-3] 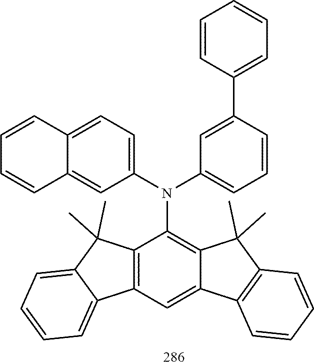 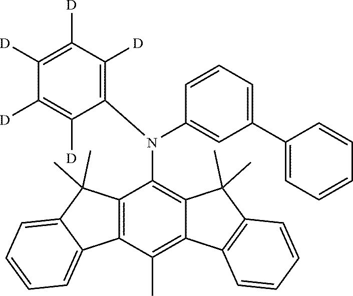 [SMILES: [2H]c1c([2H])c([2H])c(N(c2cccc(-c3ccccc3)c2)c2c3c(c(C)c4c2C(C)(C)c2ccccc2-4)-c2ccccc2C3(C)C)c([2H])c1[2H]]
.

13. An organic electroluminescent device, comprising an anode, a cathode and a functional layer located between the anode and the cathode, wherein the functional layer comprises the nitrogen-containing compound of claim 1 .

14. The organic electroluminescent device of claim 13, wherein the functional layer comprises a hole transporting layer, wherein the hole transporting layer comprises the nitrogen-containing compound.

15. The organic electroluminescent device of claim 13, wherein the functional layer comprises a luminescent layer, the luminescent layer comprises a host material and a luminescent dopant, and the host material comprises the nitrogen-containing compound.

16. An electronic apparatus, comprising the organic electroluminescent device of claim 13.

17. The organic electroluminescent device of claim 14, wherein the hole transporting layer comprises a first hole transporting layer and a second hole transporting layer stacked with the first hole transporting layer, and the first hole transporting layer is closer to the surface of the anode than the second hole transporting layer; and the first hole transporting layer and/or the second hole transporting layer contain/contains the nitrogen-containing compound.
